(19) United States
(12) Patent Application Publication
(10) Pub. No.: US 2023/0130537 A1
(43) Pub. Date: Apr. 27, 2023

(54) LIGHT EMITTING DEVICE AND AMINE COMPOUND FOR THE SAME

(71) Applicant: Samsung Display Co., Ltd., Yongin-si (KR)

(72) Inventor: Akinori YAMATANI, Yokohama (JP)

(73) Assignee: Samsung Display Co., Ltd., Yongin-si (KR)

(21) Appl. No.: 17/883,067

(22) Filed: Aug. 8, 2022

(30) Foreign Application Priority Data
Sep. 10, 2021 (KR) 10-2021-0121190
Feb. 18, 2022 (KR) 10-2022-0021678

(51) Int. Cl. H01L 51/00; C07C 211/58; C09K 11/06; C07D 307/91; C07D 333/76

(52) U.S. Cl. CPC H01L 51/006; C07C 211/58; C09K 11/06; C07D 307/91; H01L 51/0061; C07D 333/76; H01L 51/5056

(57) ABSTRACT

Provided is a light emitting device including a first electrode, a second electrode, and at least one functional layer disposed between the first electrode and the second electrode, and the at least one functional layer may include an amine compound represented by Formula E, thereby exhibiting high luminous efficiency and improved service life characteristics.

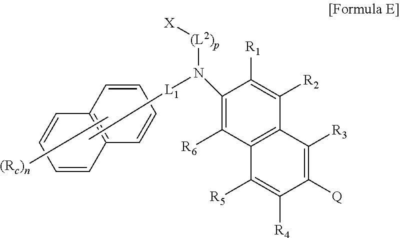

### CROSS-REFERENCE TO RELATED APPLICATION(S)

This application claims priority to and benefits of Korean Patent Application Nos. 10-2021-0121190 and 10-2022-0021678, under 35 U.S.C. § 119, filed on Sep. 10, 2021 and Feb. 18, 2022, respectively, in the Korean Intellectual Property Office, the entire contents of which are incorporated herein by reference.

### BACKGROUND

### 1. Technical Field

The disclosure relates to an amine compound used in a hole transport region and a light emitting device including the same.

### 2. Description of the Related Art

Active development continues for an organic electroluminescence display apparatus as an image display apparatus. The organic electroluminescence display apparatus includes a so-called self-luminescent light emitting device in which holes and electrons respectively injected from a first electrode and a second electrode recombine in an emission layer, so that a luminescent material of the emission layer emits light to achieve display.

In the application of a light emitting device to a display apparatus, there is a demand for a light emitting device having low driving voltage, high luminous efficiency, and a long service life, and continuous development is required on materials for a light emitting device that is capable of stably attaining such characteristics.

Further development is presently conducted on materials for a hole transport region having excellent electron blocking ability and hole transport ability in order to implement a highly efficient light emitting device.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light emitting device exhibiting excellent luminous efficiency and long service life characteristics.

The disclosure also provides an amine compound which is a material for a light emitting device having high efficiency and long service life characteristics.

An embodiment provides a light emitting device which may include a first electrode, a second electrode disposed on the first electrode, an emission layer disposed between the first electrode and the second electrode, and a hole transport region disposed between the emission layer and the first electrode and including an amine compound represented by Formula 1:

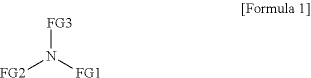

In Formula 1, FG1 may be a group represented by Formula A, FG2 may be a group represented by Formula B, and FG3 may be a group represented by any one of Formula A to Formula C:

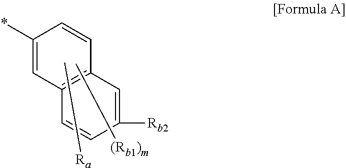

In Formula A, m may be an integer from 1 to 5; Rₐ may be a substituted or unsubstituted phenyl group, a substituted or unsubstituted dibenzofuran group, or a substituted or unsubstituted dibenzothiophene group; and R_{b1} and R_{b2} may each independently be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

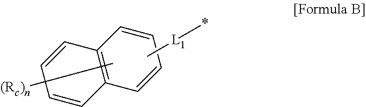

In Formula B, n may be an integer from 0 to 7; L₁ may be a substituted or unsubstituted arylene group having 6 to 30 ring-forming carbon atoms, or a substituted or unsubstituted heteroarylene group having 2 to 30 ring-forming carbon atoms; and R_{c} may be a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

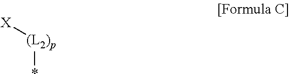

In Formula C, p may be 0 or 1; L₂ may be a substituted or unsubstituted arylene group having 6 to 30 ring-forming carbon atoms, or a substituted or unsubstituted heteroarylene group having 2 to 30 ring-forming carbon atoms, and X may be a group represented by any one of Formula D-1 to Formula D-3:

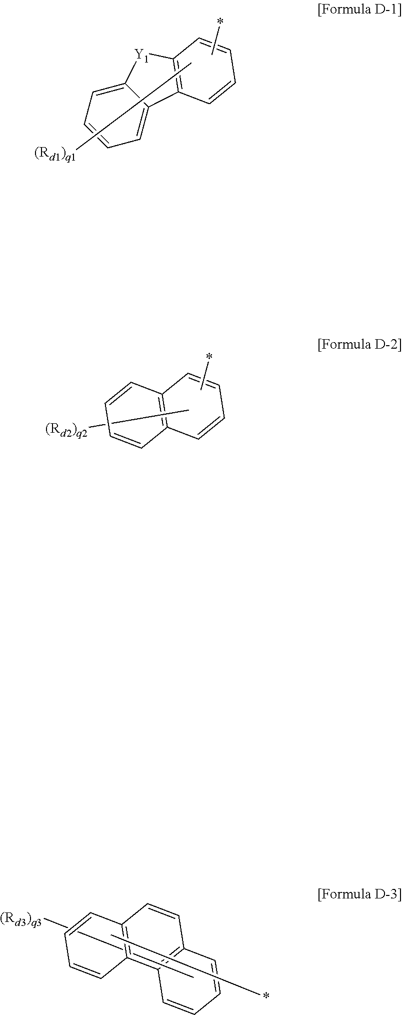

In Formula D-1, Y₁ may be O or S. In Formulae D-1 to D-3, q₁ and q₂ may each independently be an integer from 0 to 7; q₃ may be an integer from 0 to 9; and R_{d1} to R_{d3} may each independently be a deuterium atom, a halogen atom, a substituted or unsubstituted aryl group having 6 to 30 ring-forming carbon atoms, a substituted or unsubstituted heteroaryl group having 2 to 30 heteroaryl group, a substituted or unsubstituted alkenyl group having 2 to 10 carbon atoms, a substituted or unsubstituted aryloxy group, or a substituted or unsubstituted thio group, or may be bonded to an adjacent group to form a ring.

In an embodiment, the group represented by Formula A may be represented by Formula AA-1:

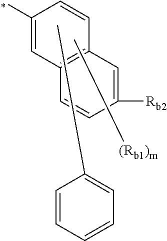

In Formula AA-1, m, R_{b1}, and R_{b2} are the same as defined in Formula A.

In an embodiment, the group represented by Formula B may be represented by any one of Formula BB-1 to Formula BB-3:

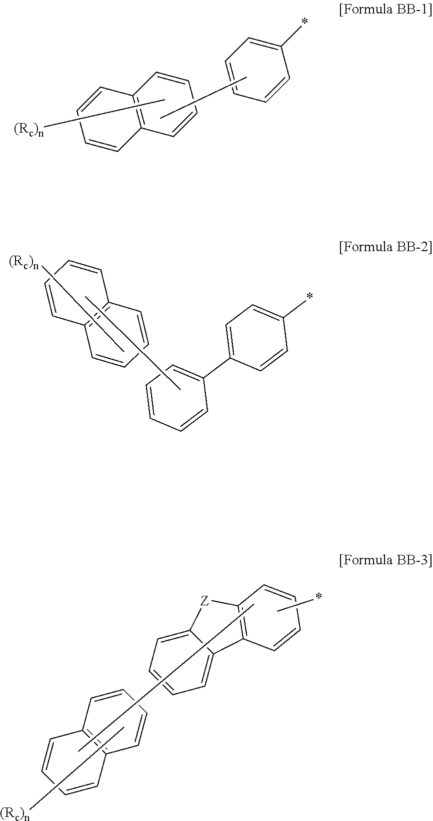

In Formula BB-3, Z may be C(R_{z1})(R_{z2}), N(R_{z3}), O, or S; and R_{z1} to R_{z3} may each independently be a substituted or unsubstituted phenyl group. In Formula BB-1 to Formula BB-3, n and R_{c} are the same as defined in Formula B.

In an embodiment, the group represented by Formula BB-3 may be represented by any one of Formula BBB-1 to Formula BBB-3:

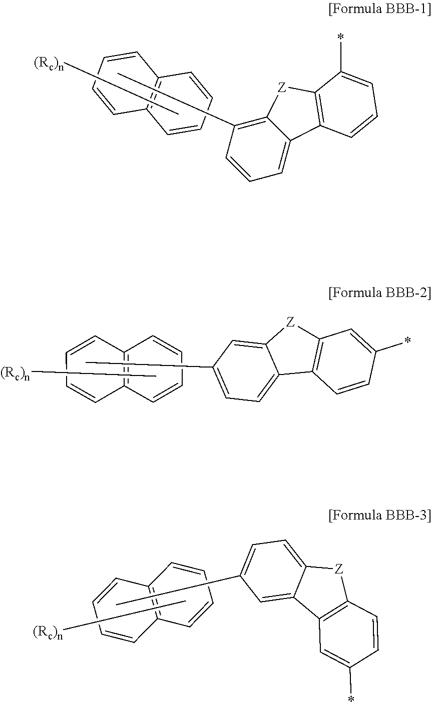

In Formula BBB-1 to Formula BBB-3, n and R_{c} are the same as defined in Formula B; and Z is the same as defined in Formula BB-3.

In an embodiment, the group represented by Formula C may be represented by any one of Formula CC-1 to Formula CC-3:

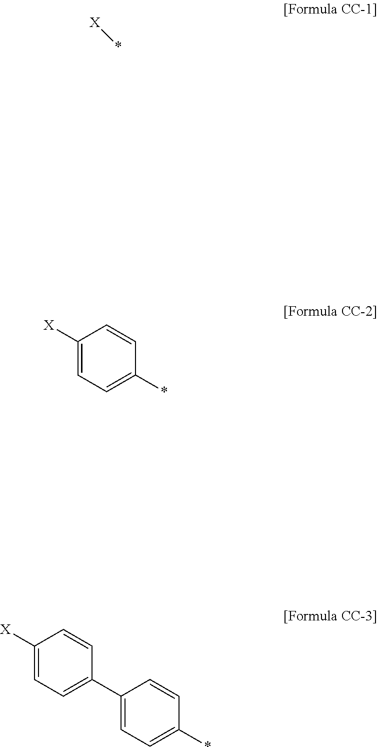

In Formula CC-1 to Formula CC-3, X is the same as defined in Formula C.

In an embodiment, the emission layer may emit blue light having a center wavelength in a range of about 450 nm to about 470 nm.

In an embodiment, the hole transport region may further include a hole injection layer disposed on the first electrode, and a hole transport layer disposed between the hole injection layer and the emission layer; and the hole transport layer may include the amine compound.

In an embodiment, in Formula 1, FG1 may be a group represented by any one of Formulae A-1 to A-75, FG2 may be a group represented by any one of Formulae B-1 to B-99 and B-101 to B-106, and FG3 may be a group represented by any one of Formulae A-1 to A-75, B-1 to B-99, B-101 to B-106, C-1 to C-77, and C-101 to C-109, wherein Formulae A-1 to A-75, B-1 to B-99, B-101 to B-106, C-1 to C-77, and C-101 to C-109 are explained below.

An embodiment provides a light emitting device which may include a first electrode, a second electrode disposed on the first electrode, an emission layer disposed between the first electrode and the second electrode, and a hole transport region disposed between the emission layer and the first electrode and including an amine compound represented by Formula E:

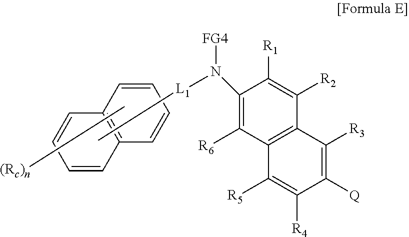

In Formula E, n may be an integer from 0 to 7; L₁ may be a substituted or unsubstituted arylene group having 6 to 30 ring-forming carbon atoms, or a substituted or unsubstituted heteroarylene group having 2 to 30 ring-forming carbon atoms; at least one of R₁ to R₆ may be a substituted or unsubstituted phenyl group, a substituted or unsubstituted dibenzofuran group, or a substituted or unsubstituted dibenzothiophene group; the remainder of R₁ to R₆ may each independently be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group; Q may be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group; R_{c} may be a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group; and FR4 may be a group represented by any one of Formula F-1 to Formula F-3:

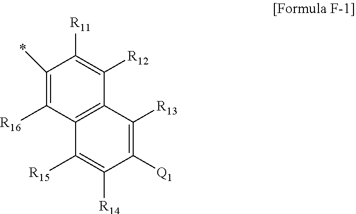

In Formula F-1, at least one of R₁₁ to R₁₆ may be a substituted or unsubstituted phenyl group, a substituted or unsubstituted dibenzofuran group, or a substituted or unsubstituted dibenzothiophene group; the remainder of R₁₁ to R₁₆ may each independently be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group; and Q₁ may be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

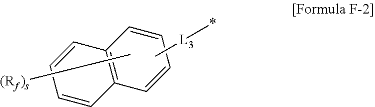

In Formula F-2, s may be an integer from 0 to 7; L₃ may be a substituted or unsubstituted arylene group having 6 to 30 ring-forming carbon atoms, or a substituted or unsubstituted heteroarylene group having 2 to 30 ring-forming carbon atoms; and R_{f} may be a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

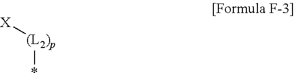

In Formula F-3, p may be 0 or 1; L₂ may be a substituted or unsubstituted arylene group having 6 to 30 ring-forming carbon atoms, or a substituted or unsubstituted heteroarylene group having 2 to 30 ring-forming carbon atoms; and X may be a group represented by any one of Formula D-1 to Formula D-3:

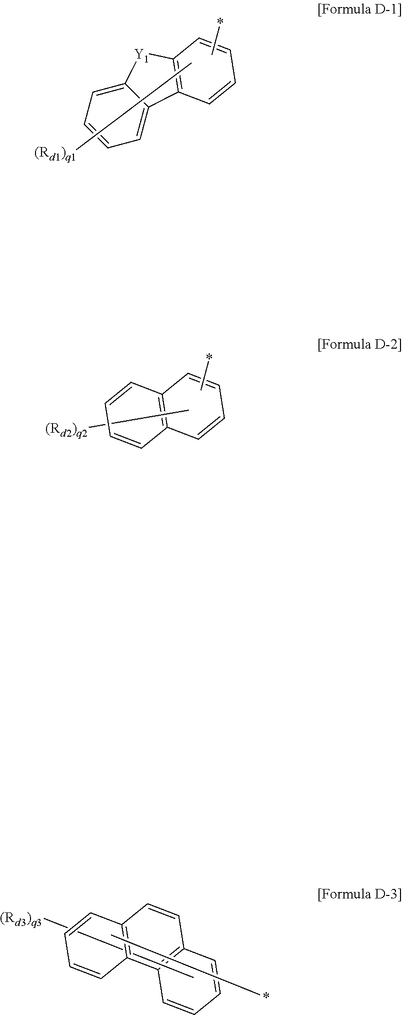

In Formula D-1, Y₁ may be O or S. In Formula D-1 to Formula D-3, q₁ and q₂ may each independently be an integer from 0 to 7; q₃ may be an integer from 0 to 9; and R_{d1} to R_{d3} may each independently be a deuterium atom, a halogen atom, a substituted or unsubstituted aryl group having 6 to 30 ring-forming carbon atoms, a substituted or unsubstituted heteroaryl group having 2 to 30 heteroaryl group, a substituted or unsubstituted alkenyl group having 2 to 10 carbon atoms, a substituted or unsubstituted aryloxy group, or a substituted or unsubstituted thio group, or may be bonded to an adjacent group to form a ring.

In an embodiment, in Formula E, at least one of R₁ to R₆ may be an unsubstituted phenyl group.

In an embodiment, the amine compound represented by Formula E may be represented by any one of Formula G-1 to Formula G-3:

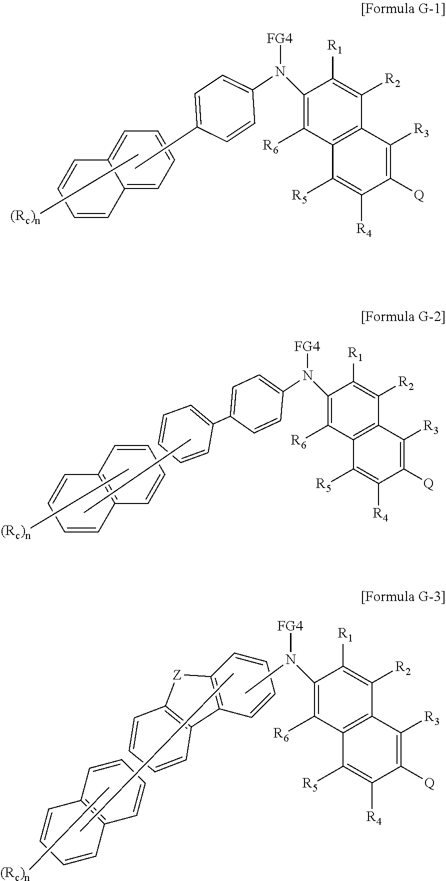

In Formula G-3, Z may be C(R_{z1})(R_{z2}), N(R_{z3}), O, or S; and R_{z1} to R_{z3} may each independently be a substituted or unsubstituted phenyl group. In Formula G-1 to Formula G-3, n, FG4, R_{c}, Q, and R₁ to R₆ are the same as defined in Formula E.

In an embodiment, the amine compound represented by Formula G-3 may be represented by any one of Formula GG-1 to Formula GG-3:

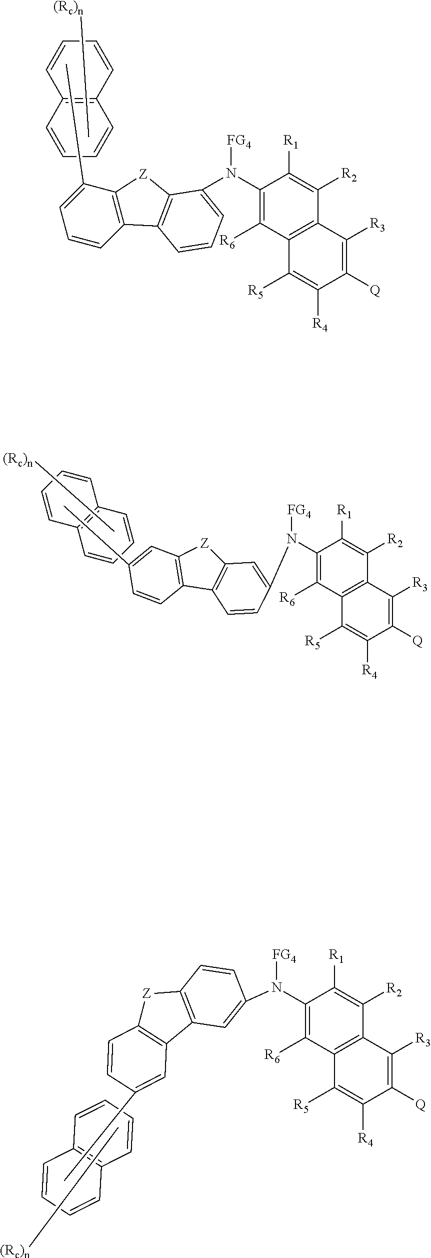

In Formula GG-1 to Formula GG-3, Z is the same as defined in Formula G-3; and n, FG4, R_{c}, Q, and R₁ to R₆ are the same as defined in Formula E.

In an embodiment, in Formula F-3, L₂ may be a substituted or unsubstituted divalent phenylene group or a substituted or unsubstituted divalent biphenyl group.

In an embodiment, the amine compound represented by Formula E may include a first substituent represented by any one of Formulae A-1 to A-75, a second substituent represented by any one of Formulae B-1 to B-99 and B-101 to B-106, and a third substituent represented by any one of Formulae A-1 to A-75, B-1 to B-99, B-101 to B-106, C-1 to C-77, and C-101 to C-109, wherein Formulae A-1 to A-75, B-1 to B-99, B-101 to B-106, C-1 to C-77, and C-101 to C-109 are explained below.

An embodiment provides an amine compound which may be represented by Formula 1:

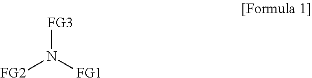

In Formula 1, FG1 may be a group represented by Formula A, FG2 may be a group represented by Formula B, and FG3 may be a group represented by any one of Formula A to Formula C:

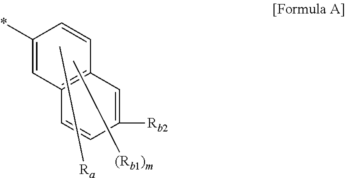

In Formula A, m may be an integer from 1 to 5; Rₐ may be a substituted or unsubstituted phenyl group, a substituted or unsubstituted dibenzofuran group, or a substituted or unsubstituted dibenzothiophene group; and R_{b1} and R_{b2} may each independently be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

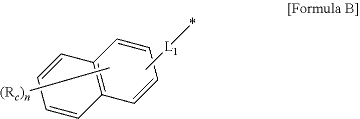

In Formula B, n may be an integer from 0 to 7; L₁ may be a substituted or unsubstituted arylene group having 6 to 30 ring-forming carbon atoms, or a substituted or unsubstituted heteroarylene group having 2 to 30 ring-forming carbon atoms; and R_{c} may be a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

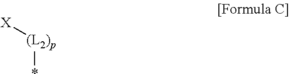

In Formula C, p may be 0 or 1; L₂ may be a substituted or unsubstituted arylene group having 6 to 30 ring-forming carbon atoms, or a substituted or unsubstituted heteroarylene group having 2 to 30 ring-forming carbon atoms; and X may be a group represented by any one of Formula D-1 to Formula D-3:

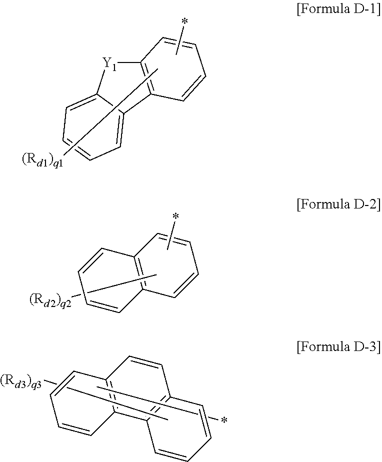

In Formula D-1, Y₁ may be O or S. In Formula D-1 to Formula D-3, q₁ and q₂ may each independently be an integer from 0 to 7; q₃ may be an integer from 0 to 9; and R_{d1} to R_{d3} may each independently be a deuterium atom, a halogen atom, a substituted or unsubstituted aryl group having 6 to 30 ring-forming carbon atoms, a substituted or unsubstituted heteroaryl group having 2 to 30 heteroaryl group, a substituted or unsubstituted alkenyl group having 2 to 10 carbon atoms, a substituted or unsubstituted aryloxy group, or a substituted or unsubstituted thio group, or may be bonded to an adjacent group to form a ring.

In an embodiment, the group represented by Formula A may be represented by Formula AA-1:

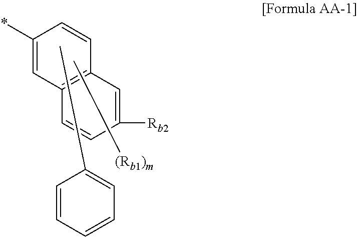

In Formula AA-1, m, R_{b1}, and R_{b2} are the same as defined in Formula A.

In an embodiment, the group represented by Formula B may be represented by any one of Formula BB-1 to Formula BB-3:

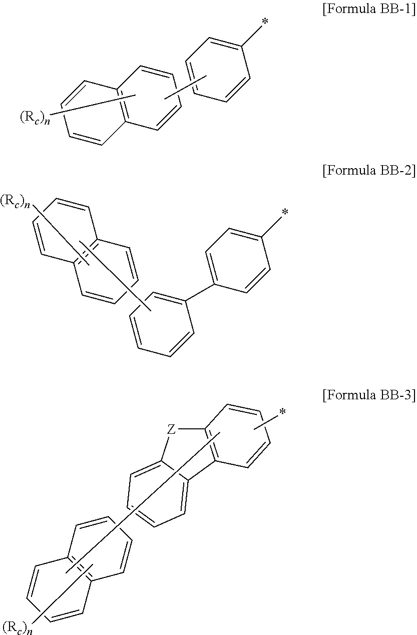

In Formula BB-3, Z may be C(R_{z1})(R_{z2}), N(R_{z3}), O, or S; and R_{z1} to R_{z3} may each independently be a substituted or unsubstituted phenyl group. In Formula BB-1 to Formula BB-3, n and R_{c} are the same as defined in Formula B.

In an embodiment, the group represented by Formula BB-3 may be represented by any one of Formula BBB-1 to Formula BBB-3:

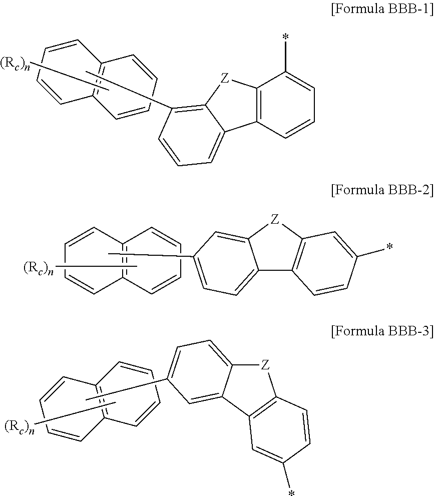

In Formula BBB-1 to Formula BBB-3, n and R_{c} are the same as defined in Formula B; and Z is the same as defined in Formula BB-3.

In an embodiment, the group represented by Formula C may be represented by any one of Formula CC-1 to Formula CC-3:

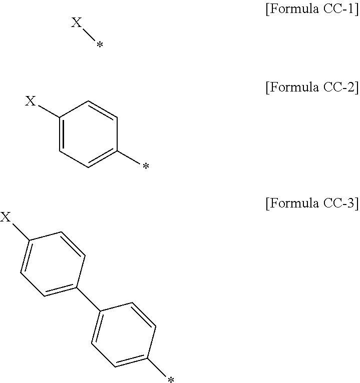

In Formula CC-1 to Formula CC-3, X is the same as defined in Formula C.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a plan view illustrating a display apparatus according to an embodiment;

FIG. 2 is a schematic cross-sectional view of a display apparatus according to an embodiment;

FIG. 3 is a schematic cross-sectional view illustrating a light emitting device according to an embodiment;

FIG. 4 is a schematic cross-sectional view illustrating a light emitting device according to an embodiment;

FIG. 5 is a schematic cross-sectional view illustrating a light emitting device according to an embodiment;

FIG. 6 is a schematic cross-sectional view illustrating a light emitting device according to an embodiment;

FIG. 7 is a schematic cross-sectional view of a display apparatus according to an embodiment; and

FIG. 8 is a schematic cross-sectional view of a display apparatus according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

The term “at least one of” is intended to include the meaning of “at least one selected from the group of” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may mean a group that is substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the specification, the phrase “bonded to an adjacent group to form a ring” may mean a group that is bonded to an adjacent group to form a substituted or unsubstituted hydrocarbon ring, or a substituted or unsubstituted heterocycle. The hydrocarbon ring may be an aliphatic hydrocarbon ring or an aromatic hydrocarbon ring. The heterocycle may be an aliphatic heterocycle or an aromatic heterocycle. The hydrocarbon ring and the heterocycle may each independently be monocyclic or polycyclic. A ring that is formed by adjacent groups being bonded to each other may itself be connected to another ring to form a spiro structure.

In the specification, the term “adjacent group” may mean a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other, and two ethyl groups in 1,1-diethylcyclopentane may be interpreted as “adjacent groups” to each other. For example, two methyl groups in 4,5-dimethylphenanthrene may be interpreted as “adjacent groups” to each other.

In the specification, examples of a halogen atom may include a fluorine atom, a chlorine atom, a bromine atom, or an iodine atom.

In the specification, an alkyl group may be linear, branched, or cyclic. The number of carbon atoms in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments are not limited thereto.

In the specification, a cycloalkyl group may be a cyclic alkyl group. The number of carbon atoms in the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments are not limited thereto.

In the specification, an alkenyl group may be a hydrocarbon group including at least one carbon-carbon double bond in the middle or at a terminus of an alkyl group having 2 or more carbon atoms. The alkenyl group may be linear or branched. The number of carbon atoms in the alkenyl group is not specifically limited, but may be 2 to 30, 2 to 20 or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the specification, an alkynyl group may be a hydrocarbon group including at least one carbon-carbon triple bond in the middle or at a terminus of an alkyl group having 2 or more carbon atoms. The alkynyl group may be linear or branched. The number of carbon atoms in the alkynyl group is not specifically limited, but may be 2 to 30, 2 to 20 or 2 to 10. Examples of the alkynyl group may include an ethynyl group, a propynyl group, etc., without limitation.

In the specification, a hydrocarbon ring group may be any functional group or substituent derived from an aliphatic hydrocarbon ring. For example, the hydrocarbon ring group may be a saturated hydrocarbon ring group having 5 to 20 ring-forming carbon atoms.

In the specification, an aryl group may be any functional group or substituent derived from an aromatic hydrocarbon ring. The aryl group may be monocyclic or polycyclic. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments are not limited thereto.

In the specification, a fluorenyl group may be substituted, and two substituents may be bonded to each other to form a spiro structure. Examples of substituted fluorenyl groups are as follows. However, embodiments are not limited thereto.

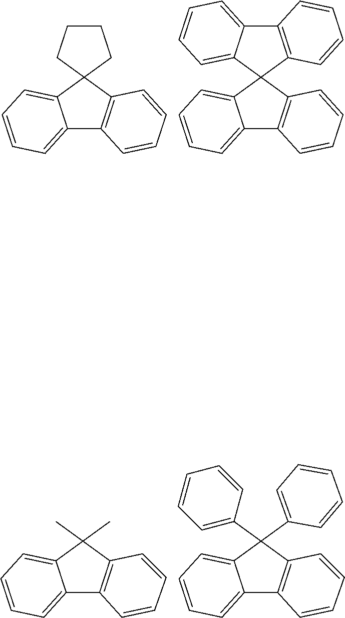

In the specification, a heterocyclic group may be any functional group or substituent derived from a ring including at least one of B, O, N, P, Si, or Se as a heteroatom. The heterocyclic group may be an aliphatic heterocyclic group or an aromatic heterocyclic group. The aromatic heterocyclic group may be a heteroaryl group. The aliphatic heterocycle and the aromatic heterocycle may each independently be monocyclic or polycyclic.

In the specification, the heterocyclic group may include at least one of B, O, N, P, Si, or S as a heteroatom. If the heterocyclic group includes two or more heteroatoms, the two or more heteroatoms may be the same as or different from each other. The heterocyclic group may be monocyclic or polycyclic, and the heterocyclic group may be a heteroaryl group. The number of ring-forming carbon atoms in the heterocyclic group may be 2 to 30, 2 to 20, or 2 to 10.

In the specification, an aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments are not limited thereto.

In the specification, the heteroaryl group may include one or more of B, O, N, P, Si, or S as a heteroatom. When the heteroaryl group contains two or more heteroatoms, the two or more heteroatoms may be the same as or different from each other. The heteroaryl group may be monocyclic or polycyclic. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a triazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments are not limited thereto.

In the specification, the above description of the aryl group may be applied to an arylene group, except that the arylene group is a divalent group. The above description of the heteroaryl group may be applied to a heteroarylene group except that the heteroarylene group is a divalent group.

In the specification, a silyl group may be an alkylsilyl group or an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments are not limited thereto.

In the specification, the number of carbon atoms in an amino group is not specifically limited, but may be 1 to 30. The amino group may be an alkyl amino group, an aryl amino group, or a heteroaryl amino group. Examples of the amino group may include a methylamino group, a dimethylamino group, a phenylamino group, a diphenylamino group, a naphthylamino group, a 9-methyl-anthracenylamino group, a triphenylamino group, etc., but are not limited thereto.

In the specification, the number of carbon atoms in a carbonyl group is not specifically limited, but may be 1 to 40, 1 to 30, or 1 to 20. For example, the carbonyl group may have one of the following structures, but embodiments are not limited thereto.

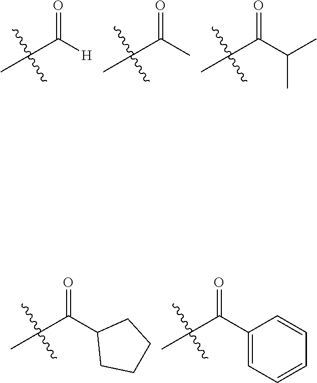

In the specification, the number of carbon atoms in a sulfinyl group or a sulfonyl group is not particularly limited, but may be 1 to 30. The sulfinyl group may be an alkyl sulfinyl group or an aryl sulfinyl group. The sulfonyl group may be an alkyl sulfonyl group or an aryl sulfonyl group.

In the specification, a thio group may be an alkylthio group or an arylthio group. The thio group may be a sulfur atom that is bonded to an alkyl group or an aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments are not limited thereto.

In the specification, an oxy group may be an oxygen atom that is bonded to an alkyl group or an aryl group as defined above. The oxy group may be an alkoxy group or an aryl oxy group. The alkoxy group may be linear, branched, or cyclic. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., but embodiments are not limited thereto.

In the specification, a boron group may be a boron atom that is bonded to an alkyl group or an aryl group as defined above. The boron group may be an alkyl boron group or an aryl boron group. Examples of the boron group may include a trimethylboron group, a triethylboron group, a t-butyldimethylboron group, a triphenylboron group, a diphenylboron group, a phenylboron group, etc., but embodiments are not limited thereto.

In the specification, the alkenyl group may be linear or branched. The number of carbon atoms in the alkenyl group is not specifically limited, but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the specification, the number of carbon atoms in an amine group is not specifically limited, but may be 1 to 30. The amine group may be an alkyl amine group or an aryl amine group. Examples of the amine group may include a methylamine group, a dimethylamine group, a phenylamine group, a diphenylamine group, a naphthylamine group, a 9-methyl-anthracenylamine group, a triphenylamine group, etc., but embodiments are not limited thereto.

In the specification, the alkyl group in an alkylthio group, an alkylsulfoxy group, an alkylaryl group, an alkylamino group, an alkyl boron group, an alkyl silyl group, and an alkyl amine group may be the same as the examples of the alkyl group described above.

In the specification, the aryl group in an aryloxy group, an arylthio group, an arylsulfoxy group, an arylamino group, an arylboron group, an arylsilyl group, and an arylamine group may be the same as the examples of the aryl group described above.

In the specification, a direct linkage herein may be a single bond.

In the specification, the symbols

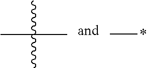

each represents a bonding site to a neighboring atom.

Hereinafter, embodiments will be described with reference to the accompanying drawings.

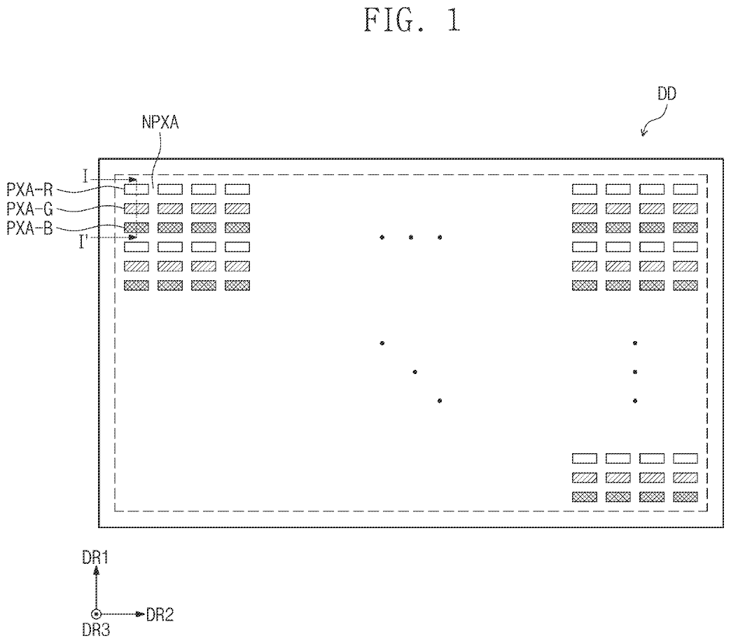

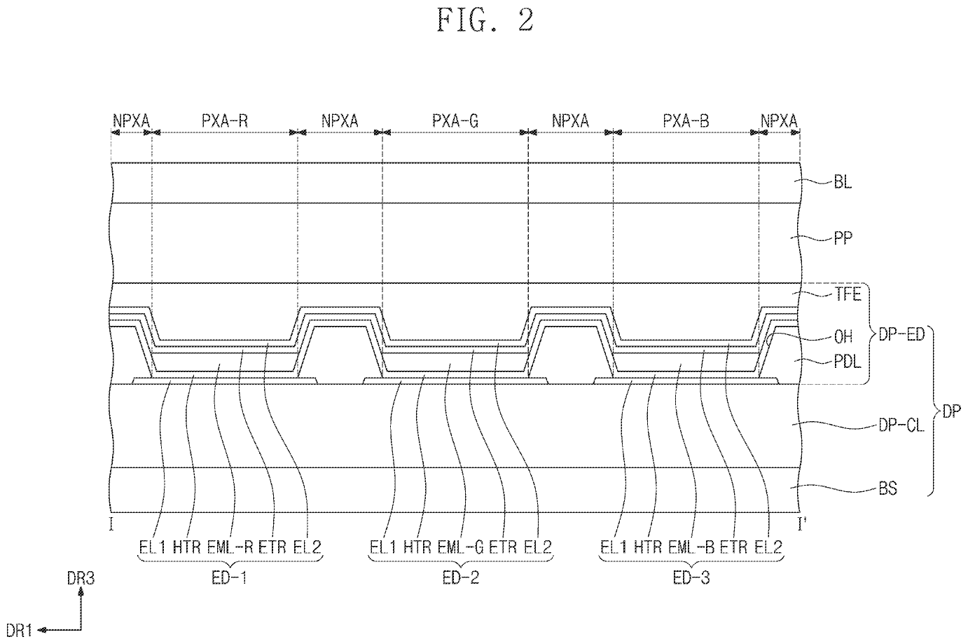

FIG. 1 is a plan view illustrating an embodiment of a display apparatus DD. FIG. 2 is a schematic cross-sectional view of the display apparatus DD according to an embodiment. FIG. 2 is a schematic cross-sectional view illustrating a portion corresponding to line I-I′ of FIG. 1.

The display apparatus DD may include a display panel DP and an optical layer PP disposed on the display panel DP. The display panel DP includes light emitting devices ED-1, ED-2, and ED-3. The display apparatus DD may include multiples of each of the light emitting devices ED-1, ED-2, and ED-3. The optical layer PP may be disposed on the display panel DP and may control light reflected at the display panel DP from an external light. The optical layer PP may include, for example, a polarization layer or a color filter layer. Although not shown in the drawings, in an embodiment, the optical layer PP may be omitted from the display apparatus DD.

A base substrate BL may be disposed on the optical layer PP. The base substrate BL may provide a base surface on which the optical layer PP disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

The display apparatus DD according to an embodiment may further include a filling layer (not shown). The filling layer (not shown) may be disposed between a display device layer DP-ED and the base substrate BL. The filling layer (not shown) may be an organic material layer. The filling layer (not shown) may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light emitting devices ED-1, ED-2, and ED-3 disposed between portions of the pixel defining film PDL, and an encapsulation layer TFE disposed on the light emitting devices ED-1, ED-2, and ED-3.

The base layer BS may provide a base surface on which the display device layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include transistors (not shown). Each of the transistors (not shown) may include a control electrode, an input electrode, and an output electrode. For example, the circuit layer DP-CL may include a switching transistor and a driving transistor in order to drive the light emitting devices ED-1, ED-2, and ED-3 of the display device layer DP-ED.

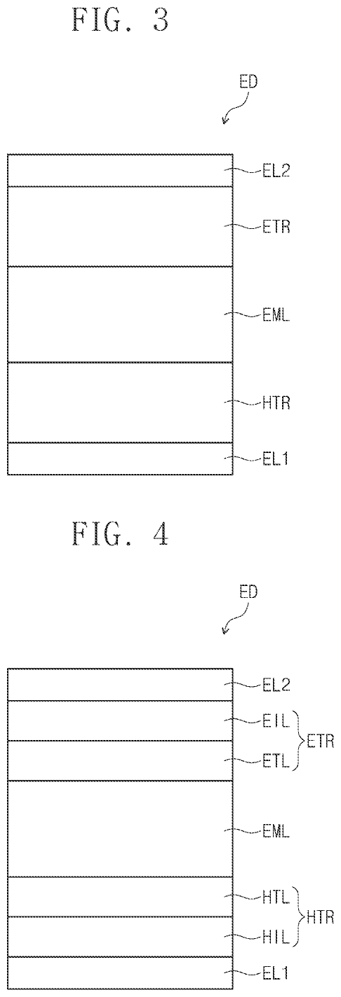

Each of the light emitting devices ED-1, ED-2, and ED-3 may have a structure of a light emitting device ED according to an embodiment in FIGS. 3 to 6, which will be described later. Each of the light emitting devices ED-1, ED-2, and ED-3 may include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting devices ED-1, ED-2, and ED-3 are disposed in openings OH defined in the pixel defining film PDL, and the hole transport region HTR, the electron transport region ETR, and the second electrode EL2 are each provided as a common layer for all of the light emitting devices ED-1, ED-2, and ED-3. However, embodiments are not limited thereto. Although not shown in FIG. 2, in an embodiment, the hole transport region HTR and the electron transport region ETR may each be provided by being patterned inside the openings OH defined in the pixel defining film PDL. For example, in an embodiment, the hole transport region HTR, the emission layers EML-R, EML-G, and EML-B, and the electron transport region ETR of the light emitting devices ED-1, ED-2, and ED-3 may each be provided by being patterned by an inkjet printing method.

The encapsulation layer TFE may cover the light emitting devices ED-1, ED-2, and ED-3. The encapsulation layer TFE may seal the display device layer DP-ED. The encapsulation layer TFE may be a thin film encapsulation layer. The encapsulation layer TFE may be formed of a single layer or of multiple layers. The encapsulation layer TFE may include at least one insulation layer. The encapsulation layer TFE according to an embodiment may include at least one inorganic film (hereinafter, an encapsulation-inorganic film). The encapsulation layer TFE according to an embodiment may include at least one organic film (hereinafter, an encapsulation-organic film) and at least one encapsulation-inorganic film.

The encapsulation-inorganic film may protect the display device layer DP-ED from moisture and/or oxygen, and the encapsulation-organic film may protect the display device layer DP-ED from foreign substances such as dust particles. The encapsulation-inorganic film may include silicon nitride, silicon oxynitride, silicon oxide, titanium oxide, aluminum oxide, or the like, but embodiments are not limited thereto. The encapsulation-organic film may include an acrylic-based compound, an epoxy-based compound, or the like. The encapsulation-organic film may include a photopolymerizable organic material, but embodiments are not limited thereto.

The encapsulation layer TFE may be disposed on the second electrode EL2 and may be disposed to fill the openings OH.

Referring to FIGS. 1 and 2, the display apparatus DD may include a non-light emitting region NPXA and light emitting regions PXA-R, PXA-G, and PXA-B. The light emitting regions PXA-R, PXA-G, and PXA-B may each be a region which emits light generated from the light emitting devices ED-1, ED-2, and ED-3, respectively. The light emitting regions PXA-R, PXA-G, and PXA-B may be spaced apart from each other in a plan view.

The light emitting regions PXA-R, PXA-G, and PXA-B may each be a region separated by the pixel defining film PDL. The non-light emitting regions NPXA may be regions between the adjacent light emitting regions PXA-R, PXA-G, and PXA-B, which correspond to portions of the pixel defining film PDL. For example, in an embodiment, each of the light emitting regions PXA-R, PXA-G, and PXA-B may correspond to a pixel. The pixel defining film PDL may separate the light emitting elements ED-1, ED-2, and ED-3. The emission layers EML-R, EML-G, and EML-B of the light emitting devices ED-1, ED-2, and ED-3 may be disposed in openings OH defined by the pixel defining film PDL and separated from each other.

The light emitting regions PXA-R, PXA-G, and PXA-B may be arranged into groups according to the color of light generated from the light emitting devices ED-1, ED-2, and ED-3. In the display apparatus DD according to an embodiment shown in FIGS. 1 and 2, three light emitting regions PXA-R, PXA-G, and PXA-B which respectively emit red light, green light, and blue light are illustrated as an example. For example, the display apparatus DD according to an embodiment may include the red light emitting region PXA-R, the green light emitting region PXA-G, and the blue light emitting region PXA-B which are separated from one another.

In the display apparatus DD according to an embodiment, the light emitting devices ED-1, ED-2, and ED-3 may emit light having wavelengths different from one another. For example, in an embodiment, the display apparatus DD may include a first light emitting device ED-1 that emits red light, a second light emitting device ED-2 that emits green light, and a third light emitting device ED-3 that emits blue light. For example, the red light emitting region PXA-R, the green light emitting region PXA-G, and the blue light emitting region PXA-B of the display apparatus DD may respectively correspond to the first light emitting device ED-1, the second light emitting device ED-2, and the third light emitting device ED-3.

However, embodiments are not limited thereto, and the first to third light emitting devices ED-1, ED-2, and ED-3 may emit light in a same wavelength range, or at least one light emitting device may emit light in a wavelength range different from the others. For example, the first to third light emitting devices ED-1, ED-2, and ED-3 may all emit blue light.

The light emitting regions PXA-R, PXA-G, and PXA-B in the display apparatus DD according to an embodiment may be arranged in a stripe configuration. Referring to FIG. 1, the red light emitting regions PXA-R, the green light emitting regions PXA-G, and the blue light emitting regions PXA-B may each be arranged along a second directional axis DR2. In another embodiment, the red light emitting region PXA-R, the green light emitting region PXA-G, and the blue light emitting region PXA-B may be arranged by turns along a first directional axis DR1.

FIGS. 1 and 2 illustrate that the light emitting regions PXA-R, PXA-G, and PXA-B have a similar area to each other, but embodiments are not limited thereto. The light emitting regions PXA-R, PXA-G, and PXA-B may have different areas from each other according to a wavelength range of the emitted light. The areas of the light emitting regions PXA-R, PXA-G, and PXA-B may be areas in a plan view that are defined by the first directional axis DR1 and the second directional axis DR2.

The arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in various combinations according to the display quality characteristics which are required in the display apparatus DD. For example, the arrangement form of the light emitting regions PXA-R, PXA-G, and PXA-B may be a PENTILE® configuration or a diamond configuration.

In an embodiment, the areas of the light emitting regions PXA-R, PXA-G, and PXA-B may be different in size from each other. For example, in an embodiment, an area of the green light emitting region PXA-G may be smaller than an area of the blue light emitting region PXA-B, but embodiments are not limited thereto.

Hereinafter, FIGS. 3 to 6 are schematic cross-sectional views illustrating light emitting devices according to embodiments. Each of the light emitting devices ED according to embodiments may include a first electrode EL1, a hole transport region HTR, an emission layer EML, an electron transport region ETR, and a second electrode EL2 that are stacked in that order.

The light emitting device ED of an embodiment may include an amine compound according to an embodiment, which will be described below. The light emitting device ED according to an embodiment may include the amine compound according to an embodiment, which will be described below, in the hole transport region HTR. The light emitting device ED according to an embodiment may include the amine compound according to an embodiment, which will be described below, in the hole transport layer ETL.

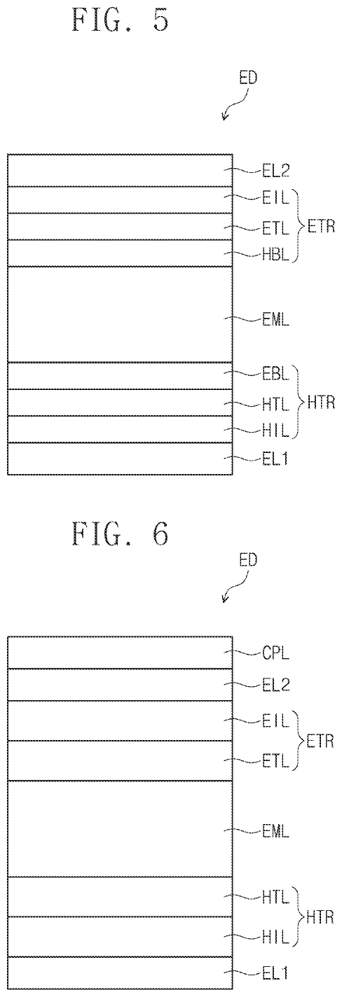

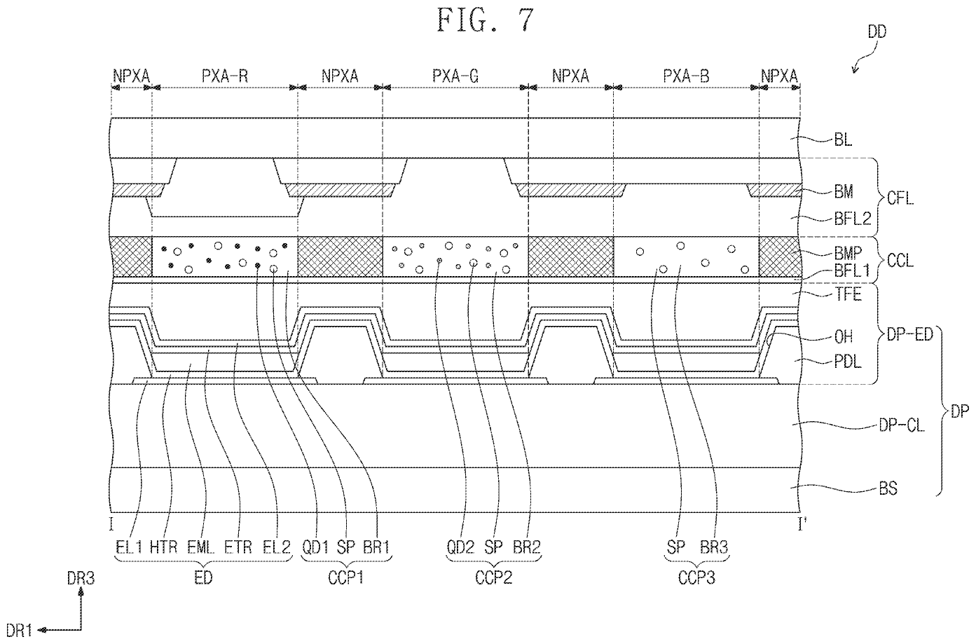

In comparison to FIG. 3, FIG. 4 illustrates a schematic cross-sectional view of a light emitting device ED according to an embodiment, in which a hole transport region HTR includes a hole injection layer HIL and a hole transport layer HTL, and an electron transport region ETR includes an electron injection layer EIL and an electron transport layer ETL. In comparison to FIG. 3, FIG. 5 illustrates a schematic cross-sectional view of a light emitting device ED according to an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 illustrates a schematic cross-sectional view of a light emitting device ED according to an embodiment, including a capping layer CPL disposed on a second electrode EL2.

The first electrode EL1 has conductivity. The first electrode EL1 may be formed of a metal material, a metal alloy, or a conductive compound. The first electrode EL1 may be an anode or a cathode. However, embodiments are not limited thereto. For example, the first electrode EL1 may be a pixel electrode. The first electrode EL1 may be a transmissive electrode, a transflective electrode, or a reflective electrode. If the first electrode EL1 is a transmissive electrode, the first electrode EL1 may be formed of a transparent metal oxide such as indium tin oxide (ITO), indium zinc oxide (IZO), zinc oxide (ZnO), and indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, W, a compound thereof, or a mixture thereof (e.g., a mixture of Ag and Mg). In another embodiment, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but embodiments are not limited thereto. However, embodiments are not limited thereto, and the first electrode EL1 may include the above-described metal materials, combinations of at least two metal materials of the above-described metal materials, oxides of the above-described metal materials, or the like. A thickness of the first electrode EL1 may be in a range of about 700 Å to about 10,000 Å. For example, the thickness of the first electrode EL1 may be in a range of about 1,000 Å to about 3,000 Å.

The hole transport region HTR is provided on the first electrode EL1. The hole transport region HTR may include at least one of a hole injection layer HIL, a hole transport layer HTL, a buffer layer (not shown), an emission-auxiliary layer (not shown), or an electron blocking layer EBL. A thickness of the hole transport region HTR may be, for example, in a range of about 50 Å to about 15,000 Å.

The hole transport region HTR may be a layer formed of a single material, a layer formed of different materials, or a structure including multiple layers formed of different materials.

In an embodiment, the hole transport region HTR may include a hole injection layer HIL and a hole transport layer HTL disposed on the hole injection layer HIL. In an embodiment, the hole transport region HTR may further include an electron blocking layer EBL disposed on the hole transport layer HTL. The hole transport layer HTL and the electron blocking layer EBL may be provided as separate layers or as a single layer.

In an embodiment, the hole transport region HTR may include the amine compound according to an embodiment, which will be described below, in at least one layer. For example, at least one layer disposed on the hole injection layer HIL may include the amine compound according to an embodiment. For example, the hole transport layer HTL or the electron blocking layer EBL may include the amine compound according to an embodiment. When the hole transport layer HTL includes the amine compound according to an embodiment, the hole transport layer HTL may perform an electron blocking function.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole transport region HTR in the light emitting device ED according to an embodiment may include an amine compound represented by Formula 1. The hole transport layer HTL in the light emitting device ED according to an embodiment may include the amine compound represented by Formula 1:

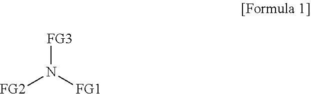

In Formula 1, FG1 may be a group represented by Formula A:

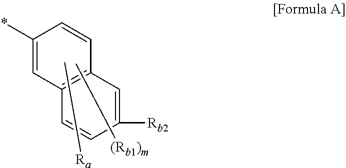

In Formula A, Rₐ may be a substituted or unsubstituted phenyl group, a substituted or unsubstituted dibenzofuran group, or a substituted or unsubstituted dibenzothiophene group.

In Formula A, m may be an integer from 1 to 5. In Formula A, R_{b1} and R_{b2} may each independently be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group. When m is 1, R_{b1} and R₂ may be the same as or different from each other. When m is 2 or greater, multiple R_{b1} groups and R_{b2} may all be the same as each other, or at least one may be different from the rest.

In Formula 1, FG2 may be a group represented by Formula B:

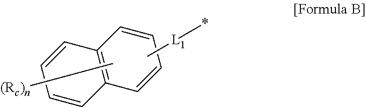

In Formula B, n may be an integer from 0 to 7. In Formula B, R_{c} may be a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group. When n is 2 or greater, multiple R_{c} groups may all be the same as each other, or at least one may be different from the rest.

In Formula B, L₁ may be a substituted or unsubstituted arylene group having 6 to 30 ring-forming carbon atoms, or a substituted or unsubstituted heteroarylene group having 2 to 30 ring-forming carbon atoms.

In Formula 1, FG3 may be a group represented by any one of Formula A, Formula B, or Formula C as described herein.

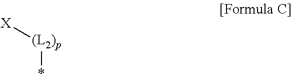

In Formula C, p may be 0 or 1. In Formula C, L₂ may be a substituted or unsubstituted arylene group having 6 to 30 ring-forming carbon atoms, or a substituted or unsubstituted heteroarylene group having 2 to 30 ring-forming carbon atoms.

In Formula C, X may be a group represented by any one of Formula D-1 to Formula D-3:

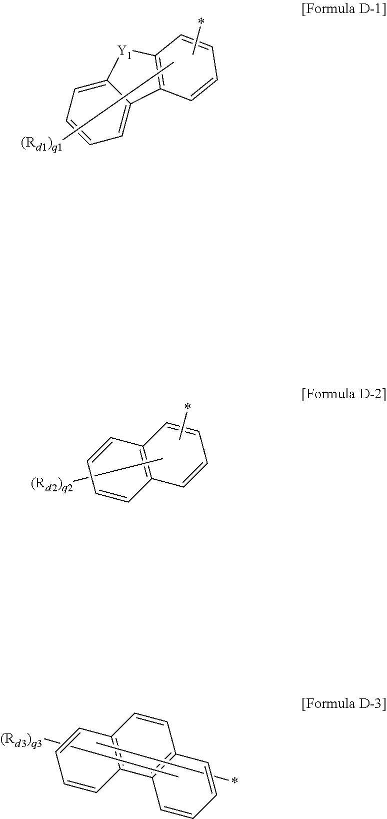

In Formula D-1, Y₁ may be O or S. In Formula D-1 to Formula D-3, q₁ and q₂ may each independently be an integer from 0 to 7. In Formula D-1 to Formula D-3, q₃ may be an integer from 0 to 9. In Formula D-1 to Formula D-3, R_{d1} to R_{d3} may each independently be a deuterium atom, a halogen atom, a substituted or unsubstituted aryl group having 6 to 30 ring-forming carbon atoms, a substituted or unsubstituted heteroaryl group having 2 to 30 ring-forming carbon atoms, a substituted or unsubstituted alkenyl group 2 to 10 carbon atoms, a substituted or unsubstituted oxy group, or a substituted or unsubstituted thio group, or may be bonded to an adjacent group to form a ring. In Formula D-1, when q1 is 2 or greater, multiple R_{d1} groups may all be the same or at least one may be different from the rest. In Formula D-2, when q2 is 2 or greater, multiple R_{d2} groups may all be the same or at least one may be different from the rest. In Formula D-3, when q3 is 2 or greater, multiple R_{d3} groups may all be the same or at least one may be different from the rest.

The amine compound represented by Formula 1 has a structure including a substituted naphthyl group directly substituted at the nitrogen atom of amine, a substituted or unsubstituted naphthyl group that is substituted, via a linker, at the nitrogen atom of amine, and a substituted or unsubstituted naphthyl group or a substituted or unsubstituted aryl group that is directly substituted at the nitrogen atom of amine or substituted via a linker. The naphthyl group directly substituted at the nitrogen atom of amine may be one at which at least one of a substituted or unsubstituted phenyl group, a substituted or unsubstituted dibenzofuran group, or a substituted or unsubstituted dibenzothiophene group is substituted. The amine group represented by Formula 1 may have structural stability because a naphthyl group or a heteroaryl group having strong electron resistance is substituted at the nitrogen atom.

In an embodiment, the group represented by Formula A may be represented by Formula AA-1. Formula AA-1 represents a case where Rₐ in Formula A is an unsubstituted phenyl group. In Formula AA-1, m, R_{b1}, and R_{b2} are the same as defined in Formula A.

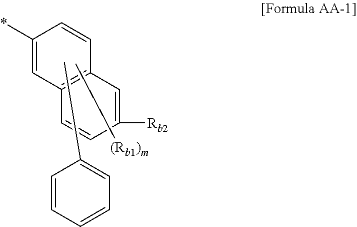

In an embodiment, the group represented by Formula B may be represented by any one of Formula BB-1 to Formula BB-3. Formula BB-1 to Formula BB-3 each represent a case where L₁ in Formula B is specified as an unsubstituted phenyl group, an unsubstituted divalent biphenyl group, and

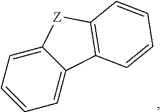

respectively. In Formula BB-1 to Formula BB-3, n and R_{c} are the same as defined in Formula B.

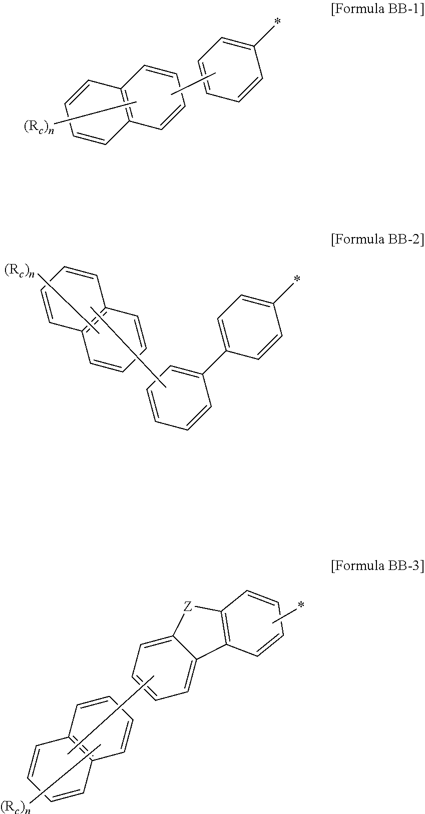

In Formula BB-3, Z may be C(R_{z1})(R_{z2}), N(R_{z3}), O, or S. In Formula BB-3, R_{z1} to R_{z3} may each independently be a substituted or unsubstituted phenyl group. R_{z} and R_{z2} may be the same as or different from each other.

In an embodiment, the group represented by Formula BB-3 may be represented by any one of Formula BBB-1 to Formula BBB-3. Formula BBB-1 to Formula BBB-3 each represent a case where in Formula BB-3, the position at which L₁ is bonded to the nitrogen atom of the amine compound represented by Formula 1, and the position at which the naphthyl group substituted with (R_{c})ₙ is bonded to L₁ are specified.

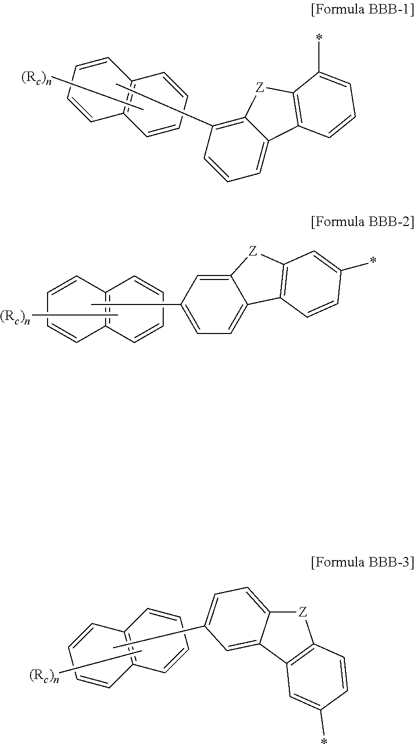

In Formula BBB-1 to Formula BBB-3, n and R_{c} are the same as defined in Formula B, and Z is the same as defined in Formula BB-3.

In an embodiment, the group represented by Formula C may be represented by any one of Formula CC-1 to Formula CC-3. Formula CC-1 represents a case where p in Formula C is 0, and Formula CC-2 and Formula CC-3 each represents a case where p in Formula C is 1. Formula CC-2 represents a case where L₂ in Formula C is an unsubstituted phenyl group. Formula CC-3 represents a case where L₂ in Formula C is an unsubstituted divalent biphenyl group.

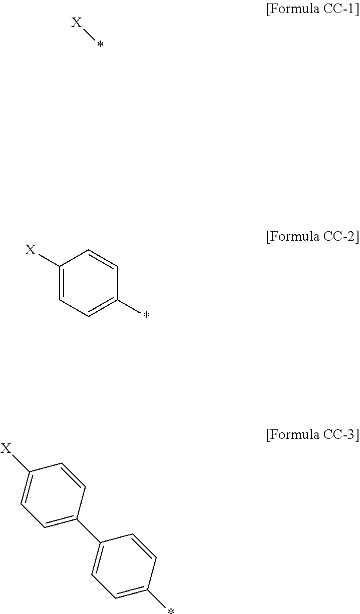

In Formula CC-1 to Formula CC-3, X is the same as defined in Formula C.

The hole transport region HTR in the light emitting device ED according to an embodiment may include the amine compound represented by Formula E. The hole transport layer HTL in the light emitting device ED according to an embodiment may include the amine compound represented by Formula E:

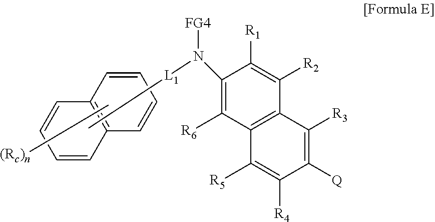

In Formula E, at least one of R₁ to R₆ may be a substituted or unsubstituted phenyl group, a substituted or unsubstituted dibenzofuran group, or a substituted or unsubstituted dibenzothiophene group. In Formula 3, the remainder of R₁ to R₆ each independently be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group. For example, in Formula E, at least one of R₁ to R₆ may be an unsubstituted phenyl group.

In Formula E, Q may be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

In Formula E, L₁ may be a substituted or unsubstituted arylene group having 6 to 30 ring-forming carbon atoms, or a substituted or unsubstituted heteroarylene group having 2 to 30 ring-forming carbon atoms.

In Formula E, n may be an integer from 0 to 7. In Formula E, R_{c} may be a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group. When n is 2 or greater, multiple R_{c} groups may all be the same or at least one may be different from the rest.

In Formula E, FG4 may be a group represented by any one of Formula F-1 to Formula F-3:

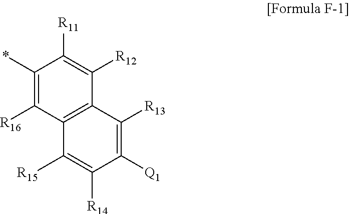

In Formula F-1, at least one of R₁₁ to R₁₆ may be a substituted or unsubstituted phenyl group, a substituted or unsubstituted dibenzofuran group, or a substituted or unsubstituted dibenzothiophene group; and the remainder of R₁₁ to R₁₆ may each independently be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group. In Formula F-1, Q₁ may be a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

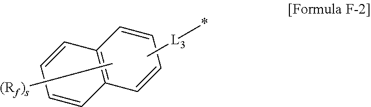

In Formula F-2, s may be an integer from 0 to 7. In Formula F-2, R_{f} may be a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group.

In Formula F-2, L₃ may be a substituted or unsubstituted arylene group having 6 to 30 ring-forming carbon atoms, or a substituted or unsubstituted heteroarylene group having 2 to 30 ring-forming carbon atoms.

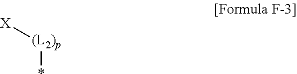

In Formula F-3, p may be 0 or 1. In Formula F-3, L₂ may be a substituted or unsubstituted arylene group having 6 to 30 ring-forming carbon atoms, or a substituted or unsubstituted heteroarylene group having 2 to 30 ring-forming carbon atoms. For example, L₂ may be a substituted or unsubstituted divalent phenylene group or a substituted or unsubstituted divalent biphenyl group. When p is 0, X may be directly bonded to the nitrogen atom represented in Formula E, and when p is 1, X may be bonded via L₂ to the nitrogen atom represented in Formula E.

In Formula F-3, X may be a group represented by any one of Formula D-1 to Formula D-3:

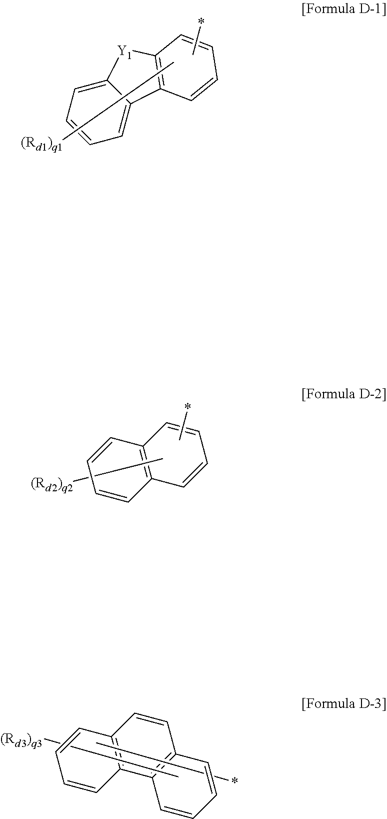

In Formula D-1, Y₁ may be O or S. In Formula D-1 to Formula D-3, q₁ and q₂ may each independently be an integer from 0 to 7. In Formula D-1 to Formula D-3, q₃ may be an integer from 0 to 9. In Formula D-1 to Formula D-3, R_{d1} to R_{d3} may each independently be a deuterium atom, a halogen atom, a substituted or unsubstituted aryl group having 6 to 30 ring-forming carbon atoms, a substituted or unsubstituted heteroaryl group having 2 to 30 ring-forming carbon atoms, a substituted or unsubstituted alkenyl group 2 to 10 carbon atoms, a substituted or unsubstituted oxy group, or a substituted or unsubstituted thio group, or may be bonded to an adjacent group to form a ring. In Formula D-1, when q1 is 2 or greater, multiple R_{d1} groups may all be the same or at least one may be different from the rest. In Formula D-2, when q2 is 2 or greater, multiple R_{d2} groups may all be the same or at least one may be different from the rest. In Formula D-3, when q₃ is 2 or greater, multiple R_{d3} groups may all be the same or at least one may be different from the rest.

In an embodiment, the anime compound represented by Formula E may be represented by any one of Formula G-1 to Formula G-3. Formula G-1 to Formula G-3 each represent a case where L₁ in Formula E is specified as an unsubstituted phenyl group, an unsubstituted divalent biphenyl group, and

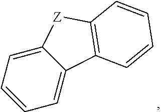

respectively. In Formula G-1 to Formula G-3, n, FG4, R_{c}, Q, and R₁ to R₆ are the same as defined in Formula E.

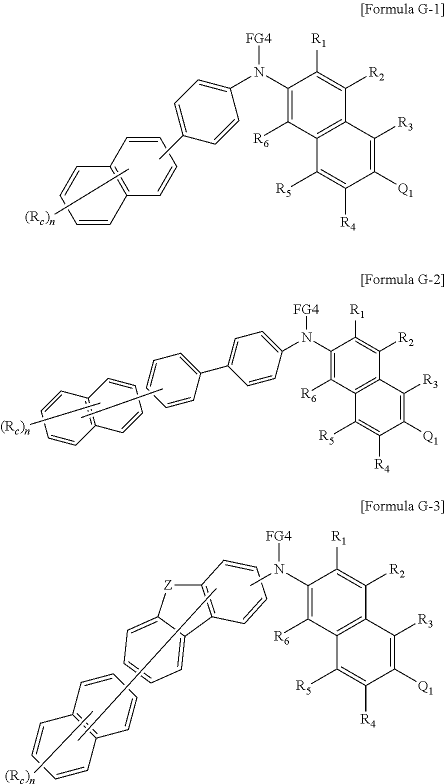

In Formula G-3, Z may be C(R_{z1})(R_{z2}), N(R_{z3}), O, or S. In Formula G-3, R_{z1} to R_{z3} may each independently be a substituted or unsubstituted phenyl group. For example, R_{z1} and R_{z2} may be the same as or different from each other.

In an embodiment, the amine compound represented by Formula G-3 may be represented by any one of Formula GG-1 to Formula GG-3. Each of Formula GG-1 to Formula GG-3 represents a case where in Formula G-3, the position at which L₁ is bonded to the nitrogen atom, and the position, at which the naphthyl group substituted with (R_{c})ₙ is bonded to L₁ are specified. In Formula GG-1 to Formula GG-3, n, FG4, R_{c}, Q, and R₁ to R₆ are the same as defined in Formula E. In Formula GG-1 to Formula GG-3, Z is the same as defined in Formula G-3.

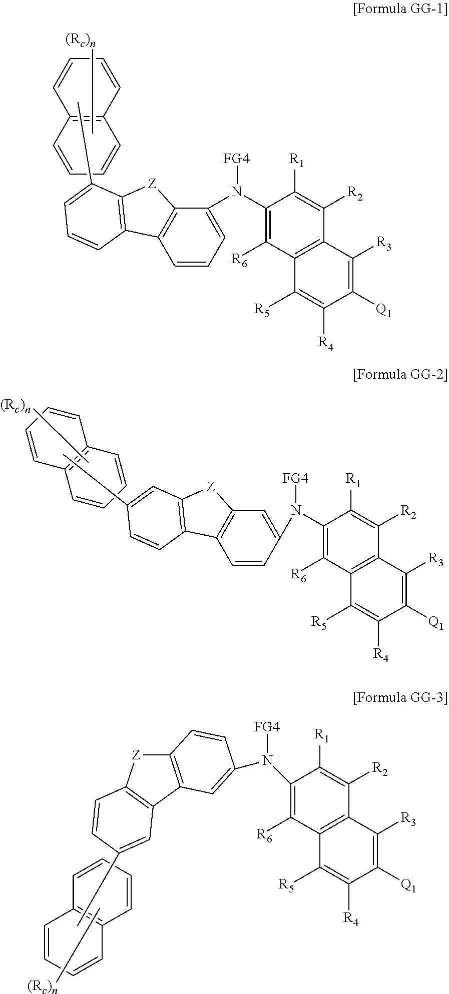

In an embodiment, the amine compound represented by Formula E may include a first substituent represented by any one of Formulae A-1 to A-75, a second substituent represented by any one of Formulae B-1 to B-99 and B-101 to B-106, and a third substituent represented by any one of Formulae A-1 to A-75, B-1 to B-99, B-101 to B-106, C-1 to C-77, and C-101 to C-109. In another embodiment, in the amine compound represented by Formula 1, FG1 may be a group represented by any one of Formulae A-1 to A-75, FG2 may be a group represented by any one of Formulae B-1 to B-99 and B-101 to B-106, and FG3 may be a group represented by any one of Formulae A-1 to A-75, B-1 to B-99, B-101 to B-106, C-1 to C-77, and C-101 to C-109.

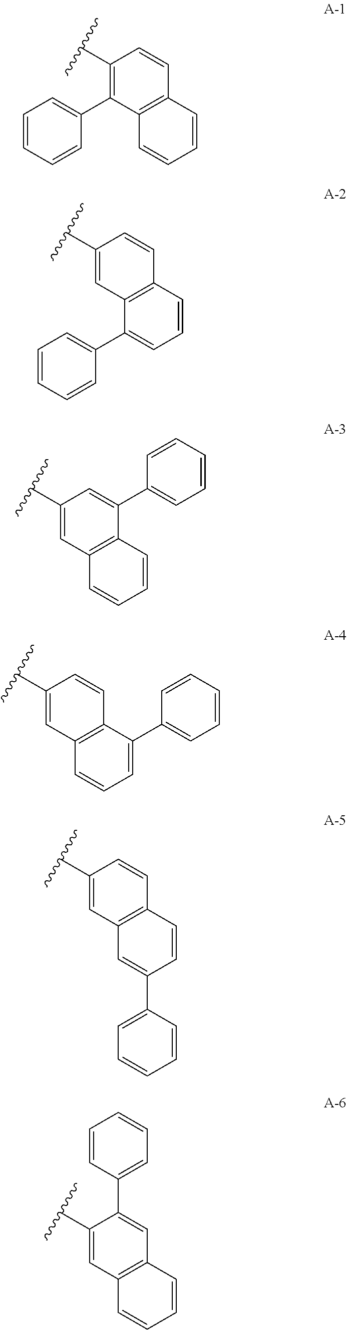

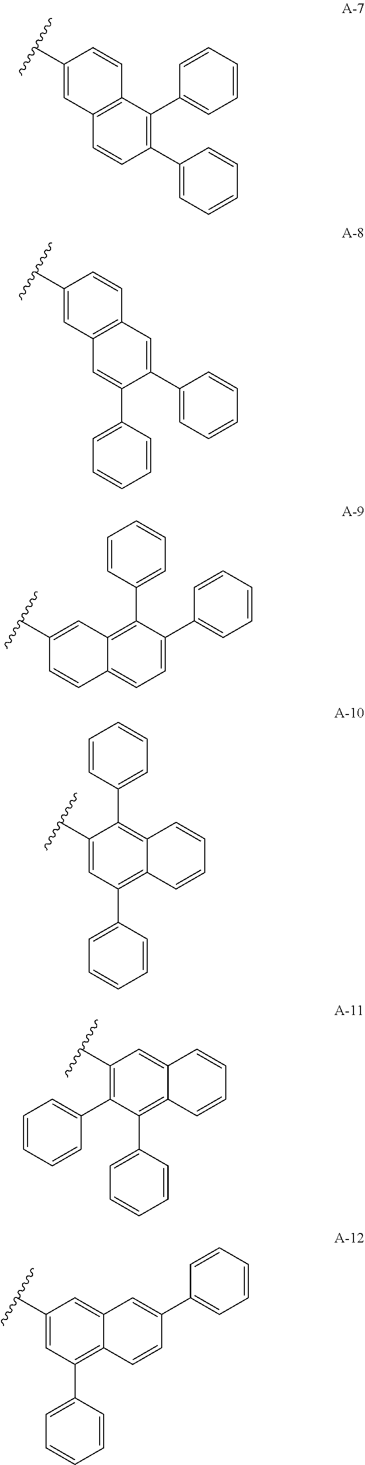

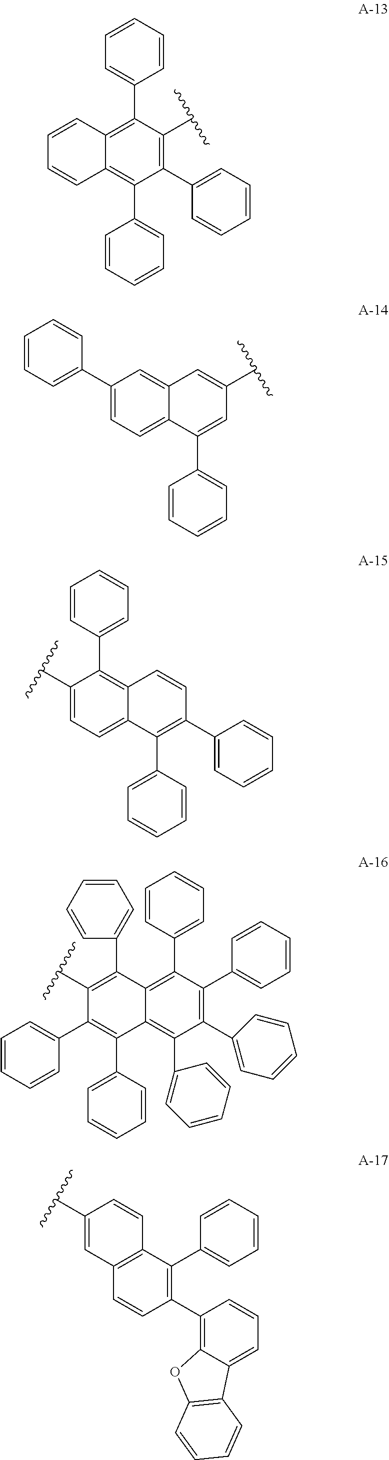

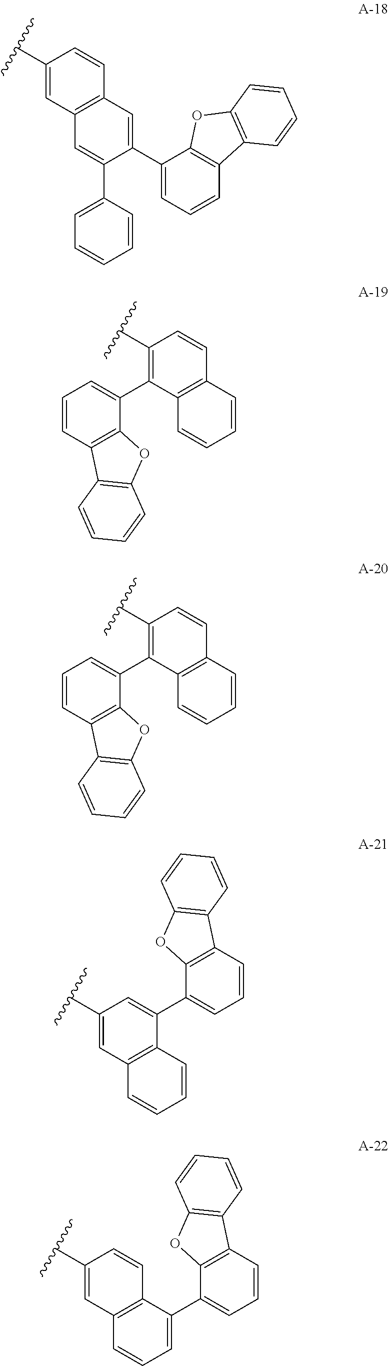 [SMILES: *c1ccc2cc(-c3cccc4c3oc3ccccc34)c(-c3ccccc3)cc2c1 ; *c1ccc2ccccc2c1-c1cccc2c1oc1ccccc12 ; *c1ccc2ccccc2c1-c1cccc2c1oc1ccccc12 ; *c1cc(-c2cccc3c2oc2ccccc23)c2ccccc2c1 ; *c1ccc2c(-c3cccc4c3oc3ccccc34)cccc2c1]

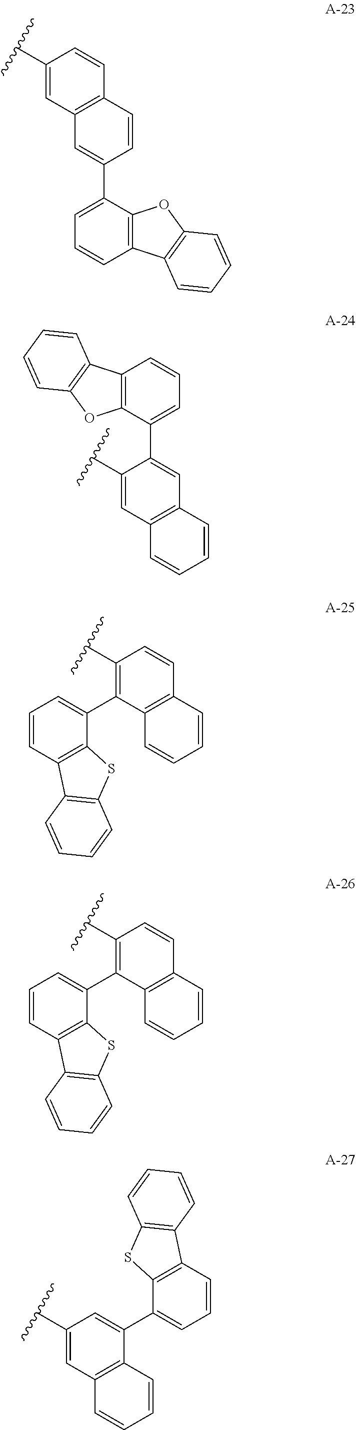 [SMILES: *c1ccc2ccc(-c3cccc4c3oc3ccccc34)cc2c1 ; *c1cc2ccccc2cc1-c1cccc2c1oc1ccccc12 ; *c1ccc2ccccc2c1-c1cccc2c1sc1ccccc12 ; *c1ccc2ccccc2c1-c1cccc2c1sc1ccccc12 ; *c1cc(-c2cccc3c2sc2ccccc23)c2ccccc2c1]

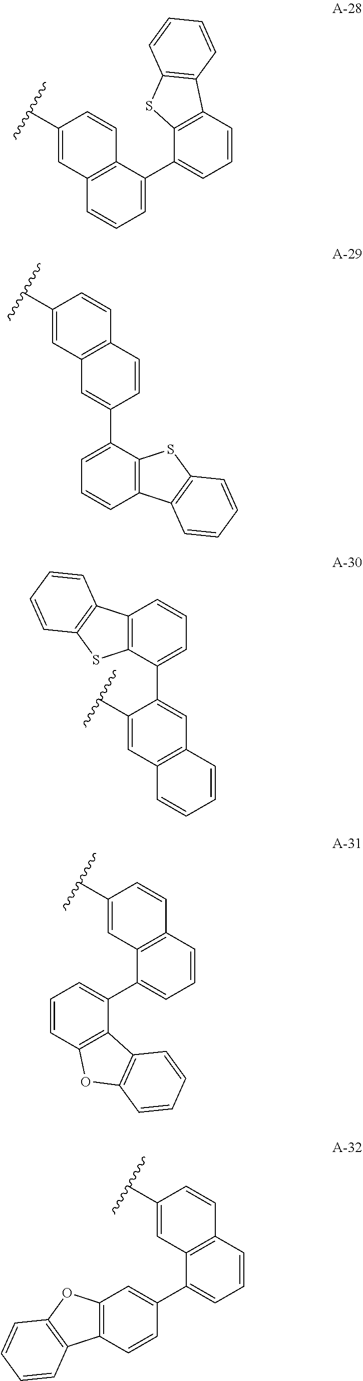 [SMILES: *c1ccc2c(-c3cccc4c3sc3ccccc34)cccc2c1 ; *c1ccc2ccc(-c3cccc4c3sc3ccccc34)cc2c1 ; *c1cc2ccccc2cc1-c1cccc2c1sc1ccccc12 ; *c1ccc2cccc(-c3cccc4oc5ccccc5c34)c2c1 ; *c1ccc2cccc(-c3ccc4c(c3)oc3ccccc34)c2c1]

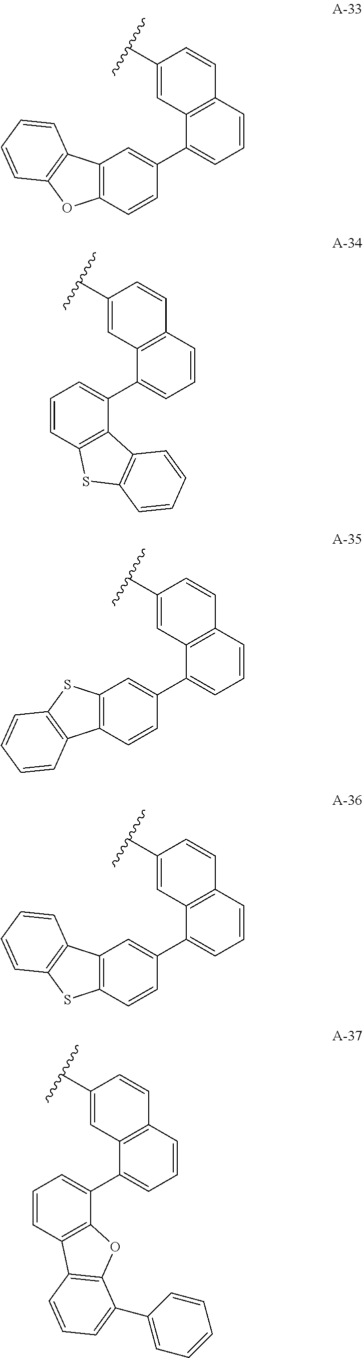 [SMILES: *c1ccc2cccc(-c3ccc4oc5ccccc5c4c3)c2c1 ; *c1ccc2cccc(-c3cccc4sc5ccccc5c34)c2c1 ; *c1ccc2cccc(-c3ccc4c(c3)sc3ccccc34)c2c1 ; *c1ccc2cccc(-c3ccc4sc5ccccc5c4c3)c2c1 ; *c1ccc2cccc(-c3cccc4c3oc3c(-c5ccccc5)cccc34)c2c1]

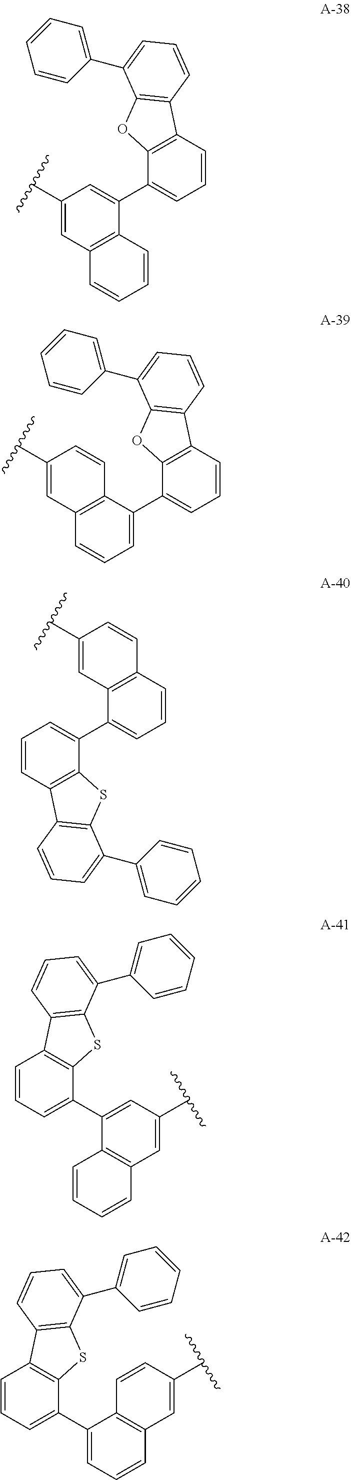

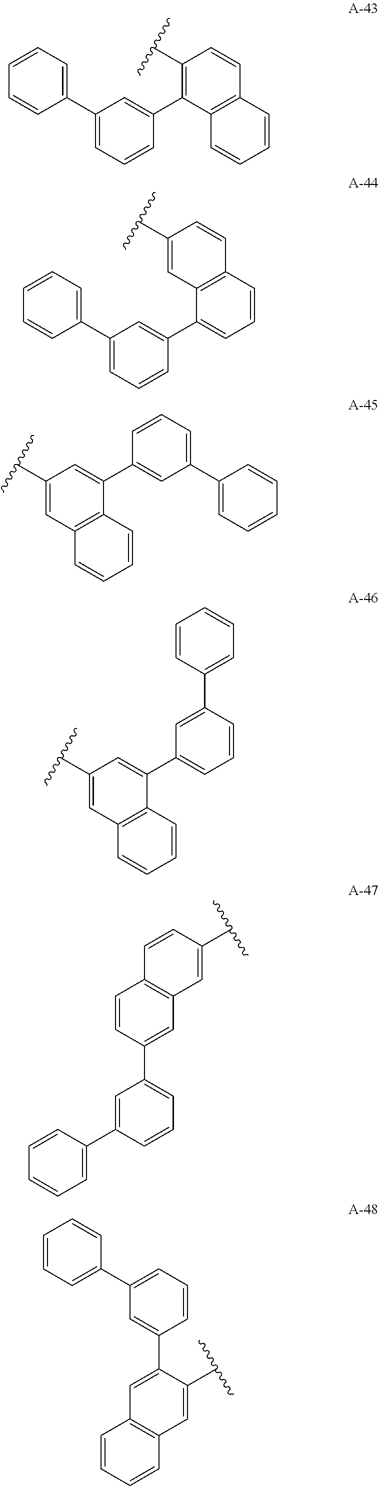

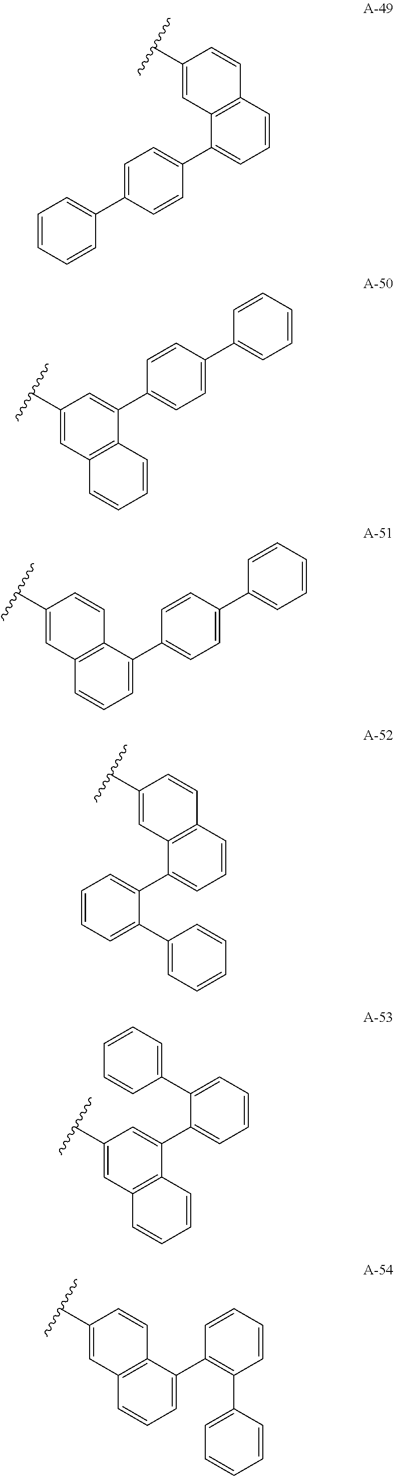 [SMILES: *c1ccc2cccc(-c3ccc(-c4ccccc4)cc3)c2c1 ; *c1cc(-c2ccc(-c3ccccc3)cc2)c2ccccc2c1 ; *c1ccc2c(-c3ccc(-c4ccccc4)cc3)cccc2c1 ; *c1ccc2cccc(-c3ccccc3-c3ccccc3)c2c1 ; *c1cc(-c2ccccc2-c2ccccc2)c2ccccc2c1 ; *c1ccc2c(-c3ccccc3-c3ccccc3)cccc2c1]

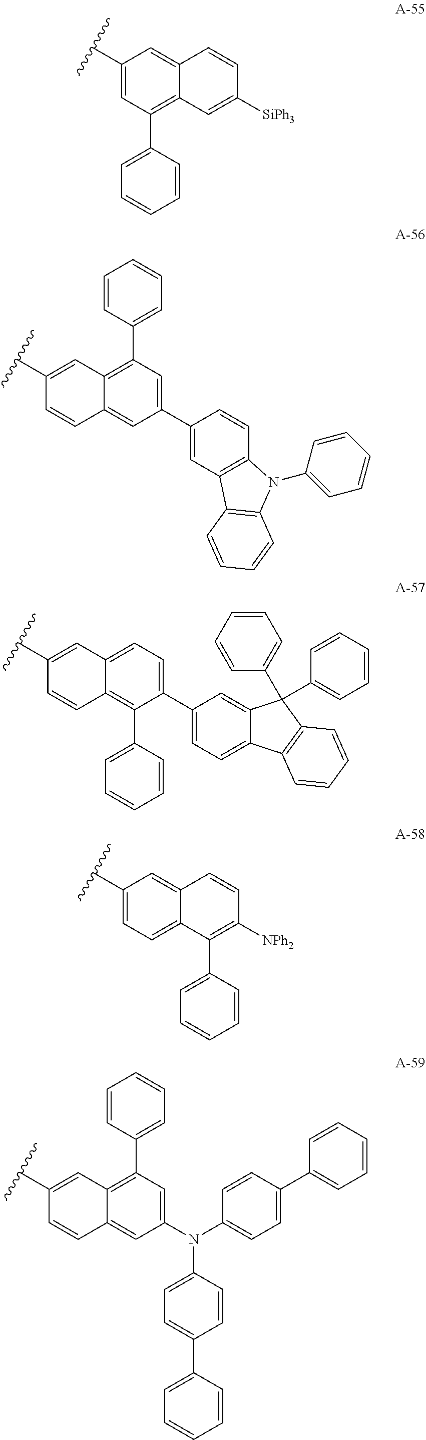

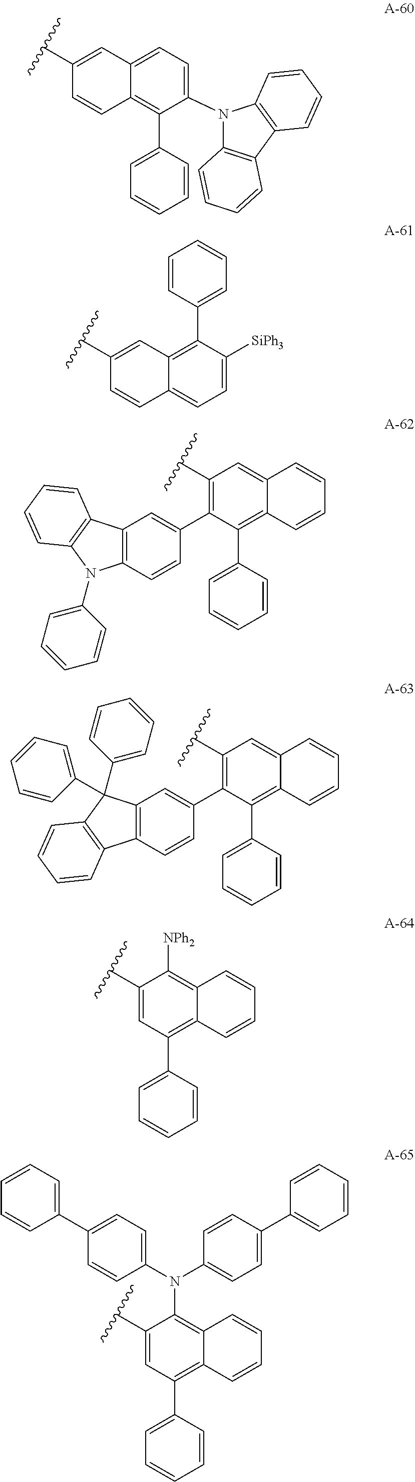

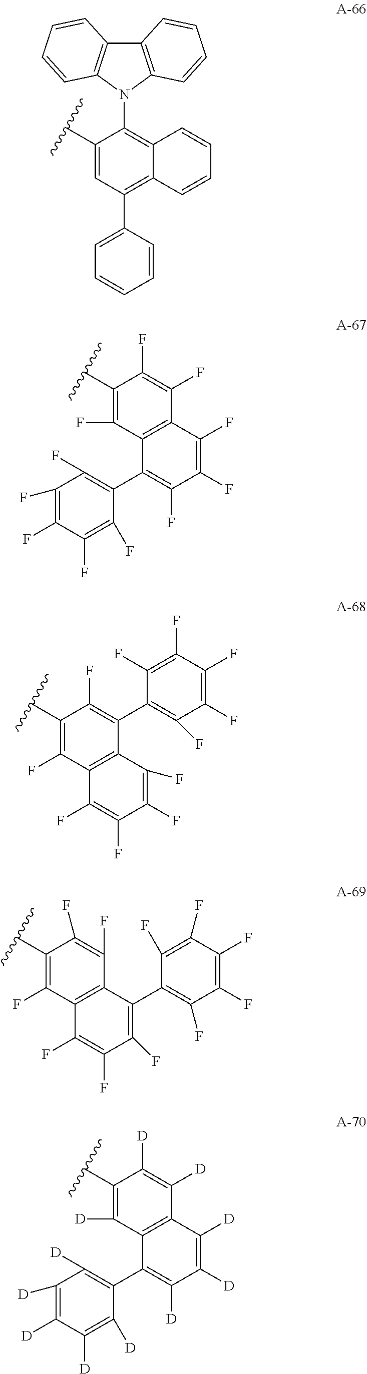 [SMILES: *c1cc(-c2ccccc2)c2ccccc2c1-n1c2ccccc2c2ccccc21 ; *c1c(F)c(F)c2c(F)c(F)c(F)c(-c3c(F)c(F)c(F)c(F)c3F)c2c1F ; *c1c(F)c(-c2c(F)c(F)c(F)c(F)c2F)c2c(F)c(F)c(F)c(F)c2c1F ; *c1c(F)c(F)c2c(-c3c(F)c(F)c(F)c(F)c3F)c(F)c(F)c(F)c2c1F ; *c1c([2H])c([2H])c2c([2H])c([2H])c([2H])c(-c3c([2H])c([2H])c([2H])c([2H])c3[2H])c2c1[2H]]

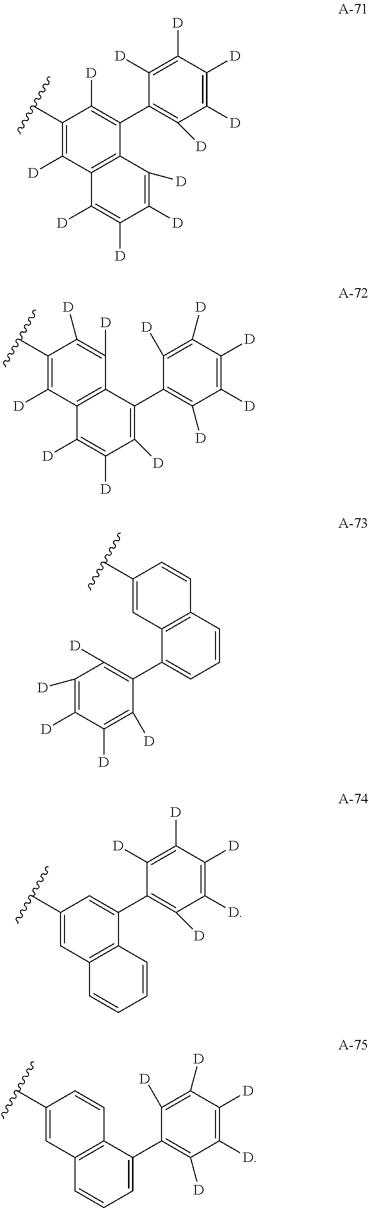

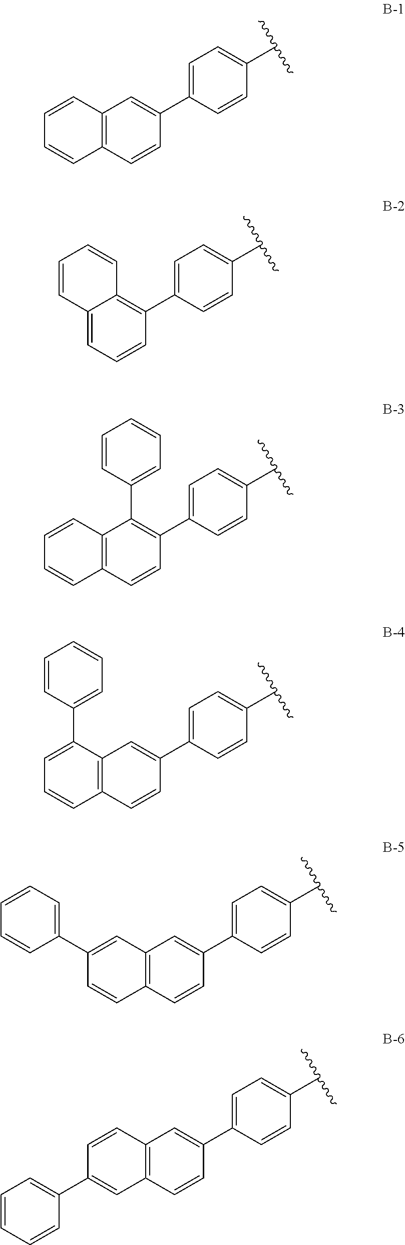 [SMILES: *c1ccc(-c2ccc3ccccc3c2)cc1 ; *c1ccc(-c2cccc3ccccc23)cc1 ; *c1ccc(-c2ccc3ccccc3c2-c2ccccc2)cc1 ; *c1ccc(-c2ccc3cccc(-c4ccccc4)c3c2)cc1 ; *c1ccc(-c2ccc3ccc(-c4ccccc4)cc3c2)cc1 ; *c1ccc(-c2ccc3cc(-c4ccccc4)ccc3c2)cc1]

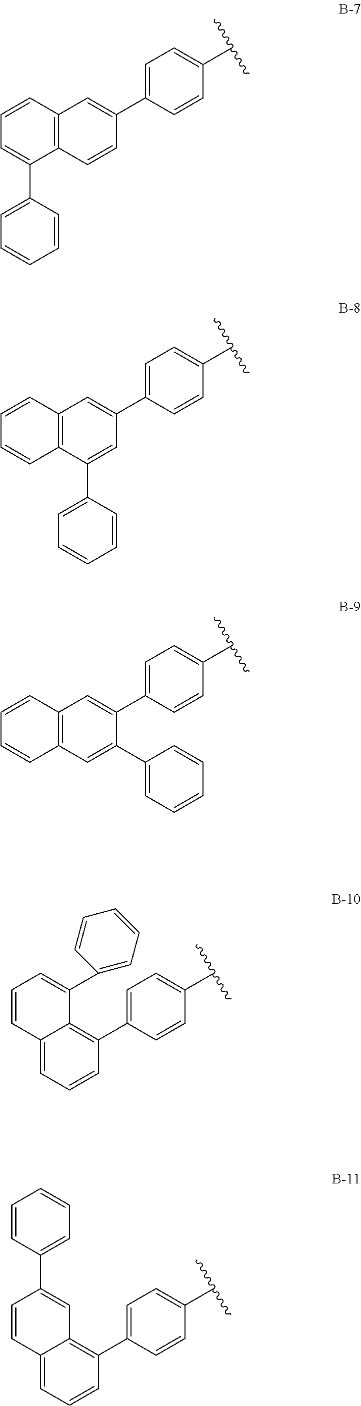 [SMILES: *c1ccc(-c2ccc3c(-c4ccccc4)cccc3c2)cc1 ; *c1ccc(-c2cc(-c3ccccc3)c3ccccc3c2)cc1 ; *c1ccc(-c2cc3ccccc3cc2-c2ccccc2)cc1 ; *c1ccc(-c2cccc3cccc(-c4ccccc4)c23)cc1 ; *c1ccc(-c2cccc3ccc(-c4ccccc4)cc23)cc1]

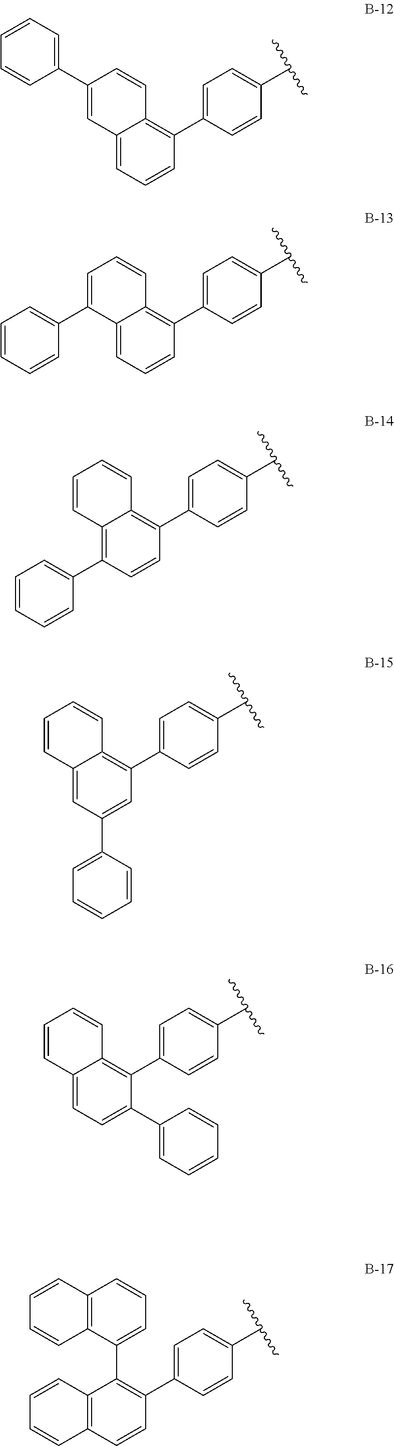 [SMILES: *c1ccc(-c2cccc3cc(-c4ccccc4)ccc23)cc1 ; *c1ccc(-c2cccc3c(-c4ccccc4)cccc23)cc1 ; *c1ccc(-c2ccc(-c3ccccc3)c3ccccc23)cc1 ; *c1ccc(-c2cc(-c3ccccc3)cc3ccccc23)cc1 ; *c1ccc(-c2c(-c3ccccc3)ccc3ccccc23)cc1 ; *c1ccc(-c2ccc3ccccc3c2-c2cccc3ccccc23)cc1]

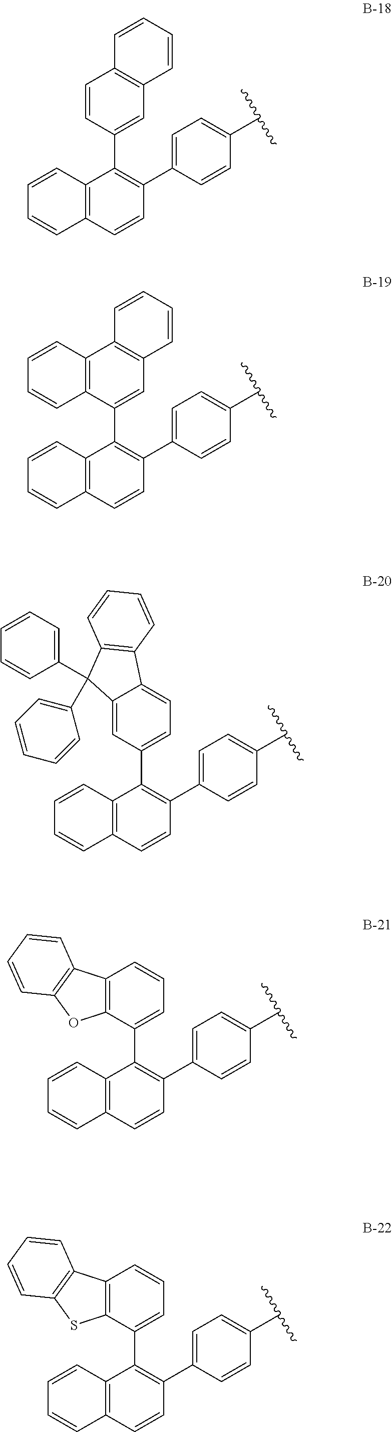 [SMILES: *c1ccc(-c2ccc3ccccc3c2-c2ccc3ccccc3c2)cc1 ; *c1ccc(-c2ccc3ccccc3c2-c2cc3ccccc3c3ccccc23)cc1 ; *c1ccc(-c2ccc3ccccc3c2-c2ccc3c(c2)C(c2ccccc2)(c2ccccc2)c2ccccc2-3)cc1 ; *c1ccc(-c2ccc3ccccc3c2-c2cccc3c2oc2ccccc23)cc1 ; *c1ccc(-c2ccc3ccccc3c2-c2cccc3c2sc2ccccc23)cc1]

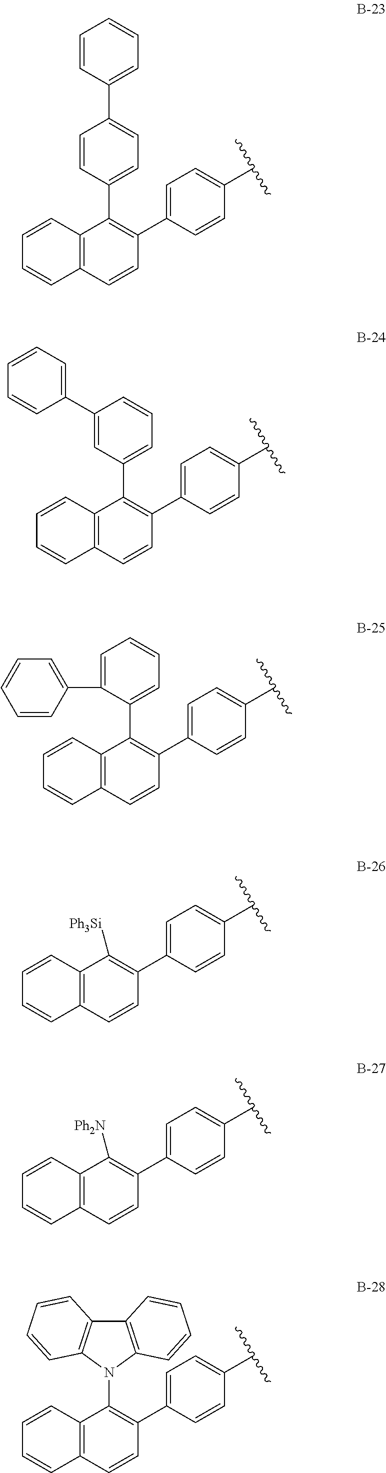

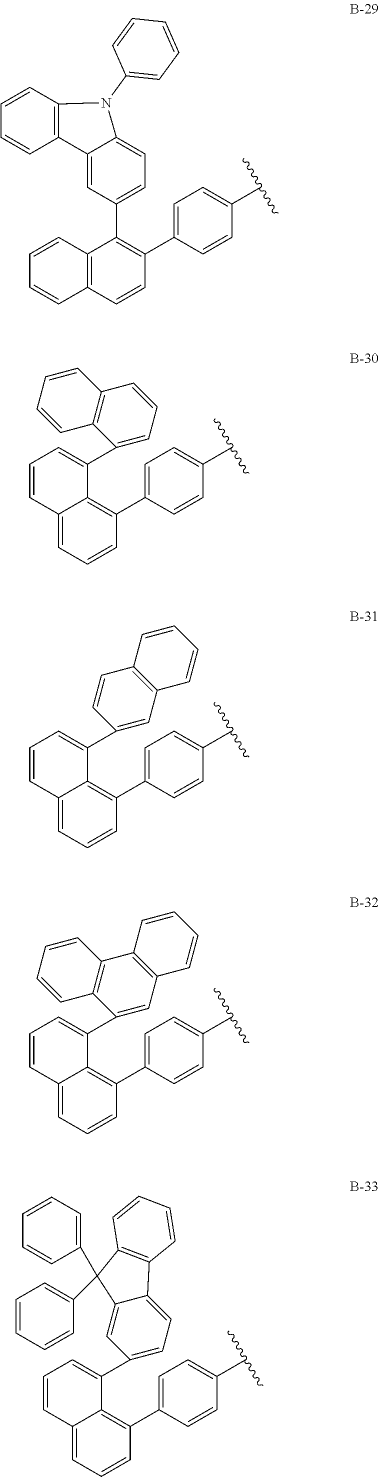 [SMILES: *c1ccc(-c2ccc3ccccc3c2-c2ccc3c(c2)c2ccccc2n3-c2ccccc2)cc1 ; *c1ccc(-c2cccc3cccc(-c4cccc5ccccc45)c23)cc1 ; *c1ccc(-c2cccc3cccc(-c4ccc5ccccc5c4)c23)cc1 ; *c1ccc(-c2cccc3cccc(-c4cc5ccccc5c5ccccc45)c23)cc1 ; *c1ccc(-c2cccc3cccc(-c4ccc5c(c4)C(c4ccccc4)(c4ccccc4)c4ccccc4-5)c23)cc1]

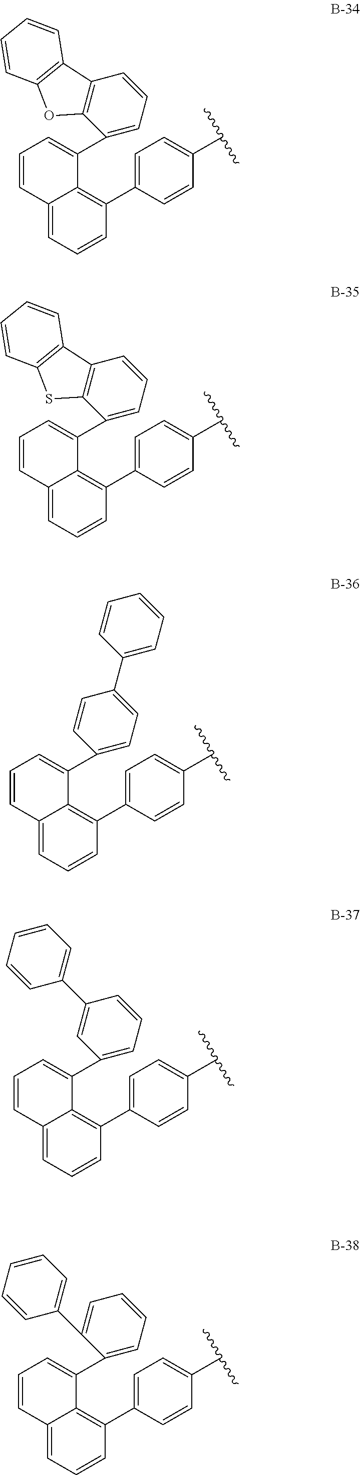 [SMILES: *c1ccc(-c2cccc3cccc(-c4cccc5c4oc4ccccc45)c23)cc1 ; *c1ccc(-c2cccc3cccc(-c4cccc5c4sc4ccccc45)c23)cc1 ; *c1ccc(-c2cccc3cccc(-c4ccc(-c5ccccc5)cc4)c23)cc1 ; *c1ccc(-c2cccc3cccc(-c4cccc(-c5ccccc5)c4)c23)cc1 ; *c1ccc(-c2cccc3cccc(-c4ccccc4-c4ccccc4)c23)cc1]

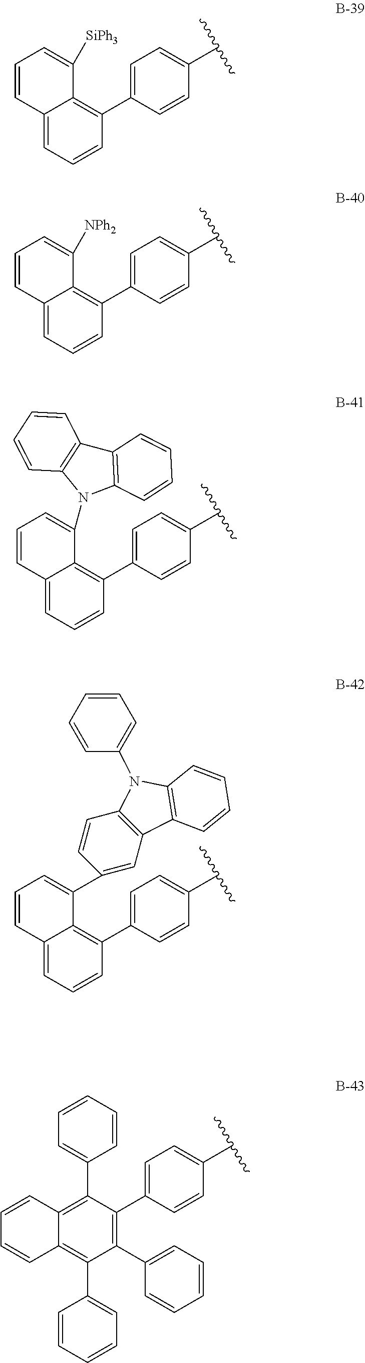

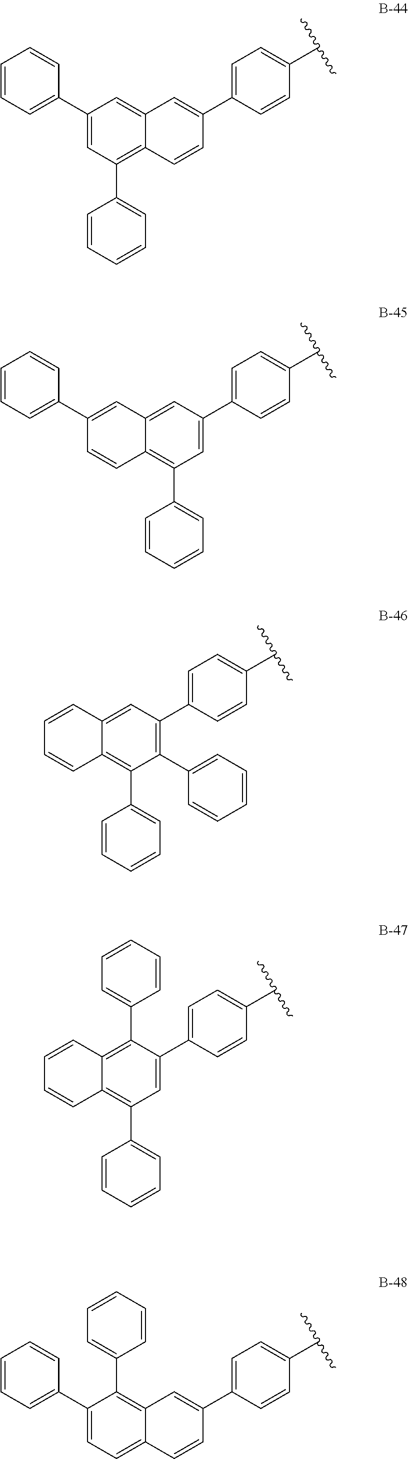 [SMILES: *c1ccc(-c2ccc3c(-c4ccccc4)cc(-c4ccccc4)cc3c2)cc1 ; *c1ccc(-c2cc(-c3ccccc3)c3ccc(-c4ccccc4)cc3c2)cc1 ; *c1ccc(-c2cc3ccccc3c(-c3ccccc3)c2-c2ccccc2)cc1 ; *c1ccc(-c2cc(-c3ccccc3)c3ccccc3c2-c2ccccc2)cc1 ; *c1ccc(-c2ccc3ccc(-c4ccccc4)c(-c4ccccc4)c3c2)cc1]

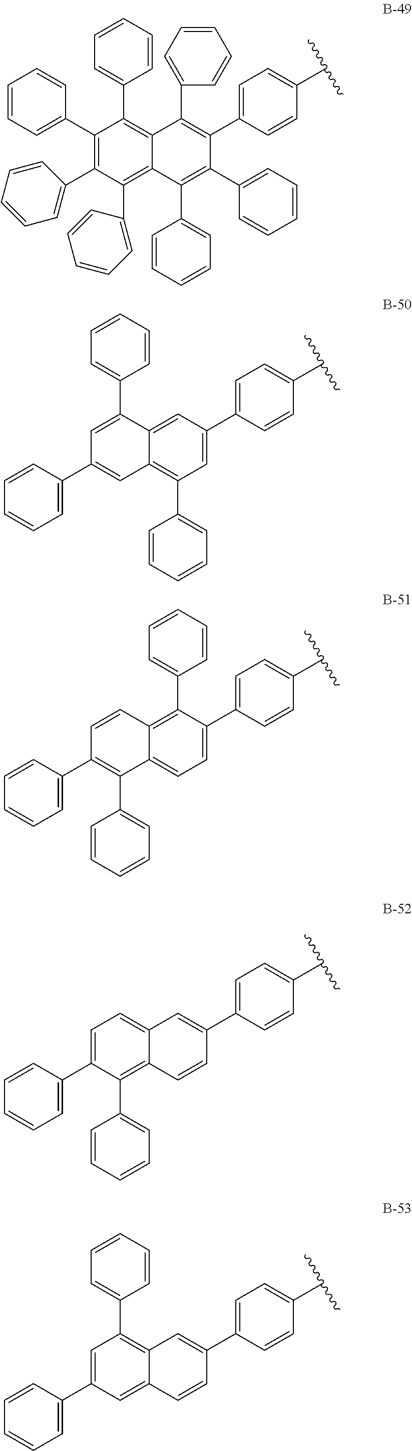

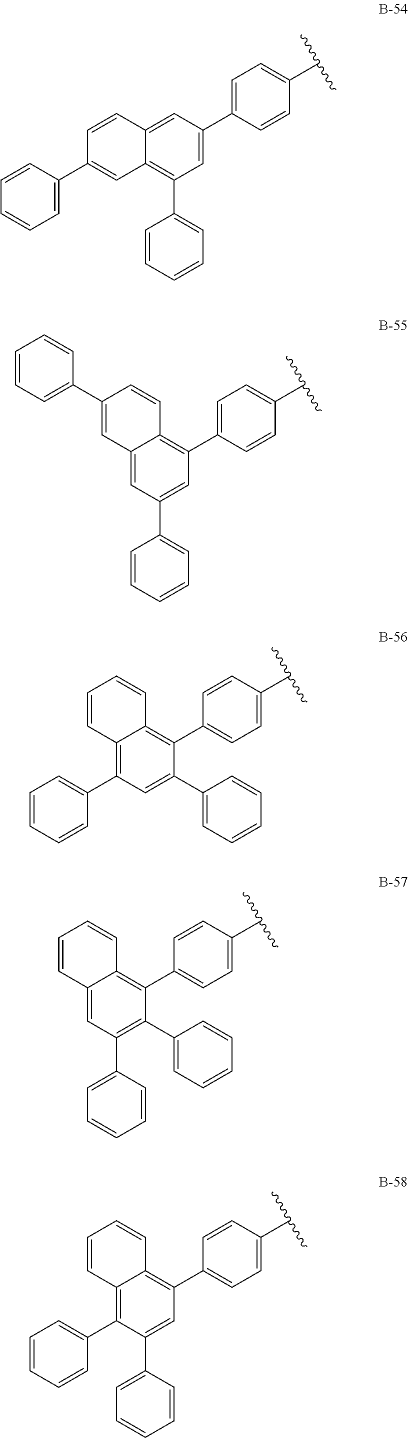 [SMILES: *c1ccc(-c2cc(-c3ccccc3)c3cc(-c4ccccc4)ccc3c2)cc1 ; *c1ccc(-c2cc(-c3ccccc3)cc3cc(-c4ccccc4)ccc23)cc1 ; *c1ccc(-c2c(-c3ccccc3)cc(-c3ccccc3)c3ccccc23)cc1 ; *c1ccc(-c2c(-c3ccccc3)c(-c3ccccc3)cc3ccccc23)cc1 ; *c1ccc(-c2cc(-c3ccccc3)c(-c3ccccc3)c3ccccc23)cc1]

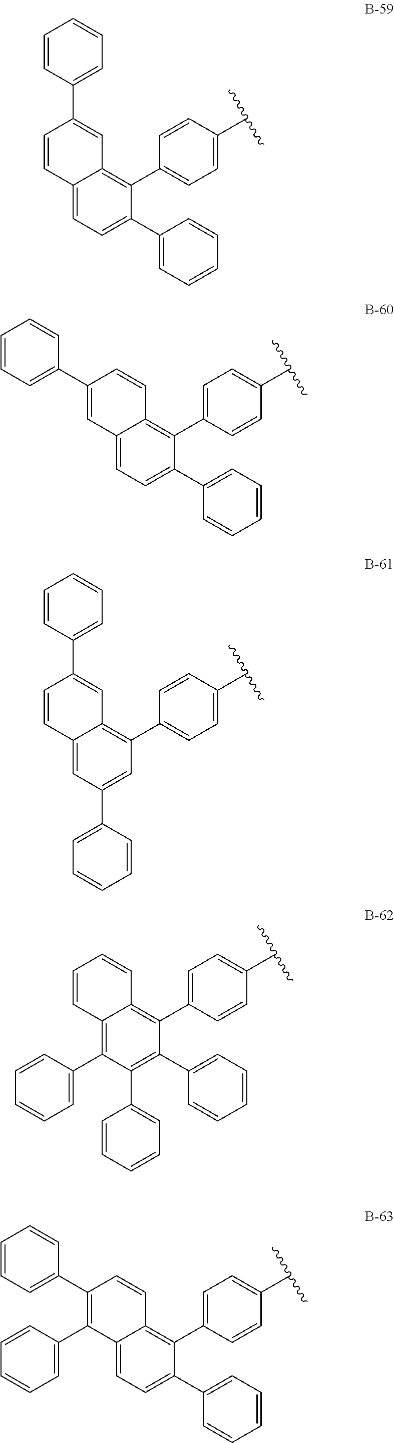 [SMILES: *c1ccc(-c2c(-c3ccccc3)ccc3ccc(-c4ccccc4)cc23)cc1 ; *c1ccc(-c2c(-c3ccccc3)ccc3cc(-c4ccccc4)ccc23)cc1 ; *c1ccc(-c2cc(-c3ccccc3)cc3ccc(-c4ccccc4)cc23)cc1 ; *c1ccc(-c2c(-c3ccccc3)c(-c3ccccc3)c(-c3ccccc3)c3ccccc23)cc1 ; *c1ccc(-c2c(-c3ccccc3)ccc3c(-c4ccccc4)c(-c4ccccc4)ccc23)cc1]

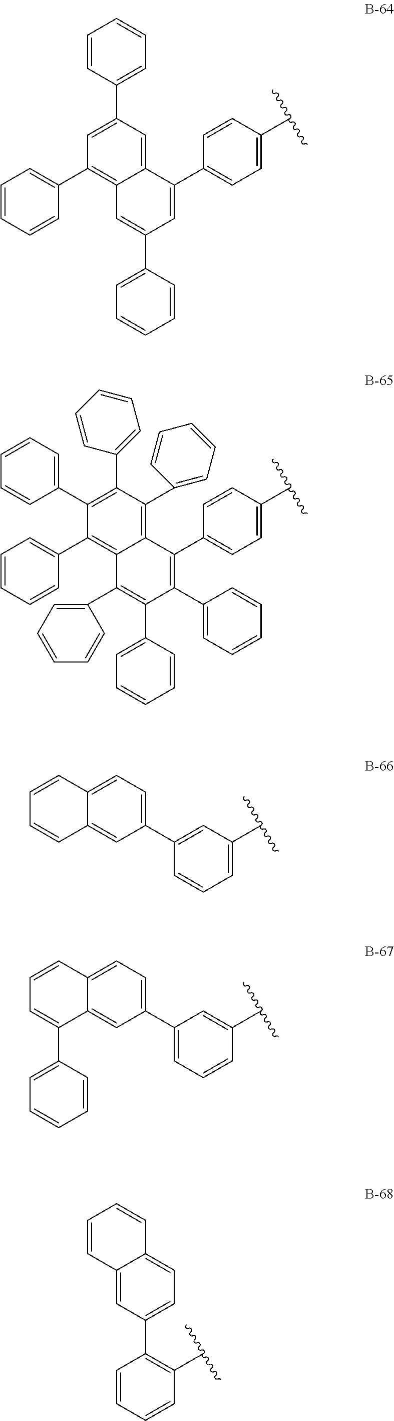 [SMILES: *c1ccc(-c2cc(-c3ccccc3)cc3c(-c4ccccc4)cc(-c4ccccc4)cc23)cc1 ; *c1ccc(-c2c(-c3ccccc3)c(-c3ccccc3)c(-c3ccccc3)c3c(-c4ccccc4)c(-c4ccccc4)c(-c4ccccc4)c(-c4ccccc4)c23)cc1 ; *c1cccc(-c2ccc3ccccc3c2)c1 ; *c1cccc(-c2ccc3cccc(-c4ccccc4)c3c2)c1 ; *c1ccccc1-c1ccc2ccccc2c1]

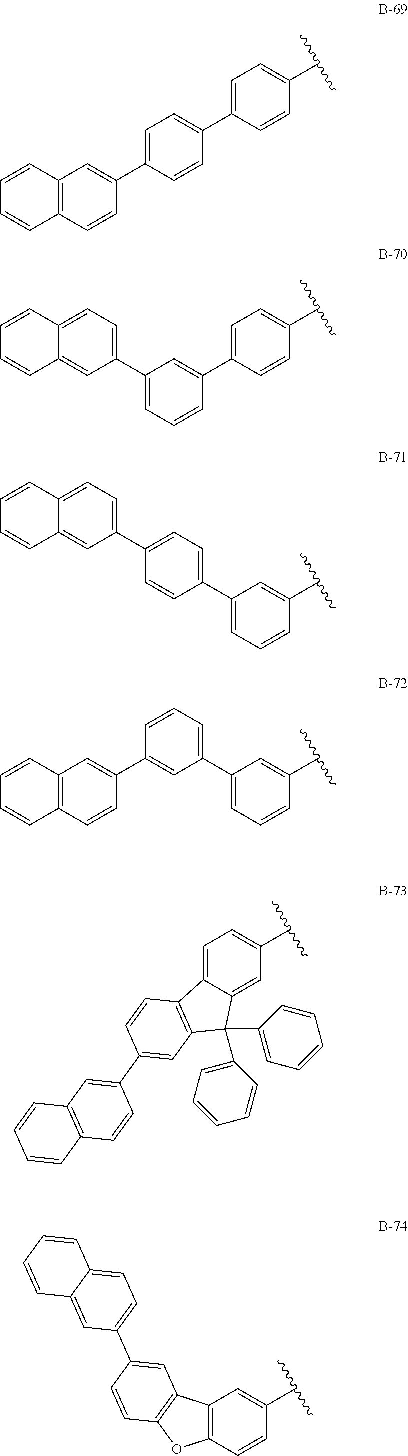 [SMILES: *c1ccc(-c2ccc(-c3ccc4ccccc4c3)cc2)cc1 ; *c1ccc(-c2cccc(-c3ccc4ccccc4c3)c2)cc1 ; *c1cccc(-c2ccc(-c3ccc4ccccc4c3)cc2)c1 ; *c1cccc(-c2cccc(-c3ccc4ccccc4c3)c2)c1 ; *c1ccc2c(c1)C(c1ccccc1)(c1ccccc1)c1cc(-c3ccc4ccccc4c3)ccc1-2 ; *c1ccc2oc3ccc(-c4ccc5ccccc5c4)cc3c2c1]

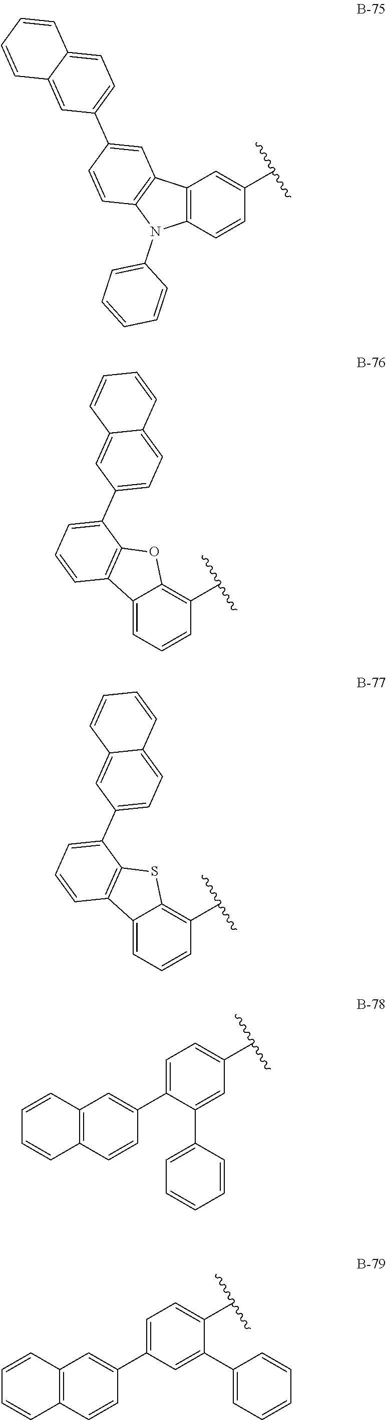 [SMILES: *c1ccc2c(c1)c1cc(-c3ccc4ccccc4c3)ccc1n2-c1ccccc1 ; *c1cccc2c1oc1c(-c3ccc4ccccc4c3)cccc12 ; *c1cccc2c1sc1c(-c3ccc4ccccc4c3)cccc12 ; *c1ccc(-c2ccc3ccccc3c2)c(-c2ccccc2)c1 ; *c1ccc(-c2ccc3ccccc3c2)cc1-c1ccccc1]

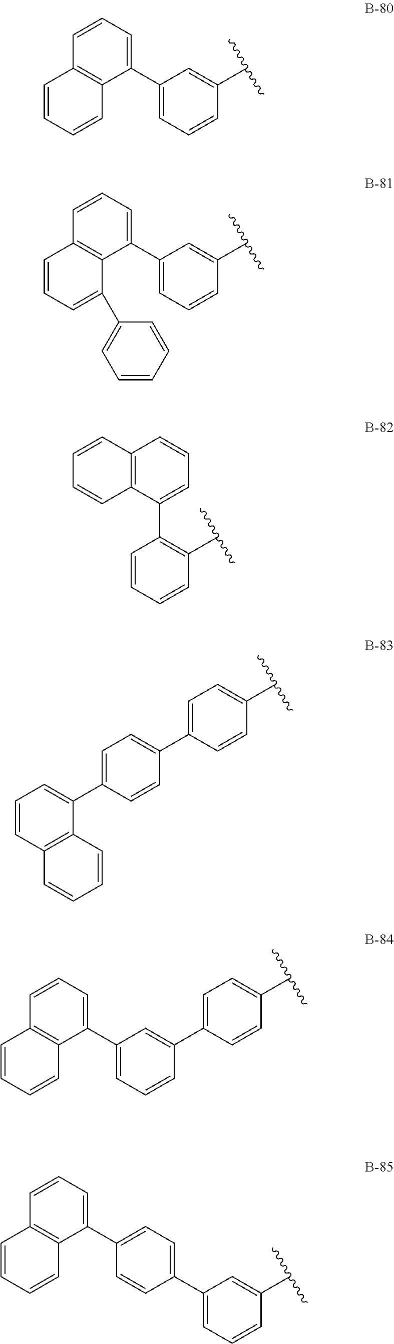 [SMILES: *c1cccc(-c2cccc3ccccc23)c1 ; *c1cccc(-c2cccc3cccc(-c4ccccc4)c23)c1 ; *c1ccccc1-c1cccc2ccccc12 ; *c1ccc(-c2ccc(-c3cccc4ccccc34)cc2)cc1 ; *c1ccc(-c2cccc(-c3cccc4ccccc34)c2)cc1 ; *c1cccc(-c2ccc(-c3cccc4ccccc34)cc2)c1]

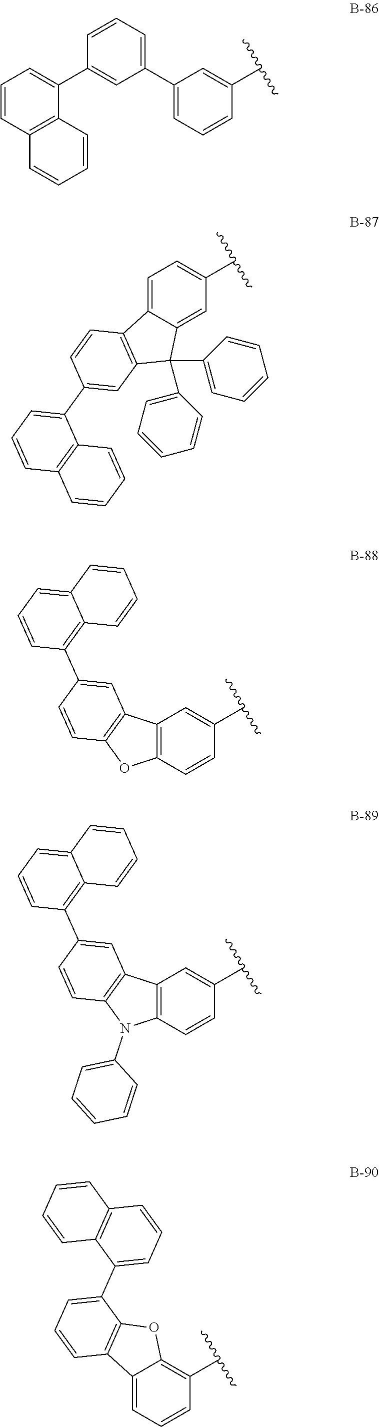 [SMILES: *c1cccc(-c2cccc(-c3cccc4ccccc34)c2)c1 ; *c1ccc2c(c1)C(c1ccccc1)(c1ccccc1)c1cc(-c3cccc4ccccc34)ccc1-2 ; *c1ccc2oc3ccc(-c4cccc5ccccc45)cc3c2c1 ; *c1ccc2c(c1)c1cc(-c3cccc4ccccc34)ccc1n2-c1ccccc1 ; *c1cccc2c1oc1c(-c3cccc4ccccc34)cccc12]

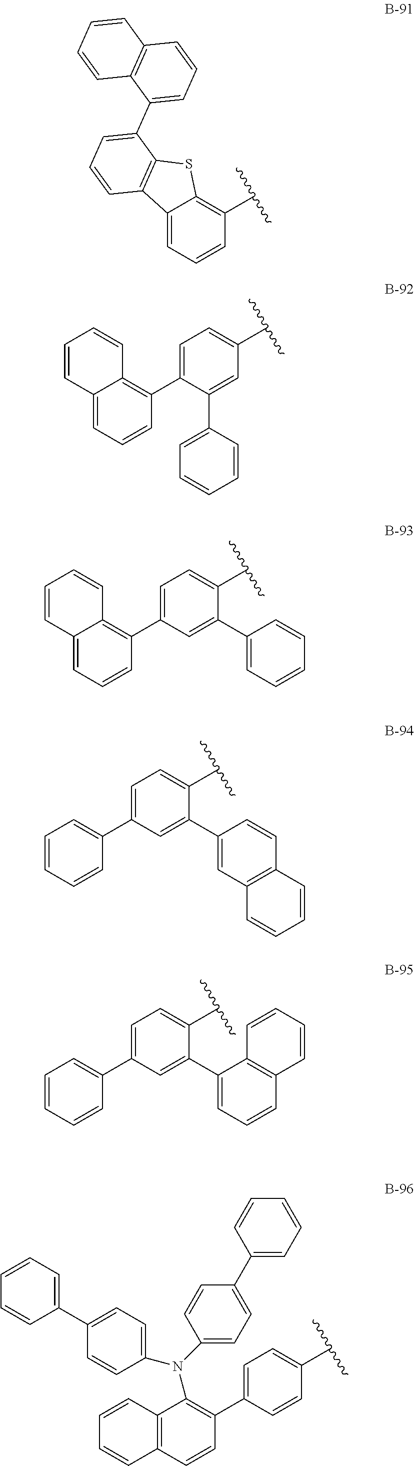 [SMILES: *c1cccc2c1sc1c(-c3cccc4ccccc34)cccc12 ; *c1ccc(-c2cccc3ccccc23)c(-c2ccccc2)c1 ; *c1ccc(-c2cccc3ccccc23)cc1-c1ccccc1 ; *c1ccc(-c2ccccc2)cc1-c1ccc2ccccc2c1 ; *c1ccc(-c2ccccc2)cc1-c1cccc2ccccc12 ; *c1ccc(-c2ccc3ccccc3c2N(c2ccc(-c3ccccc3)cc2)c2ccc(-c3ccccc3)cc2)cc1]

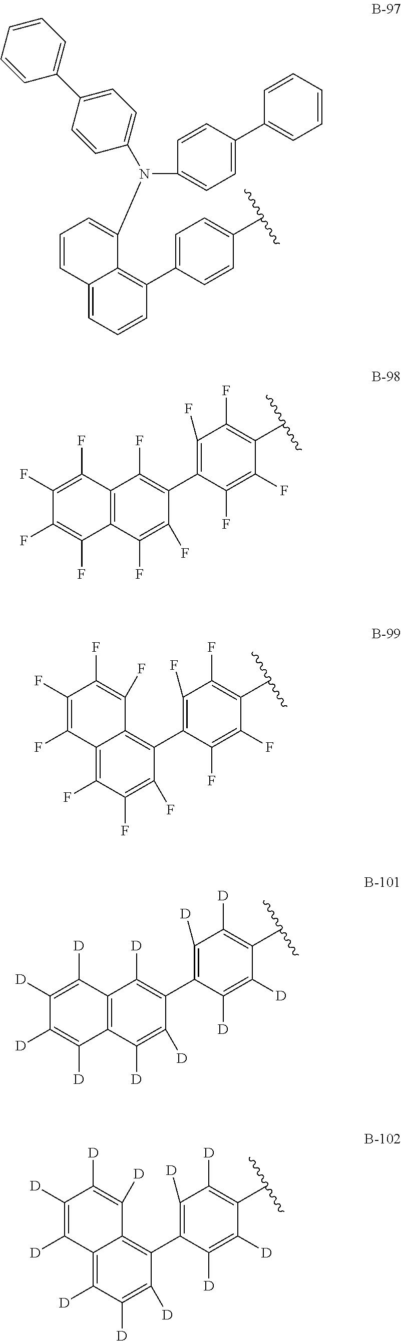 [SMILES: *c1ccc(-c2cccc3cccc(N(c4ccc(-c5ccccc5)cc4)c4ccc(-c5ccccc5)cc4)c23)cc1 ; *c1c(F)c(F)c(-c2c(F)c(F)c3c(F)c(F)c(F)c(F)c3c2F)c(F)c1F ; *c1c(F)c(F)c(-c2c(F)c(F)c(F)c3c(F)c(F)c(F)c(F)c23)c(F)c1F ; *c1c([2H])c([2H])c(-c2c([2H])c([2H])c3c([2H])c([2H])c([2H])c([2H])c3c2[2H])c([2H])c1[2H] ; *c1c([2H])c([2H])c(-c2c([2H])c([2H])c([2H])c3c([2H])c([2H])c([2H])c([2H])c23)c([2H])c1[2H]]

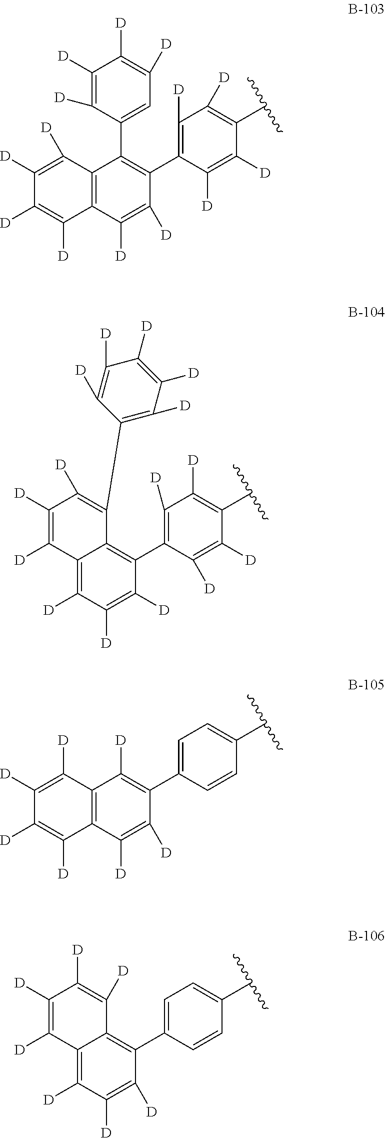 [SMILES: *c1c([2H])c([2H])c(-c2c([2H])c([2H])c3c([2H])c([2H])c([2H])c([2H])c3c2-c2cc([2H])c([2H])c([2H])c2[2H])c([2H])c1[2H] ; *c1c([2H])c([2H])c(-c2c([2H])c([2H])c([2H])c3c([2H])c([2H])c([2H])c(-c4c([2H])c([2H])c([2H])c([2H])c4[2H])c23)c([2H])c1[2H] ; *c1ccc(-c2c([2H])c([2H])c3c([2H])c([2H])c([2H])c([2H])c3c2[2H])cc1 ; *c1ccc(-c2c([2H])c([2H])c([2H])c3c([2H])c([2H])c([2H])c([2H])c23)cc1]

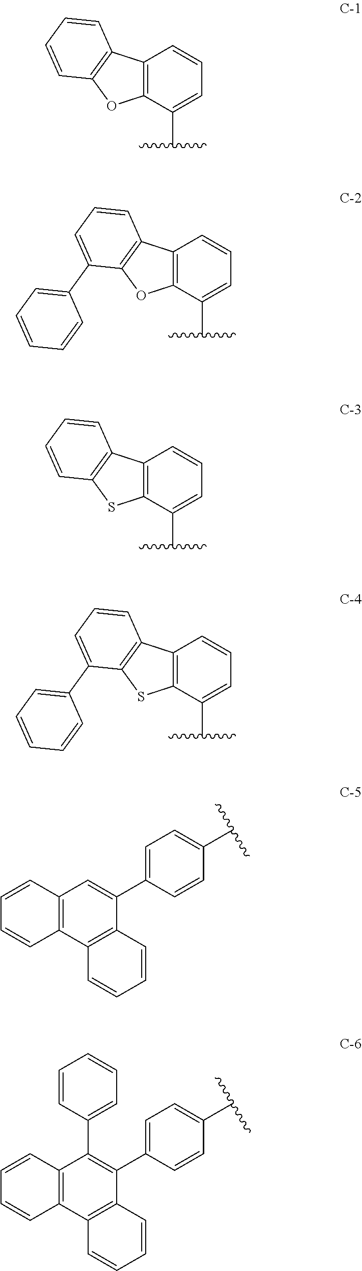 [SMILES: *c1cccc2c1oc1ccccc12 ; *c1cccc2c1oc1c(-c3ccccc3)cccc12 ; *c1cccc2c1sc1ccccc12 ; *c1cccc2c1sc1c(-c3ccccc3)cccc12 ; *c1ccc(-c2cc3ccccc3c3ccccc23)cc1 ; *c1ccc(-c2c(-c3ccccc3)c3ccccc3c3ccccc23)cc1]

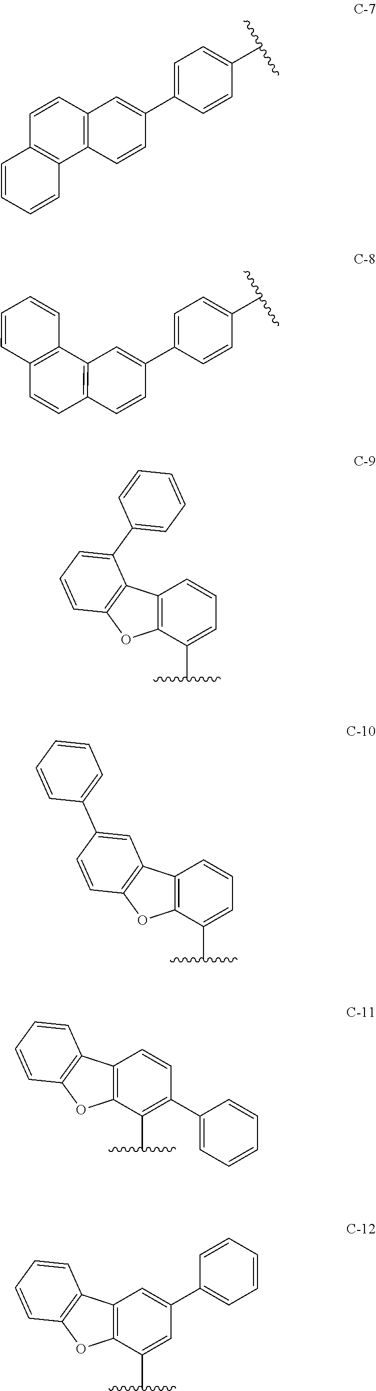 [SMILES: *c1ccc(-c2ccc3c(ccc4ccccc43)c2)cc1 ; *c1ccc(-c2ccc3ccc4ccccc4c3c2)cc1 ; *c1cccc2c1oc1cccc(-c3ccccc3)c12 ; *c1cccc2c1oc1ccc(-c3ccccc3)cc12 ; *c1c(-c2ccccc2)ccc2c1oc1ccccc12 ; *c1cc(-c2ccccc2)cc2c1oc1ccccc12]

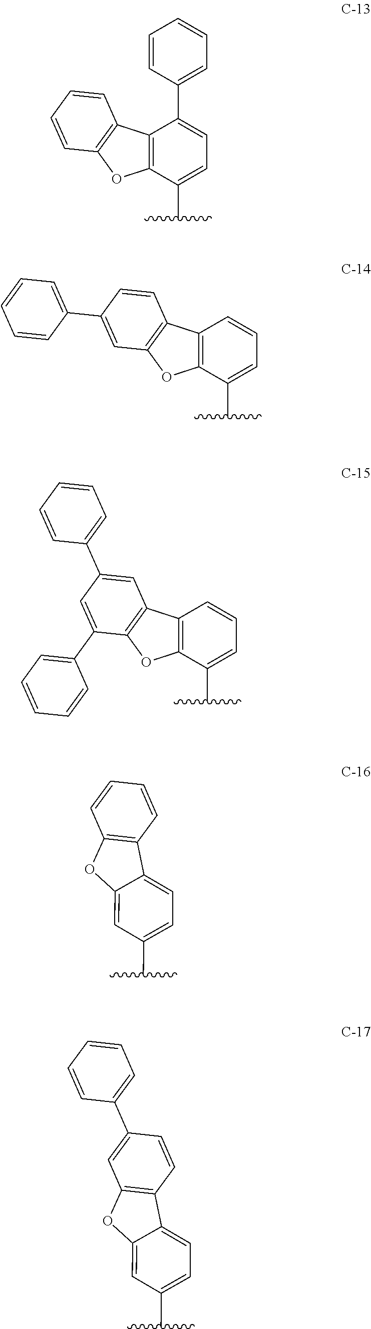 [SMILES: *c1ccc(-c2ccccc2)c2c1oc1ccccc12 ; *c1cccc2c1oc1cc(-c3ccccc3)ccc12 ; *c1cccc2c1oc1c(-c3ccccc3)cc(-c3ccccc3)cc12 ; *c1ccc2c(c1)oc1ccccc12 ; *c1ccc2c(c1)oc1cc(-c3ccccc3)ccc12]

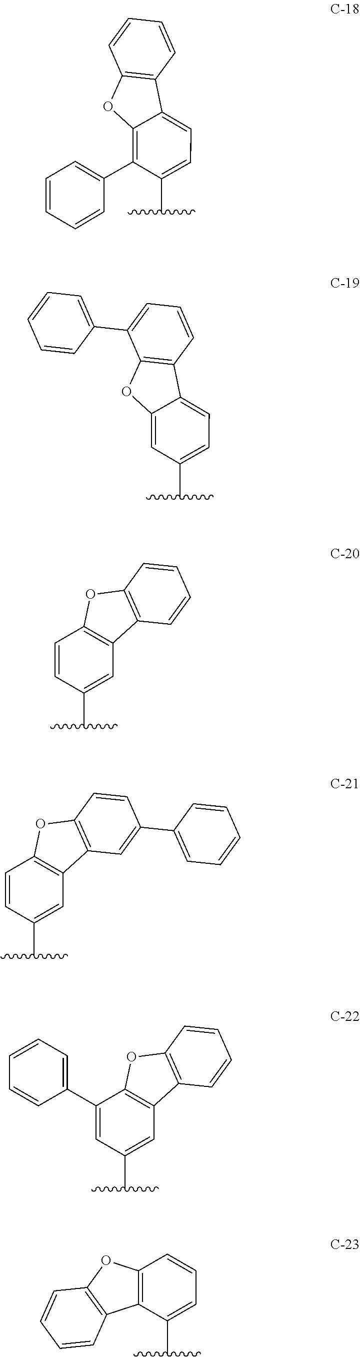 [SMILES: *c1ccc2c(oc3ccccc32)c1-c1ccccc1 ; *c1ccc2c(c1)oc1c(-c3ccccc3)cccc12 ; *c1ccc2oc3ccccc3c2c1 ; *c1ccc2oc3ccc(-c4ccccc4)cc3c2c1 ; *c1cc(-c2ccccc2)c2oc3ccccc3c2c1 ; *c1cccc2oc3ccccc3c12]

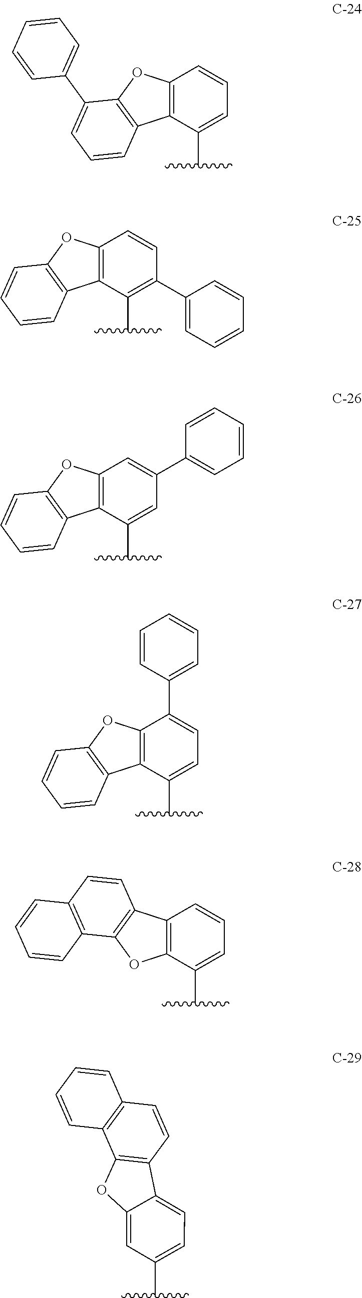 [SMILES: *c1cccc2oc3c(-c4ccccc4)cccc3c12 ; *c1c(-c2ccccc2)ccc2oc3ccccc3c12 ; *c1cc(-c2ccccc2)cc2oc3ccccc3c12 ; *c1ccc(-c2ccccc2)c2oc3ccccc3c12 ; *c1cccc2c1oc1c3ccccc3ccc21 ; *c1ccc2c(c1)oc1c3ccccc3ccc21]

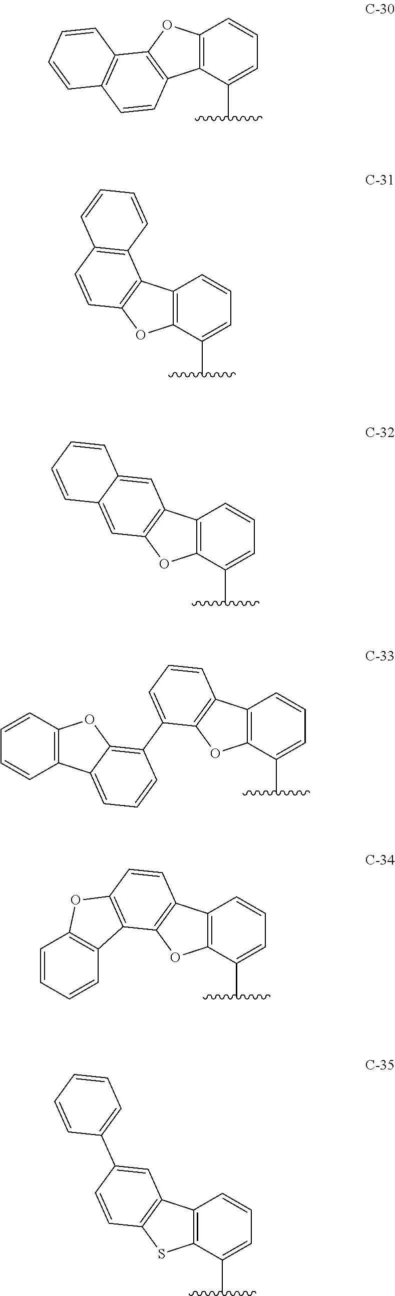 [SMILES: *c1cccc2oc3c4ccccc4ccc3c12 ; *c1cccc2c1oc1ccc3ccccc3c12 ; *c1cccc2c1oc1cc3ccccc3cc12 ; *c1cccc2c1oc1c(-c3cccc4c3oc3ccccc34)cccc12 ; *c1cccc2c1oc1c2ccc2oc3ccccc3c21 ; *c1cccc2c1sc1ccc(-c3ccccc3)cc12]

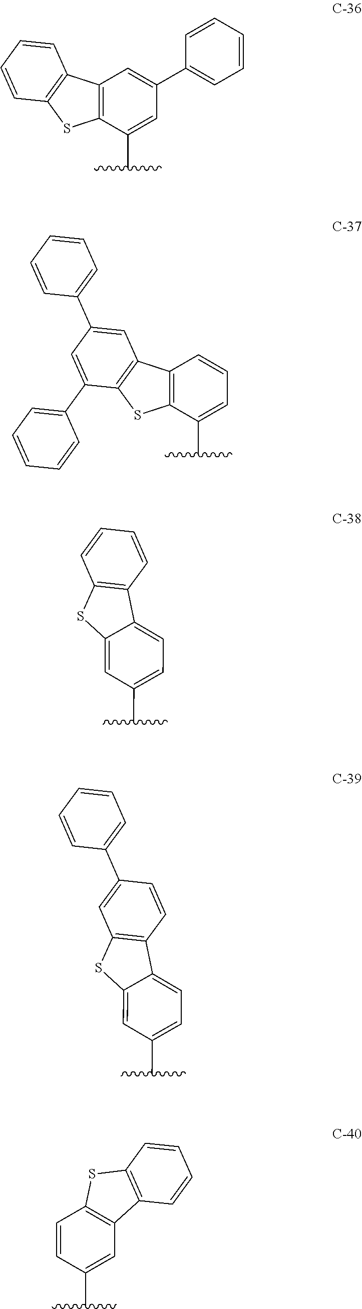 [SMILES: *c1cc(-c2ccccc2)cc2c1sc1ccccc12 ; *c1cccc2c1sc1c(-c3ccccc3)cc(-c3ccccc3)cc12 ; *c1ccc2c(c1)sc1ccccc12 ; *c1ccc2c(c1)sc1cc(-c3ccccc3)ccc12 ; *c1ccc2sc3ccccc3c2c1]

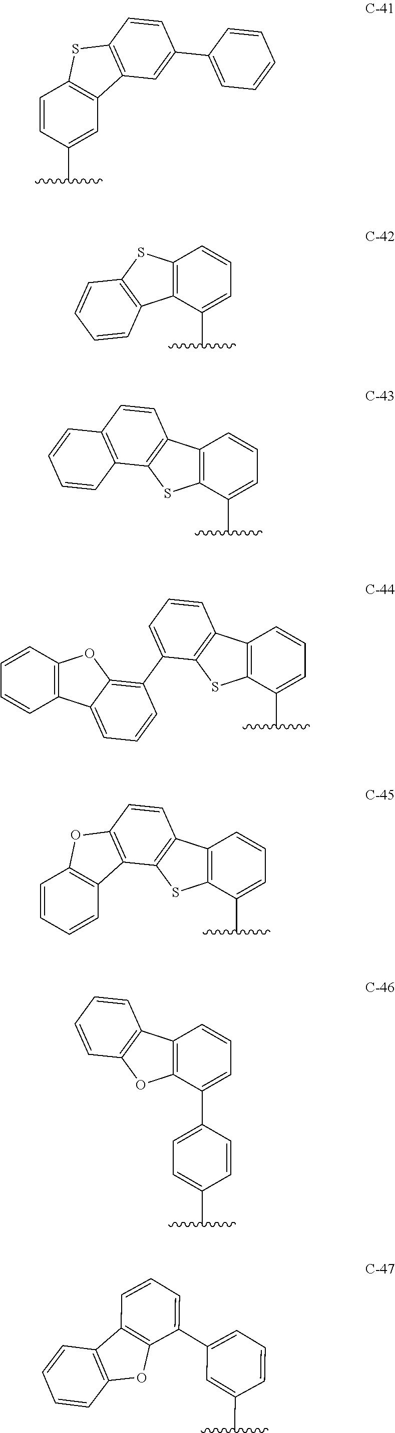

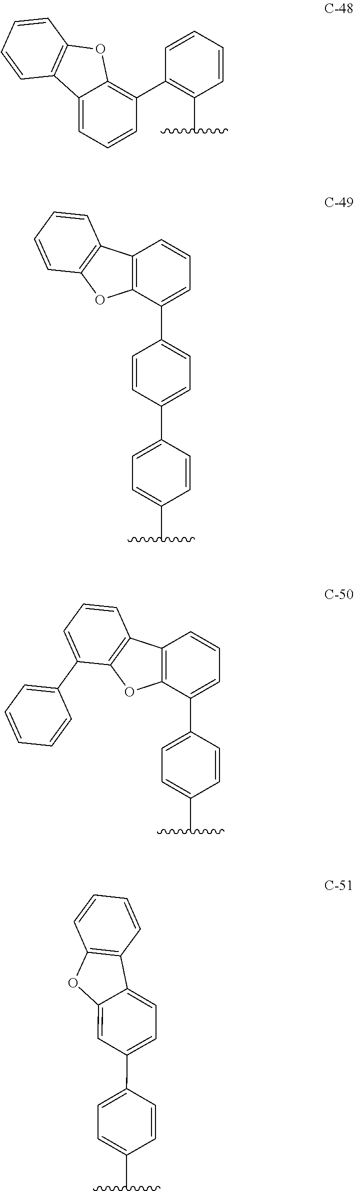 [SMILES: *c1ccccc1-c1cccc2c1oc1ccccc12 ; *c1ccc(-c2ccc(-c3cccc4c3oc3ccccc34)cc2)cc1 ; *c1ccc(-c2cccc3c2oc2c(-c4ccccc4)cccc23)cc1 ; *c1ccc(-c2ccc3c(c2)oc2ccccc23)cc1]

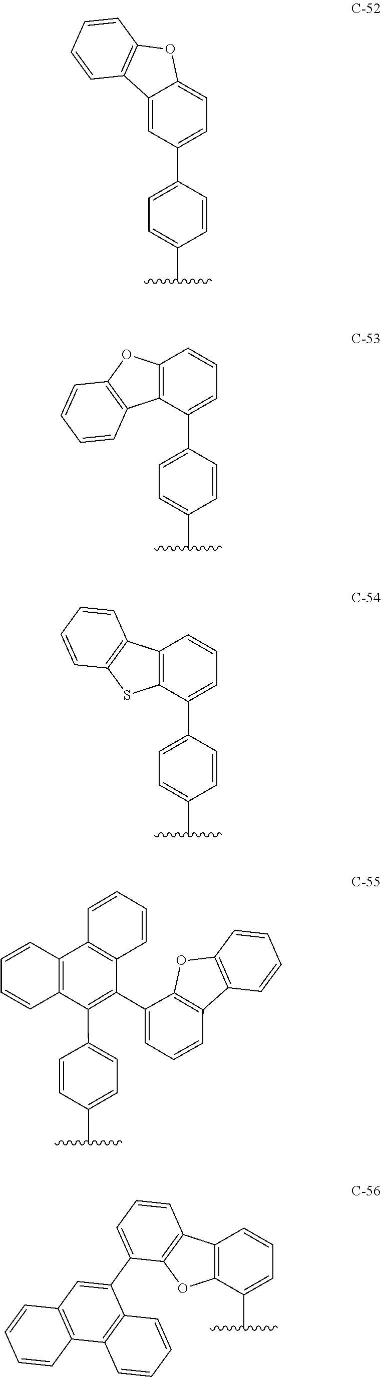 [SMILES: *c1ccc(-c2ccc3oc4ccccc4c3c2)cc1 ; *c1ccc(-c2cccc3oc4ccccc4c23)cc1 ; *c1ccc(-c2cccc3c2sc2ccccc23)cc1 ; *c1ccc(-c2c(-c3cccc4c3oc3ccccc34)c3ccccc3c3ccccc23)cc1 ; *c1cccc2c1oc1c(-c3cc4ccccc4c4ccccc34)cccc12]

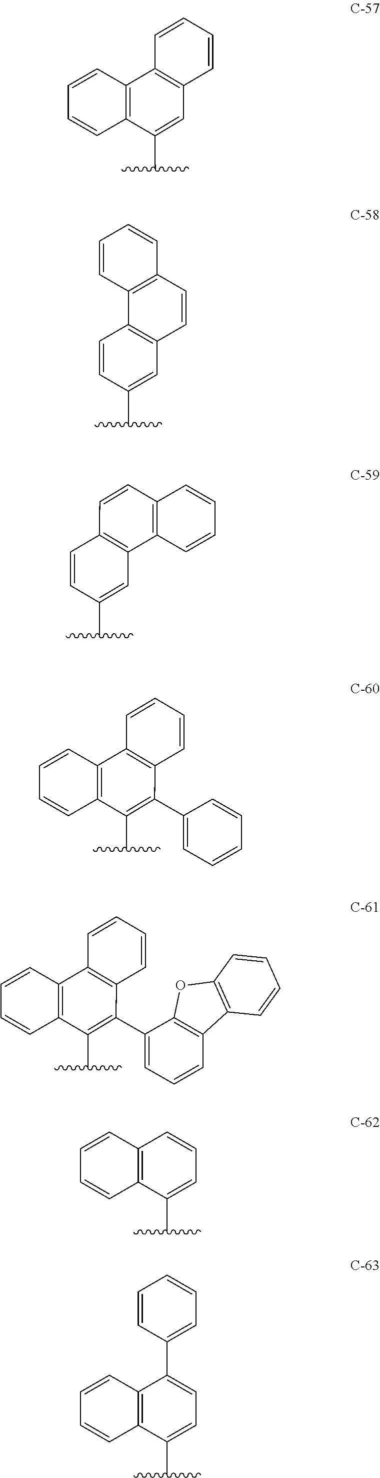

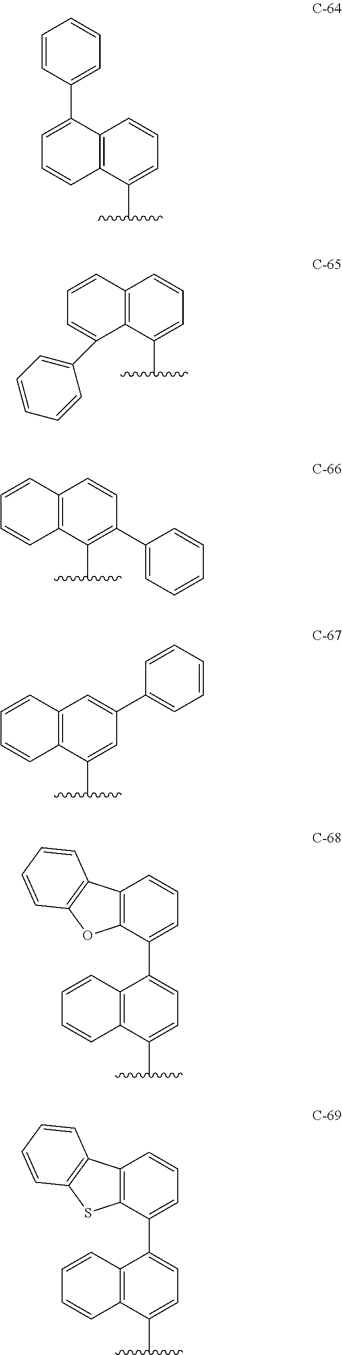 [SMILES: *c1cccc2c(-c3ccccc3)cccc12 ; *c1cccc2cccc(-c3ccccc3)c12 ; *c1c(-c2ccccc2)ccc2ccccc12 ; *c1cc(-c2ccccc2)cc2ccccc12 ; *c1ccc(-c2cccc3c2oc2ccccc23)c2ccccc12 ; *c1ccc(-c2cccc3c2sc2ccccc23)c2ccccc12]

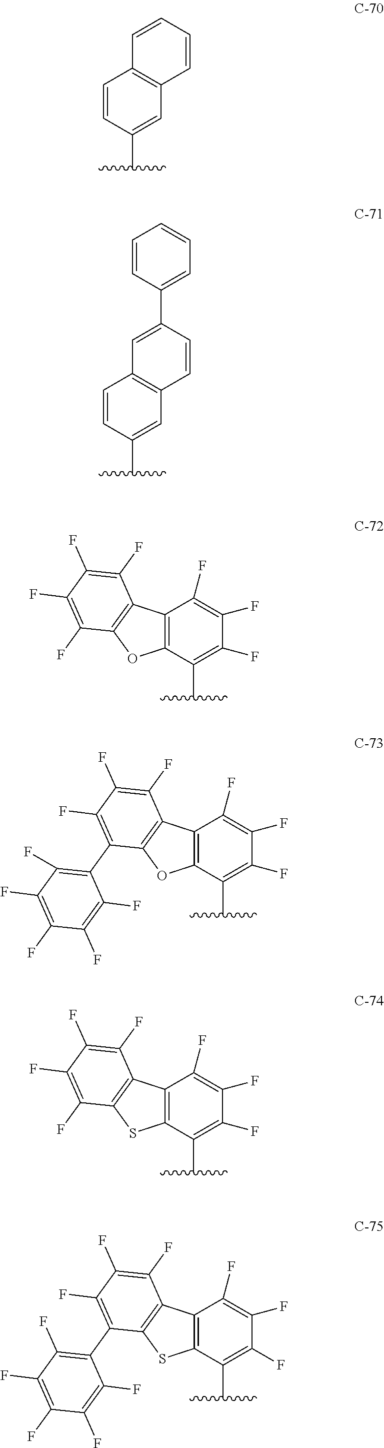 [SMILES: *c1ccc2ccccc2c1 ; *c1ccc2cc(-c3ccccc3)ccc2c1 ; *c1c(F)c(F)c(F)c2c1oc1c(F)c(F)c(F)c(F)c12 ; *c1c(F)c(F)c(F)c2c1oc1c(-c3c(F)c(F)c(F)c(F)c3F)c(F)c(F)c(F)c12 ; *c1c(F)c(F)c(F)c2c1sc1c(F)c(F)c(F)c(F)c12 ; *c1c(F)c(F)c(F)c2c1sc1c(-c3c(F)c(F)c(F)c(F)c3F)c(F)c(F)c(F)c12]

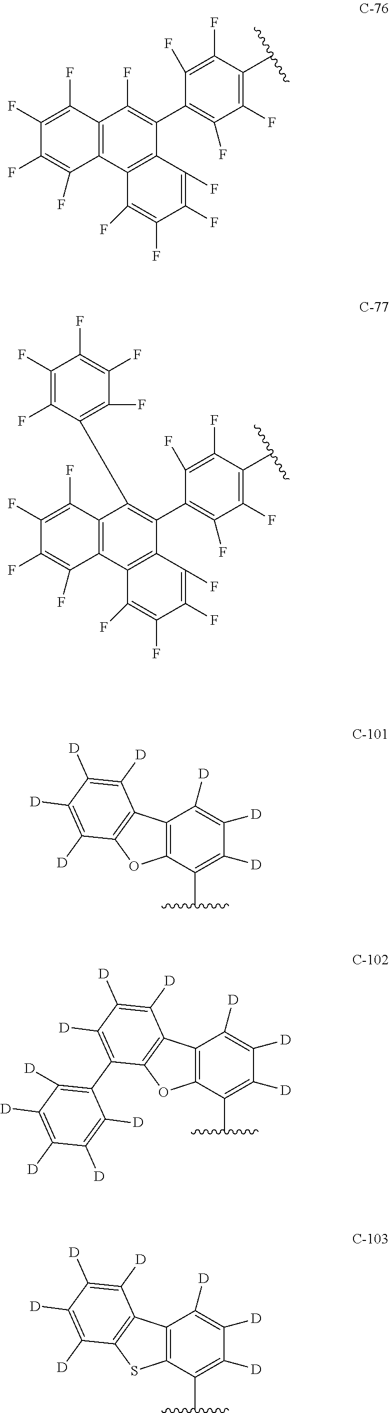 [SMILES: *c1c(F)c(F)c(-c2c(F)c3c(F)c(F)c(F)c(F)c3c3c(F)c(F)c(F)c(F)c23)c(F)c1F ; *c1c(F)c(F)c(-c2c(-c3c(F)c(F)c(F)c(F)c3F)c3c(F)c(F)c(F)c(F)c3c3c(F)c(F)c(F)c(F)c23)c(F)c1F ; *c1c([2H])c([2H])c([2H])c2c1oc1c([2H])c([2H])c([2H])c([2H])c12 ; *c1c([2H])c([2H])c([2H])c2c1oc1c(-c3c([2H])c([2H])c([2H])c([2H])c3[2H])c([2H])c([2H])c([2H])c12 ; *c1c([2H])c([2H])c([2H])c2c1sc1c([2H])c([2H])c([2H])c([2H])c12]

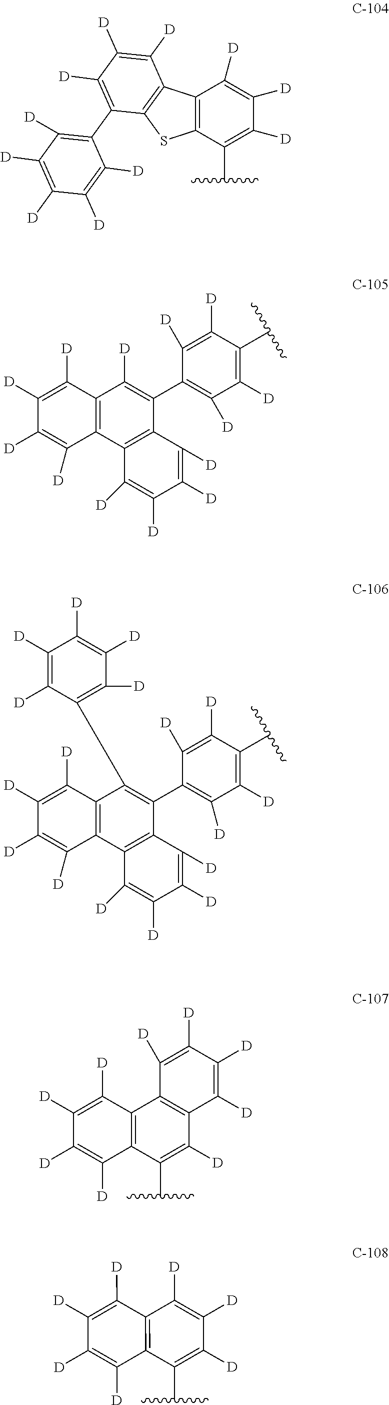 [SMILES: *c1c([2H])c([2H])c([2H])c2c1sc1c(-c3c([2H])c([2H])c([2H])c([2H])c3[2H])c([2H])c([2H])c([2H])c12 ; *c1c([2H])c([2H])c(-c2c([2H])c3c([2H])c([2H])c([2H])c([2H])c3c3c([2H])c([2H])c([2H])c([2H])c23)c([2H])c1[2H] ; *c1c([2H])c([2H])c(-c2c(-c3c([2H])c([2H])c([2H])c([2H])c3[2H])c3c([2H])c([2H])c([2H])c([2H])c3c3c([2H])c([2H])c([2H])c([2H])c23)c([2H])c1[2H] ; *c1c([2H])c2c([2H])c([2H])c([2H])c([2H])c2c2c([2H])c([2H])c([2H])c([2H])c12 ; *c1c([2H])c([2H])c([2H])c2c([2H])c([2H])c([2H])c([2H])c12]

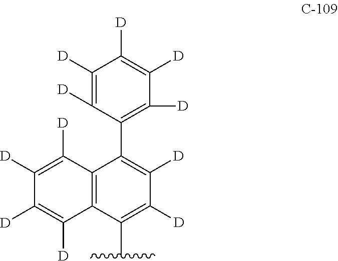 [SMILES: *c1c([2H])c([2H])c(-c2c([2H])c([2H])c([2H])c([2H])c2[2H])c2c([2H])c([2H])c([2H])c([2H])c12]

In an embodiment, the amine compound represented by Formula 1 may have a compound structure according to combinations of FG1 represented by any one of Formulae A-1 to A-75, FG2 represented by any one of Formulae B-1 to B-99 and B-101 to B-106, and FG3 represented by any one of Formulae A-1 to A-75, B-1 to B-99, B-101 to B-106, C-1 to C-77, and C-101 to C-109. In the amine compound represented by Formula 1 according to an embodiment, the combinations of FG1, FG2, and FG3 are shown in the Table 1.

TABLE 1

| Compound | | | |
|---|---|---|---|
| number | A | B | C |
| 1 | A-1 | B-1 | B-1 |
| 2 | A-1 | B-1 | B-2 |
| 3 | A-1 | B-1 | B-3 |
| 4 | A-1 | B-1 | C-1 |
| 5 | A-1 | B-1 | C-2 |
| 6 | A-1 | B-1 | C-3 |
| 7 | A-1 | B-1 | C-4 |
| 8 | A-1 | B-1 | C-5 |
| 9 | A-1 | B-1 | C-6 |
| 10 | A-1 | B-1 | C-13 |
| 11 | A-1 | B-1 | C-19 |
| 12 | A-1 | B-1 | C-27 |
| 13 | A-1 | B-1 | C-28 |
| 14 | A-1 | B-1 | C-57 |
| 15 | A-1 | B-1 | C-62 |
| 16 | A-1 | B-1 | C-43 |
| 17 | A-1 | B-2 | B-2 |
| 18 | A-1 | B-2 | B-3 |
| 19 | A-1 | B-2 | C-1 |
| 20 | A-1 | B-2 | C-2 |
| 21 | A-1 | B-2 | C-3 |
| 22 | A-1 | B-2 | C-4 |
| 23 | A-1 | B-2 | C-5 |
| 24 | A-1 | B-2 | C-6 |
| 25 | A-1 | B-2 | C-13 |
| 26 | A-1 | B-2 | C-19 |
| 27 | A-1 | B-2 | C-27 |
| 28 | A-1 | B-2 | C-28 |
| 29 | A-1 | B-2 | C-57 |
| 30 | A-1 | B-2 | C-62 |
| 31 | A-1 | B-2 | C-43 |
| 32 | A-1 | B-3 | B-3 |
| 33 | A-1 | B-3 | C-1 |
| 34 | A-1 | B-3 | C-2 |
| 35 | A-1 | B-3 | C-3 |
| 36 | A-1 | B-3 | C-4 |
| 37 | A-1 | B-3 | C-5 |
| 38 | A-1 | B-3 | C-6 |
| 39 | A-1 | B-3 | C-13 |
| 40 | A-1 | B-3 | C-19 |
| 41 | A-1 | B-3 | C-27 |
| 42 | A-1 | B-3 | C-28 |
| 43 | A-1 | B-3 | C-57 |
| 44 | A-1 | B-3 | C-62 |
| 45 | A-1 | B-3 | C-43 |
| 46 | A-1 | B-4 | B-1 |
| 47 | A-1 | B-4 | B-2 |
| 48 | A-1 | B-4 | B-3 |
| 49 | A-1 | B-4 | C-1 |
| 50 | A-1 | B-4 | C-2 |
| 51 | A-1 | B-4 | C-3 |
| 52 | A-1 | B-4 | C-4 |
| 53 | A-1 | B-4 | C-5 |
| 54 | A-1 | B-4 | C-6 |
| 55 | A-1 | B-4 | C-13 |
| 56 | A-1 | B-4 | C-19 |
| 57 | A-1 | B-4 | C-27 |
| 58 | A-1 | B-4 | C-28 |
| 59 | A-1 | B-4 | C-57 |
| 60 | A-1 | B-4 | C-62 |
| 61 | A-1 | B-4 | C-43 |
| 62 | A-1 | B-10 | B-1 |
| 63 | A-1 | B-10 | B-2 |
| 64 | A-1 | B-10 | B-3 |
| 65 | A-1 | B-10 | C-1 |
| 66 | A-1 | B-10 | C-2 |
| 67 | A-1 | B-10 | C-3 |
| 68 | A-1 | B-10 | C-4 |
| 69 | A-1 | B-10 | C-5 |
| 70 | A-1 | B-10 | C-6 |
| 71 | A-1 | B-10 | C-13 |
| 72 | A-1 | B-10 | C-19 |
| 73 | A-1 | B-10 | C-27 |
| 74 | A-1 | B-10 | C-28 |
| 75 | A-1 | B-10 | C-57 |
| 76 | A-1 | B-10 | C-62 |
| 77 | A-1 | B-10 | C-43 |
| 78 | A-1 | B-11 | B-1 |
| 79 | A-1 | B-11 | B-2 |
| 80 | A-1 | B-11 | B-3 |
| 81 | A-1 | B-11 | C-1 |
| 82 | A-1 | B-11 | C-2 |
| 83 | A-1 | B-11 | C-3 |
| 84 | A-1 | B-11 | C-4 |
| 85 | A-1 | B-11 | C-5 |
| 86 | A-1 | B-11 | C-6 |
| 87 | A-1 | B-11 | C-13 |
| 88 | A-1 | B-11 | C-19 |
| 89 | A-1 | B-11 | C-27 |
| 90 | A-1 | B-11 | C-28 |
| 91 | A-1 | B-11 | C-57 |
| 92 | A-1 | B-11 | C-62 |
| 93 | A-1 | B-11 | C-43 |
| 94 | A-2 | B-1 | B-1 |
| 95 | A-2 | B-1 | B-2 |
| 96 | A-2 | B-1 | B-3 |
| 97 | A-2 | B-1 | C-1 |
| 98 | A-2 | B-1 | C-2 |
| 99 | A-2 | B-1 | C-3 |
| 100 | A-2 | B-1 | C-4 |
| 101 | A-2 | B-1 | C-5 |
| 102 | A-2 | B-1 | C-6 |
| 103 | A-2 | B-1 | C-13 |
| 104 | A-2 | B-1 | C-19 |
| 105 | A-2 | B-1 | C-27 |
| 106 | A-2 | B-1 | C-28 |
| 107 | A-2 | B-1 | C-57 |
| 108 | A-2 | B-1 | C-62 |
| 109 | A-2 | B-1 | C-43 |
| 110 | A-2 | B-2 | B-2 |
| 111 | A-2 | B-2 | B-3 |
| 112 | A-2 | B-2 | C-1 |
| 113 | A-2 | B-2 | C-2 |
| 114 | A-2 | B-2 | C-3 |
| 115 | A-2 | B-2 | C-4 |
| 116 | A-2 | B-2 | C-5 |
| 117 | A-2 | B-2 | C-6 |
| 118 | A-2 | B-2 | C-13 |
| 119 | A-2 | B-2 | C-19 |
| 120 | A-2 | B-2 | C-27 |
| 121 | A-2 | B-2 | C-28 |
| 122 | A-2 | B-2 | C-57 |
| 123 | A-2 | B-2 | C-62 |
| 124 | A-2 | B-2 | C-43 |
| 125 | A-2 | B-3 | B-3 |
| 126 | A-2 | B-3 | C-1 |
| 127 | A-2 | B-3 | C-2 |
| 128 | A-2 | B-3 | C-3 |
| 129 | A-2 | B-3 | C-4 |
| 130 | A-2 | B-3 | C-5 |
| 131 | A-2 | B-3 | C-6 |
| 132 | A-2 | B-3 | C-13 |
| 133 | A-2 | B-3 | C-19 |
| 134 | A-2 | B-3 | C-27 |
| 135 | A-2 | B-3 | C-28 |
| 136 | A-2 | B-3 | C-57 |
| 137 | A-2 | B-3 | C-62 |
| 138 | A-2 | B-3 | C-43 |
| 139 | A-2 | B-4 | B-1 |
| 140 | A-2 | B-4 | B-2 |
| 141 | A-2 | B-4 | B-3 |
| 142 | A-2 | B-4 | C-1 |
| 143 | A-2 | B-4 | C-2 |
| 144 | A-2 | B-4 | C-3 |
| 145 | A-2 | B-4 | C-4 |
| 146 | A-2 | B-4 | C-5 |
| 147 | A-2 | B-4 | C-6 |
| 148 | A-2 | B-4 | C-13 |
| 149 | A-2 | B-4 | C-19 |
| 150 | A-2 | B-4 | C-27 |
| 151 | A-2 | B-4 | C-28 |
| 152 | A-2 | B-4 | C-57 |
| 153 | A-2 | B-4 | C-62 |
| 154 | A-2 | B-4 | C-43 |
| 155 | A-2 | B-10 | B-1 |
| 156 | A-2 | B-10 | B-2 |
| 157 | A-2 | B-10 | B-3 |
| 158 | A-2 | B-10 | C-1 |
| 159 | A-2 | B-10 | C-2 |
| 160 | A-2 | B-10 | C-3 |
| 161 | A-2 | B-10 | C-4 |
| 162 | A-2 | B-10 | C-5 |
| 163 | A-2 | B-10 | C-6 |
| 164 | A-2 | B-10 | C-13 |
| 165 | A-2 | B-10 | C-19 |
| 166 | A-2 | B-10 | C-27 |
| 167 | A-2 | B-10 | C-28 |
| 168 | A-2 | B-10 | C-57 |
| 169 | A-2 | B-10 | C-62 |
| 170 | A-2 | B-10 | C-43 |
| 171 | A-2 | B-11 | B-1 |
| 172 | A-2 | B-11 | B-2 |
| 173 | A-2 | B-11 | B-3 |
| 174 | A-2 | B-11 | C-1 |
| 175 | A-2 | B-11 | C-2 |
| 176 | A-2 | B-11 | C-3 |
| 177 | A-2 | B-11 | C-4 |
| 178 | A-2 | B-11 | C-5 |
| 179 | A-2 | B-11 | C-6 |
| 180 | A-2 | B-11 | C-13 |
| 181 | A-2 | B-11 | C-19 |
| 182 | A-2 | B-11 | C-27 |
| 183 | A-2 | B-11 | C-28 |
| 184 | A-2 | B-11 | C-57 |
| 185 | A-2 | B-11 | C-62 |
| 186 | A-2 | B-11 | C-43 |
| 187 | A-3 | B-1 | B-1 |
| 188 | A-3 | B-1 | B-2 |
| 189 | A-3 | B-1 | B-3 |
| 190 | A-3 | B-1 | C-1 |
| 191 | A-3 | B-1 | C-2 |
| 192 | A-3 | B-1 | C-3 |
| 193 | A-3 | B-1 | C-4 |
| 194 | A-3 | B-1 | C-5 |
| 195 | A-3 | B-1 | C-6 |
| 196 | A-3 | B-1 | C-13 |
| 197 | A-3 | B-1 | C-19 |
| 198 | A-3 | B-1 | C-27 |
| 199 | A-3 | B-1 | C-28 |
| 200 | A-3 | B-1 | C-57 |
| 201 | A-3 | B-1 | C-62 |
| 202 | A-3 | B-1 | C-43 |
| 203 | A-3 | B-2 | B-2 |
| 204 | A-3 | B-2 | B-3 |
| 205 | A-3 | B-2 | C-1 |
| 206 | A-3 | B-2 | C-2 |
| 207 | A-3 | B-2 | C-3 |
| 208 | A-3 | B-2 | C-4 |
| 209 | A-3 | B-2 | C-5 |
| 210 | A-3 | B-2 | C-6 |
| 211 | A-3 | B-2 | C-13 |
| 212 | A-3 | B-2 | C-19 |
| 213 | A-3 | B-2 | C-27 |
| 214 | A-3 | B-2 | C-28 |
| 215 | A-3 | B-2 | C-57 |
| 216 | A-3 | B-2 | C-62 |
| 217 | A-3 | B-2 | C-43 |
| 218 | A-3 | B-3 | B-3 |
| 219 | A-3 | B-3 | C-1 |
| 220 | A-3 | B-3 | C-2 |
| 221 | A-3 | B-3 | C-3 |
| 222 | A-3 | B-3 | C-4 |
| 223 | A-3 | B-3 | C-5 |
| 224 | A-3 | B-3 | C-6 |
| 225 | A-3 | B-3 | C-13 |
| 226 | A-3 | B-3 | C-19 |
| 227 | A-3 | B-3 | C-27 |
| 228 | A-3 | B-3 | C-28 |
| 229 | A-3 | B-3 | C-57 |
| 230 | A-3 | B-3 | C-62 |
| 231 | A-3 | B-3 | C-43 |
| 232 | A-3 | B-4 | B-1 |
| 233 | A-3 | B-4 | B-2 |
| 234 | A-3 | B-4 | B-3 |
| 235 | A-3 | B-4 | C-1 |
| 236 | A-3 | B-4 | C-2 |
| 237 | A-3 | B-4 | C-3 |
| 238 | A-3 | B-4 | C-4 |
| 239 | A-3 | B-4 | C-5 |
| 240 | A-3 | B-4 | C-6 |
| 241 | A-3 | B-4 | C-13 |
| 242 | A-3 | B-4 | C-19 |
| 243 | A-3 | B-4 | C-27 |
| 244 | A-3 | B-4 | C-28 |
| 245 | A-3 | B-4 | C-57 |
| 246 | A-3 | B-4 | C-62 |
| 247 | A-3 | B-4 | C-43 |
| 248 | A-3 | B-10 | B-1 |
| 249 | A-3 | B-10 | B-2 |
| 250 | A-3 | B-10 | B-3 |
| 251 | A-3 | B-10 | C-1 |
| 252 | A-3 | B-10 | C-2 |
| 253 | A-3 | B-10 | C-3 |
| 254 | A-3 | B-10 | C-4 |
| 255 | A-3 | B-10 | C-5 |
| 256 | A-3 | B-10 | C-6 |
| 257 | A-3 | B-10 | C-13 |
| 258 | A-3 | B-10 | C-19 |
| 259 | A-3 | B-10 | C-27 |
| 260 | A-3 | B-10 | C-28 |
| 261 | A-3 | B-10 | C-57 |
| 262 | A-3 | B-10 | C-62 |
| 263 | A-3 | B-10 | C-43 |
| 264 | A-3 | B-11 | B-1 |
| 265 | A-3 | B-11 | B-2 |
| 266 | A-3 | B-11 | B-3 |
| 267 | A-3 | B-11 | C-1 |
| 268 | A-3 | B-11 | C-2 |
| 269 | A-3 | B-11 | C-3 |
| 270 | A-3 | B-11 | C-4 |
| 271 | A-3 | B-11 | C-5 |
| 272 | A-3 | B-11 | C-6 |
| 273 | A-3 | B-11 | C-13 |
| 274 | A-3 | B-11 | C-19 |
| 275 | A-3 | B-11 | C-27 |
| 276 | A-3 | B-11 | C-28 |
| 277 | A-3 | B-11 | C-57 |
| 278 | A-3 | B-11 | C-62 |
| 279 | A-3 | B-11 | C-43 |
| 280 | A-4 | B-1 | B-1 |
| 281 | A-4 | B-1 | B-2 |
| 282 | A-4 | B-1 | B-3 |
| 283 | A-4 | B-1 | C-1 |
| 284 | A-4 | B-1 | C-2 |
| 285 | A-4 | B-1 | C-3 |
| 286 | A-4 | B-1 | C-4 |
| 287 | A-4 | B-1 | C-5 |
| 288 | A-4 | B-1 | C-6 |
| 289 | A-4 | B-1 | C-13 |
| 290 | A-4 | B-1 | C-19 |
| 291 | A-4 | B-1 | C-27 |
| 292 | A-4 | B-1 | C-28 |
| 293 | A-4 | B-1 | C-57 |
| 294 | A-4 | B-1 | C-62 |
| 295 | A-4 | B-1 | C-43 |
| 296 | A-4 | B-2 | B-2 |
| 297 | A-4 | B-2 | B-3 |
| 298 | A-4 | B-2 | C-1 |
| 299 | A-4 | B-2 | C-2 |
| 300 | A-4 | B-2 | C-3 |
| 301 | A-4 | B-2 | C-4 |
| 302 | A-4 | B-2 | C-5 |
| 303 | A-4 | B-2 | C-6 |
| 304 | A-4 | B-2 | C-13 |
| 305 | A-4 | B-2 | C-19 |
| 306 | A-4 | B-2 | C-27 |
| 307 | A-4 | B-2 | C-28 |
| 308 | A-4 | B-2 | C-57 |
| 309 | A-4 | B-2 | C-62 |
| 310 | A-4 | B-2 | C-43 |
| 311 | A-4 | B-3 | B-3 |
| 312 | A-4 | B-3 | C-1 |
| 313 | A-4 | B-3 | C-2 |
| 314 | A-4 | B-3 | C-3 |
| 315 | A-4 | B-3 | C-4 |
| 316 | A-4 | B-3 | C-5 |
| 317 | A-4 | B-3 | C-6 |
| 318 | A-4 | B-3 | C-13 |
| 319 | A-4 | B-3 | C-19 |
| 320 | A-4 | B-3 | C-27 |
| 321 | A-4 | B-3 | C-28 |
| 322 | A-4 | B-3 | C-57 |
| 323 | A-4 | B-3 | C-62 |
| 324 | A-4 | B-3 | C-43 |
| 325 | A-4 | B-4 | B-1 |
| 326 | A-4 | B-4 | B-2 |
| 327 | A-4 | B-4 | B-3 |
| 328 | A-4 | B-4 | C-1 |
| 329 | A-4 | B-4 | C-2 |
| 330 | A-4 | B-4 | C-3 |
| 331 | A-4 | B-4 | C-4 |
| 332 | A-4 | B-4 | C-5 |
| 333 | A-4 | B-4 | C-6 |
| 334 | A-4 | B-4 | C-13 |
| 335 | A-4 | B-4 | C-19 |
| 336 | A-4 | B-4 | C-27 |
| 337 | A-4 | B-4 | C-28 |
| 338 | A-4 | B-4 | C-57 |
| 339 | A-4 | B-4 | C-62 |
| 340 | A-4 | B-4 | C-43 |
| 341 | A-4 | B-10 | B-1 |
| 342 | A-4 | B-10 | B-2 |
| 343 | A-4 | B-10 | B-3 |
| 344 | A-4 | B-10 | C-1 |
| 345 | A-4 | B-10 | C-2 |
| 346 | A-4 | B-10 | C-3 |
| 347 | A-4 | B-10 | C-4 |
| 348 | A-4 | B-10 | C-5 |
| 349 | A-4 | B-10 | C-6 |
| 350 | A-4 | B-10 | C-13 |
| 351 | A-4 | B-10 | C-19 |
| 352 | A-4 | B-10 | C-27 |
| 353 | A-4 | B-10 | C-28 |
| 354 | A-4 | B-10 | C-57 |
| 355 | A-4 | B-10 | C-62 |
| 356 | A-4 | B-10 | C-43 |
| 357 | A-4 | B-11 | B-1 |
| 358 | A-4 | B-11 | B-2 |
| 359 | A-4 | B-11 | B-3 |
| 360 | A-4 | B-11 | C-1 |
| 361 | A-4 | B-11 | C-2 |
| 362 | A-4 | B-11 | C-3 |
| 363 | A-4 | B-11 | C-4 |
| 364 | A-4 | B-11 | C-5 |
| 365 | A-4 | B-11 | C-6 |
| 366 | A-4 | B-11 | C-13 |
| 367 | A-4 | B-11 | C-19 |
| 368 | A-4 | B-11 | C-27 |
| 369 | A-4 | B-11 | C-28 |
| 370 | A-4 | B-11 | C-57 |
| 371 | A-4 | B-11 | C-62 |
| 372 | A-4 | B-11 | C-43 |
| 373 | A-5 | B-1 | B-1 |
| 374 | A-5 | B-1 | B-2 |
| 375 | A-5 | B-1 | B-3 |
| 376 | A-5 | B-1 | C-1 |
| 377 | A-5 | B-1 | C-2 |
| 378 | A-5 | B-1 | C-3 |
| 379 | A-5 | B-1 | C-4 |
| 380 | A-5 | B-1 | C-5 |
| 381 | A-5 | B-1 | C-6 |
| 382 | A-5 | B-1 | C-13 |
| 383 | A-5 | B-1 | C-19 |
| 384 | A-5 | B-1 | C-27 |
| 385 | A-5 | B-1 | C-28 |
| 386 | A-5 | B-1 | C-57 |
| 387 | A-5 | B-1 | C-62 |
| 388 | A-5 | B-1 | C-43 |
| 389 | A-5 | B-2 | B-2 |
| 390 | A-5 | B-2 | B-3 |
| 391 | A-5 | B-2 | C-1 |
| 392 | A-5 | B-2 | C-2 |
| 393 | A-5 | B-2 | C-3 |
| 394 | A-5 | B-2 | C-4 |
| 395 | A-5 | B-2 | C-5 |
| 396 | A-5 | B-2 | C-6 |
| 397 | A-5 | B-2 | C-13 |
| 398 | A-5 | B-2 | C-19 |
| 399 | A-5 | B-2 | C-27 |
| 400 | A-5 | B-2 | C-28 |
| 401 | A-5 | B-2 | C-57 |
| 402 | A-5 | B-2 | C-62 |
| 403 | A-5 | B-2 | C-43 |
| 404 | A-5 | B-3 | B-3 |
| 405 | A-5 | B-3 | C-1 |
| 406 | A-5 | B-3 | C-2 |
| 407 | A-5 | B-3 | C-3 |
| 408 | A-5 | B-3 | C-4 |
| 409 | A-5 | B-3 | C-5 |
| 410 | A-5 | B-3 | C-6 |
| 411 | A-5 | B-3 | C-13 |
| 412 | A-5 | B-3 | C-19 |
| 413 | A-5 | B-3 | C-27 |
| 414 | A-5 | B-3 | C-28 |
| 415 | A-5 | B-3 | C-57 |
| 416 | A-5 | B-3 | C-62 |
| 417 | A-5 | B-3 | C-43 |
| 418 | A-5 | B-4 | B-1 |
| 419 | A-5 | B-4 | B-2 |
| 420 | A-5 | B-4 | B-3 |
| 421 | A-5 | B-4 | C-1 |
| 422 | A-5 | B-4 | C-2 |
| 423 | A-5 | B-4 | C-3 |
| 424 | A-5 | B-4 | C-4 |
| 425 | A-5 | B-4 | C-5 |
| 426 | A-5 | B-4 | C-6 |
| 427 | A-5 | B-4 | C-13 |
| 428 | A-5 | B-4 | C-19 |
| 429 | A-5 | B-4 | C-27 |
| 430 | A-5 | B-4 | C-28 |
| 431 | A-5 | B-4 | C-57 |
| 432 | A-5 | B-4 | C-62 |
| 433 | A-5 | B-4 | C-43 |
| 434 | A-5 | B-10 | B-1 |
| 435 | A-5 | B-10 | B-2 |
| 436 | A-5 | B-10 | B-3 |
| 437 | A-5 | B-10 | C-1 |
| 438 | A-5 | B-10 | C-2 |
| 439 | A-5 | B-10 | C-3 |
| 440 | A-5 | B-10 | C-4 |
| 441 | A-5 | B-10 | C-5 |
| 442 | A-5 | B-10 | C-6 |
| 443 | A-5 | B-10 | C-13 |
| 444 | A-5 | B-10 | C-19 |
| 445 | A-5 | B-10 | C-27 |
| 446 | A-5 | B-10 | C-28 |
| 447 | A-5 | B-10 | C-57 |
| 448 | A-5 | B-10 | C-62 |
| 449 | A-5 | B-10 | C-43 |
| 450 | A-5 | B-11 | B-1 |
| 451 | A-5 | B-11 | B-2 |
| 452 | A-5 | B-11 | B-3 |
| 453 | A-5 | B-11 | C-1 |
| 454 | A-5 | B-11 | C-2 |
| 455 | A-5 | B-11 | C-3 |
| 456 | A-5 | B-11 | C-4 |
| 457 | A-5 | B-11 | C-5 |
| 458 | A-5 | B-11 | C-6 |
| 459 | A-5 | B-11 | C-13 |
| 460 | A-5 | B-11 | C-19 |
| 461 | A-5 | B-11 | C-27 |
| 462 | A-5 | B-11 | C-28 |
| 463 | A-5 | B-11 | C-57 |
| 464 | A-5 | B-11 | C-62 |
| 465 | A-5 | B-11 | C-43 |
| 466 | A-6 | B-1 | B-1 |
| 467 | A-6 | B-1 | B-2 |
| 468 | A-6 | B-1 | B-3 |
| 469 | A-6 | B-1 | C-1 |
| 470 | A-6 | B-1 | C-2 |
| 471 | A-6 | B-1 | C-3 |
| 472 | A-6 | B-1 | C-4 |
| 473 | A-6 | B-1 | C-5 |
| 474 | A-6 | B-1 | C-6 |
| 475 | A-6 | B-1 | C-13 |
| 476 | A-6 | B-1 | C-19 |
| 477 | A-6 | B-1 | C-27 |
| 478 | A-6 | B-1 | C-28 |
| 479 | A-6 | B-1 | C-57 |
| 480 | A-6 | B-1 | C-62 |
| 481 | A-6 | B-1 | C-43 |
| 482 | A-6 | B-2 | B-2 |
| 483 | A-6 | B-2 | B-3 |
| 484 | A-6 | B-2 | C-1 |
| 485 | A-6 | B-2 | C-2 |
| 486 | A-6 | B-2 | C-3 |
| 487 | A-6 | B-2 | C-4 |
| 488 | A-6 | B-2 | C-5 |
| 489 | A-6 | B-2 | C-6 |
| 490 | A-6 | B-2 | C-13 |
| 491 | A-6 | B-2 | C-19 |
| 492 | A-6 | B-2 | C-27 |
| 493 | A-6 | B-2 | C-28 |
| 494 | A-6 | B-2 | C-57 |
| 495 | A-6 | B-2 | C-62 |
| 496 | A-6 | B-2 | C-43 |
| 497 | A-6 | B-3 | B-3 |
| 498 | A-6 | B-3 | C-1 |
| 499 | A-6 | B-3 | C-2 |
| 500 | A-6 | B-3 | C-3 |
| 501 | A-6 | B-3 | C-4 |
| 502 | A-6 | B-3 | C-5 |
| 503 | A-6 | B-3 | C-6 |
| 504 | A-6 | B-3 | C-13 |
| 505 | A-6 | B-3 | C-19 |
| 506 | A-6 | B-3 | C-27 |
| 507 | A-6 | B-3 | C-28 |
| 508 | A-6 | B-3 | C-57 |
| 509 | A-6 | B-3 | C-62 |
| 510 | A-6 | B-3 | C-43 |
| 511 | A-6 | B-4 | B-1 |
| 512 | A-6 | B-4 | B-2 |
| 513 | A-6 | B-4 | B-3 |
| 514 | A-6 | B-4 | C-1 |
| 515 | A-6 | B-4 | C-2 |
| 516 | A-6 | B-4 | C-3 |
| 517 | A-6 | B-4 | C-4 |
| 518 | A-6 | B-4 | C-5 |
| 519 | A-6 | B-4 | C-6 |
| 520 | A-6 | B-4 | C-13 |
| 521 | A-6 | B-4 | C-19 |
| 522 | A-6 | B-4 | C-27 |
| 523 | A-6 | B-4 | C-28 |
| 524 | A-6 | B-4 | C-57 |
| 525 | A-6 | B-4 | C-62 |
| 526 | A-6 | B-4 | C-43 |
| 527 | A-6 | B-10 | B-1 |
| 528 | A-6 | B-10 | B-2 |
| 529 | A-6 | B-10 | B-3 |
| 530 | A-6 | B-10 | C-1 |
| 531 | A-6 | B-10 | C-2 |
| 532 | A-6 | B-10 | C-3 |
| 533 | A-6 | B-10 | C-4 |
| 534 | A-6 | B-10 | C-5 |
| 535 | A-6 | B-10 | C-6 |
| 536 | A-6 | B-10 | C-13 |
| 537 | A-6 | B-10 | C-19 |
| 538 | A-6 | B-10 | C-27 |
| 539 | A-6 | B-10 | C-28 |
| 540 | A-6 | B-10 | C-57 |
| 541 | A-6 | B-10 | C-62 |
| 542 | A-6 | B-10 | C-43 |
| 543 | A-6 | B-11 | B-1 |
| 544 | A-6 | B-11 | B-2 |
| 545 | A-6 | B-11 | B-3 |
| 546 | A-6 | B-11 | C-1 |
| 547 | A-6 | B-11 | C-2 |
| 548 | A-6 | B-11 | C-3 |
| 549 | A-6 | B-11 | C-4 |
| 550 | A-6 | B-11 | C-5 |
| 551 | A-6 | B-11 | C-6 |
| 552 | A-6 | B-11 | C-13 |
| 553 | A-6 | B-11 | C-19 |
| 554 | A-6 | B-11 | C-27 |
| 555 | A-6 | B-11 | C-28 |
| 556 | A-6 | B-11 | C-57 |
| 557 | A-6 | B-11 | C-62 |
| 558 | A-6 | B-11 | C-43 |
| 559 | A-3 | B-1 | C-7 |
| 560 | A-3 | B-2 | C-8 |
| 561 | A-4 | B-1 | C-9 |
| 562 | A-4 | B-2 | C-10 |
| 563 | A-5 | B-1 | C-11 |
| 564 | A-5 | B-2 | C-12 |
| 565 | A-3 | B-1 | C-14 |
| 566 | A-3 | B-2 | C-15 |
| 567 | A-4 | B-1 | C-16 |
| 568 | A-4 | B-2 | C-17 |
| 569 | A-5 | B-1 | C-18 |
| 570 | A-5 | B-2 | C-20 |
| 571 | A-3 | B-1 | C-21 |
| 572 | A-3 | B-2 | C-22 |
| 573 | A-4 | B-1 | C-23 |
| 574 | A-4 | B-2 | C-24 |
| 575 | A-5 | B-1 | C-25 |
| 576 | A-5 | B-2 | C-26 |
| 577 | A-3 | B-1 | C-29 |
| 578 | A-3 | B-2 | C-30 |
| 579 | A-4 | B-1 | C-31 |
| 580 | A-4 | B-2 | C-32 |
| 581 | A-5 | B-1 | C-33 |
| 582 | A-5 | B-2 | C-34 |
| 583 | A-3 | B-1 | C-35 |
| 584 | A-3 | B-2 | C-36 |
| 585 | A-4 | B-1 | C-37 |
| 586 | A-4 | B-2 | C-38 |
| 587 | A-5 | B-1 | C-39 |
| 588 | A-5 | B-2 | C-40 |
| 589 | A-3 | B-1 | C-41 |
| 590 | A-3 | B-2 | C-42 |
| 591 | A-4 | B-1 | C-44 |
| 592 | A-4 | B-2 | C-45 |
| 593 | A-5 | B-1 | C-46 |
| 594 | A-5 | B-2 | C-47 |
| 595 | A-3 | B-1 | C-48 |
| 596 | A-3 | B-2 | C-49 |
| 597 | A-4 | B-1 | C-50 |
| 598 | A-4 | B-2 | C-51 |
| 599 | A-5 | B-1 | C-52 |
| 600 | A-5 | B-2 | C-53 |
| 601 | A-3 | B-1 | C-54 |
| 602 | A-3 | B-2 | C-55 |
| 603 | A-4 | B-1 | C-56 |
| 604 | A-4 | B-2 | C-58 |
| 605 | A-5 | B-1 | C-59 |
| 606 | A-5 | B-2 | C-60 |
| 607 | A-3 | B-1 | C-61 |
| 608 | A-3 | B-2 | C-63 |
| 609 | A-4 | B-1 | C-64 |
| 610 | A-4 | B-2 | C-65 |
| 611 | A-5 | B-1 | C-66 |
| 612 | A-5 | B-2 | C-67 |
| 613 | A-3 | B-1 | C-68 |
| 614 | A-3 | B-2 | C-69 |
| 615 | A-4 | B-1 | C-70 |
| 616 | A-4 | B-2 | C-71 |
| 617 | A-5 | B-1 | C-72 |
| 618 | A-5 | B-2 | C-101 |
| 619 | A-3 | B-1 | C-102 |
| 620 | A-3 | B-2 | C-103 |
| 621 | A-4 | B-1 | C-104 |
| 622 | A-4 | B-2 | C-105 |
| 623 | A-5 | B-1 | C-106 |
| 624 | A-5 | B-2 | C-107 |
| 625 | A-3 | B-1 | C-108 |
| 626 | A-3 | B-2 | C-109 |
| 627 | A-4 | B-1 | B-4 |
| 628 | A-4 | B-2 | B-5 |
| 629 | A-5 | B-1 | B-6 |
| 630 | A-5 | B-2 | B-7 |
| 631 | A-3 | B-1 | B-8 |
| 632 | A-3 | B-2 | B-9 |
| 633 | A-4 | B-1 | B-10 |
| 634 | A-4 | B-2 | B-11 |
| 635 | A-5 | B-1 | B-12 |
| 636 | A-5 | B-2 | B-13 |
| 637 | A-3 | B-1 | B-14 |
| 638 | A-3 | B-2 | B-15 |
| 639 | A-4 | B-1 | B-16 |
| 640 | A-4 | B-2 | B-17 |
| 641 | A-5 | B-1 | B-18 |
| 642 | A-5 | B-2 | B-19 |
| 643 | A-3 | B-1 | B-20 |
| 644 | A-3 | B-2 | B-21 |
| 645 | A-4 | B-1 | B-22 |
| 646 | A-4 | B-2 | B-23 |
| 647 | A-5 | B-1 | B-24 |
| 648 | A-5 | B-2 | B-25 |
| 649 | A-3 | B-1 | B-26 |
| 650 | A-3 | B-2 | B-27 |
| 651 | A-4 | B-1 | B-28 |
| 652 | A-4 | B-2 | B-29 |
| 653 | A-5 | B-1 | B-30 |
| 654 | A-5 | B-2 | B-31 |
| 655 | A-3 | B-1 | B-32 |
| 656 | A-3 | B-2 | B-33 |
| 657 | A-4 | B-1 | B-34 |
| 658 | A-4 | B-2 | B-35 |
| 659 | A-5 | B-1 | B-36 |
| 660 | A-5 | B-2 | B-37 |
| 661 | A-3 | B-1 | B-38 |
| 662 | A-3 | B-2 | B-39 |
| 663 | A-4 | B-1 | B-40 |
| 664 | A-4 | B-2 | B-41 |
| 665 | A-5 | B-1 | B-42 |
| 666 | A-5 | B-2 | B-43 |
| 667 | A-3 | B-1 | B-44 |
| 668 | A-3 | B-2 | B-45 |
| 669 | A-4 | B-1 | B-46 |
| 670 | A-4 | B-2 | B-47 |
| 671 | A-5 | B-1 | B-48 |
| 672 | A-5 | B-2 | B-49 |
| 673 | A-3 | B-1 | B-50 |
| 674 | A-3 | B-2 | B-51 |
| 675 | A-4 | B-1 | B-52 |
| 676 | A-4 | B-2 | B-53 |
| 677 | A-5 | B-1 | B-54 |
| 678 | A-5 | B-2 | B-55 |
| 679 | A-3 | B-1 | B-56 |
| 680 | A-3 | B-2 | B-57 |
| 681 | A-4 | B-1 | B-58 |
| 682 | A-4 | B-2 | B-59 |
| 683 | A-5 | B-1 | B-60 |
| 684 | A-5 | B-2 | B-61 |
| 685 | A-3 | B-1 | B-62 |
| 686 | A-3 | B-2 | B-63 |
| 687 | A-4 | B-1 | B-64 |
| 688 | A-4 | B-2 | B-65 |
| 689 | A-5 | B-1 | B-66 |
| 690 | A-5 | B-2 | B-67 |
| 691 | A-3 | B-1 | B-68 |
| 692 | A-3 | B-2 | B-69 |
| 693 | A-4 | B-1 | B-70 |
| 694 | A-4 | B-2 | B-71 |
| 695 | A-5 | B-1 | B-72 |
| 696 | A-5 | B-2 | B-73 |
| 697 | A-3 | B-1 | B-74 |
| 698 | A-3 | B-2 | B-75 |
| 699 | A-4 | B-1 | B-76 |
| 700 | A-4 | B-2 | B-77 |
| 701 | A-5 | B-1 | B-78 |
| 702 | A-5 | B-2 | B-79 |
| 703 | A-3 | B-1 | B-80 |
| 704 | A-3 | B-2 | B-81 |
| 705 | A-4 | B-1 | B-82 |
| 706 | A-4 | B-2 | B-83 |
| 707 | A-5 | B-1 | B-84 |
| 708 | A-5 | B-2 | B-85 |
| 709 | A-3 | B-1 | B-86 |
| 710 | A-3 | B-2 | B-87 |
| 711 | A-4 | B-1 | B-88 |
| 712 | A-4 | B-2 | B-89 |
| 713 | A-5 | B-1 | B-90 |
| 714 | A-5 | B-2 | B-91 |
| 715 | A-3 | B-1 | B-92 |
| 716 | A-3 | B-2 | B-93 |
| 717 | A-4 | B-1 | B-94 |
| 718 | A-4 | B-2 | B-95 |
| 719 | A-5 | B-1 | B-96 |
| 720 | A-5 | B-2 | B-97 |
| 721 | A-3 | B-1 | B-98 |
| 722 | A-3 | B-2 | B-99 |
| 723 | A-4 | B-1 | B-101 |
| 724 | A-4 | B-2 | B-102 |
| 725 | A-5 | B-1 | B-103 |
| 726 | A-5 | B-2 | B-104 |
| 727 | A-3 | B-1 | B-105 |
| 728 | A-3 | B-2 | B-106 |
| 729 | A-3 | B-1 | A-1 |
| 730 | A-3 | B-2 | A-2 |
| 731 | A-4 | B-1 | A-3 |
| 732 | A-4 | B-2 | A-4 |
| 733 | A-5 | B-1 | A-5 |
| 734 | A-5 | B-2 | A-6 |
| 735 | A-3 | B-1 | A-7 |
| 736 | A-3 | B-2 | A-8 |
| 737 | A-4 | B-1 | A-9 |
| 738 | A-4 | B-2 | A-10 |
| 739 | A-5 | B-1 | A-11 |
| 740 | A-5 | B-2 | A-12 |
| 741 | A-3 | B-1 | A-13 |
| 742 | A-3 | B-2 | A-14 |
| 743 | A-4 | B-1 | A-15 |
| 744 | A-4 | B-2 | A-16 |
| 745 | A-5 | B-1 | A-17 |
| 746 | A-5 | B-2 | A-18 |
| 747 | A-3 | B-1 | A-19 |
| 748 | A-3 | B-2 | A-20 |
| 749 | A-4 | B-1 | A-21 |
| 750 | A-4 | B-2 | A-22 |
| 751 | A-5 | B-1 | A-23 |
| 752 | A-5 | B-2 | A-24 |
| 753 | A-3 | B-1 | A-25 |
| 754 | A-3 | B-2 | A-26 |
| 755 | A-4 | B-1 | A-27 |
| 756 | A-4 | B-2 | A-28 |
| 757 | A-5 | B-1 | A-29 |
| 758 | A-5 | B-2 | A-30 |
| 759 | A-3 | B-1 | A-31 |
| 760 | A-3 | B-2 | A-32 |
| 761 | A-4 | B-1 | A-33 |
| 762 | A-4 | B-2 | A-34 |
| 763 | A-5 | B-1 | A-35 |
| 764 | A-5 | B-2 | A-36 |
| 765 | A-3 | B-1 | A-37 |
| 766 | A-3 | B-2 | A-38 |
| 767 | A-4 | B-1 | A-39 |
| 768 | A-4 | B-2 | A-40 |
| 769 | A-5 | B-1 | A-41 |
| 770 | A-5 | B-2 | A-42 |
| 771 | A-3 | B-1 | A-43 |
| 772 | A-3 | B-2 | A-44 |
| 773 | A-4 | B-1 | A-45 |
| 774 | A-4 | B-2 | A-46 |
| 775 | A-5 | B-1 | A-47 |
| 776 | A-5 | B-2 | A-48 |
| 777 | A-3 | B-1 | A-49 |
| 778 | A-3 | B-2 | A-50 |
| 779 | A-4 | B-1 | A-51 |
| 780 | A-4 | B-2 | A-52 |
| 781 | A-5 | B-1 | A-53 |
| 782 | A-5 | B-2 | A-54 |
| 783 | A-3 | B-1 | A-55 |
| 784 | A-3 | B-2 | A-56 |
| 785 | A-4 | B-1 | A-57 |
| 786 | A-4 | B-2 | A-58 |
| 787 | A-5 | B-1 | A-59 |
| 788 | A-5 | B-2 | A-60 |
| 789 | A-3 | B-1 | A-61 |
| 790 | A-3 | B-2 | A-62 |
| 791 | A-4 | B-1 | A-63 |
| 792 | A-4 | B-2 | A-64 |
| 793 | A-5 | B-1 | A-65 |
| 794 | A-5 | B-2 | A-66 |
| 795 | A-3 | B-1 | A-67 |
| 796 | A-3 | B-2 | A-68 |
| 797 | A-4 | B-1 | A-69 |
| 798 | A-4 | B-2 | A-101 |
| 799 | A-5 | B-1 | A-102 |
| 800 | A-5 | B-2 | A-103 |
| 801 | A-3 | B-1 | A-104 |
| 802 | A-3 | B-2 | A-105 |
| 803 | A-4 | B-1 | A-106 |
| 804 | A-3 | B-5 | B-1 |
| 805 | A-3 | B-6 | B-2 |
| 806 | A-4 | B-7 | B-1 |
| 807 | A-4 | B-8 | B-2 |
| 808 | A-5 | B-9 | B-1 |
| 809 | A-5 | B-12 | B-2 |
| 810 | A-3 | B-13 | B-1 |
| 811 | A-3 | B-14 | B-2 |
| 812 | A-4 | B-15 | B-1 |
| 813 | A-4 | B-16 | B-2 |
| 814 | A-5 | B-17 | B-1 |
| 815 | A-5 | B-18 | B-2 |
| 816 | A-3 | B-19 | B-1 |
| 817 | A-3 | B-20 | B-2 |
| 818 | A-4 | B-21 | B-1 |
| 819 | A-4 | B-22 | B-2 |
| 820 | A-5 | B-23 | B-1 |
| 821 | A-5 | B-24 | B-2 |
| 822 | A-3 | B-25 | B-1 |
| 823 | A-3 | B-26 | B-2 |
| 824 | A-4 | B-27 | B-1 |
| 825 | A-4 | B-28 | B-2 |
| 826 | A-5 | B-29 | B-1 |
| 827 | A-5 | B-30 | B-2 |
| 828 | A-3 | B-31 | B-1 |
| 829 | A-3 | B-32 | B-2 |
| 830 | A-4 | B-33 | B-1 |
| 831 | A-4 | B-34 | B-2 |
| 832 | A-5 | B-35 | B-1 |
| 833 | A-5 | B-36 | B-2 |
| 834 | A-3 | B-37 | B-1 |
| 835 | A-3 | B-38 | B-2 |
| 836 | A-4 | B-39 | B-1 |
| 837 | A-4 | B-40 | B-2 |
| 838 | A-5 | B-41 | B-1 |
| 839 | A-5 | B-42 | B-2 |
| 840 | A-3 | B-43 | B-1 |
| 841 | A-3 | B-44 | B-2 |
| 842 | A-4 | B-45 | B-1 |
| 843 | A-4 | B-46 | B-2 |
| 844 | A-5 | B-47 | B-1 |
| 845 | A-5 | B-48 | B-2 |
| 846 | A-3 | B-49 | B-1 |
| 847 | A-3 | B-50 | B-2 |
| 848 | A-4 | B-51 | B-1 |
| 849 | A-4 | B-52 | B-2 |
| 850 | A-5 | B-53 | B-1 |
| 851 | A-5 | B-54 | B-2 |
| 852 | A-3 | B-55 | B-1 |
| 853 | A-3 | B-56 | B-2 |
| 854 | A-4 | B-57 | B-1 |
| 855 | A-4 | B-58 | B-2 |
| 856 | A-5 | B-59 | B-1 |
| 857 | A-5 | B-60 | B-2 |
| 858 | A-3 | B-61 | B-1 |
| 859 | A-3 | B-62 | B-2 |
| 860 | A-4 | B-63 | B-1 |
| 861 | A-4 | B-64 | B-2 |
| 862 | A-5 | B-65 | B-1 |
| 863 | A-5 | B-66 | B-2 |
| 864 | A-3 | B-67 | B-1 |
| 865 | A-3 | B-68 | B-2 |
| 866 | A-4 | B-69 | B-1 |
| 867 | A-4 | B-70 | B-2 |
| 868 | A-5 | B-71 | B-1 |
| 869 | A-5 | B-72 | B-2 |
| 870 | A-3 | B-73 | B-1 |
| 871 | A-3 | B-74 | B-2 |
| 872 | A-4 | B-75 | B-1 |
| 873 | A-4 | B-76 | B-2 |
| 874 | A-5 | B-77 | B-1 |
| 875 | A-5 | B-78 | B-2 |
| 876 | A-3 | B-79 | B-1 |
| 877 | A-3 | B-80 | B-2 |
| 878 | A-4 | B-81 | B-1 |
| 879 | A-4 | B-82 | B-2 |
| 880 | A-5 | B-83 | B-1 |
| 881 | A-5 | B-84 | B-2 |
| 882 | A-3 | B-85 | B-1 |
| 883 | A-3 | B-86 | B-2 |
| 884 | A-4 | B-87 | B-1 |
| 885 | A-4 | B-88 | B-2 |
| 886 | A-5 | B-89 | B-1 |
| 887 | A-5 | B-90 | B-2 |
| 888 | A-3 | B-91 | B-1 |
| 889 | A-3 | B-92 | B-2 |
| 890 | A-4 | B-93 | B-1 |
| 891 | A-4 | B-94 | B-2 |
| 892 | A-5 | B-95 | B-1 |
| 893 | A-5 | B-96 | B-2 |
| 894 | A-3 | B-97 | B-1 |
| 895 | A-3 | B-98 | B-2 |
| 896 | A-4 | B-99 | B-1 |
| 897 | A-4 | B-101 | B-1 |
| 898 | A-5 | B-102 | B-2 |
| 899 | A-5 | B-103 | B-1 |
| 900 | A-3 | B-104 | B-2 |
| 901 | A-3 | B-105 | B-1 |
| 902 | A-3 | B-106 | B-2 |
| 903 | A-4 | B-5 | C-1 |
| 904 | A-4 | B-6 | C-2 |
| 905 | A-5 | B-7 | C-1 |
| 906 | A-5 | B-8 | C-2 |
| 907 | A-3 | B-9 | C-1 |
| 908 | A-3 | B-12 | C-2 |
| 909 | A-4 | B-13 | C-1 |
| 910 | A-4 | B-14 | C-2 |
| 911 | A-5 | B-15 | C-1 |
| 912 | A-5 | B-16 | C-2 |
| 913 | A-3 | B-17 | C-1 |
| 914 | A-3 | B-18 | C-2 |
| 915 | A-4 | B-19 | C-1 |
| 916 | A-4 | B-20 | C-2 |
| 917 | A-5 | B-21 | C-1 |
| 918 | A-5 | B-22 | C-2 |
| 919 | A-3 | B-23 | C-1 |
| 920 | A-3 | B-24 | C-2 |
| 921 | A-4 | B-25 | C-1 |
| 922 | A-4 | B-26 | C-2 |
| 923 | A-5 | B-27 | C-1 |
| 924 | A-5 | B-28 | C-2 |
| 925 | A-3 | B-29 | C-1 |
| 926 | A-3 | B-30 | C-2 |
| 927 | A-4 | B-31 | C-1 |
| 928 | A-4 | B-32 | C-2 |
| 929 | A-5 | B-33 | C-1 |
| 930 | A-5 | B-34 | C-2 |
| 931 | A-3 | B-35 | C-1 |
| 932 | A-3 | B-36 | C-2 |
| 933 | A-4 | B-37 | C-1 |
| 934 | A-4 | B-38 | C-2 |
| 935 | A-5 | B-39 | C-1 |
| 936 | A-5 | B-40 | C-2 |
| 937 | A-3 | B-41 | C-1 |
| 938 | A-3 | B-42 | C-2 |
| 939 | A-4 | B-43 | C-1 |
| 940 | A-4 | B-44 | C-2 |
| 941 | A-5 | B-45 | C-1 |
| 942 | A-5 | B-46 | C-2 |
| 943 | A-3 | B-47 | C-1 |
| 944 | A-3 | B-48 | C-2 |
| 945 | A-4 | B-49 | C-1 |
| 946 | A-4 | B-50 | C-2 |
| 947 | A-5 | B-51 | C-1 |
| 948 | A-5 | B-52 | C-2 |
| 949 | A-3 | B-53 | C-1 |
| 950 | A-3 | B-54 | C-2 |
| 951 | A-4 | B-55 | C-1 |
| 952 | A-4 | B-56 | C-2 |
| 953 | A-5 | B-57 | C-1 |
| 954 | A-5 | B-58 | C-2 |
| 955 | A-3 | B-59 | C-1 |
| 956 | A-3 | B-60 | C-2 |
| 957 | A-4 | B-61 | C-1 |
| 958 | A-4 | B-62 | C-2 |
| 959 | A-5 | B-63 | C-1 |
| 960 | A-5 | B-64 | C-2 |
| 961 | A-3 | B-65 | C-1 |
| 962 | A-3 | B-66 | C-2 |
| 963 | A-4 | B-67 | C-1 |
| 964 | A-4 | B-68 | C-2 |
| 965 | A-5 | B-69 | C-1 |
| 966 | A-5 | B-70 | C-2 |
| 967 | A-3 | B-71 | C-1 |
| 968 | A-3 | B-72 | C-2 |
| 969 | A-4 | B-73 | C-1 |
| 970 | A-4 | B-74 | C-2 |
| 971 | A-5 | B-75 | C-1 |
| 972 | A-5 | B-76 | C-2 |
| 973 | A-3 | B-77 | C-1 |
| 974 | A-3 | B-78 | C-2 |
| 975 | A-4 | B-79 | C-1 |
| 976 | A-4 | B-80 | C-2 |
| 977 | A-5 | B-81 | C-1 |
| 978 | A-5 | B-82 | C-2 |
| 979 | A-3 | B-83 | C-1 |
| 980 | A-3 | B-84 | C-2 |
| 981 | A-4 | B-85 | C-1 |
| 982 | A-4 | B-86 | C-2 |
| 983 | A-5 | B-87 | C-1 |
| 984 | A-5 | B-88 | C-2 |
| 985 | A-3 | B-89 | C-1 |
| 986 | A-3 | B-90 | C-2 |
| 987 | A-4 | B-91 | C-1 |
| 988 | A-4 | B-92 | C-2 |
| 989 | A-5 | B-93 | C-1 |
| 990 | A-5 | B-94 | C-2 |
| 991 | A-3 | B-95 | C-1 |
| 992 | A-3 | B-96 | C-2 |
| 993 | A-4 | B-97 | C-1 |
| 994 | A-4 | B-98 | C-2 |
| 995 | A-5 | B-99 | C-1 |
| 996 | A-5 | B-101 | C-2 |
| 997 | A-3 | B-102 | C-1 |
| 998 | A-3 | B-103 | C-2 |
| 999 | A-4 | B-104 | C-1 |
| 1000 | A-4 | B-105 | C-2 |
| 1001 | A-5 | B-106 | C-1 |
| 1002 | A-7 | B-1 | C-1 |
| 1003 | A-8 | B-1 | C-2 |
| 1004 | A-9 | B-2 | C-1 |
| 1005 | A-10 | B-2 | C-2 |
| 1006 | A-11 | B-3 | C-1 |
| 1007 | A-12 | B-3 | C-2 |
| 1008 | A-13 | B-10 | C-1 |
| 1009 | A-14 | B-10 | C-2 |
| 1010 | A-15 | B-1 | C-1 |
| 1011 | A-16 | B-1 | C-2 |
| 1012 | A-17 | B-2 | C-1 |
| 1013 | A-18 | B-2 | C-2 |
| 1014 | A-19 | B-3 | C-1 |
| 1015 | A-20 | B-3 | C-2 |
| 1016 | A-21 | B-10 | C-1 |
| 1017 | A-22 | B-10 | C-2 |
| 1018 | A-23 | B-1 | C-1 |
| 1019 | A-24 | B-1 | C-2 |
| 1020 | A-25 | B-2 | C-1 |
| 1021 | A-26 | B-2 | C-2 |
| 1022 | A-27 | B-3 | C-1 |
| 1023 | A-28 | B-3 | C-2 |
| 1024 | A-29 | B-10 | C-1 |
| 1025 | A-30 | B-10 | C-2 |
| 1026 | A-31 | B-1 | C-1 |
| 1027 | A-32 | B-1 | C-2 |
| 1028 | A-33 | B-2 | C-1 |
| 1029 | A-34 | B-2 | C-2 |
| 1030 | A-35 | B-3 | C-1 |
| 1031 | A-36 | B-3 | C-2 |
| 1032 | A-37 | B-10 | C-1 |
| 1033 | A-38 | B-10 | C-2 |
| 1034 | A-39 | B-1 | C-1 |
| 1035 | A-40 | B-1 | C-2 |
| 1036 | A-41 | B-2 | C-1 |
| 1037 | A-42 | B-2 | C-2 |
| 1038 | A-43 | B-3 | C-1 |
| 1039 | A-44 | B-3 | C-2 |
| 1040 | A-45 | B-10 | C-1 |
| 1041 | A-46 | B-10 | C-2 |
| 1042 | A-47 | B-1 | C-1 |
| 1043 | A-48 | B-1 | C-2 |
| 1044 | A-49 | B-2 | C-1 |
| 1045 | A-50 | B-2 | C-2 |
| 1046 | A-51 | B-3 | C-1 |
| 1047 | A-52 | B-3 | C-2 |
| 1048 | A-53 | B-10 | C-1 |
| 1049 | A-54 | B-10 | C-2 |
| 1050 | A-55 | B-1 | C-1 |
| 1051 | A-56 | B-1 | C-2 |
| 1052 | A-57 | B-2 | C-1 |
| 1053 | A-58 | B-2 | C-2 |
| 1054 | A-59 | B-3 | C-1 |
| 1055 | A-60 | B-3 | C-2 |
| 1056 | A-61 | B-10 | C-1 |
| 1057 | A-62 | B-10 | C-2 |
| 1058 | A-63 | B-1 | C-1 |
| 1059 | A-64 | B-1 | C-2 |
| 1060 | A-65 | B-2 | C-1 |
| 1061 | A-66 | B-2 | C-2 |
| 1062 | A-67 | B-3 | C-1 |
| 1063 | A-68 | B-3 | C-2 |
| 1064 | A-69 | B-10 | C-1 |
| 1065 | A-101 | B-10 | C-1 |
| 1066 | A-102 | B-1 | C-2 |
| 1067 | A-103 | B-1 | C-1 |
| 1068 | A-104 | B-2 | C-2 |
| 1069 | A-105 | B-2 | C-1 |
| 1070 | A-106 | B-1 | C-2 |
| 1071 | A-7 | B-2 | B-10 |
| 1072 | A-8 | B-1 | B-1 |
| 1073 | A-9 | B-1 | B-2 |
| 1074 | A-10 | B-2 | B-2 |
| 1075 | A-11 | B-1 | B-3 |
| 1076 | A-12 | B-1 | B-10 |
| 1077 | A-13 | B-2 | B-3 |
| 1078 | A-14 | B-2 | B-10 |
| 1079 | A-15 | B-1 | B-1 |
| 1080 | A-16 | B-1 | B-2 |
| 1081 | A-17 | B-2 | B-2 |
| 1082 | A-18 | B-1 | B-3 |
| 1083 | A-19 | B-1 | B-10 |
| 1084 | A-20 | B-2 | B-3 |
| 1085 | A-21 | B-2 | B-10 |
| 1086 | A-22 | B-1 | B-1 |
| 1087 | A-23 | B-1 | B-2 |
| 1088 | A-24 | B-2 | B-2 |
| 1089 | A-25 | B-1 | B-3 |
| 1090 | A-26 | B-1 | B-10 |
| 1091 | A-27 | B-2 | B-3 |
| 1092 | A-28 | B-2 | B-10 |
| 1093 | A-29 | B-1 | B-1 |
| 1094 | A-30 | B-1 | B-2 |
| 1095 | A-31 | B-2 | B-2 |
| 1096 | A-32 | B-1 | B-3 |
| 1097 | A-33 | B-1 | B-10 |
| 1098 | A-34 | B-2 | B-3 |
| 1099 | A-35 | B-2 | B-10 |
| 1100 | A-36 | B-1 | B-1 |
| 1101 | A-37 | B-1 | B-2 |
| 1102 | A-38 | B-2 | B-2 |
| 1103 | A-39 | B-1 | B-3 |
| 1104 | A-40 | B-1 | B-10 |
| 1105 | A-41 | B-2 | B-3 |
| 1106 | A-42 | B-2 | B-10 |
| 1107 | A-43 | B-1 | B-1 |
| 1108 | A-44 | B-1 | B-2 |
| 1109 | A-45 | B-2 | B-2 |
| 1110 | A-46 | B-1 | B-3 |
| 1111 | A-47 | B-1 | B-10 |
| 1112 | A-48 | B-2 | B-3 |
| 1113 | A-49 | B-2 | B-10 |
| 1114 | A-50 | B-1 | B-1 |
| 1115 | A-51 | B-1 | B-2 |
| 1116 | A-52 | B-2 | B-2 |
| 1117 | A-53 | B-1 | B-3 |
| 1118 | A-54 | B-1 | B-10 |
| 1119 | A-55 | B-2 | B-3 |
| 1120 | A-56 | B-2 | B-10 |
| 1121 | A-57 | B-1 | B-1 |
| 1122 | A-58 | B-1 | B-2 |
| 1123 | A-59 | B-2 | B-2 |
| 1124 | A-60 | B-1 | B-3 |
| 1125 | A-61 | B-1 | B-10 |
| 1126 | A-62 | B-2 | B-3 |
| 1127 | A-63 | B-2 | B-10 |
| 1128 | A-64 | B-1 | B-1 |
| 1129 | A-65 | B-1 | B-2 |
| 1130 | A-66 | B-2 | B-2 |
| 1131 | A-67 | B-1 | B-3 |
| 1132 | A-68 | B-1 | B-10 |
| 1133 | A-69 | B-2 | B-3 |
| 1134 | A-101 | B-2 | B-10 |
| 1135 | A-102 | B-1 | B-1 |
| 1136 | A-103 | B-1 | B-2 |
| 1137 | A-104 | B-2 | B-2 |
| 1138 | A-105 | B-1 | B-3 |
| 1139 | A-106 | B-1 | B-10 |
| 1140 | A-1 | B-1 | A-1 |
| 1141 | A-2 | B-2 | A-2 |
| 1142 | A-3 | B-3 | A-3 |
| 1143 | A-4 | B-10 | A-4 |
| 1144 | A-5 | B-1 | A-5 |
| 1145 | A-6 | B-2 | A-6 |
| 1146 | A-7 | B-3 | A-7 |
| 1147 | A-8 | B-10 | A-8 |
| 1148 | A-9 | B-1 | A-9 |
| 1149 | A-10 | B-2 | A-10 |
| 1150 | A-11 | B-3 | A-11 |
| 1151 | A-12 | B-10 | A-12 |
| 1152 | A-13 | B-1 | A-13 |
| 1153 | A-14 | B-2 | A-14 |
| 1154 | A-15 | B-3 | A-15 |
| 1155 | A-16 | B-10 | A-16 |
| 1156 | A-17 | B-1 | A-17 |
| 1157 | A-18 | B-2 | A-18 |
| 1158 | A-19 | B-3 | A-19 |
| 1159 | A-20 | B-10 | A-20 |
| 1160 | A-21 | B-1 | A-21 |
| 1161 | A-22 | B-2 | A-22 |
| 1162 | A-23 | B-3 | A-23 |
| 1163 | A-24 | B-10 | A-24 |
| 1164 | A-25 | B-1 | A-25 |
| 1165 | A-26 | B-2 | A-26 |
| 1166 | A-27 | B-3 | A-27 |
| 1167 | A-28 | B-10 | A-28 |
| 1168 | A-29 | B-1 | A-29 |
| 1169 | A-30 | B-2 | A-30 |
| 1170 | A-31 | B-3 | A-31 |
| 1171 | A-32 | B-10 | A-32 |
| 1172 | A-33 | B-1 | A-33 |
| 1173 | A-34 | B-2 | A-34 |
| 1174 | A-35 | B-3 | A-35 |
| 1175 | A-36 | B-10 | A-36 |
| 1176 | A-37 | B-1 | A-37 |
| 1177 | A-38 | B-2 | A-38 |
| 1178 | A-39 | B-3 | A-39 |
| 1179 | A-40 | B-10 | A-40 |
| 1180 | A-41 | B-1 | A-41 |
| 1181 | A-42 | B-2 | A-42 |
| 1182 | A-43 | B-3 | A-43 |
| 1183 | A-44 | B-10 | A-44 |
| 1184 | A-45 | B-1 | A-45 |
| 1185 | A-46 | B-2 | A-46 |
| 1186 | A-47 | B-3 | A-47 |
| 1187 | A-48 | B-10 | A-48 |
| 1188 | A-49 | B-1 | A-49 |
| 1189 | A-50 | B-2 | A-50 |
| 1190 | A-51 | B-3 | A-51 |
| 1191 | A-52 | B-10 | A-52 |
| 1192 | A-53 | B-1 | A-53 |
| 1193 | A-54 | B-2 | A-54 |
| 1194 | A-55 | B-3 | A-55 |
| 1195 | A-56 | B-10 | A-56 |
| 1196 | A-57 | B-1 | A-57 |
| 1197 | A-58 | B-2 | A-58 |
| 1198 | A-59 | B-3 | A-59 |
| 1199 | A-60 | B-10 | A-60 |
| 1200 | A-61 | B-1 | A-61 |
| 1201 | A-62 | B-2 | A-62 |
| 1202 | A-63 | B-3 | A-63 |
| 1203 | A-64 | B-10 | A-64 |
| 1204 | A-65 | B-1 | A-65 |
| 1205 | A-66 | B-2 | A-66 |
| 1206 | A-67 | B-3 | A-67 |
| 1207 | A-68 | B-10 | A-68 |
| 1208 | A-69 | B-1 | A-69 |
| 1209 | A-101 | B-2 | A-101 |
| 1210 | A-102 | B-3 | A-102 |
| 1211 | A-103 | B-10 | A-103 |
| 1212 | A-104 | B-1 | A-104 |
| 1213 | A-105 | B-2 | A-105 |
| 1214 | A-106 | B-3 | A-106 |
| 1215 | A-3 | B-4 | B-4 |
| 1216 | A-4 | B-5 | B-5 |
| 1217 | A-5 | B-6 | B-6 |
| 1218 | A-3 | B-7 | B-7 |
| 1219 | A-4 | B-8 | B-8 |
| 1220 | A-5 | B-9 | B-9 |
| 1221 | A-3 | B-10 | B-10 |
| 1222 | A-4 | B-11 | B-11 |
| 1223 | A-5 | B-12 | B-12 |
| 1224 | A-3 | B-13 | B-13 |
| 1225 | A-4 | B-14 | B-14 |
| 1226 | A-5 | B-15 | B-15 |
| 1227 | A-3 | B-16 | B-16 |
| 1228 | A-4 | B-17 | B-17 |
| 1229 | A-5 | B-18 | B-18 |
| 1230 | A-3 | B-19 | B-19 |
| 1231 | A-4 | B-20 | B-20 |
| 1232 | A-5 | B-21 | B-21 |
| 1233 | A-3 | B-22 | B-22 |
| 1234 | A-4 | B-23 | B-23 |
| 1235 | A-5 | B-24 | B-24 |
| 1236 | A-3 | B-25 | B-25 |
| 1237 | A-4 | B-26 | B-26 |
| 1238 | A-5 | B-27 | B-27 |
| 1239 | A-3 | B-28 | B-28 |
| 1240 | A-4 | B-29 | B-29 |
| 1241 | A-5 | B-30 | B-30 |
| 1242 | A-3 | B-31 | B-31 |
| 1243 | A-4 | B-32 | B-32 |
| 1244 | A-5 | B-33 | B-33 |
| 1245 | A-3 | B-34 | B-34 |
| 1246 | A-4 | B-35 | B-35 |
| 1247 | A-5 | B-36 | B-36 |
| 1248 | A-3 | B-37 | B-37 |
| 1249 | A-4 | B-38 | B-38 |
| 1250 | A-5 | B-39 | B-39 |
| 1251 | A-3 | B-40 | B-40 |
| 1252 | A-4 | B-41 | B-41 |
| 1253 | A-5 | B-42 | B-42 |
| 1254 | A-3 | B-43 | B-43 |
| 1255 | A-4 | B-44 | B-44 |
| 1256 | A-5 | B-45 | B-45 |
| 1257 | A-3 | B-46 | B-46 |
| 1258 | A-4 | B-47 | B-47 |
| 1259 | A-5 | B-48 | B-48 |
| 1260 | A-3 | B-49 | B-49 |
| 1261 | A-4 | B-50 | B-50 |
| 1262 | A-5 | B-51 | B-51 |
| 1263 | A-3 | B-52 | B-52 |
| 1264 | A-4 | B-53 | B-53 |
| 1265 | A-5 | B-54 | B-54 |
| 1266 | A-3 | B-55 | B-55 |
| 1267 | A-4 | B-56 | B-56 |
| 1268 | A-5 | B-57 | B-57 |
| 1269 | A-3 | B-58 | B-58 |
| 1270 | A-4 | B-59 | B-59 |
| 1271 | A-5 | B-60 | B-60 |
| 1272 | A-3 | B-61 | B-61 |
| 1273 | A-4 | B-62 | B-62 |
| 1274 | A-5 | B-63 | B-63 |
| 1275 | A-3 | B-64 | B-64 |
| 1276 | A-4 | B-65 | B-65 |
| 1277 | A-5 | B-66 | B-66 |
| 1278 | A-3 | B-67 | B-67 |
| 1279 | A-4 | B-68 | B-68 |
| 1280 | A-5 | B-69 | B-69 |
| 1281 | A-3 | B-70 | B-70 |
| 1282 | A-4 | B-71 | B-71 |
| 1283 | A-5 | B-72 | B-72 |
| 1284 | A-3 | B-73 | B-73 |
| 1285 | A-4 | B-74 | B-74 |
| 1286 | A-5 | B-75 | B-75 |
| 1287 | A-3 | B-76 | B-76 |
| 1288 | A-4 | B-77 | B-77 |
| 1289 | A-5 | B-78 | B-78 |
| 1290 | A-3 | B-79 | B-79 |
| 1291 | A-4 | B-80 | B-80 |
| 1292 | A-5 | B-81 | B-81 |
| 1293 | A-3 | B-82 | B-82 |
| 1294 | A-4 | B-83 | B-83 |
| 1295 | A-5 | B-84 | B-84 |
| 1296 | A-3 | B-85 | B-85 |
| 1297 | A-4 | B-86 | B-86 |
| 1298 | A-5 | B-87 | B-87 |
| 1299 | A-3 | B-88 | B-88 |
| 1300 | A-4 | B-89 | B-89 |
| 1301 | A-5 | B-90 | B-90 |
| 1302 | A-3 | B-91 | B-91 |
| 1303 | A-4 | B-92 | B-92 |
| 1304 | A-5 | B-93 | B-93 |
| 1305 | A-3 | B-94 | B-94 |
| 1306 | A-4 | B-95 | B-95 |
| 1307 | A-5 | B-96 | B-96 |
| 1308 | A-3 | B-97 | B-97 |
| 1309 | A-4 | B-98 | B-98 |
| 1310 | A-5 | B-99 | B-99 |
| 1311 | A-6 | B-101 | B-101 |
| 1312 | A-5 | B-102 | B-102 |
| 1313 | A-3 | B-103 | B-103 |
| 1314 | A-4 | B-104 | B-104 |
| 1315 | A-5 | B-105 | B-105 |
| 1316 | A-6 | B-106 | B-106 |
| 1317 | A-67 | B-98 | C-72 |
| 1318 | A-68 | B-99 | C-73 |
| 1319 | A-69 | B-98 | C-74 |
| 1320 | A-67 | B-99 | C-75 |
| 1321 | A-68 | B-98 | C-76 |
| 1322 | A-69 | B-99 | C-77 |
| 1323 | A-67 | B-98 | B-98 |
| 1324 | A-68 | B-98 | B-99 |
| 1325 | A-69 | B-99 | B-99 |
| 2001 | A-101 | B-101 | B-101 |
| 2002 | A-102 | B-101 | B-102 |
| 2003 | A-103 | B-101 | B-103 |
| 2004 | A-101 | B-101 | B-104 |
| 2005 | A-102 | B-102 | B-102 |
| 2006 | A-103 | B-102 | B-103 |
| 2007 | A-101 | B-102 | B-104 |
| 2008 | A-102 | B-103 | B-103 |
| 2009 | A-103 | B-103 | B-104 |
| 2010 | A-101 | B-104 | B-104 |
| 2011 | A-102 | B-101 | C-101 |
| 2012 | A-103 | B-102 | C-102 |
| 2013 | A-101 | B-103 | C-103 |
| 2014 | A-102 | B-104 | C-104 |
| 2015 | A-103 | B-101 | C-105 |
| 2016 | A-101 | B-102 | C-106 |
| 2017 | A-102 | B-103 | C-107 |
| 2018 | A-103 | B-104 | C-108 |
| 2019 | A-101 | B-101 | C-109 |

The light emitting device ED according to an embodiment may further include materials for the hole transport region, which will be described below, in the hole transport region HTR, in addition to the above-described amine compound of an embodiment.

The hole transport region HTR may include a compound represented by Formula H-1:

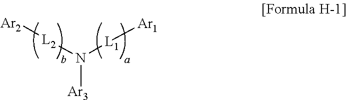

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms; and a and b may each independently be an integer from 0 to 10. When a or b is 2 or greater, multiple L₁ groups and multiple L₂ groups may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In Formula H-1, Ar₃ may be a substituted or unsubstituted aryl group having 6 to 30 ring-forming carbon atoms.

In an embodiment, the compound represented by Formula H-1 may be a monoamine compound. In another embodiment, the compound represented by Formula H-1 may be a diamine compound in which at least one of Ar₁ to Ar₃ includes an amine group as a substituent. In still another embodiment, the compound represented by Formula H-1 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be any one selected from Compound Group H. However, the compounds listed in Compound Group H are only examples, and the compounds represented by Formula H-1 are not limited to Compound Group H:

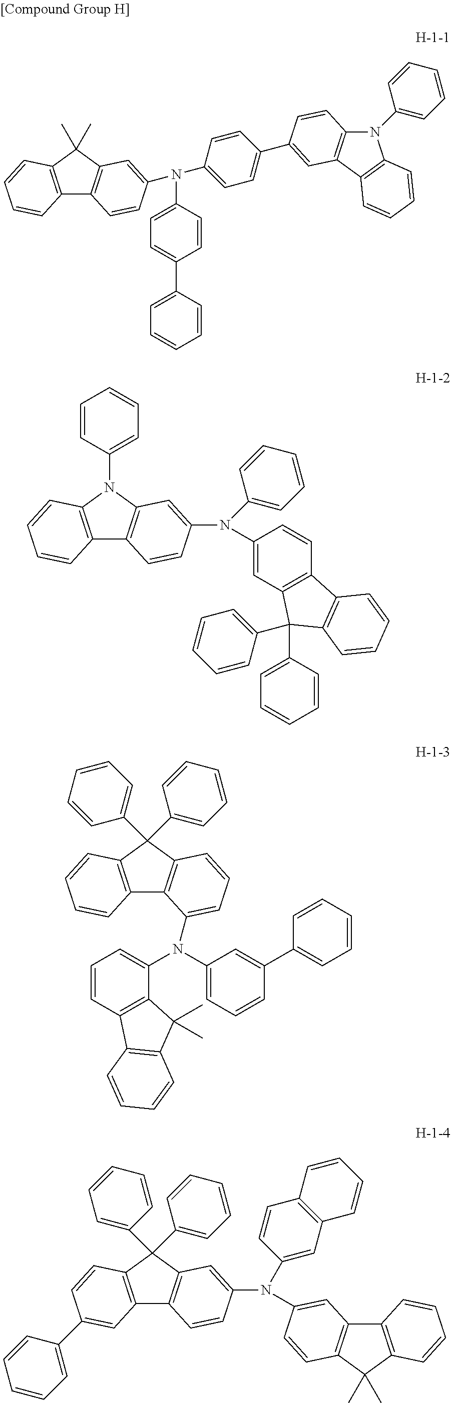 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

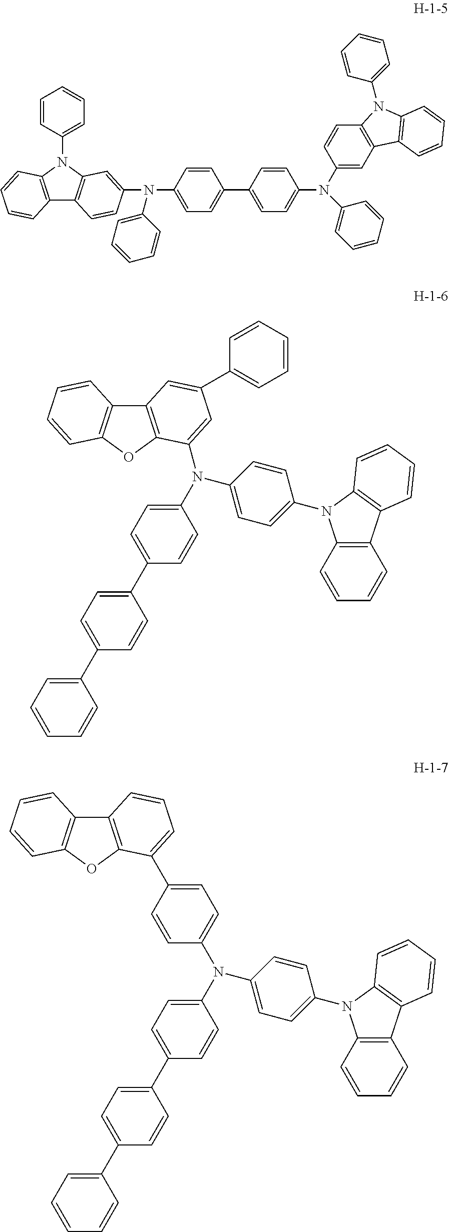 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

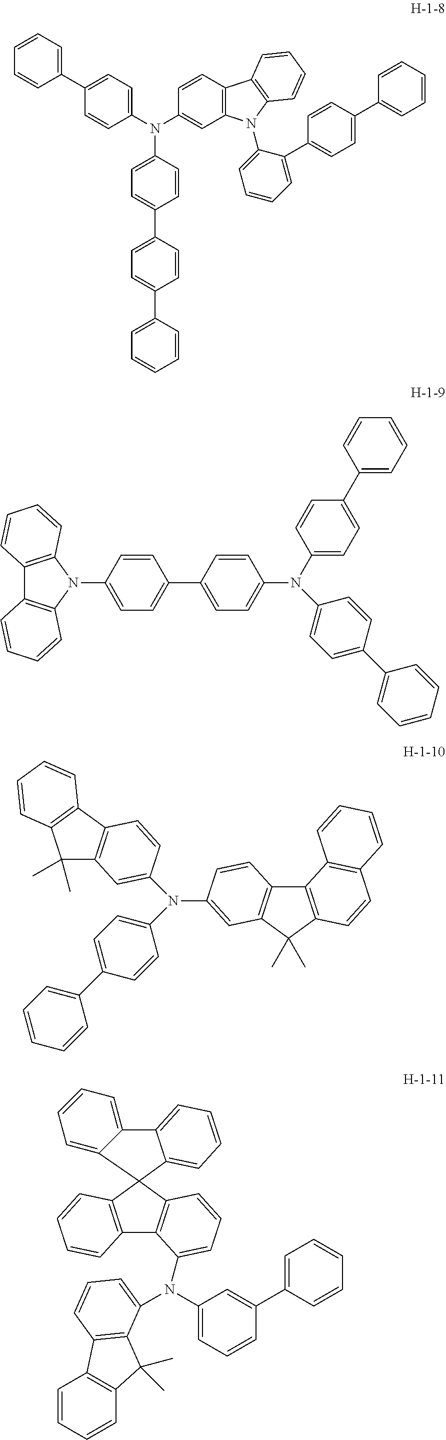

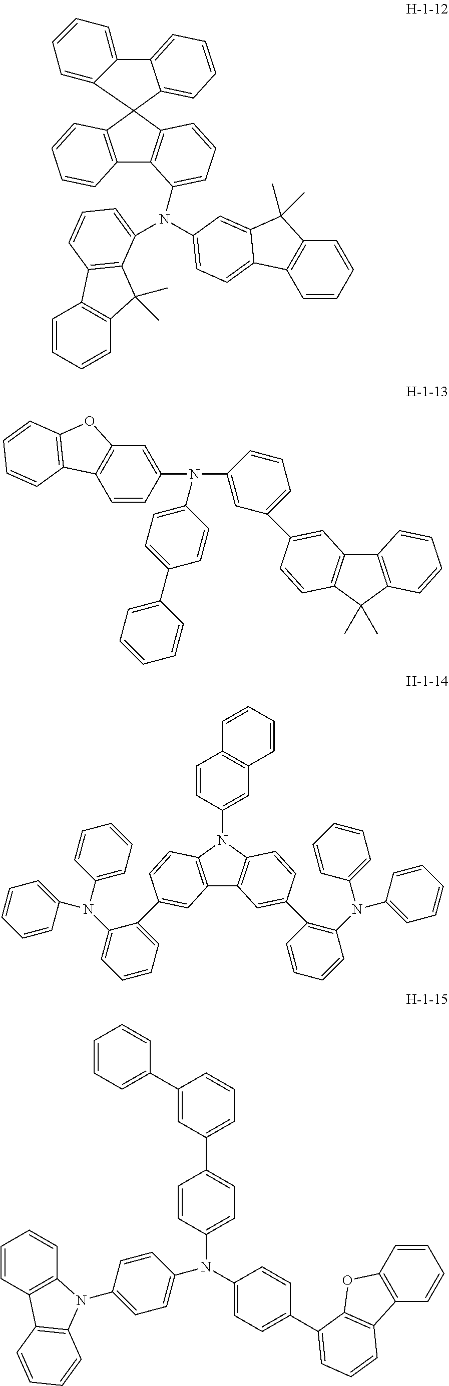

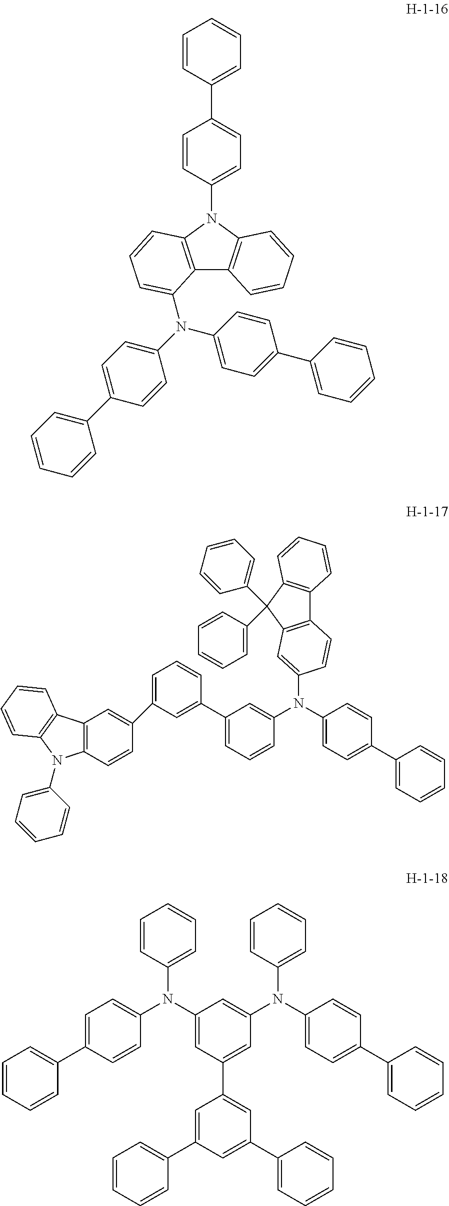 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

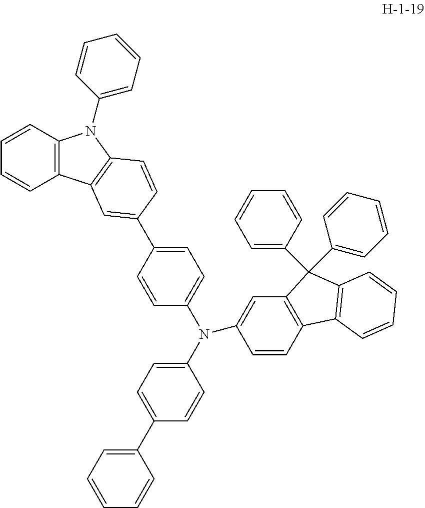 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), 4,4′,4″-tris[N(1-naphthyl)-N-phenylamino]-triphenylamine (1-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include a carbazole-based derivative such as N-phenyl carbazole and polyvinyl carbazole, a fluorene-based derivative, N,N′-bis(3-methylphenyl)-N,N′-diphenyl-[1,1-biphenyl]-4,4′-diamine (TPD), a triphenylamine-based derivative such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one of a hole injection layer HIL or a hole transport layer HTL.

A thickness of the hole transport region HTR may be in a range of about 100 Å to about 10,000 Å. For example, the thickness of the hole transport region HTR may be in a range of about 100 Å to about 5,000 Å. When the hole transport region HTR includes a hole injection layer HIL, the hole injection layer HIL may have, for example, a thickness in a range of about 30 Å to about 1,000 Å. When the hole transport region HTR includes a hole transport layer HTL, the hole transport layer HTL may have a thickness in a range of about 30 Å to about 1,000 Å. When the hole transport region HTR includes an electron blocking layer EBL, the electron blocking layer EBL may have a thickness in a range of about 10 Å to about 1,000 Å. If the thicknesses of the hole transport region HTR, the hole injection layer HIL, the hole transport layer HTL, and the electron blocking layer EBL satisfy the above-described ranges, satisfactory hole transport properties may be achieved without a substantial increase in driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but embodiments are not limited thereto. For example, the p-dopant may include a metal halide compound such as CuI and RbI, a quinone derivative such as tetracyanoquinodimethane (TCNQ) and 2,3,5,6-tetrafluoro-7,7′8,8-tetracyanoquinodimethane (F4-TCNQ), a metal oxide such as tungsten oxide and molybdenum oxide, a cyano group-containing compound such as dipyrazino[2,3-f: 2′,3′-h] quinoxaline-2,3,6,7,10,11-hexacarbonitrile (HATCN), 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments are not limited thereto.

As described above, the hole transport region HTR may further include at least one of a buffer layer (not shown) or an electron blocking layer EBL, in addition to the hole injection layer HIL and the hole transport layer HTL. The buffer layer (not shown) may compensate for a resonance distance according to a wavelength of light emitted from the emission layer EML and may thus increase light emission efficiency. A material that may be included in the hole transport region HTR may be used as a material to be included in the buffer layer (not shown). The electron blocking layer EBL may prevent the injection of electrons from the electron transport region ETR to the hole transport region HTR.

The emission layer EML is provided on the hole transport region HTR. The emission layer EML may have a thickness, for example, in a range of about 100 Å to about 1,000 Å. For example, the emission layer EML may have a thickness in a range of about 100 Å to about 300 Å. The emission layer EML may be a layer formed of a single material, a layer formed of different materials, or a structure including multiple layers formed of different materials.

In the light emitting device ED according to an embodiment, the emission layer EML may include an anthracene derivative, a pyrene derivative, a fluoranthene derivative, a chrysene derivative, a dihydrobenzanthracene derivative, or a triphenylene derivative. For example, the emission layer EML may include an anthracene derivative or a pyrene derivative.

In each light emitting device ED according to embodiments illustrated in FIGS. 3 to 6, the emission layer EML may include a host and a dopant, and the emission layer EML may include a compound represented by Formula E-1. The compound represented by Formula E-1 may be used as a fluorescence host material.

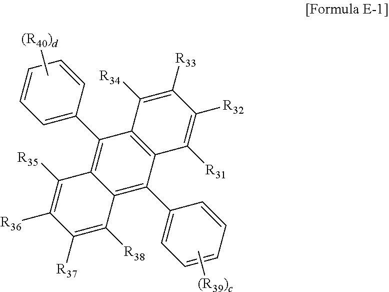

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 1 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer from 0 to 5.

The compound represented by Formula E-1 may be any one selected from Compound E1 to Compound E19:

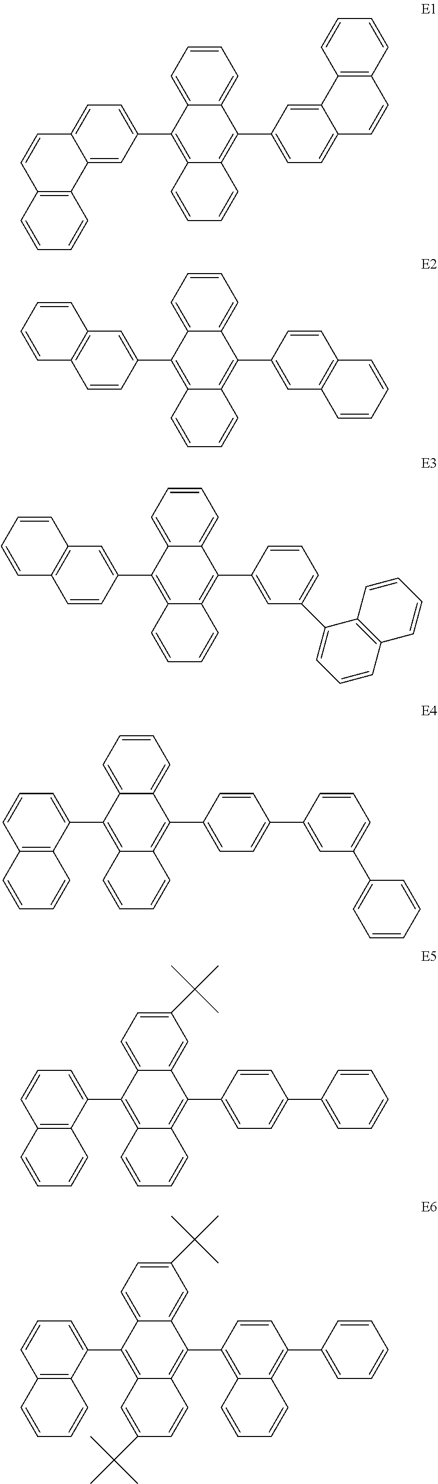

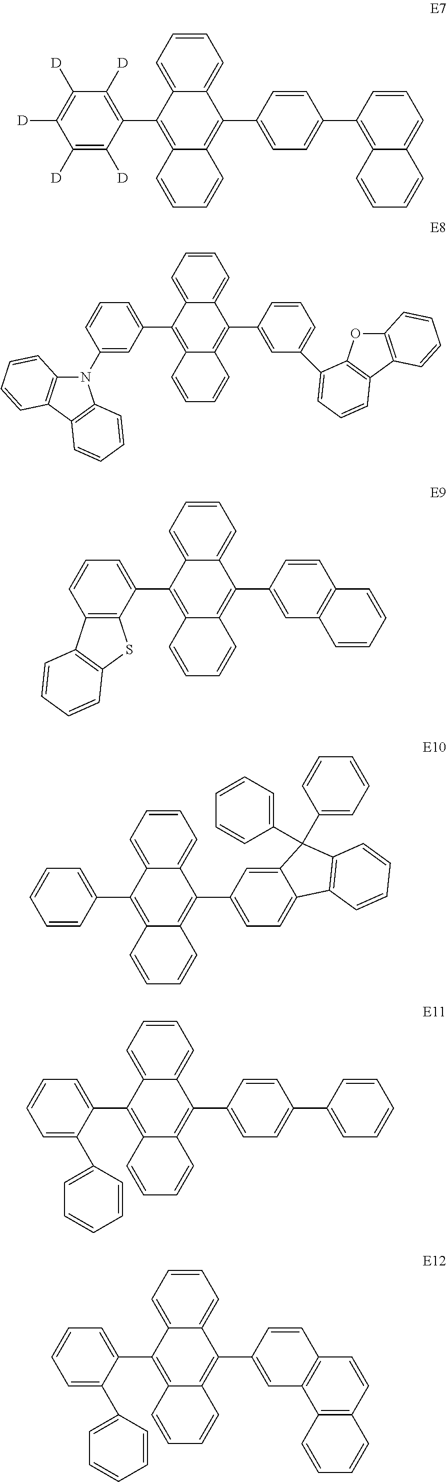

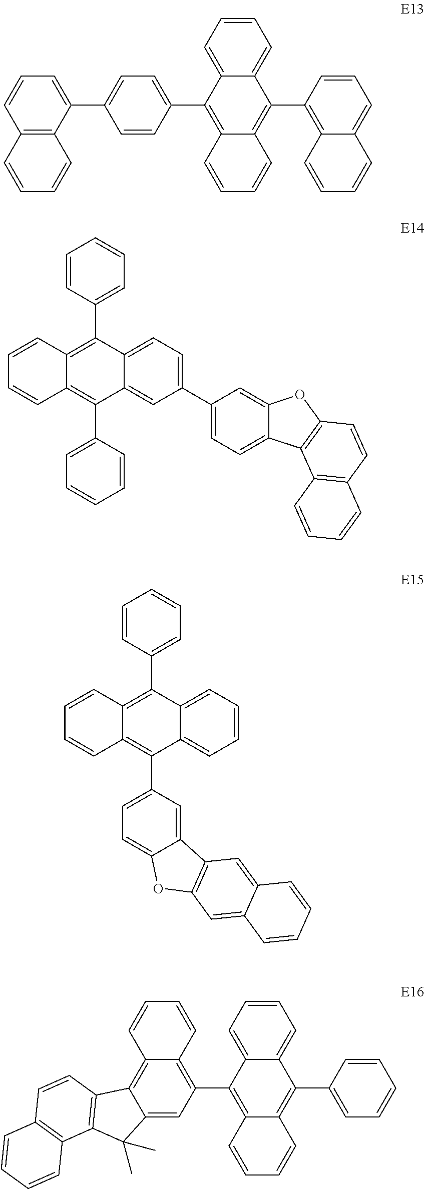

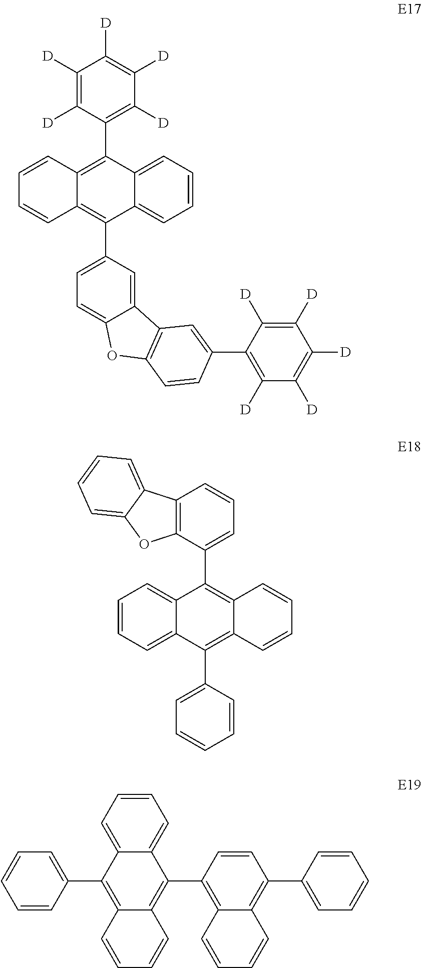

In an embodiment, the emission layer EML may include a compound represented by Formula E-2a or Formula E-2b. The compound represented by Formula E-2a or Formula E-2b may be used as a phosphorescence host material.

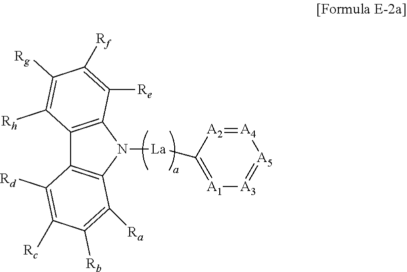

In Formula E-2a, a may be an integer from 0 to 10; and Lₐ may be a direct linkage, a substituted or unsubstituted arylene group having 6 to 30 ring-forming carbon atoms, or a substituted or unsubstituted heteroarylene group having 2 to 30 ring-forming carbon atoms. When a is 2 or more, multiple Lₐ groups may each independently be a substituted or unsubstituted arylene group having 6 to 30 ring-forming carbon atoms, or a substituted or unsubstituted heteroarylene group having 2 to 30 ring-forming carbon atoms.

In Formula E-2a, A₁ to A₅ may each independently be N or C(Rᵢ). In Formula E-2a, Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. For example, Rₐ to Rᵢ may be bonded to an adjacent group to form a hydrocarbon ring or a heterocycle containing N, O, S, etc. as a ring-forming atom.

In Formula E-2a, two or three of A₁ to A₅ may be N, and the remainder of Aᵢ to A₅ may be C(Rᵢ).

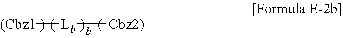

In Formula E-2b, Cbz1 and Cbz2 may each independently be an unsubstituted carbazole group, or a carbazole group substituted with an aryl group having 6 to 30 ring-forming carbon atoms. In Formula E-2b, L_{b} may be a direct linkage, a substituted or unsubstituted arylene group having 6 to 30 ring-forming carbon atoms, or a substituted or unsubstituted heteroarylene group having 2 to 30 ring-forming carbon atoms. In Formula E-2b, b may be an integer from 0 to 10, and when b is 2 or more, multiple L_{b} groups may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be any one selected from Compound Group E-2. However, the compounds listed in Compound Group E-2 are only examples, and the compound represented by Formula E-2a or Formula E-2b is not limited to Compound Group E-2.

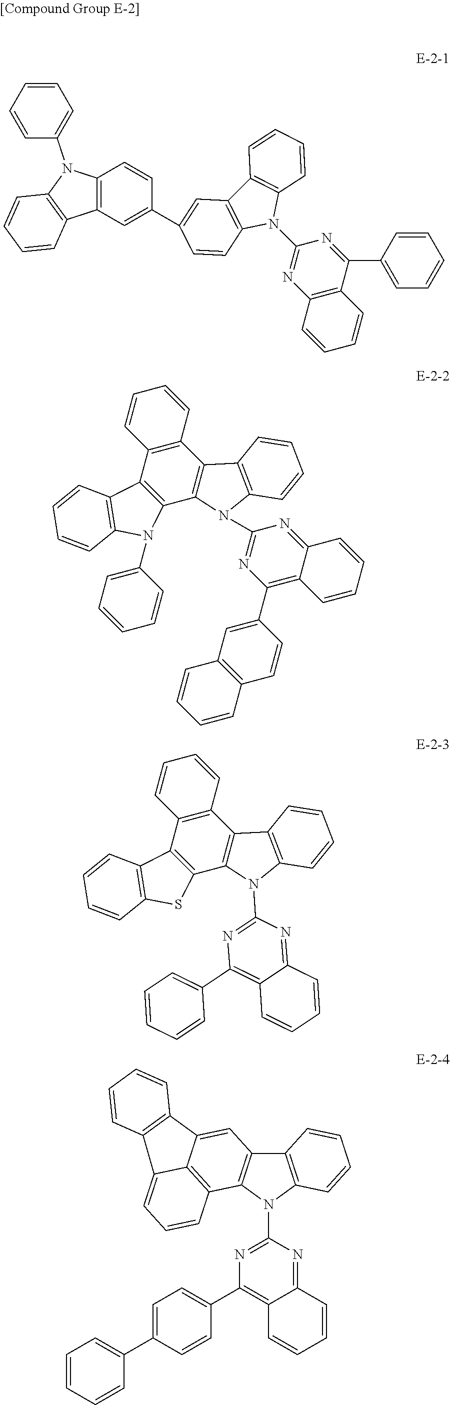

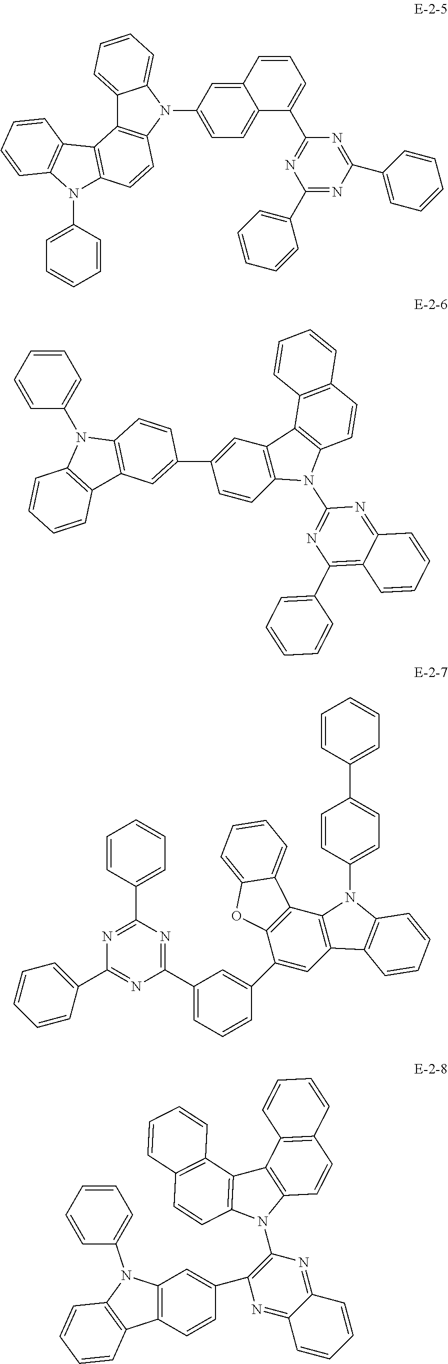 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

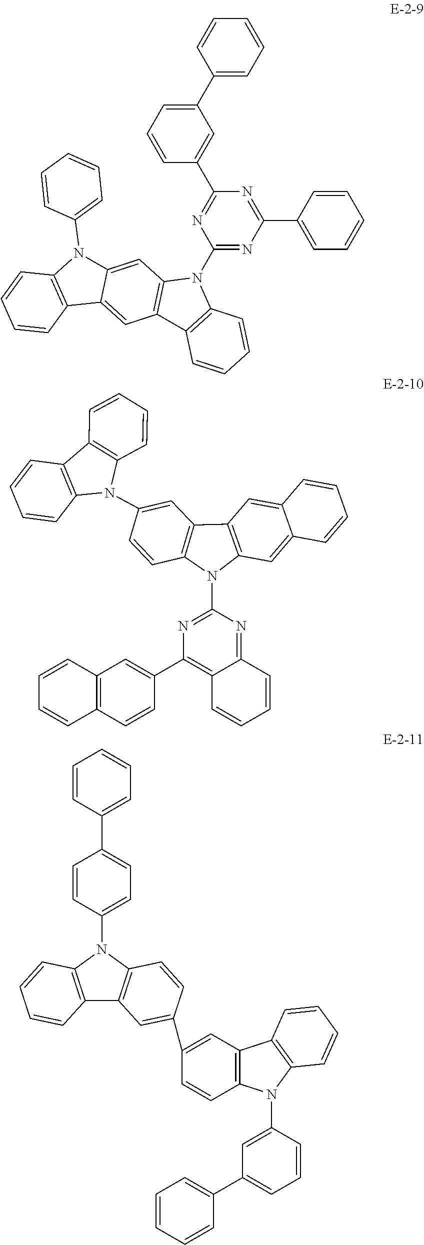

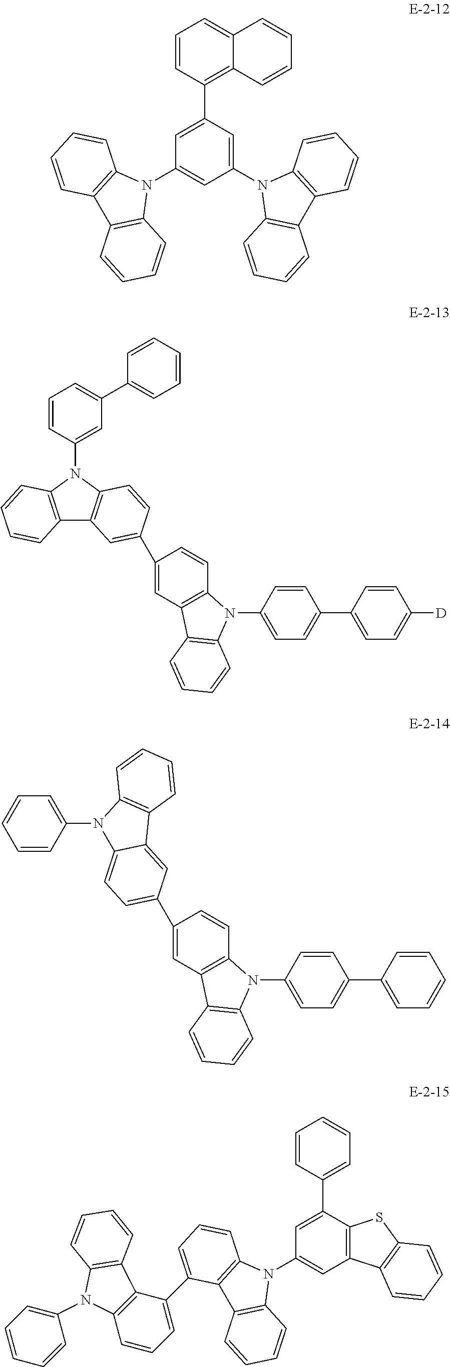 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

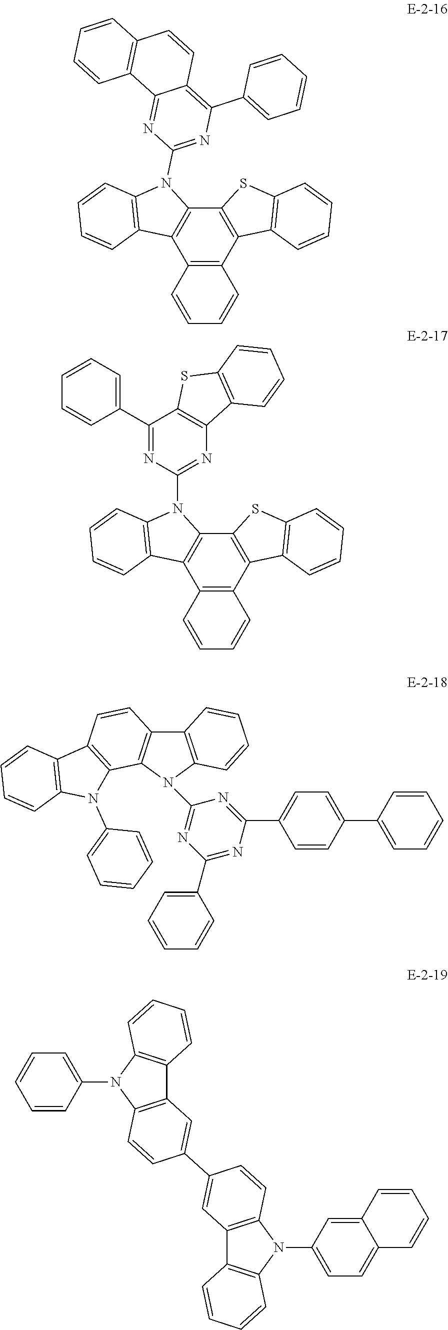

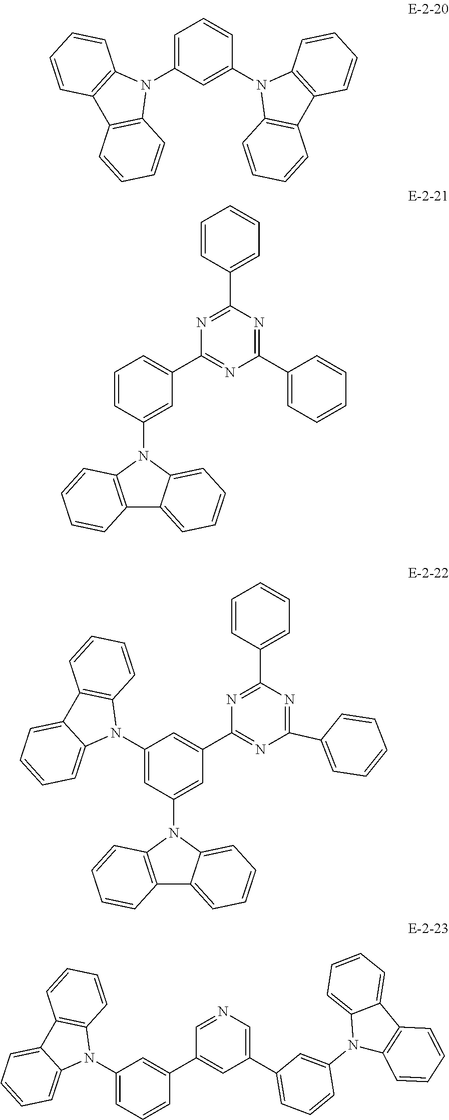

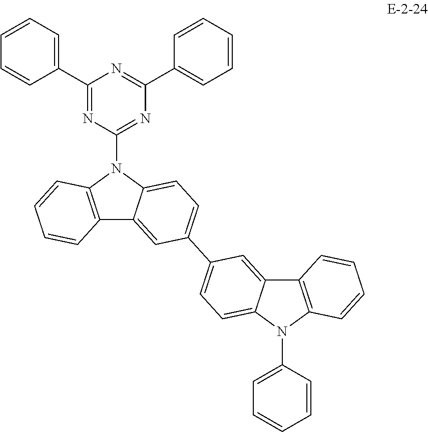 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material of the related art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, embodiments are not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as a host material.

The emission layer EML may include a compound represented by Formula M-a or Formula M-b. The compound represented by Formula M-a or Formula M-b may be used as a phosphorescence dopant material.

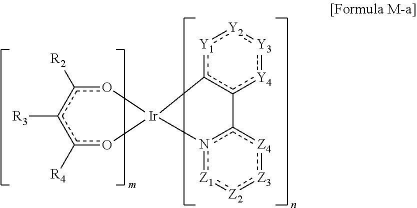

In Formula M-a, Y₁ to Y₄ and Z₁ to Z₄ may each independently be C(R₁) or N; and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m may be 0 or 1, and n may be 2 or 3. In Formula M-a, when m is 0, n may be 3, and when m is 1, n may be 2.

The compound represented by Formula M-a may be used as a phosphorescence dopant material.

The compound represented by Formula M-a may be any one selected from Compound M-a1 to Compound M-a21. However, Compounds M-a1 to M-a21 are only examples, and the compound represented by Formula M-a is not limited to Compounds M-a1 to M-a21.

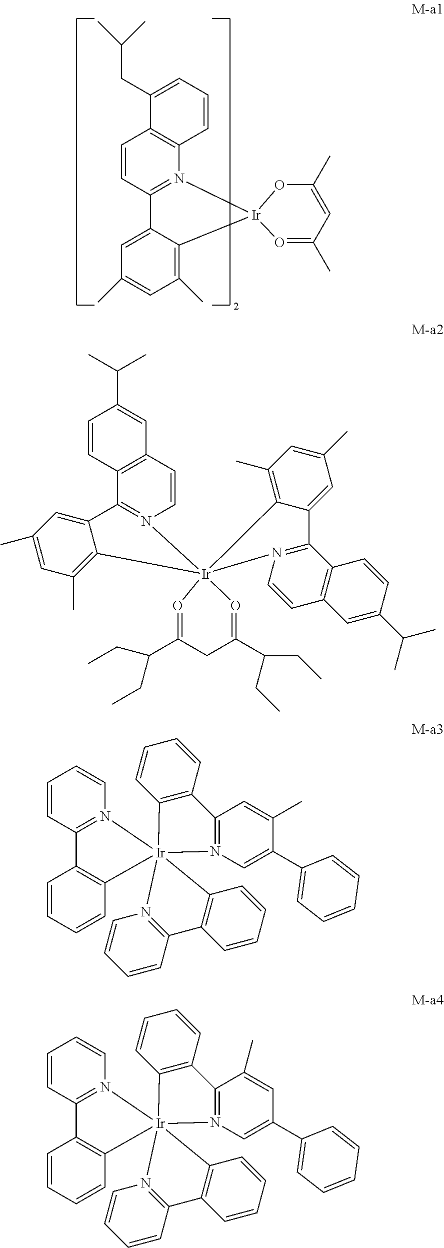

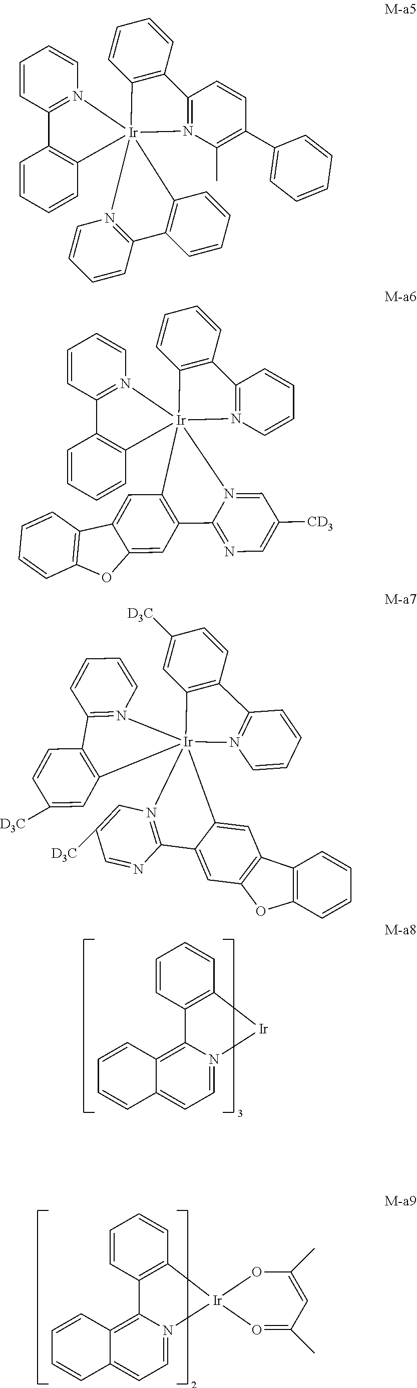

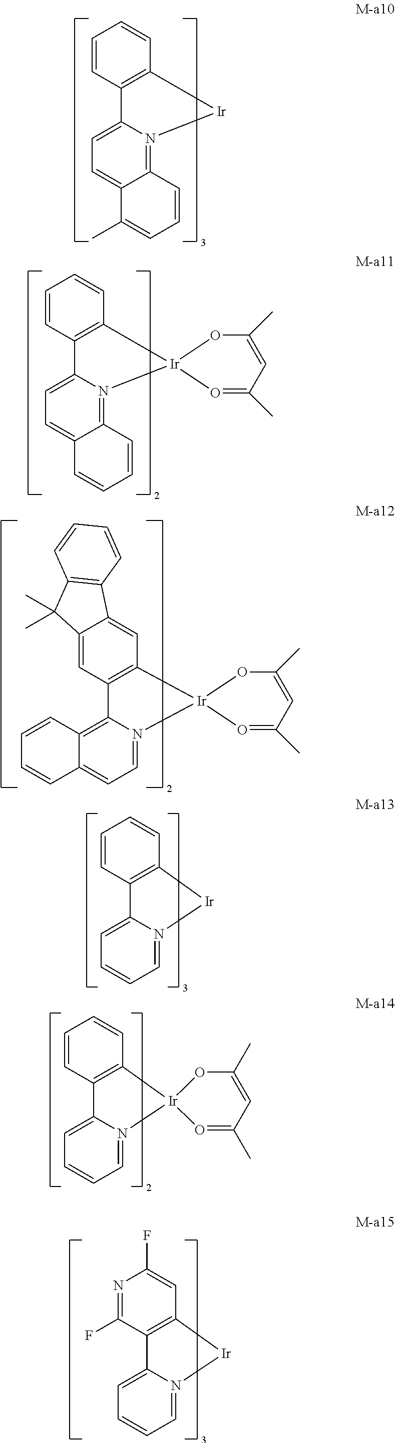

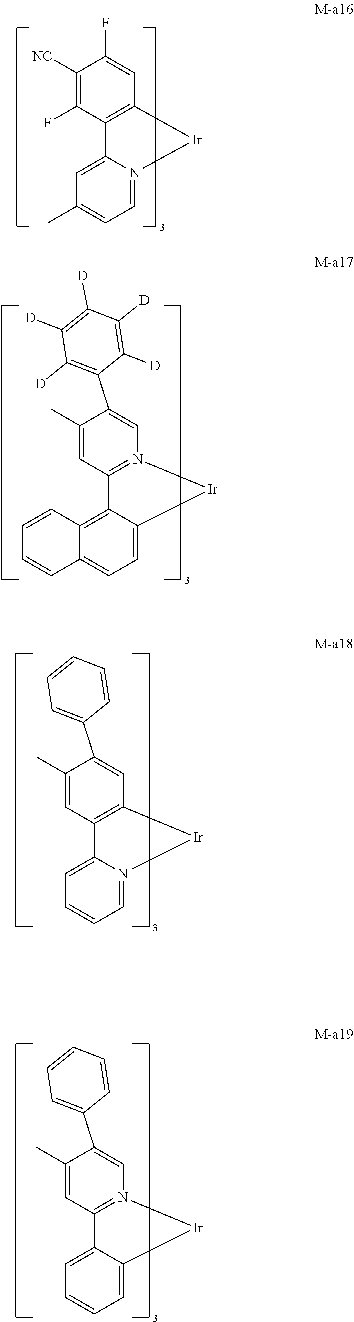

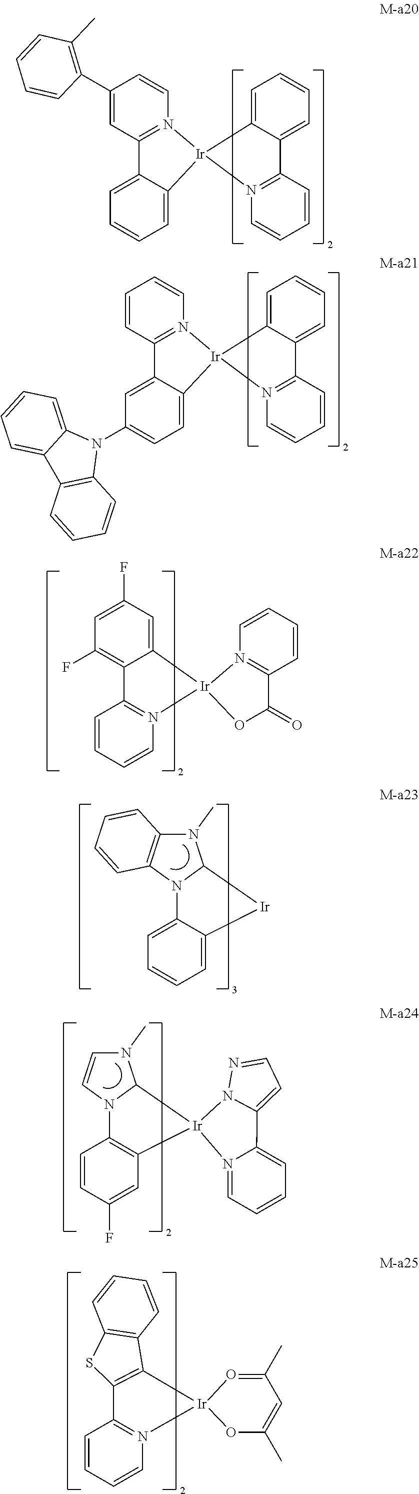

Compound M-a1 and Compound M-a2 may be used as a red dopant material, and Compound M-a3 to Compound M-a7 may be used as a green dopant material.

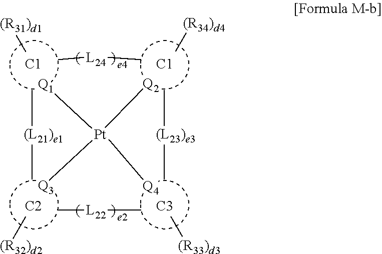

In Formula M-b, Q₁ to Q₄ may each independently be C or N; C1 to C4 may each independently be a substituted or unsubstituted hydrocarbon ring having 5 to 30 ring-forming carbon atoms, or a substituted or unsubstituted heterocycle having 2 to 30 ring-forming carbon atoms; L₂₁ to L₂₄ may each independently be a direct linkage,

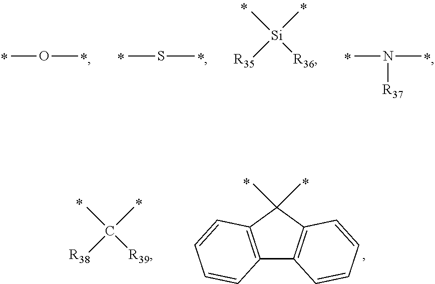

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms; and e1 to e4 may each independently be 0 or 1. In Formula M-b, R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring; and d1 to d4 may each independently be an integer from 0 to 4.

The compound represented by Formula M-b may be used as a blue phosphorescence dopant or a green phosphorescence dopant.

The compound represented by Formula M-b may be any one selected from Compounds M-b-1 to M-b-11. However, Compounds M-b-1 to M-b-11 are only examples, and the compound represented by Formula M-b is not limited to Compounds M-b-1 to M-b-11:

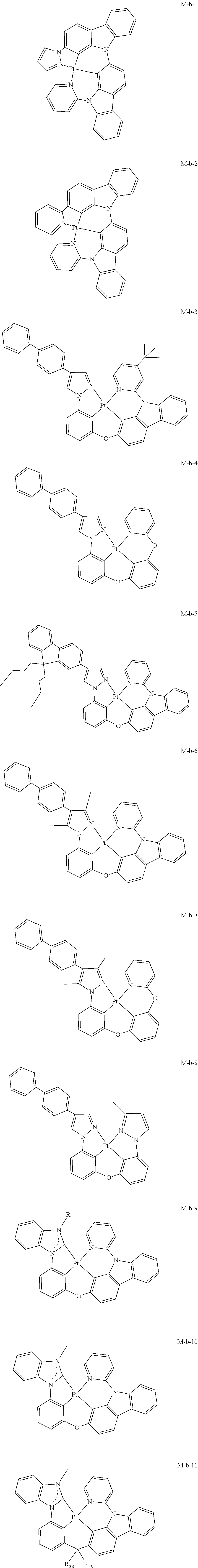

In Compounds M-b-1 to M-b-11, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML, may include a compound represented by any one of Formula F-a to Formula F-c. The compound represented by Formula F-a to Formula F-c may be used as a fluorescence dopant material.

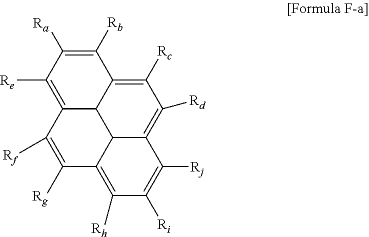

In Formula F-a, two of Rₐ to Rⱼ may each independently be substituted with a group represented by

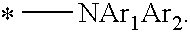

The remainder of Rₐ to Rⱼ may which are not substituted with the group represented by

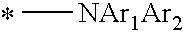

may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In the group represented by

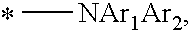

Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

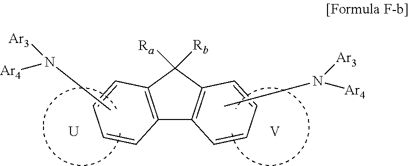

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, in Formula F-b, when the number of U or V is 1, a condensed ring may be present at a portion indicated by U or V, and when the number of U or V is 0, a condensed ring may not be present at the portion indicated by U or V. When the number of U is 0 and the number of V is 1, or when the number of U is 1 and the number of V is 0, a condensed ring having a fluorene core in Formula F-b may be a polycyclic compound having four rings. When the number of U and V is each 0, a condensed ring having a fluorene core of Formula F-b may be a polycyclic compound having three rings. When the number of U and V is each 1, a condensed ring having a fluorene core of Formula F-b may be a polycyclic compound having five rings.

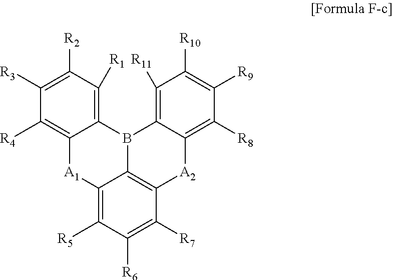

In Formula F-c, A₁ and A₂ may each independently be O, S, Se, or N(Rₘ); and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-c, A₁ and A₂ may each independently be bonded to substituents of an adjacent ring to form a condensed ring. For example, when A₁ and A₂ are each independently N(Rₘ), A₁ may be bonded to R₄ or R₅ to form a ring. For example, A₂ may be bonded to R₇ or R₈ to form a ring.

In an embodiment, the emission layer EML may include, as a dopant material of the related art, a styryl derivative (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzena mine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1-dipyrene, 1,4-dipyrenylbenzene, 1,4-bis(N,N-diphenylamino)pyrene), etc.

The emission layer EML may include a phosphorescence dopant material of the related art. For example, a metal complex including iridium (Ir), platinum (Pt), osmium (Os), aurum (Au), titanium (Ti), zirconium (Zr), hafnium (Hf), europium (Eu), terbium (Tb), or thulium (Tm) may be used as a phosphorescence dopant. For example, iridium(III) bis(4,6-difluorophenylpyridinato-N,C2′) (FIrpic), bis(2,4-difluorophenylpyridinato)-tetrakis(1-pyrazolyl)borate iridium(III) (Fir6), or platinum octaethyl porphyrin (PtOEP) may be used as a phosphorescence dopant. However, embodiments are not limited thereto.

The emission layer EML may include a quantum dot material. The quantum dot may be selected from a Group II-VI compound, a Group III-VI compound, a Group compound, a Group III-V compound, a Group III-II-V compound, a Group IV-VI compound, a Group IV element, a Group IV compound, or a combination thereof.

The Group II-VI compound may be selected from: a binary compound selected from the group consisting of CdSe, CdTe, CdS, ZnS, ZnSe, ZnTe, ZnO, HgS, HgSe, HgTe, MgSe, MgS, and a mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof; a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof; or any combination thereof.

The Group III-VI compound may be selected from: a binary compound such as In₂S₃ and In₂Se₃; a ternary compound such as InGaS₃ and InGaSe₃; or any combination thereof.

The Group compound may be selected from: a ternary compound selected from the group consisting of AgInS, AgInS₂, CuInS, CuInS₂, AgGaS₂, CuGaS₂ CuGaO₂, AgGaO₂, AgAlO₂, and a mixture thereof; a quaternary compound such as AgInGaS₂ and CuInGaS₂; or any combination thereof.

The Group III-V compound may be selected from: a binary compound selected from the group consisting of GaN, GaP, GaAs, GaSb, AlN, AlP, AlAs, AlSb, InN, InP, InAs, InSb, and a mixture thereof; a ternary compound selected from the group consisting of GaNP, GaNAs, GaNSb, GaPAs, GaPSb, AlNP, AINAs, AlNSb, AlPAs, AlPSb, InGaP, InAlP, InNP, InNAs, InNSb, InPAs, InPSb, and a mixture thereof; a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof; or any combination thereof. The Group III-V compound may further include a Group II metal. For example, InZnP, etc. may be selected as a Group III-II-V compound.

The Group IV-VI compound may be selected from: a binary compound selected from the group consisting of SnS, SnSe, SnTe, PbS, PbSe, PbTe, and a mixture thereof; a ternary compound selected from the group consisting of SnSeS, SnSeTe, SnSTe, PbSeS, PbSeTe, PbSTe, SnPbS, SnPbSe, SnPbTe, and a mixture thereof; a quaternary compound selected from the group consisting of SnPbSSe, SnPbSeTe, SnPbSTe, and a mixture thereof; or any combination thereof. The Group IV element may be selected from the group consisting of Si, Ge, and a mixture thereof. The Group IV compound may be a binary compound selected from the group consisting of SiC, SiGe, and a mixture thereof.

A binary compound, a ternary compound, or a quaternary compound may be present in a particle at a uniform concentration distribution, or may be present in a particle at a partially different concentration distribution. In an embodiment, the quantum dot may have a core/shell structure in which a quantum dot surrounds another quantum dot. A quantum dot having a core/shell structure may have a concentration gradient in which the concentration of an element that is present in the shell decreases toward the core.

In embodiments, a quantum dot may have the above-described core-shell structure including a core containing nanocrystals and a shell surrounding the core. The shell of the quantum dot may serve as a protection layer to prevent the chemical deformation of the core so as to maintain semiconductor properties, and/or may serve as a charging layer to impart electrophoresis properties to the quantum dot. The shell may be a single layer or a multilayer. An example of the shell of the quantum dot may include a metal oxide, a non-metal oxide, a semiconductor compound, or a combination thereof.

Examples of the metal oxide or the non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and NiO, or a ternary compound such as MgAl₂O₄, CoFe₂O₄, NiFe₂O₄, and CoMn₂O₄, but embodiments are not limited thereto.

Examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width of half maximum (FWHM) of a light emission wavelength spectrum equal to or less than about 45 nm. For example, the quantum dot may have a FWHM of a light emission wavelength spectrum equal to or less than about 40 nm. For example, the quantum dot may have a FWHM of a light emission wavelength spectrum equal to or less than about 30 nm. Color purity or color reproducibility may be improved in the above ranges. Light emitted through a quantum dot may be emitted in all directions, so that a wide viewing angle may be improved.

The form of a quantum dot is not limited and may be any form that is used in the related art. For example, the quantum dot may have a spherical shape, a pyramidal shape, a multi-arm shape, or a cubic shape, or the quantum dot may be in the form of nanoparticles, nanotubes, nanowires, nanofibers, nanoparticles, etc.

The quantum dot may control the color of emitted light according to a particle size thereof, and accordingly, the quantum dot may have various emission colors such as blue, red, or green.

In the light emitting device ED according to embodiments illustrated in FIGS. 3 to 6, the electron transport region ETR is provided on the emission layer EML. The electron transport region ETR may include at least one of a hole blocking layer HBL, an electron transport layer ETL, or an electron injection layer EIL, but embodiments are not limited thereto.

The electron transport region ETR may be a layer formed of a single material, a layer formed of different materials, or a structure including multiple layers formed of different materials.

For example, the electron transport region ETR may have a single layer structure of the electron injection layer EIL or the electron transport layer ETL, or may have a single layer structure formed of an electron injection material and an electron transport material. In other embodiments, the electron transport region ETR may have a single layer structure formed of different materials, or may have a structure in which an electron transport layer ETL/electron injection layer EIL, or a hole blocking layer HBL/electron transport layer ETL/electron injection layer EIL are stacked in its respective stated order from the emission layer EML, but embodiments are not limited thereto. The electron transport region ETR may have a thickness, for example, in a range of about 1,000 Å to about 1,500 Å.

The electron transport region ETR may be formed by using various methods such as a vacuum deposition method, a spin coating method, a cast method, a Langmuir-Blodgett (LB) method, an inkjet printing method, a laser printing method, a laser induced thermal imaging (LITI) method, etc.

The electron transport region ETR may include a compound represented by Formula ET-1:

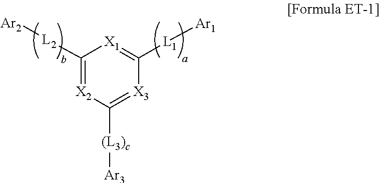

In Formula ET-1, at least one of X₁ to X₃ may be N, and the remainder of X₁ to X₃ may be C(Rₐ). In Formula ET-1, Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula ET-1, Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. When a to c are 2 or more, multiple groups of L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-Bis[3,5-di(pyridin-3-yl)phenyl]benzene (BmPyPhB), or a mixture thereof.

The electron transport region ETR may include at least one of Compound ET1 to Compound ET36:

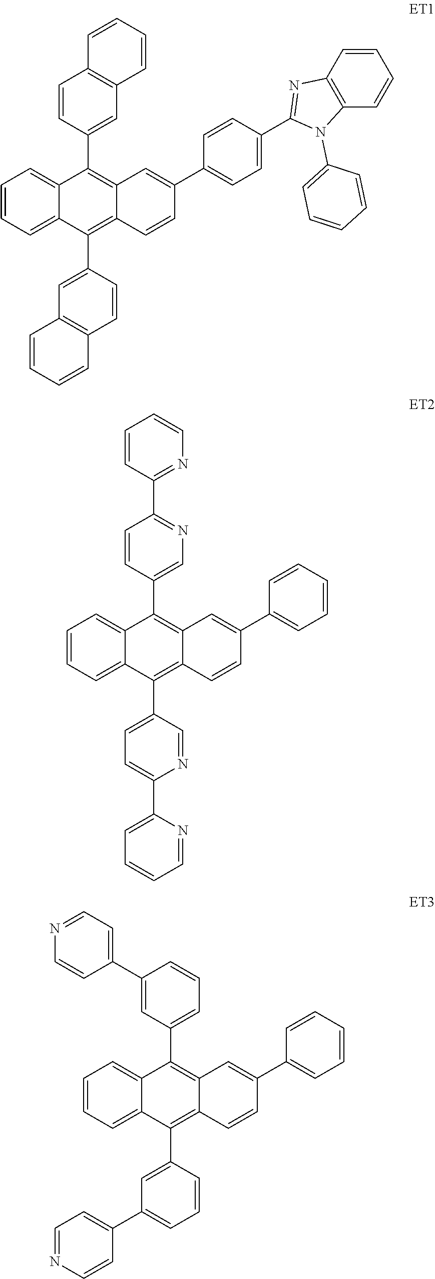 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

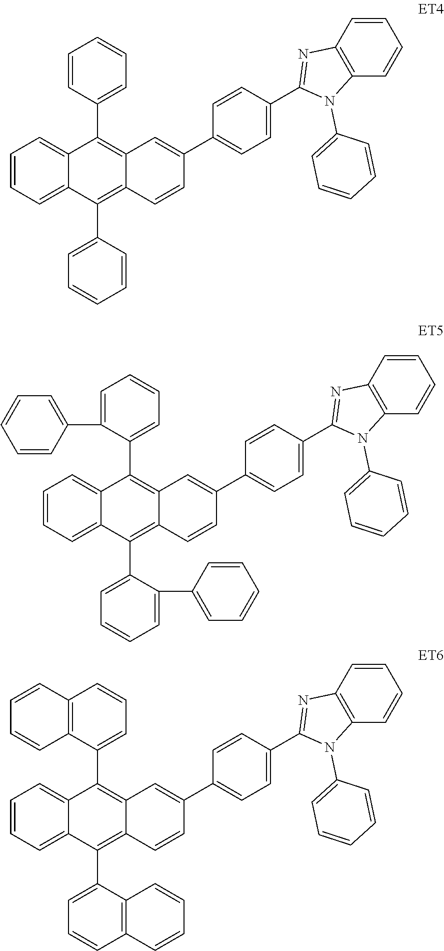 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

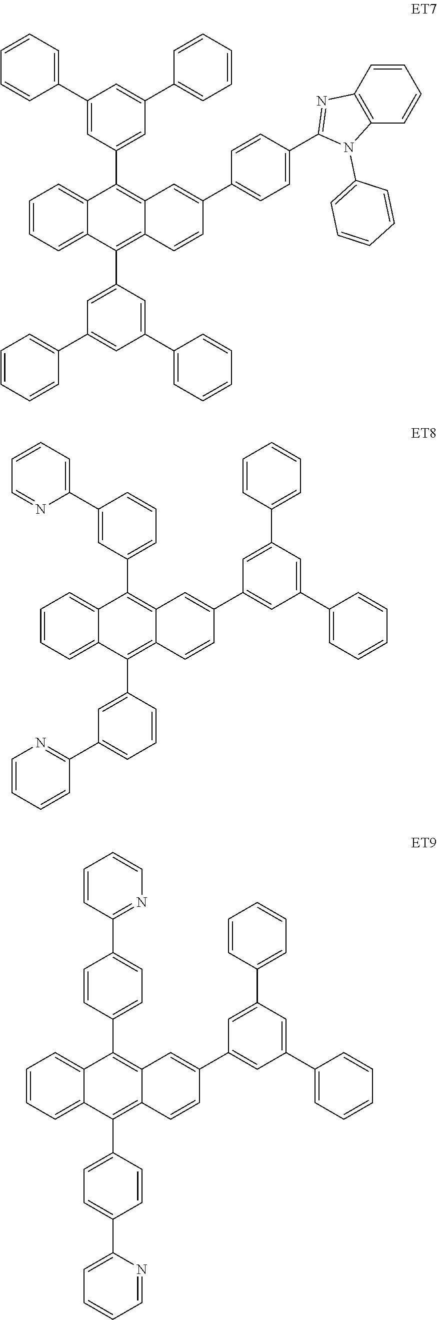

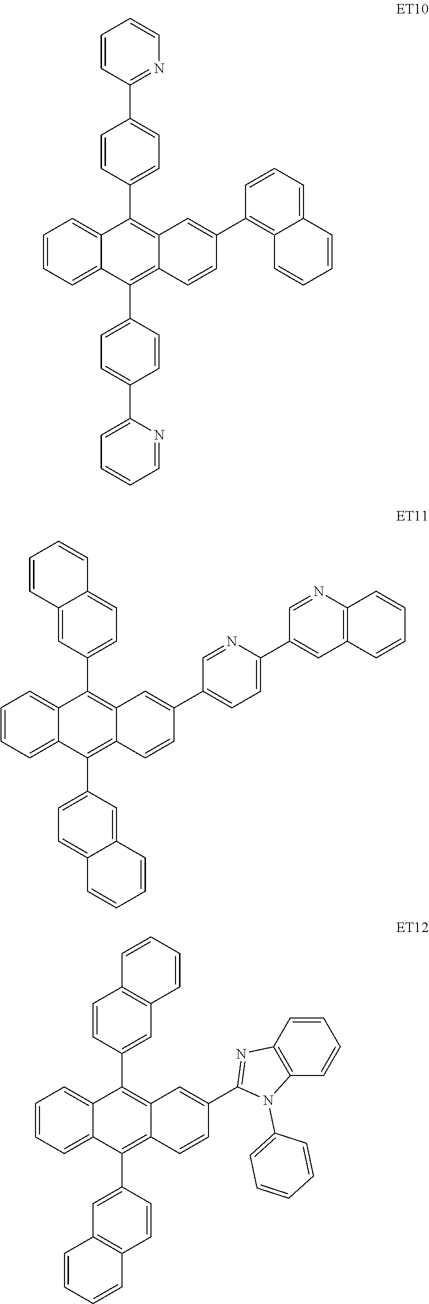

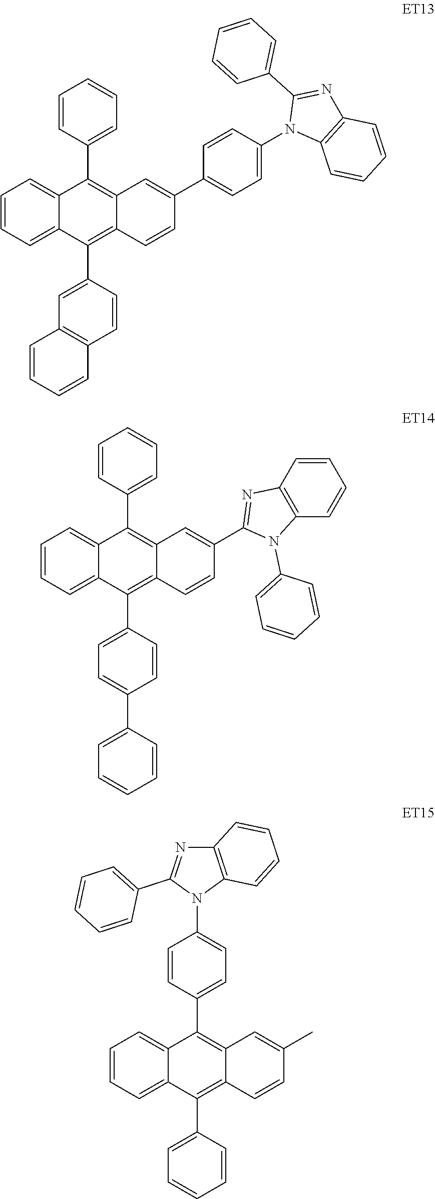 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

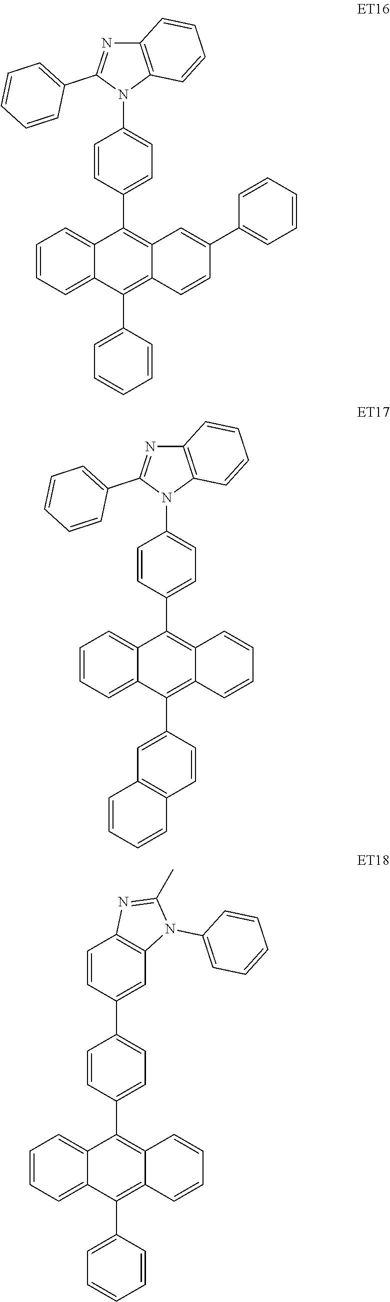

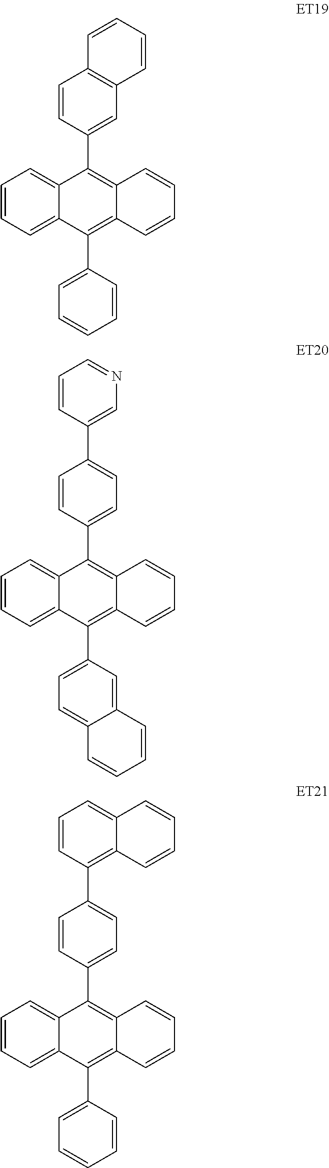

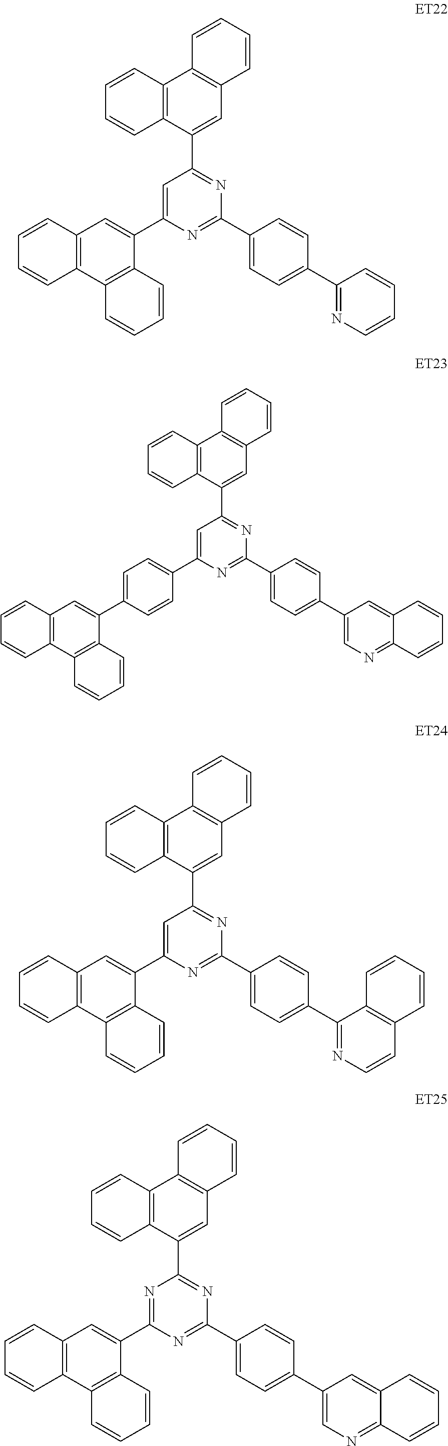 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

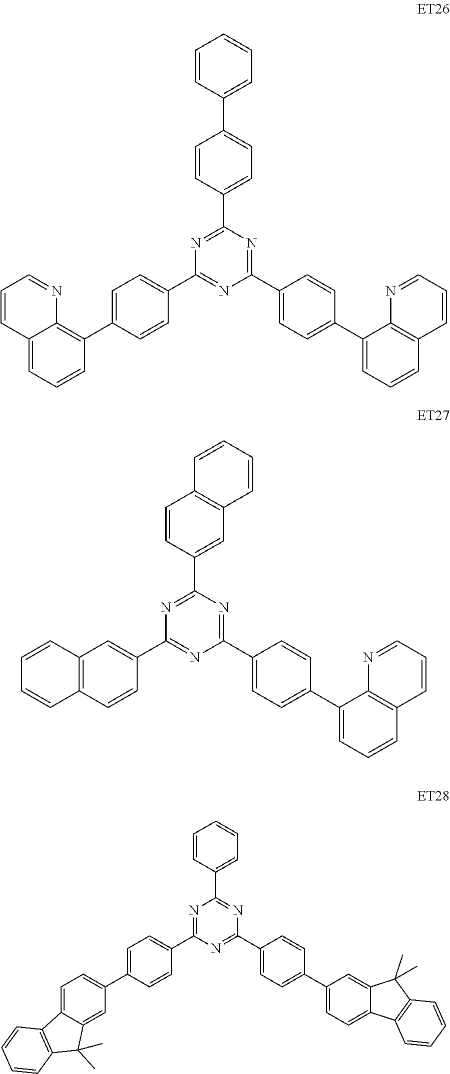 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

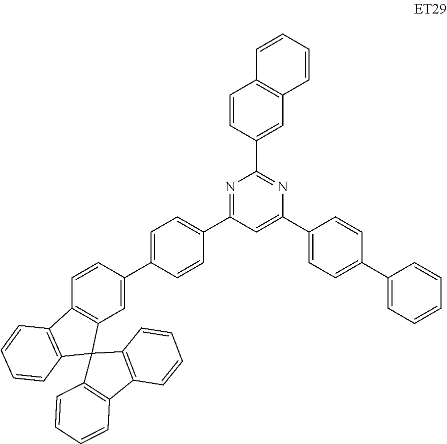 [SMILES: c1ccc(-c2ccc(-c3cc(-c4ccc(-c5ccc6c(c5)C5(c7ccccc7-c7ccccc75)c5ccccc5-6)cc4)nc(-c4ccc5ccccc5c4)n3)cc2)cc1]

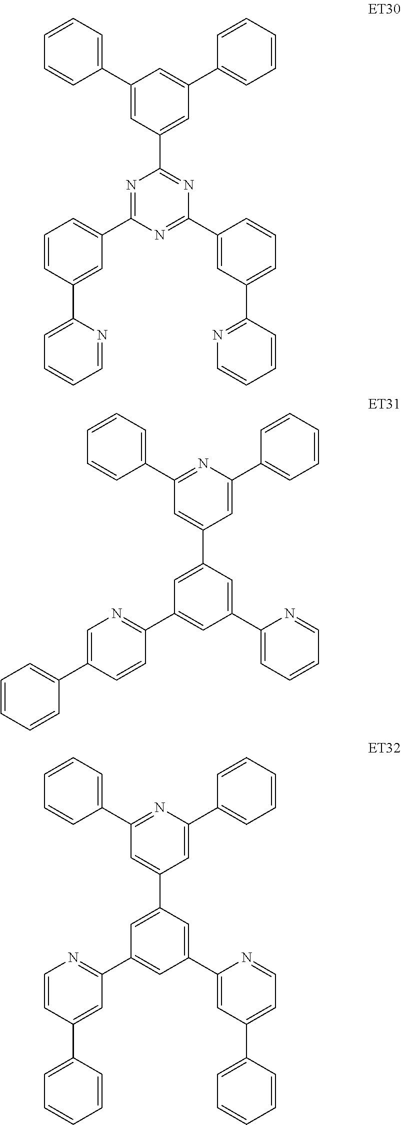

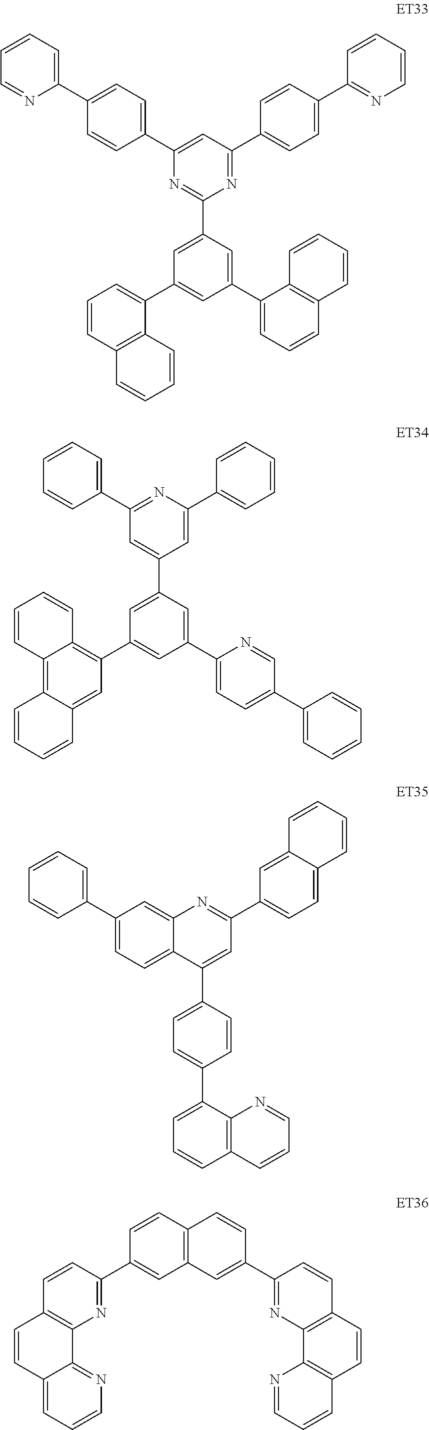

The electron transport region ETR may include a metal halide such as LiF, NaCl, CsF, RbCl, RbI, CuI, or KI; a lanthanide metal such as Yb; or a co-deposited material of the metal halide and the lanthanide metal. For example, the electron transport region ETR may include KI:Yb, RbI:Yb, LiF:Yb, etc. as a co-deposited material. The electron transport region ETR may be formed of a metal oxide such as Li₂O or BaO, or 8-hydroxyl-lithium quinolate (Liq), etc., but embodiments are not limited thereto. The electron transport region ETR may also be formed of a mixture material of an electron transport material and an insulating organometallic salt. The organometallic salt may be a material having an energy band gap equal to or greater than about 4 eV. For example, the organometallic salt may include a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenyl silyl)phenyl)phosphine oxide (TSPO1), or 4,7-diphenyl-1,10-phenanthroline (Bphen) in addition to the above-described materials, but embodiments are not limited thereto.

The electron transport region ETR may include the above-described compounds of the hole transport region in at least one of an electron injection layer EIL, an electron transport layer ETL, or a hole blocking layer HBL.

When the electron transport region ETR includes an electron transport layer ETL, the electron transport layer ETL may have a thickness in a range of about 100 Å to about 1,000 Å. For example, the electron transport layer ETL may have a thickness in a range of about 150 Å to about 500 Å. If the thickness of the electron transport layer ETL satisfies the aforementioned range, satisfactory electron transport characteristics may be obtained without a substantial increase in driving voltage. When the electron transport region ETR includes an electron injection layer EIL, the electron injection layer EIL may have a thickness in a range of about 1 Å to about 100 Å. For example, the electron injection layer EIL may have a thickness in a range of about 3 Å to about 90 Å. If the thickness of the electron injection layer EIL satisfies the above-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but embodiments are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may include a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgAg). In another embodiment, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, or the like.

Although not shown in the drawings, the second electrode EL2 may be electrically connected to an auxiliary electrode. If the second electrode EL2 is electrically connected to the auxiliary electrode, the resistance of the second electrode EL2 may decrease.

In an embodiment, the light emitting device ED may further include a capping layer CPL disposed on the second electrode EL2. The capping layer CPL may be a multilayer or a single layer.

In an embodiment, the capping layer CPL may include an organic layer or an inorganic layer. For example, when the capping layer CPL includes an inorganic material, the inorganic material may include an alkaline metal compound (for example, LiF), an alkaline earth metal compound (for example, MgF₂), SiON, SiNₓ, SiOy, etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol sol-9-yl)triphenylamine (TCTA), etc., or may include an epoxy resin, or an acrylate such as a methacrylate. However, embodiments are not limited thereto, and the capping layer CPL may include at least one of Compounds P1 to P5:

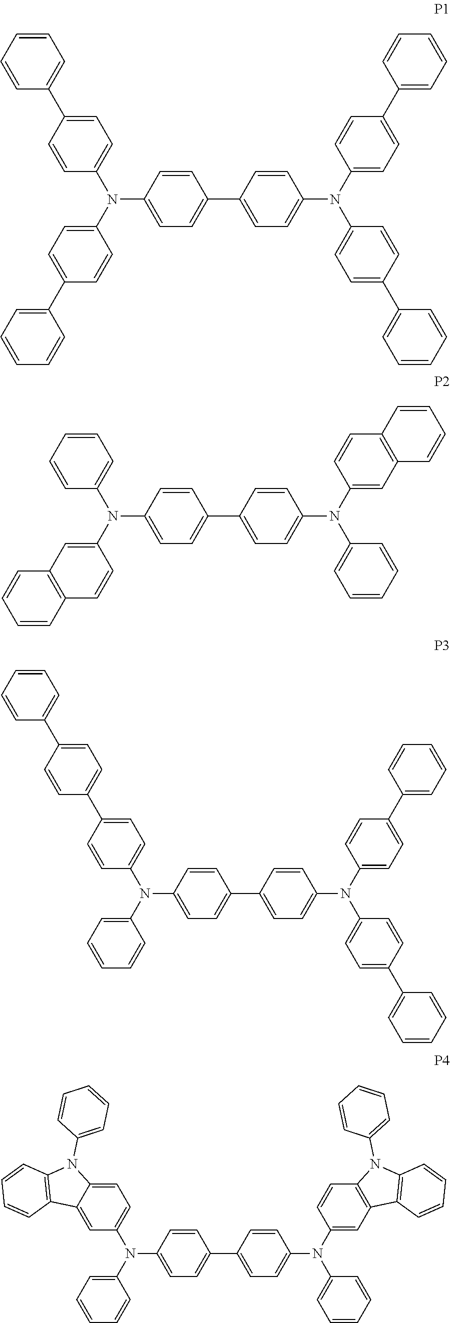 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

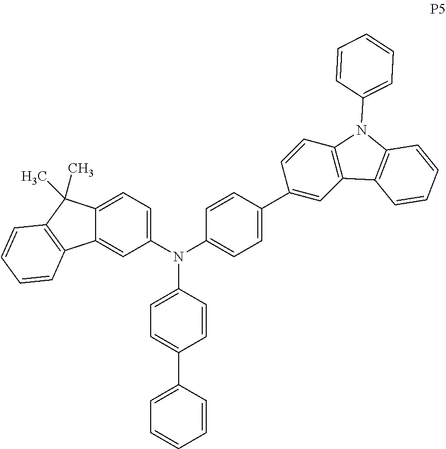 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6 with respect to light in a wavelength range of about 550 nm to about 660 nm.

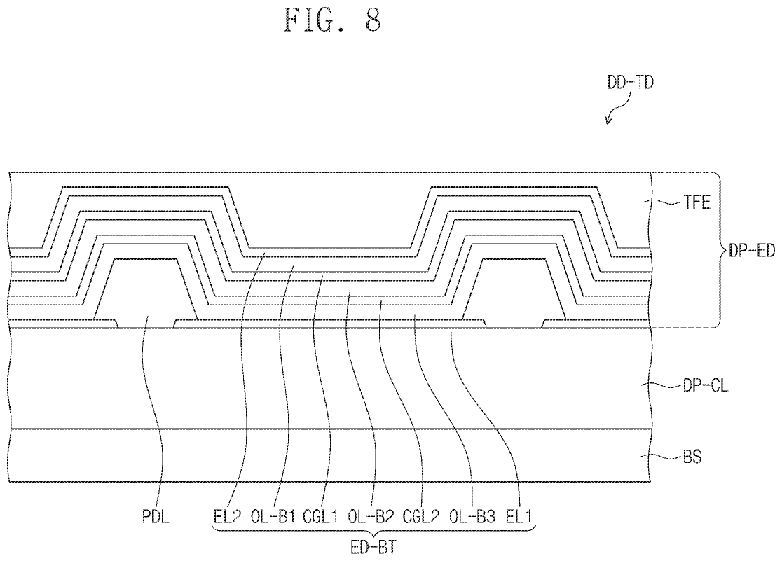

FIGS. 7 and 8 each are a schematic cross-sectional view of a display apparatus according to embodiments. In the description of the display apparatus according to embodiments with reference to FIGS. 7 and 8, the features which have been described with respect to FIGS. 1 to 6 will not be explained again, and the disclosure will describe the differing features.

Referring to FIG. 7, a display apparatus DD according to an embodiment may include a display panel DP including a display device layer DP-ED, a light control layer CCL disposed on the display panel DP, and a color filter layer CFL.

In an embodiment illustrated in FIG. 7, the display panel DP may include a base layer BS, a circuit layer DP-CL provided on the base layer BS, and the display device layer DP-ED, and the display device layer DP-ED may include a light emitting device ED.

The light emitting device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. A structure of the light emitting device ED shown in FIG. 7 may be the same as a structure of a light emitting device according to one of FIGS. 3 to 6.

Referring to FIG. 7, the emission layer EML may be disposed in openings OH defined in a pixel defining film PDL. For example, the emission layer EML which is separated by the pixel defining film PDL and provided corresponding to each light emitting regions PXA-R, PXA-G, and PXA-B, may emit light in a same wavelength range. In the display apparatus DD according to an embodiment, the emission layer EML may emit blue light. Although not shown in the drawings, in an embodiment, the emission layer EML may be provided as a common layer for all of the light emitting regions PXA-R, PXA-G, and PXA-B.

The light control layer CCL may be disposed on the display panel DP. The light control layer CCL may include a light conversion body. The light conversion body may be a quantum dot, a phosphor, or the like. The light conversion body may convert the wavelength of a provided light and may emit the resulting light. For example, the light control layer CCL may a layer containing a quantum dot or a layer containing a phosphor.

The light control layer CCL may include light control parts CCP1, CCP2, and CCP3. The light control parts CCP1, CCP2, and CCP3 may be spaced apart from one another.

Referring to FIG. 7, divided patterns BMP may be disposed between the light control parts CCP1, CCP2, and CCP3 which are spaced apart from each other, but embodiments are not limited thereto. FIG. 7 illustrates that the divided patterns BMP do not overlap the light control parts CCP1, CCP2, and CCP3, but at least a portion of the edges of the light control parts CCP1, CCP2, and CCP3 may overlap the divided patterns BMP.

The light control layer CCL may include a first light control part CCP1 containing a first quantum dot QD1 which converts first color light provided from the light emitting device ED into second color light, a second light control part CCP2 containing a second quantum dot QD2 which converts the first color light into third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting device ED. For example, the first quantum dot QD1 may be a red quantum dot, and the second quantum dot QD2 may be a green quantum dot. The same descriptions of quantum dots as provided above may be applied to the quantum dots QD1 and QD2.

The light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include any quantum dot but may include the scatterer SP.

The scatterer SP may be inorganic particles. For example, the scatterer SP may include at least one of TiO₂, ZnO, Al₂O₃, SiO₂, or hollow silica. The scatterer SP may include any one of TiO₂, ZnO, Al₂O₃, SiO₂, or hollow silica, or the scatterer SP may be a mixture of at least two materials selected from TiO₂, ZnO, Al₂O₃, SiO₂, and hollow silica.

The first light control part CCP1, the second light control part CCP2, and the third light control part CCP3 may each include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 may each be a medium in which the quantum dots QD1 and QD2 and the scatterer SP are dispersed, and may be formed of various resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 each may be the same as or different from each other.

The light control layer CCL may include a barrier layer BFL1. The barrier layer BFL1 may prevent the penetration of moisture and/or oxygen (hereinafter, referred to as ‘moisture/oxygen’). The barrier layer BFL1 may be disposed on the light control parts CCP1, CCP2, and CCP3 to block the light control parts CCP1, CCP2, and CCP3 from being exposed to moisture/oxygen. The barrier layer BFL1 may cover the light control parts CCP1, CCP2, and CCP3. A barrier layer BFL2 may be provided between the light control parts CCP1, CCP2, and CCP3 and the color filter layer CFL.

The barrier layers BFL1 and BFL2 may each independently include at least one inorganic layer. For example, the barrier layers BFL1 and BFL2 may each include an inorganic material. For example, the barrier layers BFL1 and BFL2 may each independently include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. The barrier layers BFL1 and BFL2 may further include an organic film. The barrier layers BFL1 and BFL2 may be formed of a single layer or of multiple layers.

In the display apparatus DD according to an embodiment, the color filter layer CFL may be disposed on the light control layer CCL. In an embodiment, the color filter layer CFL may be directly disposed on the light control layer CCL. For example, the barrier layer BFL2 may be omitted.

The color filter layer CFL may include a light shielding part BM and color filters CF1, CF2, and CF3. The color filter layer CFL may include a first filter CF1 that transmits the second color light, a second filter CF2 that transmits the third color light, and a third filter CF3 that transmits the first color light. For example, the first filter CF1 may be a red filter, the second filter CF2 may be a green filter, and the third filter CF3 may be a blue filter. The filters CF1, CF2, and CF3 may each include a polymeric photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include a pigment or dye. The third filter CF3 may include a polymeric photosensitive resin and may not include a pigment or a dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may not be separated but may be provided as one filter.

The light shielding part BM may be a black matrix. The light shielding part BM may include an organic light shielding material or an inorganic light shielding material containing a black pigment or dye. The light shielding part BM may prevent light leakage, and may separate boundaries between the adjacent filters CF1, CF2, and CF3. In an embodiment, the light shielding part BM may be formed of a blue filter.

The first to third filters CF1, CF2, and CF3 may be respectively disposed corresponding to the red light emitting region PXA-R, the green light emitting region PXA-G, and the blue light emitting region PXA-B.

A base substrate BL may be disposed on the color filter layer CFL. The base substrate BL may provide a base surface on which the color filter layer CFL, the light control layer CCL, and the like are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

FIG. 8 is a schematic cross-sectional view illustrating a part of a display apparatus according to an embodiment. FIG. 8 illustrates a schematic cross-sectional view of a part corresponding to the display panel DP of FIG. 7. In the display apparatus DD-TD according to an embodiment, the light emitting device ED-BT may include light emitting structures OL-B1, OL-B2, and OL-B3. The light emitting device ED-BT may include a first electrode EL1 and a second electrode EL2 which face each other, and the light emitting structures OL-B1, OL-B2, and OL-B3 stacked in a thickness direction between the first electrode EL1 and the second electrode EL2. The light emitting structures OL-B1, OL-B2, and OL-B3 may each include an emission layer EML (FIG. 7) and a hole transport region HTR and an electron transport region ETR disposed with the emission layer EML (FIG. 7) therebetween.

For example, the light emitting device ED-BT included in the display apparatus DD-TD according to an embodiment may be a light emitting device having a tandem structure and including multiple emission layers.

In an embodiment illustrated in FIG. 8, light emitted from each of the light emitting structures OL-B1, OL-B2, and OL-B3 may be blue light. However, embodiments are not limited thereto, and the light emitted from the light emitting structures OL-B1, OL-B2, and OL-B3 may have wavelength ranges different from each other. For example, the light emitting device ED-BT including the light emitting structures OL-B1, OL-B2, and OL-B3 which emit light having wavelength ranges different from each other may emit white light.

Charge generation layers CGL1 and CGL2 may be disposed between neighboring light emitting structures OL-B1, OL-B2, and OL-B3. The charge generation layers CGL1 and CGL2 may each independently include a p-type charge generation layer and/or an n-type charge generation layer.

Hereinafter, an amine compound according to an embodiment and a light emitting device according to an embodiment will be described with reference to the Examples and the Comparative Examples. The Examples described below are only provided as illustrations to assist in understanding the disclosure, and the scope thereof is not limited thereto.

### EXAMPLES

1. Synthesis of Amine Compound

A synthesis method of an amine compound according to embodiments will be described by describing the synthesis methods of Compounds 1, 17, 18, 19, 21, 96, 98, 106, 133, 189, 191, 282, 284, 424, 425, 449, and 461. The synthesis methods of the amine compound explained in the following description are provided only as examples, but the synthesis method according to embodiments is not limited to the Examples below.

<Synthetic Method of Compounds>

In an argon atmosphere, Intermediate Compound P (10.0 mmol), Intermediate Compound Q (11.0 mmol), Pd(dba)₂ (0.058 g, 0.10 mmol), P(tBu)₃*HBF₄ (0.117 g, 0.40 mmol), and NaOtBu (1.16 g, 12.0 mmol) were added in a 200 mL three-neck flask, and stirred in 50 mL of toluene solvent at about 110° C. for about 8 hours. The stirred mixture was cooled, and cleaned with water to separate an organic layer. The separated organic layer was purified by silica gel column chromatography to obtain target compounds. Table 2 shows the structure and added mass of Intermediate Compound P, the structure and added mass of Intermediate Compound Q, and the mass, yield, and FAB-MS of the resulting target compounds. Table 3 shows H-NMR of the target compounds.

TABLE 2

| | | Used | | Used | | |
|---|---|---|---|---|---|---|
| Target | Intermediate | amount | Intermediate | amount | | |
| compound | Compound P | (mass) | Compound Q | (mass) | Yield | FAB-MS |
| a01 (282) | 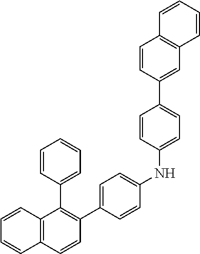 [SMILES: c1ccc(-c2c(-c3ccc(Nc4ccc(-c5ccc6ccccc6c5)cc4)cc3)ccc3ccccc23)cc1] | 4.98 g | 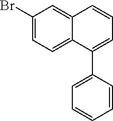 [SMILES: Brc1ccc2c(-c3ccccc3)cccc2c1] | 3.11 g | 91% | 699 |
| a02 (189) | 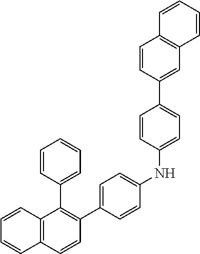 [SMILES: c1ccc(-c2c(-c3ccc(Nc4ccc(-c5ccc6ccccc6c5)cc4)cc3)ccc3ccccc23)cc1] | 4.98 g | 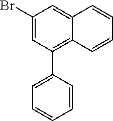 [SMILES: Brc1cc(-c2ccccc2)c2ccccc2c1] | 3.11 g | 92% | 699 |
| a03 (96) | 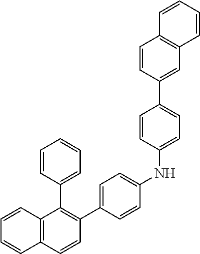 [SMILES: c1ccc(-c2c(-c3ccc(Nc4ccc(-c5ccc6ccccc6c5)cc4)cc3)ccc3ccccc23)cc1] | 4.98 g | 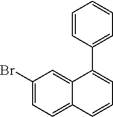 [SMILES: Brc1ccc2cccc(-c3ccccc3)c2c1] | 3.11 g | 89% | 699 |
| a04 (284) | 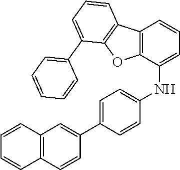 [SMILES: c1ccc(-c2cccc3c2oc2c(Nc4ccc(-c5ccc6ccccc6c5)cc4)cccc23)cc1] | 4.62 g | 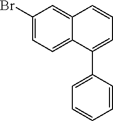 [SMILES: Brc1ccc2c(-c3ccccc3)cccc2c1] | 3.11 g | 93% | 663 |
| a05 (191) | 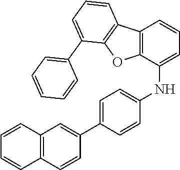 [SMILES: c1ccc(-c2cccc3c2oc2c(Nc4ccc(-c5ccc6ccccc6c5)cc4)cccc23)cc1] | 4.62 g | 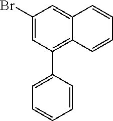 [SMILES: Brc1cc(-c2ccccc2)c2ccccc2c1] | 3.11 g | 96% | 663 |
| a06 (98) | 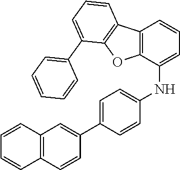 [SMILES: c1ccc(-c2cccc3c2oc2c(Nc4ccc(-c5ccc6ccccc6c5)cc4)cccc23)cc1] | 4.62 g | 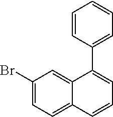 [SMILES: Brc1ccc2cccc(-c3ccccc3)c2c1] | 3.11 g | 85% | 663 |
| a07 (1) | 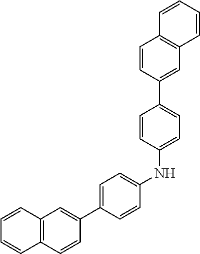 [SMILES: c1ccc2cc(-c3ccc(Nc4ccc(-c5ccc6ccccc6c5)cc4)cc3)ccc2c1] | 4.22 g | 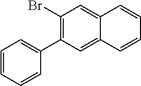 [SMILES: Brc1cc2ccccc2cc1-c1ccccc1] | 3.11 g | 59% | 623 |
| a08 (466) | 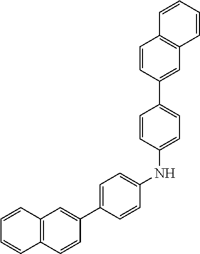 [SMILES: c1ccc2cc(-c3ccc(Nc4ccc(-c5ccc6ccccc6c5)cc4)cc3)ccc2c1] | 4.22 g | 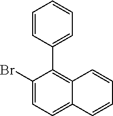 [SMILES: Brc1ccc2ccccc2c1-c1ccccc1] | 3.11 g | 64% | 623 |
| a09 (425) | 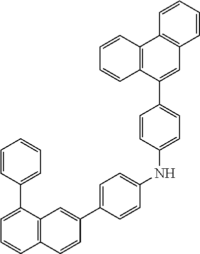 [SMILES: c1ccc(-c2cccc3ccc(-c4ccc(Nc5ccc(-c6cc7ccccc7c7ccccc67)cc5)cc4)cc23)cc1] | 5.48 g | 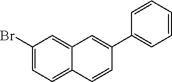 [SMILES: Brc1ccc2ccc(-c3ccccc3)cc2c1] | 3.11 g | 79% | 749 |
| a10 (424) | 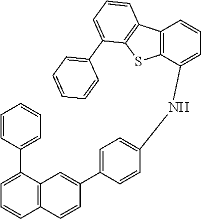 [SMILES: c1ccc(-c2cccc3ccc(-c4ccc(Nc5cccc6c5sc5c(-c7ccccc7)cccc56)cc4)cc23)cc1] | 5.54 g | 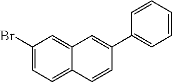 [SMILES: Brc1ccc2ccc(-c3ccccc3)cc2c1] | 3.11 g | 82% | 755 |
| a11 (17) | 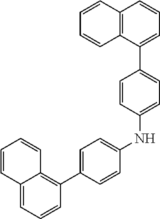 [SMILES: c1ccc2c(-c3ccc(Nc4ccc(-c5cccc6ccccc56)cc4)cc3)cccc2c1] | 4.22 g | 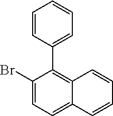 [SMILES: Brc1ccc2ccccc2c1-c1ccccc1] | 3.11 g | 52% | 623 |
| a12 (449) | 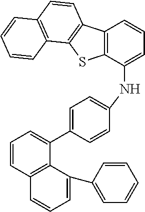 [SMILES: c1ccc(-c2cccc3cccc(-c4ccc(Nc5cccc6c5sc5c7ccccc7ccc65)cc4)c23)cc1] | 5.28 g | 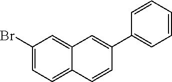 [SMILES: Brc1ccc2ccc(-c3ccccc3)cc2c1] | 3.11 g | 77% | 729 |
| a13 (461) | 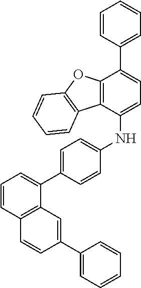 [SMILES: c1ccc(-c2ccc3cccc(-c4ccc(Nc5ccc(-c6ccccc6)c6oc7ccccc7c56)cc4)c3c2)cc1] | 5.38 g | 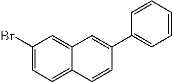 [SMILES: Brc1ccc2ccc(-c3ccccc3)cc2c1] | 3.11 g | 69% | 739 |
| a14 (19) | 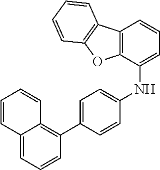 [SMILES: c1ccc2c(-c3ccc(Nc4cccc5c4oc4ccccc45)cc3)cccc2c1] | 3.85 g | 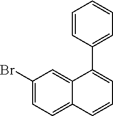 [SMILES: Brc1ccc2cccc(-c3ccccc3)c2c1] | 3.11 g | 50% | 587 |
| a15 (21) | 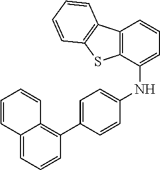 [SMILES: c1ccc2c(-c3ccc(Nc4cccc5c4sc4ccccc45)cc3)cccc2c1] | 4.02 g | 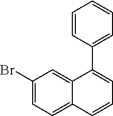 [SMILES: Brc1ccc2cccc(-c3ccccc3)c2c1] | 3.′11 g | 49% | 603 |
| a16 (133) | 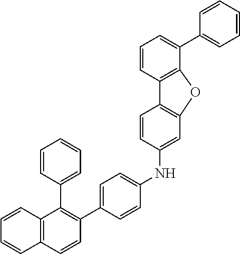 [SMILES: c1ccc(-c2c(-c3ccc(Nc4ccc5c(c4)oc4c(-c6ccccc6)cccc45)cc3)ccc3ccccc23)cc1] | 5.38 g | 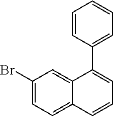 [SMILES: Brc1ccc2cccc(-c3ccccc3)c2c1] | 3.11 g | 60% | 739 |
| a17 (106) | 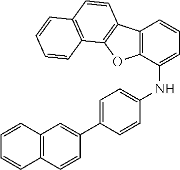 [SMILES: c1ccc2cc(-c3ccc(Nc4cccc5c4oc4c6ccccc6ccc54)cc3)ccc2c1] | 4.35 g | 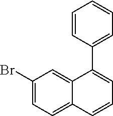 [SMILES: Brc1ccc2cccc(-c3ccccc3)c2c1] | 3.11 g | 89% | 637 |

TABLE 3

| Target | |
|---|---|
| compound | H-NMR |
| a01 | 8.03(1H), 7.93-7.84(3H), 7.81-7.72(4H), |
| (282) | 7.61-7.18(28H), 6.98(1H) |
| a02 | 8.05-8.00(2H), 7.93-7.84(2H), 7.81-7.67(4H), |
| (189) | 7.61-7.17(27H), 7.04-6.95(2H) |
| a03 | 8.01(1H), 7.97-7.69(9H), 7.65-7.57(4H), |
| (96) | 7.54-7.12(19H), 7.05-6.94(4H) |
| a04 | 7.91-7.72(7H), 7.61-7.51(3H), 7.46-7.15(21H), |
| (284) | 7.09-6.96(2H) |
| a05 | 8.02(1H), 7.91-7.77(6H), 7.69(2H), |
| (191) | 7.56-7.16(21H), 7.04-6.95(3H) |
| a06 | 7.91-7.77(7H), 7.68-7.57(3H), 7.49-7.16(21H), |
| (98) | 7.09-6.99(2H) |
| a07 | 7.91-7.77(6H), 7.68-7.55(2H), 7.52-7.14(23H), |
| (1) | 6.98(2H) |
| a08 | 8.00(1H), 7.91-7.67(8H), 7.56(1H), |
| (466) | 7.45-7.15(21H), 7.07-6.98(2H) |
| a09 | 8.20-8.12(2H), 7.94-7.78(7H), 7.71-7.15(28H), |
| (425) | 7.11-7.07(2H) |
| a10 | 8.07-7.90(4H), 7.87-7.70(3H), 7.61-7.15(28H), |
| (424) | 7.11-7.07(2H) |
| a11 | 8.17-8.08(3H), 8.00(2H), 7.92-7.54(9H), |
| (17) | 7.47-7.15(17H), 7.07-6.98(2H) |
| a12 | 8.05-8.01(2H), 7.98-7.79(6H), 7.65-7.53(6H), |
| (449) | 7.46-7.15(19H), 7.01-6.97(2H) |
| a13 | 8.04-8.00(2H), 7.97-7.86(3H), 7.76-7.63(7H), |
| (461) | 7.55-7.09(22H), 7.01-6.87(3H) |
| a14 | 8.07-7.98(2H), 7.90(1H), 7.82-7.54(7H), |
| (19) | 7.46-7.08(18H), 6.97(1H) |
| a15 | 8.07-7.98(2H), 7.92-7.83(2H), 7.72-7.54(8H), |
| (21) | 7.48-7.15(15H), 7.07-6.98(2H) |
| a16 | 8.13(1H), 8.03-7.91(3H), 7.90-7.78(4H), |
| (133) | 7.68-7.15(27H), 7.03-6.99(2H) |
| a17 | 8.04-8.00(2H), 7.94-7.74(10H), 7.66(2H), |
| (106) | 7.59(1H), 7.53-7.28(10H), 7.20-6.90(6H) |

### Example Compounds

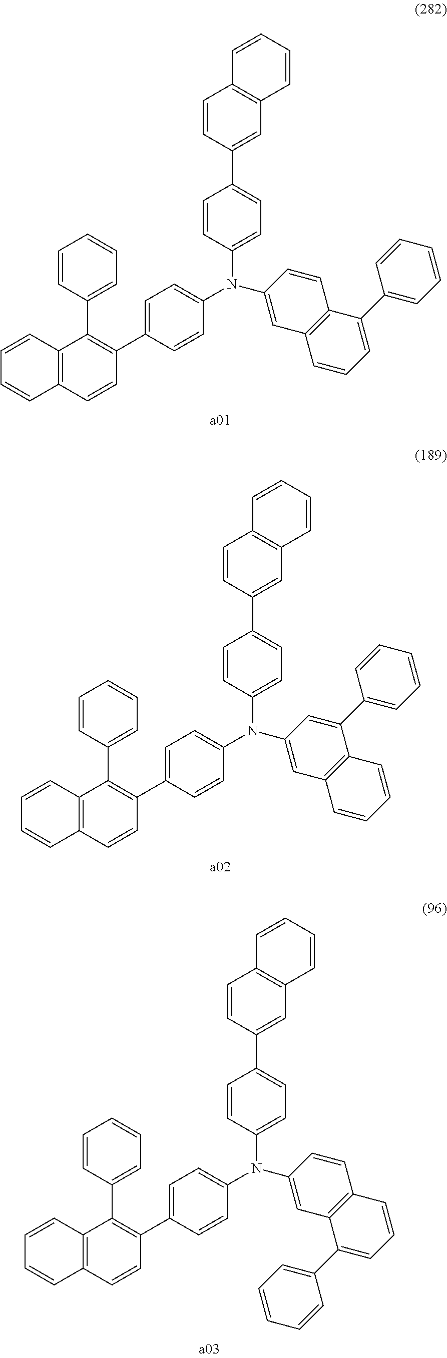 [SMILES: c1ccc(-c2cccc3cc(N(c4ccc(-c5ccc6ccccc6c5)cc4)c4ccc(-c5ccc6ccccc6c5-c5ccccc5)cc4)ccc23)cc1 ; c1ccc(-c2cc(N(c3ccc(-c4ccc5ccccc5c4)cc3)c3ccc(-c4ccc5ccccc5c4-c4ccccc4)cc3)cc3ccccc23)cc1 ; c1ccc(-c2cccc3ccc(N(c4ccc(-c5ccc6ccccc6c5)cc4)c4ccc(-c5ccc6ccccc6c5-c5ccccc5)cc4)cc23)cc1]

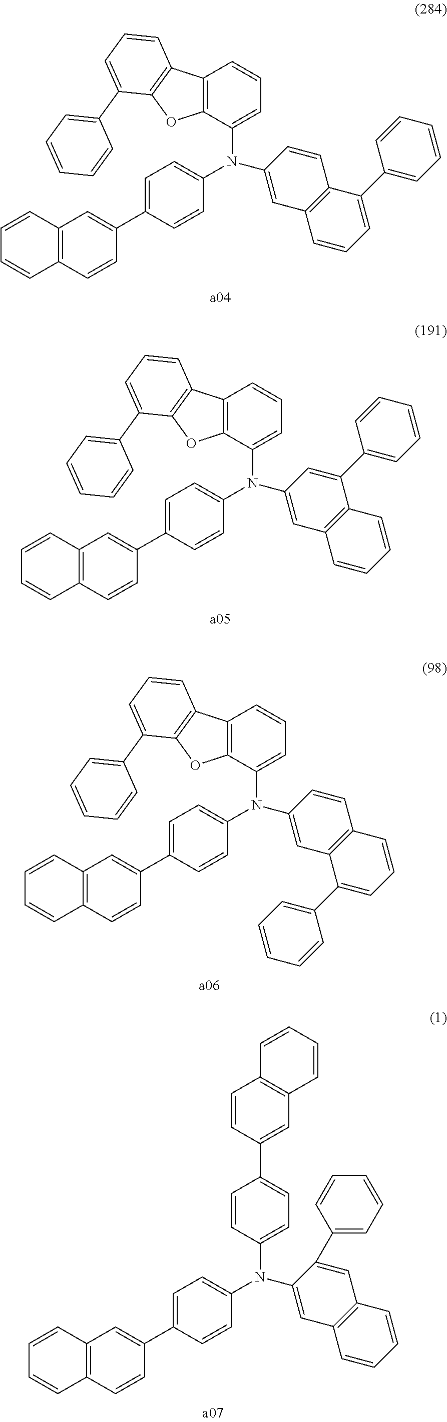 [SMILES: c1ccc(-c2cccc3cc(N(c4ccc(-c5ccc6ccccc6c5)cc4)c4cccc5c4oc4c(-c6ccccc6)cccc45)ccc23)cc1 ; c1ccc(-c2cc(N(c3ccc(-c4ccc5ccccc5c4)cc3)c3cccc4c3oc3c(-c5ccccc5)cccc34)cc3ccccc23)cc1 ; c1ccc(-c2cccc3ccc(N(c4ccc(-c5ccc6ccccc6c5)cc4)c4cccc5c4oc4c(-c6ccccc6)cccc45)cc23)cc1 ; c1ccc(-c2cc3ccccc3cc2N(c2ccc(-c3ccc4ccccc4c3)cc2)c2ccc(-c3ccc4ccccc4c3)cc2)cc1]

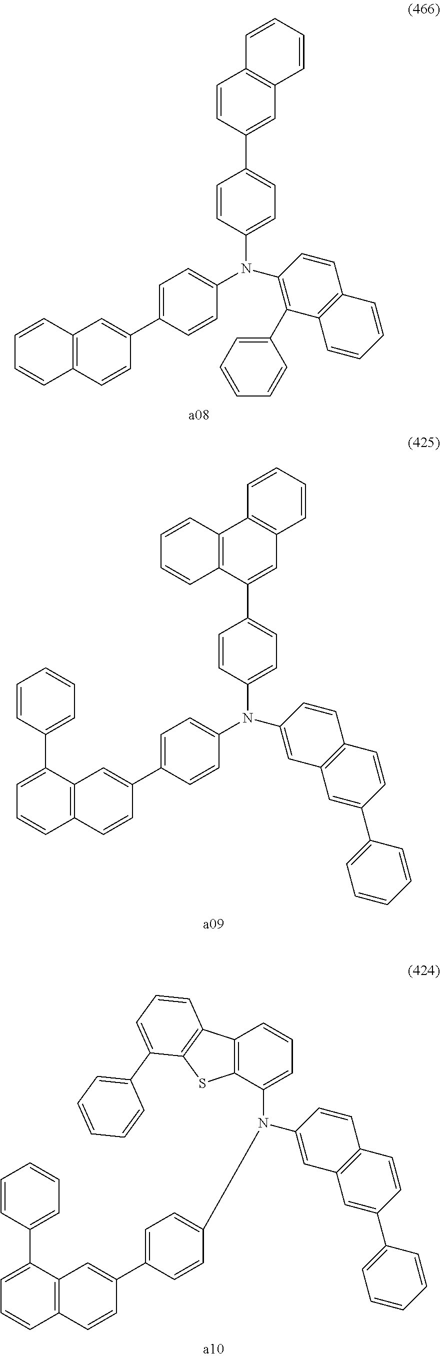 [SMILES: c1ccc(-c2c(N(c3ccc(-c4ccc5ccccc5c4)cc3)c3ccc(-c4ccc5ccccc5c4)cc3)ccc3ccccc23)cc1 ; c1ccc(-c2ccc3ccc(N(c4ccc(-c5ccc6cccc(-c7ccccc7)c6c5)cc4)c4ccc(-c5cc6ccccc6c6ccccc56)cc4)cc3c2)cc1 ; c1ccc(-c2ccc3ccc(N(c4ccc(-c5ccc6cccc(-c7ccccc7)c6c5)cc4)c4cccc5c4sc4c(-c6ccccc6)cccc45)cc3c2)cc1]

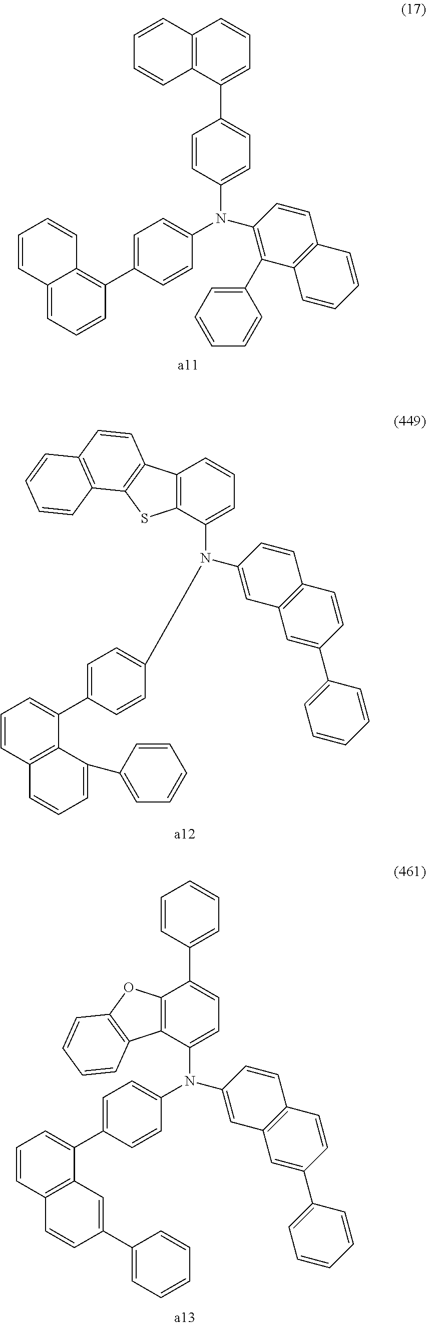 [SMILES: c1ccc(-c2c(N(c3ccc(-c4cccc5ccccc45)cc3)c3ccc(-c4cccc5ccccc45)cc3)ccc3ccccc23)cc1 ; c1ccc(-c2ccc3ccc(N(c4ccc(-c5cccc6cccc(-c7ccccc7)c56)cc4)c4cccc5c4sc4c6ccccc6ccc54)cc3c2)cc1 ; c1ccc(-c2ccc3ccc(N(c4ccc(-c5cccc6ccc(-c7ccccc7)cc56)cc4)c4ccc(-c5ccccc5)c5oc6ccccc6c45)cc3c2)cc1]

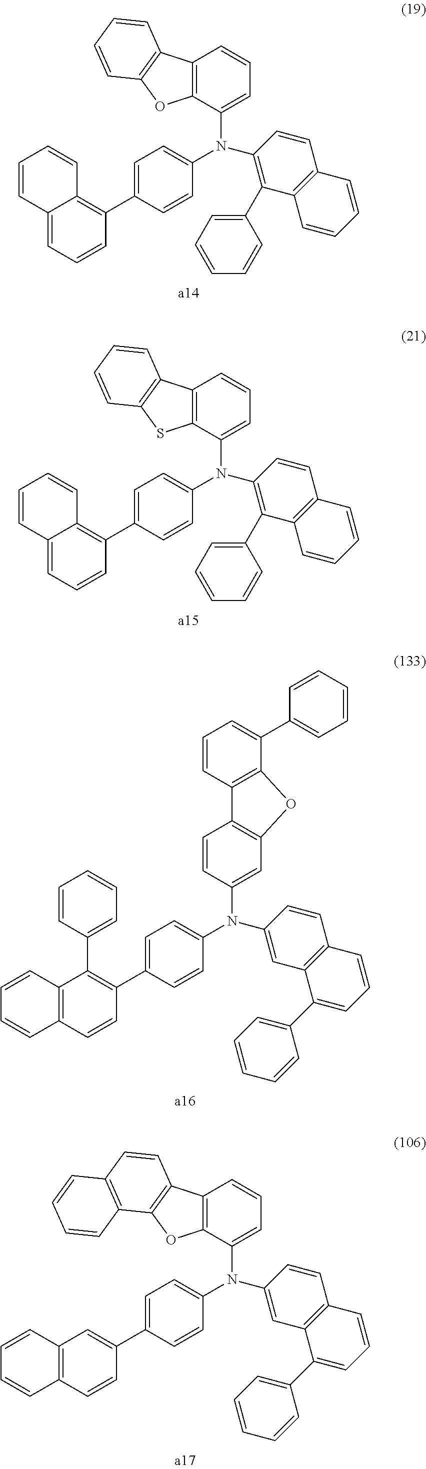 [SMILES: c1ccc(-c2c(N(c3ccc(-c4cccc5ccccc45)cc3)c3cccc4c3oc3ccccc34)ccc3ccccc23)cc1 ; c1ccc(-c2c(N(c3ccc(-c4cccc5ccccc45)cc3)c3cccc4c3sc3ccccc34)ccc3ccccc23)cc1 ; c1ccc(-c2cccc3ccc(N(c4ccc(-c5ccc6ccccc6c5-c5ccccc5)cc4)c4ccc5c(c4)oc4c(-c6ccccc6)cccc45)cc23)cc1 ; c1ccc(-c2cccc3ccc(N(c4ccc(-c5ccc6ccccc6c5)cc4)c4cccc5c4oc4c6ccccc6ccc54)cc23)cc1]

1. Manufacture and Evaluation of Light Emitting Device

(Manufacture of Light Emitting Device)

The light emitting device of an embodiment including the amine compound of an embodiment in a hole transport layer was manufactured as follows.

Compounds 1, 17, 18, 19, 21, 96, 98, 106, 133, 189, 191, 282, 284, 424, 425, 449, and 461 as described above were used as a hole transport layer material to manufacture the light emitting devices of Examples 1 to 17, respectively.

Compounds b01 to b10 were used as a hole transport layer material to manufacture the light emitting devices of Comparative Examples 1 to 10, respectively.

Comparative Example Compounds used to manufacture the devices are shown below:

### Comparative Example Compound

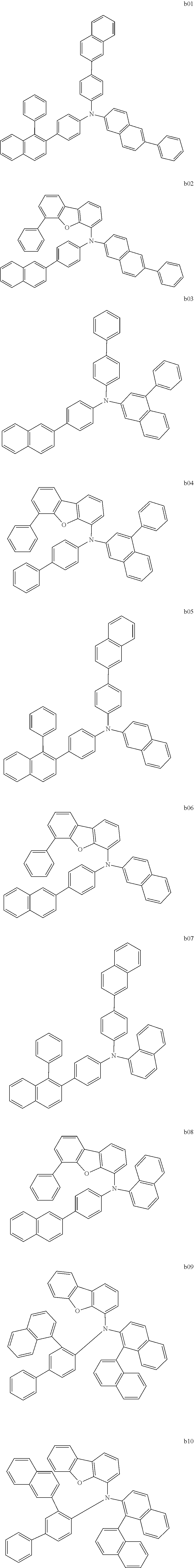

(Other Compounds Used to Manufacture Devices)

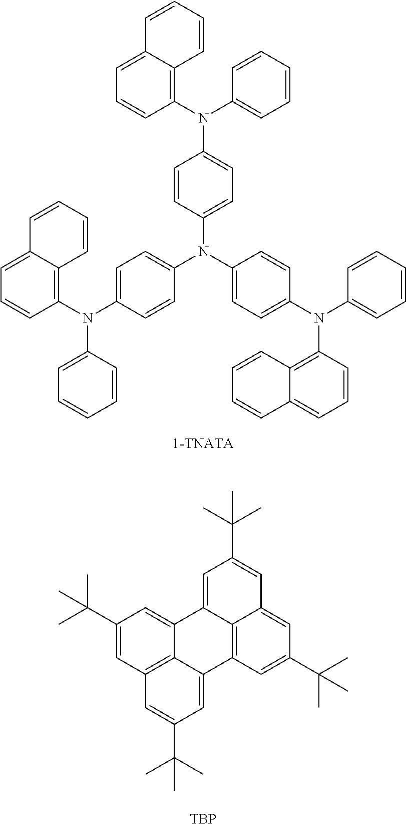

A glass substrate on which a 1,500 Å-thick ITO had been patterned was ultrasonically washed by using isopropyl alcohol and pure water for about 5 minutes each. After ultrasonic washing, the glass substrate was irradiated with UN rays for about 30 minutes and treated with ozone. 1-TNATA was deposited to form an 800 Å-thick hole injection layer. In Examples 1 to 17 and Comparative Examples 1 to 10, an Example Compound or a Comparative Example Compound was deposited to form a 100 Å-thick hole transport layer. ADN and TBP that is a blue fluorescence dopant were co-deposited at a weight ratio of 3:97 to form a 250 Å-thick emission layer. Alq3 was deposited to form a 250 Å-thick electron transport layer, and LiF was deposited to form a 10 Å-thick electron injection layer. Al was provided to form a 3,000 Å-thick second electrode.

In the Examples, the hole injection layer, the hole transport layer, the emission layer, the electron transport layer, the electron injection layer, and the second electrode were formed by using a vacuum deposition apparatus.

(Evaluation of Light Emitting Device Characteristics)

Evaluation results of the light emitting devices of Examples 1 to 17 and Comparative Examples 1 to 10 are listed in Table 4. In the evaluation results of the characteristics for the Examples and the Comparative Examples listed in Table 4, the luminous efficiency shows an efficiency value at a current density of 10 mA/cm², and the half service life shows a brightness half-life at 1,000 cd/m². Film purity shows a value in which the purity of a hole transport layer material deposited on a substrate after depositing each hole transport layer material at 0.2 nm/s is measured by HPLC. The purities of all the hole transport layer materials before being deposited were 99.9%. It was confirmed that the manufactured devices all show blue emission colors.

TABLE 4

| | Hole | | Service | Hole |
|---|---|---|---|---|
| Examples of | transport | Luminous | life | transport |
| manufactured | layer | efficiency | LT50 | layer film |
| devices | material | (cd/A) | (h) | purity |
| Example 1 | a01(282) | 6.9 | 2200 | 99.9% |
| Example 2 | a02(189) | 7.0 | 2200 | 99.9% |
| Example 3 | a03(96) | 6.8 | 2300 | 99.9% |
| Example 4 | a04(284) | 7.0 | 2300 | 99.9% |
| Example 5 | a05(191) | 6.9 | 2300 | 99.9% |
| Example 6 | a06(98) | 6.8 | 2400 | 99.9% |
| Example 7 | a07(l) | 6.8 | 2200 | 99.9% |
| Example 8 | a08(466) | 6.8 | 2200 | 99.9% |
| Example 9 | a09(425) | 6.8 | 2400 | 99.9% |
| Example 10 | a10(424) | 6.7 | 2400 | 99.9% |
| Example 11 | a1(17) | 7.0 | 2000 | 99.9% |
| Example 12 | a12(449) | 6.7 | 2400 | 99.9% |
| Example 13 | a13(461) | 6.7 | 2400 | 99.9% |
| Example 14 | a14(19) | 7.0 | 1900 | 99.9% |
| Example 15 | a15(21) | 7.0 | 2100 | 99.9% |
| Example 16 | a16(133) | 6.8 | 2500 | 99.9% |
| Example 17 | a17(106) | 7.0 | 2000 | 99.9% |
| Comparative | b01 | 6.2 | 1200 | 99.7% |
| Example 1 | | | | |
| Comparative | b02 | 6.1 | 1300 | 99.7% |
| Example 2 | | | | |
| Comparative | b03 | 6.5 | 1700 | 99.9% |
| Example 3 | | | | |
| Comparative | b04 | 6.5 | 1800 | 99.9% |
| Example 4 | | | | |
| Comparative | b05 | 6.4 | 1500 | 99.9% |
| Example 5 | | | | |
| Comparative | b06 | 6.3 | 1600 | 99.9% |
| Example 6 | | | | |
| Comparative | b07 | 6.6 | 1000 | 99.2% |
| Example 7 | | | | |
| Comparative | b08 | 6.6 | 900 | 99.1% |
| Example 8 | | | | |
| Comparative | b09 | 6.2 | 800 | 99.8% |
| Example 9 | | | | |
| Comparative | b10 | 6.2 | 800 | 99.8% |
| Example 10 | | | | |

Referring to the results of Table 4, it may be seen that Examples of the light emitting devices using the amine compounds according to embodiments as a hole transport layer material exhibit excellent luminous efficiency and long service life characteristics.

Referring to Table 4, it may be confirmed that the devices of Examples 1 to 17 exhibit high efficiency, long service life, and high purity characteristics compared to those of Comparative Examples 1 to 10.

The devices of Examples 1 to 17 have long service life characteristics compared to those of Comparative Examples 1 and 2. Compounds a01 to a17 of Examples and b01 and b02 of Comparative Examples 1 and 2 have a difference of the position at which the phenyl group is substituted at the naphthyl group directly bonded to the nitrogen atom of amine. Unlike Compounds a01 to a17, Compounds b01 and b02 have a structure in which the phenyl group is substituted at 6-position of the naphthyl group directly bonded to the nitrogen atom of amine. Compounds b01 and b02 may absorb light having a long wavelength because the phenyl group is substituted at 6-position of the naphthyl group directly bonded to the nitrogen atom of amine. Accordingly, Compounds b01 and b02 absorb a portion of light emitted from the emission layer and the hole transport layer material is excited, thereby making service lives short. It may be confirmed that the devices of Examples 1 to 17 exhibit long service life characteristics by including compounds having higher molecular stability in the hole transport layer compared to those of Comparative Examples 1 and 2.

The devices of Examples 1 to 17 have longer service life characteristics compared to those of Comparative Examples 3 and 4. For Compounds a01 to a17 of the Examples, all the substituents substituted at the nitrogen atom of amine are naphthyl groups or heteroaryl groups having relatively large electron resistance while, for Compounds b03 and b04 of Comparative Examples, one of the substituents substituted at the nitrogen atom of amine is a biphenyl group having a relatively small electron resistance. It may be confirmed that the devices of Examples 1 to 17 exhibit long service life characteristics by including compounds having relatively higher electron resistance in the hole transport layer compared to those of Comparative Examples 3 and 4.

The devices of Examples 1 to 17 have longer service life characteristics compared to those of Comparative Examples 5 and 6. There is a difference in that Compounds a01 to a17 of the Examples include a 2-naphthyl group substituted with a phenyl group directly bonded to the nitrogen atom of amine while Compounds b05 and b06 of the Comparative Examples include an unsubstituted 2-naphtyl group directly bonded to the nitrogen atom of amine. The substituted 2-naphthyl group has electron resistance greater than the unsubstituted 2-naphthyl group. It may be confirmed that the devices of Examples 1 to 17 exhibit long service life characteristics by including compounds having relatively higher electron resistance in the hole transport layer compared to those of Comparative Examples 5 and 6.

The devices of Examples 1 to 17 have longer service life characteristics compared to those of Comparative Examples 7 and 8. There is a difference in that Compounds a01 to a17 of the Examples include a 2-naphthyl group substituted with a phenyl group directly bonded to the nitrogen atom of amine while Compounds b07 and b08 of the Comparative Examples include an unsubstituted 1-naphtyl group directly bonded to the nitrogen atom of amine. A 1-naphtyl group has relatively low heat resistance compared to a 2-naphthyl group, and thus there is a limitation in that materials are deteriorated when deposited. Referring to Table 4, this may be confirmed through the fact that Comparative Examples 7 and 8 exhibit the film purity of 99.1% and 99.2% of the hole transport layer, respectively. It may be confirmed that the devices of Examples 1 to 17 include the amine compound having relatively high heat resistance in the hole transport layer compared to the devices of Comparative Examples 7 and 8 to thus have low rate of deterioration of materials when deposited, and as a result, exhibit long service life characteristics compared to those of Comparative Examples 7 and 8.

The devices of Examples 1 to 17 have longer service life characteristics compared to those of Comparative Examples 9 and 10. There is a difference in that Compounds a01 to a17 of the Examples have a phenyl group substituted at a naphthyl group directly bonded to the nitrogen atom of amine while Compounds b09 and b10 of the Comparative Examples have a naphthyl group substituted at a naphthyl group directly bonded to the nitrogen atom of amine. The naphthyl group substituted with a naphthyl group has a relatively low energy level of Lowest Unoccupied Molecular Orbital (LUMO) compared to the naphthyl group substituted with a phenyl group, and thus the function as an electron blocking layer may be weak. It may be confirmed that the devices of Examples 1 to 17 have a relatively high energy level of LUMO in the hole transport layer compared to those of Comparative Examples 9 and 10, and include the amine compounds having excellent electron blocking characteristics, thereby exhibiting excellent luminous efficiency and long service life characteristics compared to those of Comparative Examples 9 and 10.

Thus, Examples 1 to 18 show results of simultaneously improving the luminous efficiency and the light emitting service life compared to Comparative Examples 1 to 10. The light emitting devices of Examples may have the device efficiency and the device service life simultaneously improved because each of three substituents substituted at the nitrogen atom in the hole transport layer is a naphthyl group or a heteroaryl group, and the naphthyl group directly bonded to the nitrogen atom includes the amine compound having a structure in which a phenyl group is substituted at a particular position.

The amine compound according to embodiments may contribute to long service life and high efficiency characteristics of the light emitting device because each of three substituents substituted at the nitrogen atom is a naphthyl group or a heteroaryl group, and the naphthyl group directly bonded to the nitrogen atom has a compound structure in which a phenyl group is substituted at a particular position. The light emitting device according to embodiments may include the amine compound according to embodiments, thereby exhibiting long service life and high efficiency characteristics simultaneously.

The light emitting device according to embodiments may include the amine compound of an embodiment in the hole transport region, thereby exhibiting high efficiency and long service life characteristics.

The amine compound according to embodiments may improve luminous efficiency and a device service life of the light emitting device.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the following claims.

What is claimed is:

1. A light emitting device comprising:
a first electrode;
a second electrode disposed on the first electrode;
an emission layer disposed between the first electrode and the second electrode; and
a hole transport region disposed between the emission layer and the first electrode and comprising an amine compound represented by Formula 1: 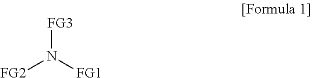
wherein in Formula 1,
FG1 is a group represented by Formula A,
FG2 is a group represented by Formula B, and
FG3 is a group represented by one of Formula A to Formula C: 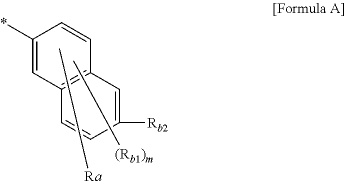
wherein in Formula A,
m is an integer from 1 to 5,
Rₐ is a substituted or unsubstituted phenyl group, a substituted or unsubstituted dibenzofuran group, or a substituted or unsubstituted dibenzothiophene group, and
R_{b1} and R_{b2} are each independently a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group, 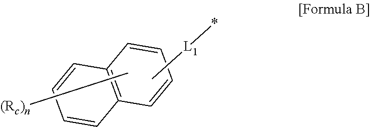
wherein in Formula B,
n is an integer from 0 to 7,
L₁ is a substituted or unsubstituted arylene group having 6 to 30 ring-forming carbon atoms, or a substituted or unsubstituted heteroarylene group having 2 to 30 ring-forming carbon atoms, and
R_{c} is a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group, 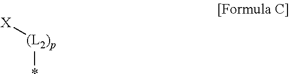
wherein in Formula C,
p is 0 or 1,
L₂ is a substituted or unsubstituted arylene group having 6 to 30 ring-forming carbon atoms, or a substituted or unsubstituted heteroarylene group having 2 to 30 ring-forming carbon atoms, and
X is a group represented by one of Formula D-1 to Formula D-3: 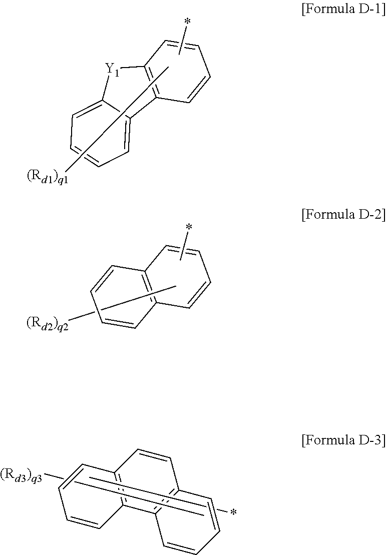
wherein in Formula D-1,
Y₁ is O or S,
wherein in Formula D-1 to Formula D-3,
q₁ and q₂ are each independently an integer from 0 to 7,
q₃ is an integer from 0 to 9, and
R_{d1} to R_{d3} are each independently a deuterium atom, a halogen atom, a substituted or unsubstituted aryl group having 6 to 30 ring-forming carbon atoms, a substituted or unsubstituted heteroaryl group having 2 to 30 ring-forming carbon atoms, a substituted or unsubstituted alkenyl group 2 to 10 carbon atoms, a substituted or unsubstituted oxy group, or a substituted or unsubstituted thio group, or are bonded to an adjacent group to form a ring.

2. The light emitting device of claim 1, wherein the group represented by Formula A is represented by Formula AA-1: 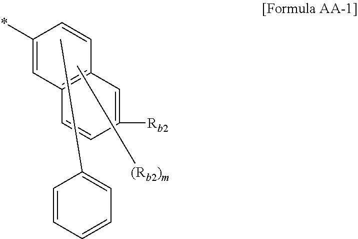
wherein in Formula AA-1,
m, R_{b1}, and R_{b2} are the same as defined in Formula A.

3. The light emitting device of claim 1, wherein the group represented by Formula B is represented by one of Formula BB-1 to Formula BB-3: 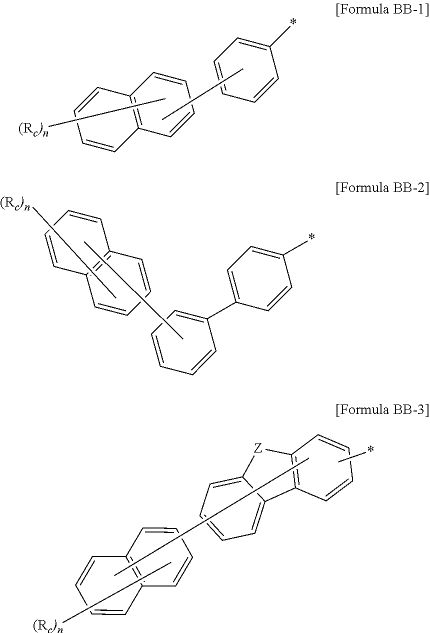
wherein in Formula BB-3,
Z is C(R_{z1})(R_{z2}), N(R_{z3}), O, or S,
R_{z1} to R_{z3} are each independently a substituted or unsubstituted phenyl group, and
wherein in Formula BB-1 to Formula BB-3,
n and R_{c} are the same as defined in Formula B.

4. The light emitting device of claim 3, wherein the group represented by Formula BB-3 is represented by one of Formula BBB-1 to Formula BBB-3: 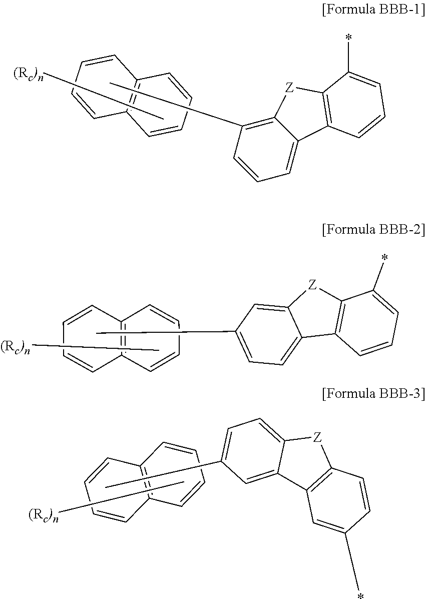
wherein in Formula BBB-1 to Formula BBB-3,
n and R_{c} are the same as defined in Formula B, and
Z is the same as defined in Formula BB-3.

5. The light emitting device of claim 1, wherein the group represented by Formula C is represented by one of Formula CC-1 to Formula CC-3: 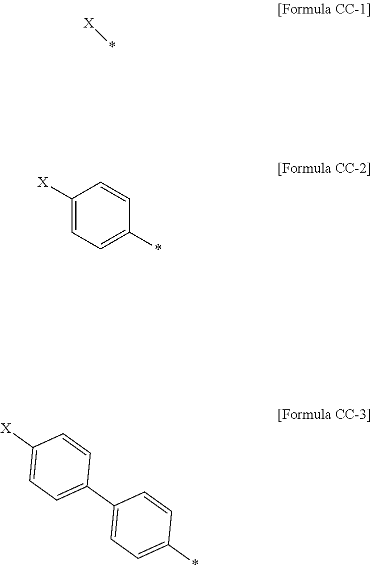
wherein in Formula CC-1 to Formula CC-3,
X is the same as defined in Formula C.

6. The light emitting device of claim 1, wherein the emission layer emits blue light having a center wavelength in a range of about 450 nm to about 470 nm.

7. The light emitting device of claim 1, wherein
the hole transport region further comprises:
a hole injection layer disposed on the first electrode; and
a hole transport layer disposed between the hole injection layer and the emission layer, and
the hole transport layer comprises the amine compound.

8. The light emitting device of claim 1, wherein in Formula 1,
FG1 is a group represented by one of Formulae A-1 to A-75,
FG2 is a group represented by one of Formulae B-1 to B-99 and B-101 to B-106, and
FG3 is a group represented by one of Formulae A-1 to A-75, B-1 to B-99, B-101 to B-106, C-1 to C-77, and C-101 to C-109:

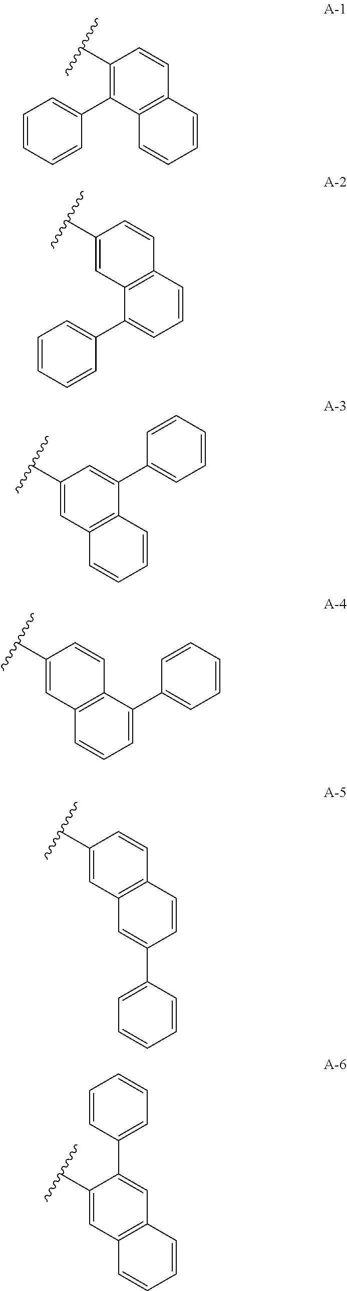

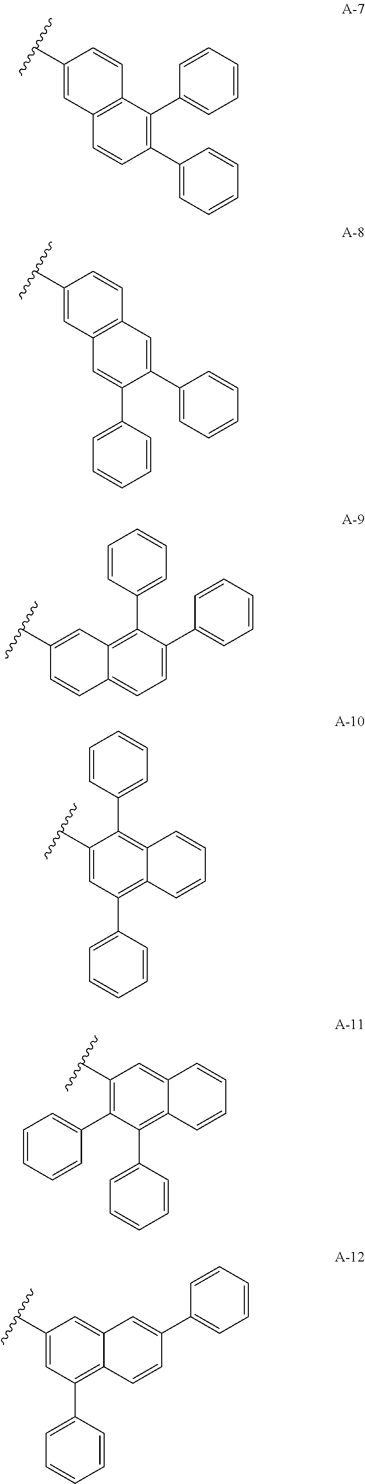

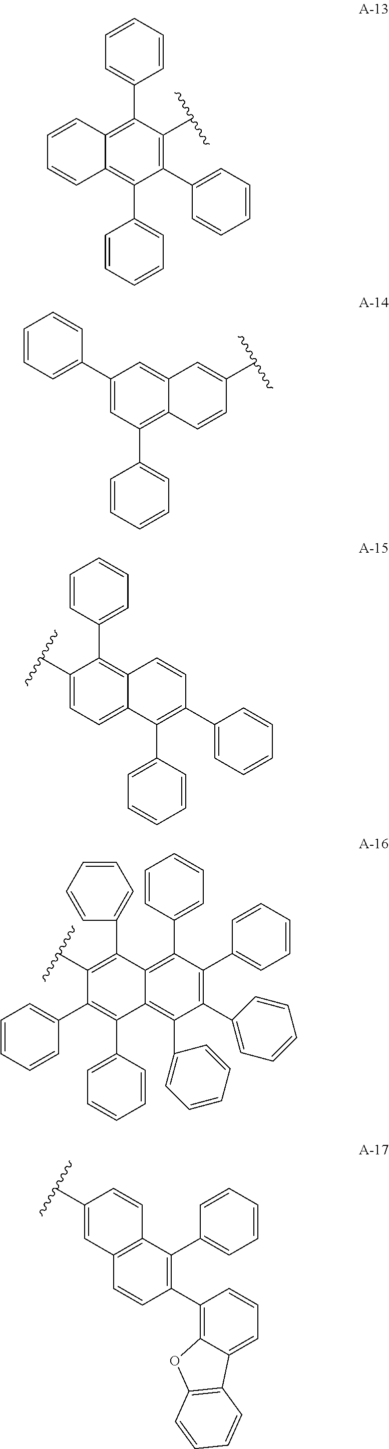

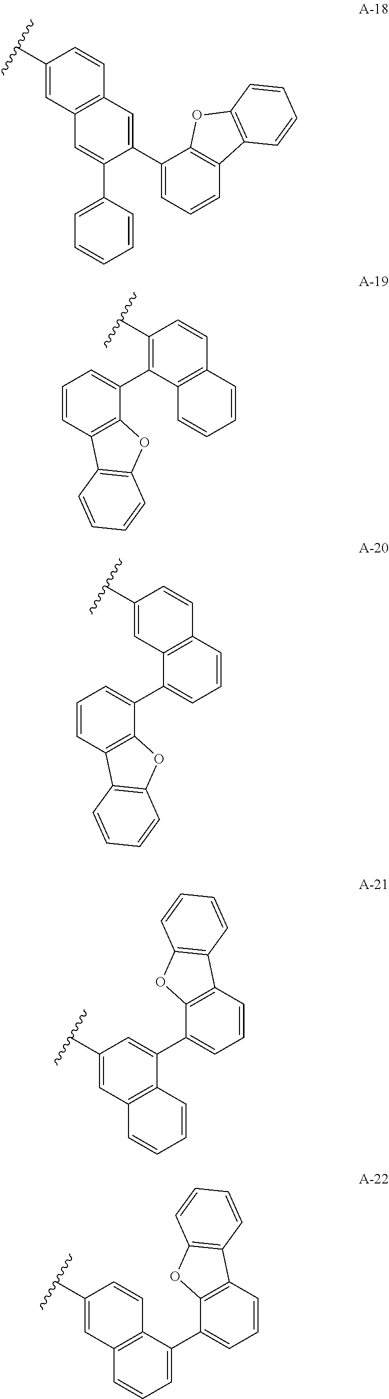 [SMILES: *c1ccc2cc(-c3cccc4c3oc3ccccc34)c(-c3ccccc3)cc2c1 ; *c1ccc2ccccc2c1-c1cccc2c1oc1ccccc12 ; *c1ccc2cccc(-c3cccc4c3oc3ccccc34)c2c1 ; *c1cc(-c2cccc3c2oc2ccccc23)c2ccccc2c1 ; *c1ccc2c(-c3cccc4c3oc3ccccc34)cccc2c1]

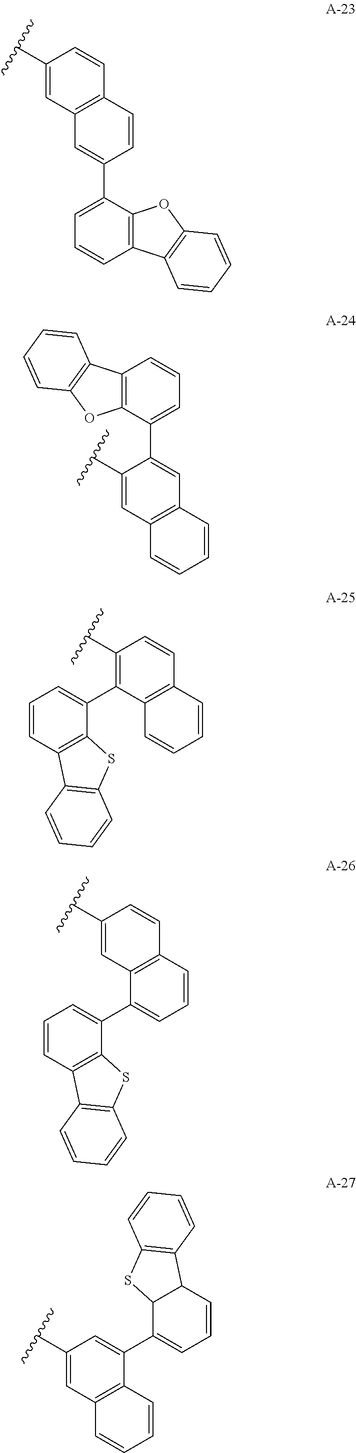 [SMILES: *c1ccc2ccc(-c3cccc4c3oc3ccccc34)cc2c1 ; *c1cc2ccccc2cc1-c1cccc2c1oc1ccccc12 ; *c1ccc2ccccc2c1-c1cccc2c1sc1ccccc12 ; *c1ccc2cccc(-c3cccc4c3sc3ccccc34)c2c1 ; *c1cc(C2=CC=CC3c4ccccc4SC23)c2ccccc2c1]

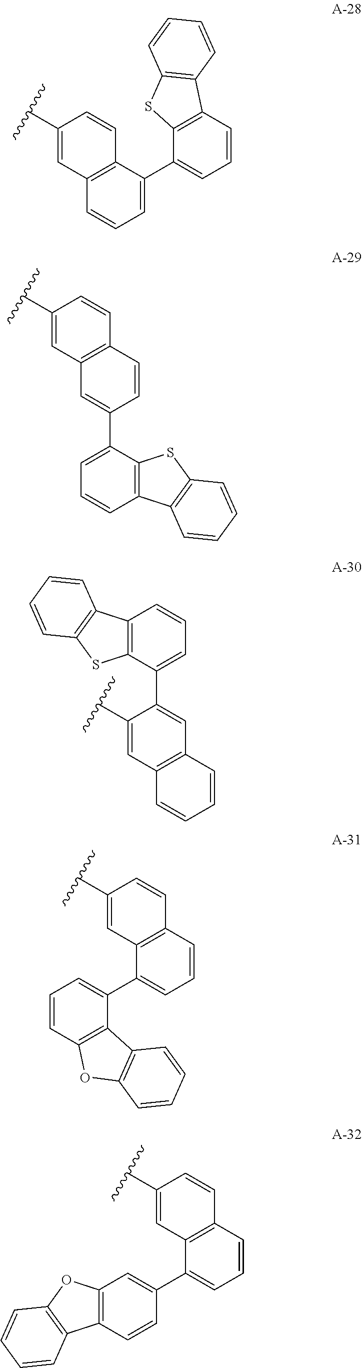 [SMILES: *c1ccc2c(-c3cccc4c3sc3ccccc34)cccc2c1 ; *c1ccc2ccc(-c3cccc4c3sc3ccccc34)cc2c1 ; *c1cc2ccccc2cc1-c1cccc2c1sc1ccccc12 ; *c1ccc2cccc(-c3cccc4oc5ccccc5c34)c2c1 ; *c1ccc2cccc(-c3ccc4c(c3)oc3ccccc34)c2c1]

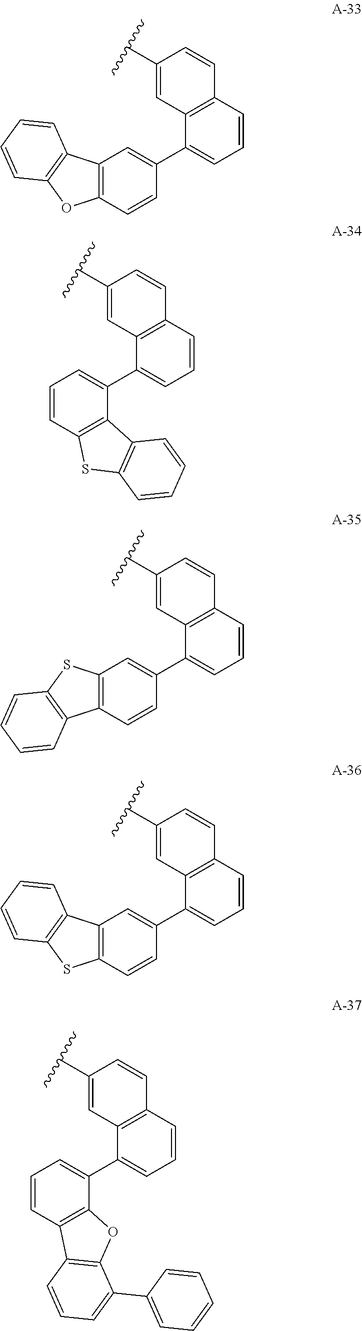 [SMILES: *c1ccc2cccc(-c3ccc4oc5ccccc5c4c3)c2c1 ; *c1ccc2cccc(-c3cccc4sc5ccccc5c34)c2c1 ; *c1ccc2cccc(-c3ccc4c(c3)sc3ccccc34)c2c1 ; *c1ccc2cccc(-c3ccc4sc5ccccc5c4c3)c2c1 ; *c1ccc2cccc(-c3cccc4c3oc3c(-c5ccccc5)cccc34)c2c1]

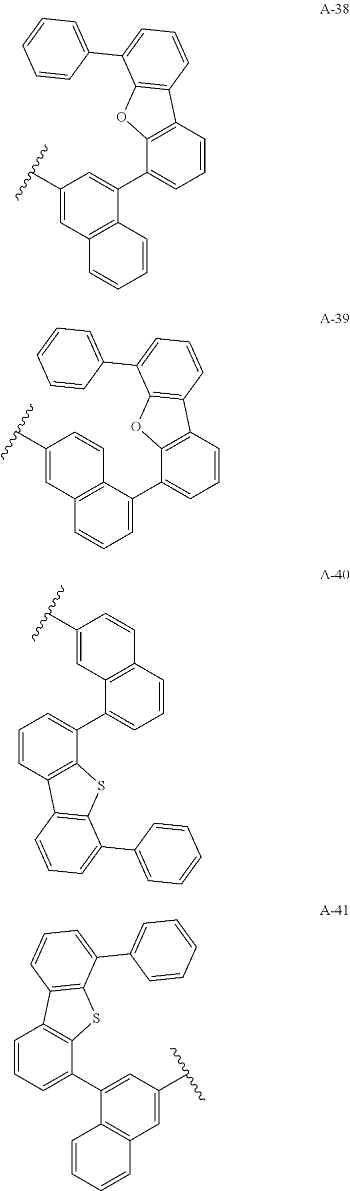

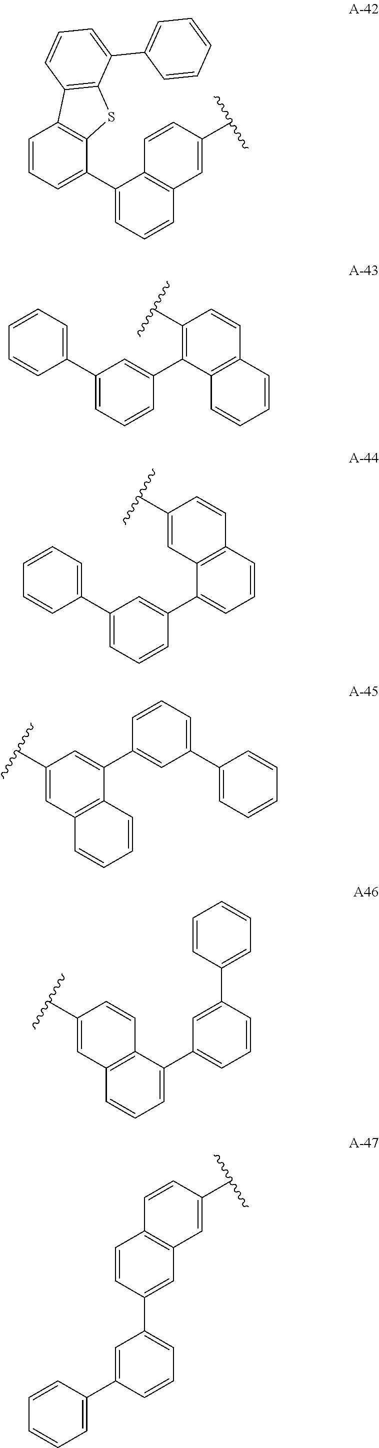

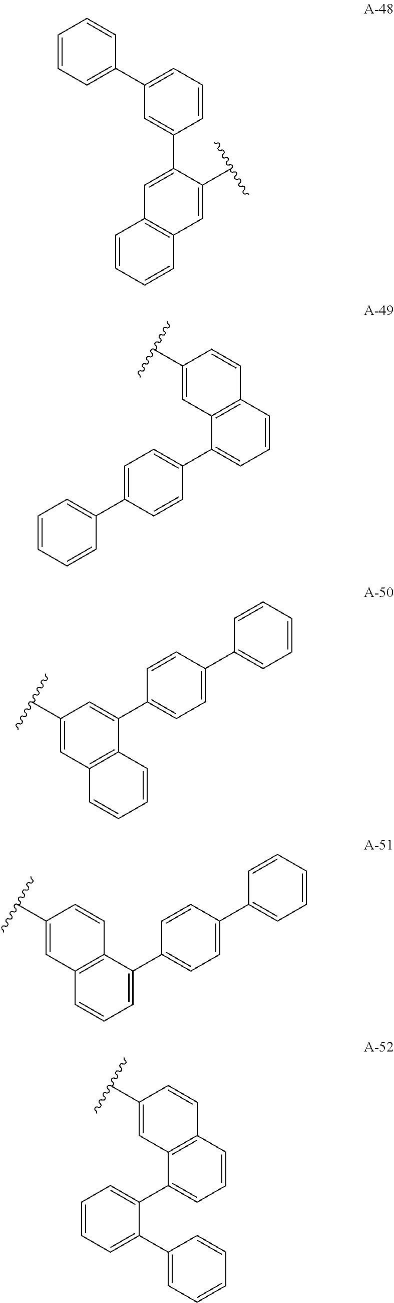 [SMILES: *c1cc2ccccc2cc1-c1cccc(-c2ccccc2)c1 ; *c1ccc2cccc(-c3ccc(-c4ccccc4)cc3)c2c1 ; *c1cc(-c2ccc(-c3ccccc3)cc2)c2ccccc2c1 ; *c1ccc2c(-c3ccc(-c4ccccc4)cc3)cccc2c1 ; *c1ccc2cccc(-c3ccccc3-c3ccccc3)c2c1]

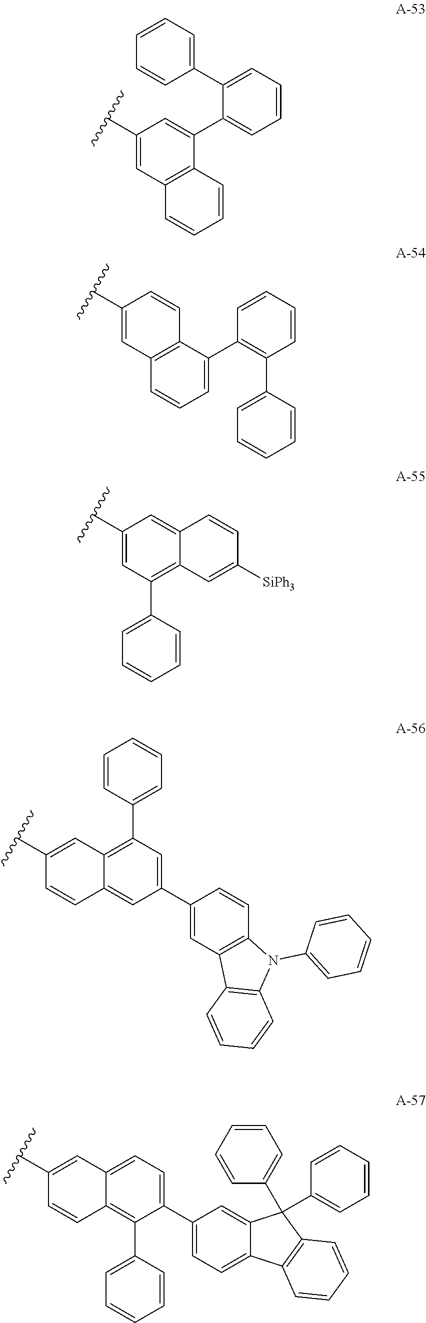

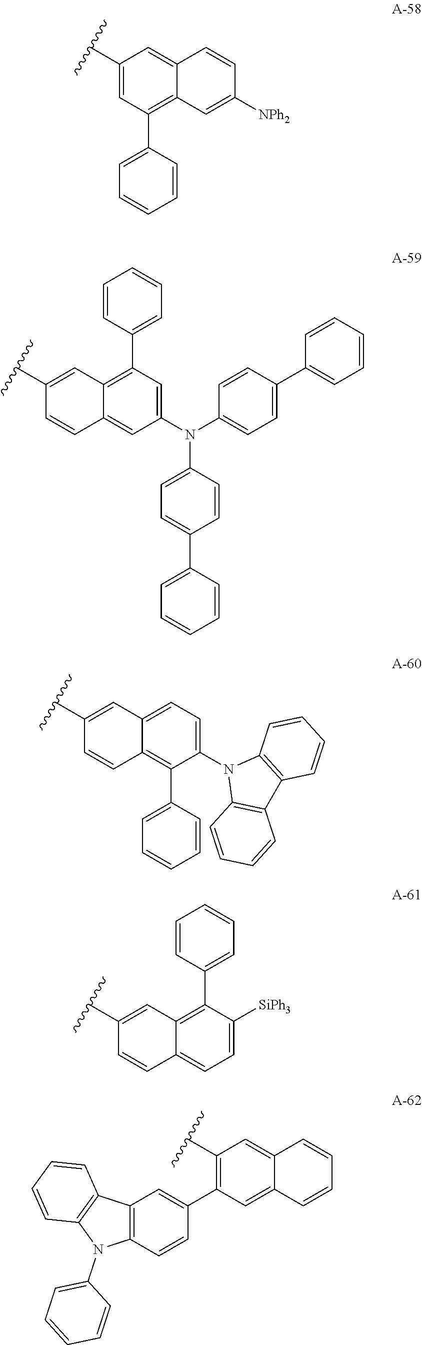

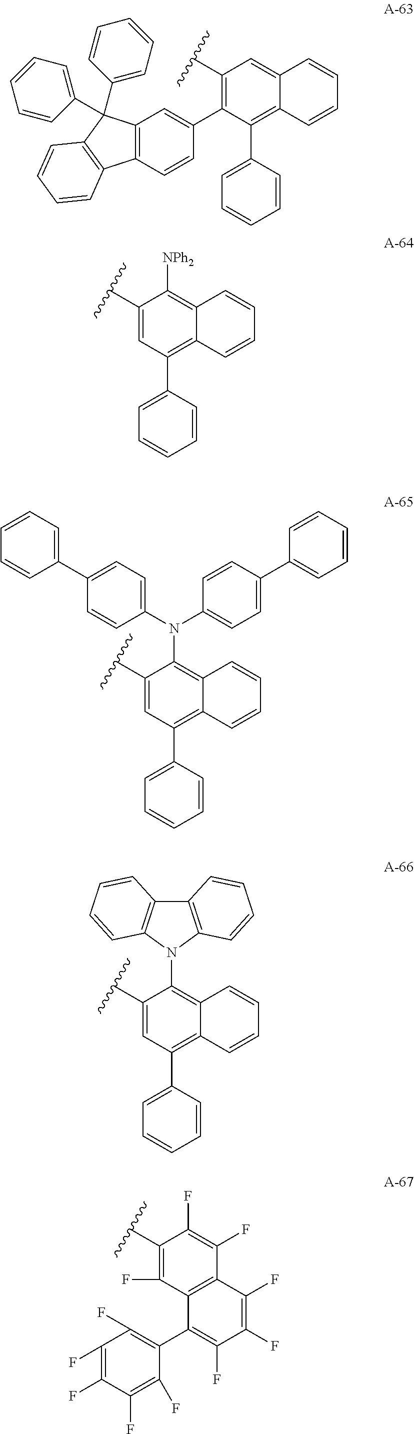

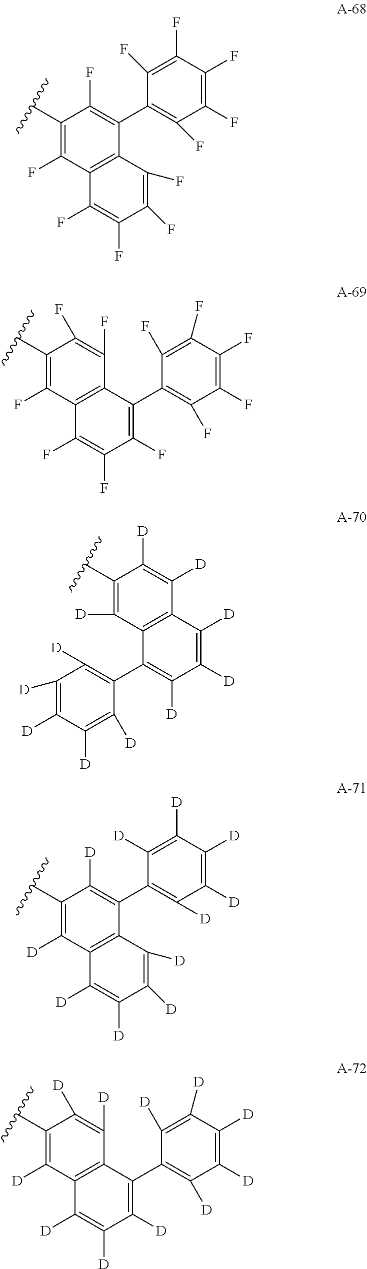 [SMILES: *c1c(F)c(-c2c(F)c(F)c(F)c(F)c2F)c2c(F)c(F)c(F)c(F)c2c1F ; *c1c(F)c(F)c2c(-c3c(F)c(F)c(F)c(F)c3F)c(F)c(F)c(F)c2c1F ; *c1c([2H])c([2H])c2c([2H])c([2H])c([2H])c(-c3c([2H])c([2H])c([2H])c([2H])c3[2H])c2c1[2H] ; *c1c([2H])c(-c2c([2H])c([2H])c([2H])c([2H])c2[2H])c2c([2H])c([2H])c([2H])c([2H])c2c1[2H] ; *c1c([2H])c([2H])c2c(-c3c([2H])c([2H])c([2H])c([2H])c3[2H])c([2H])c([2H])c([2H])c2c1[2H]]

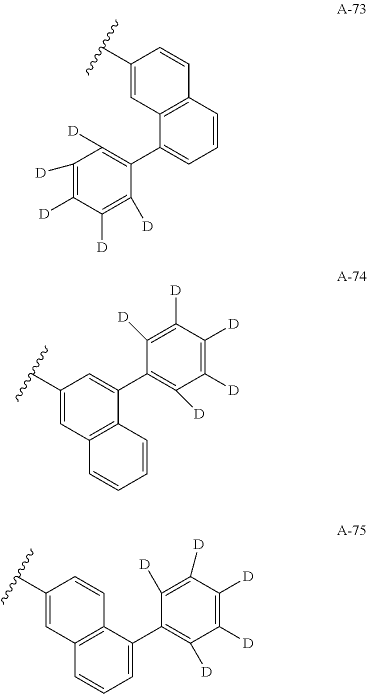 [SMILES: *c1ccc2cccc(-c3c([2H])c([2H])c([2H])c([2H])c3[2H])c2c1 ; *c1cc(-c2c([2H])c([2H])c([2H])c([2H])c2[2H])c2ccccc2c1 ; *c1ccc2c(-c3c([2H])c([2H])c([2H])c([2H])c3[2H])cccc2c1]

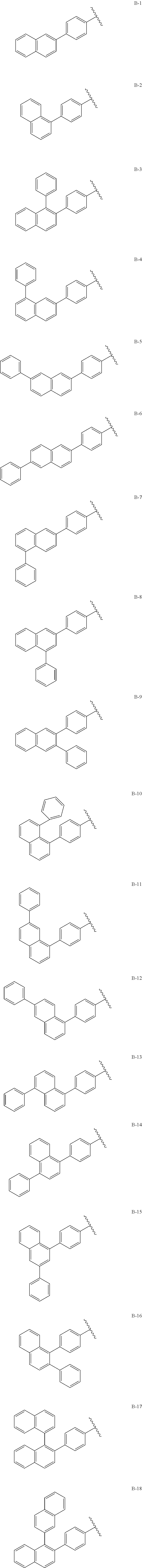

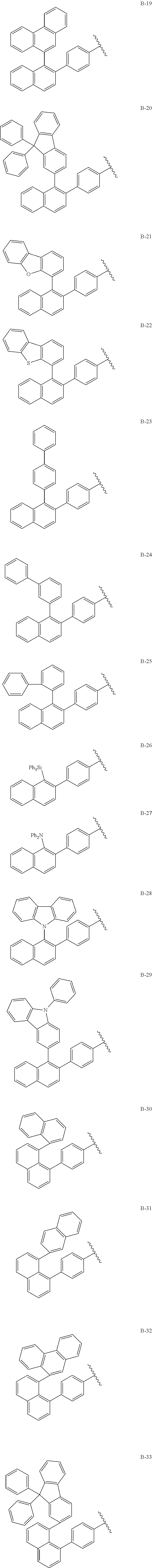

[chemical structure]

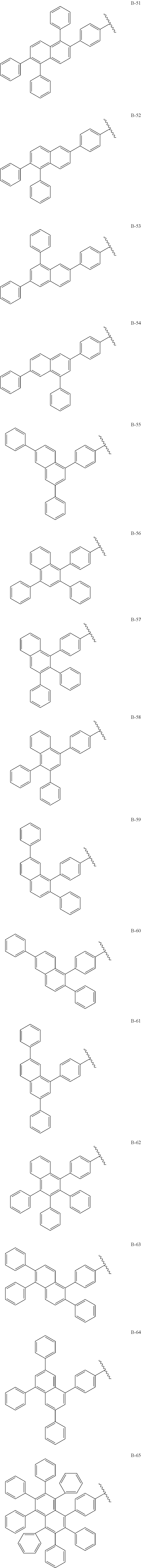

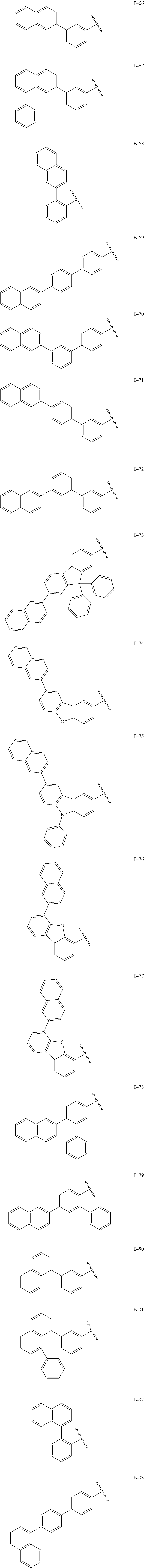

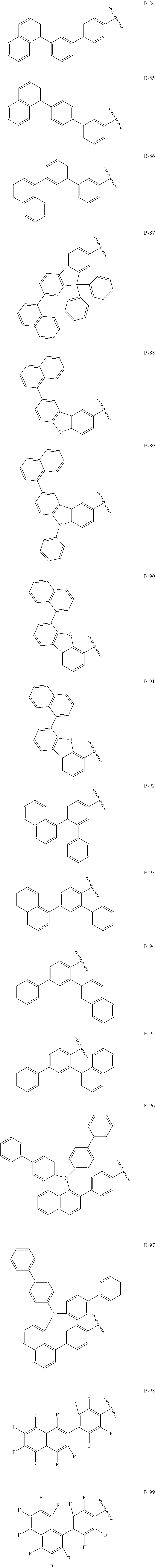

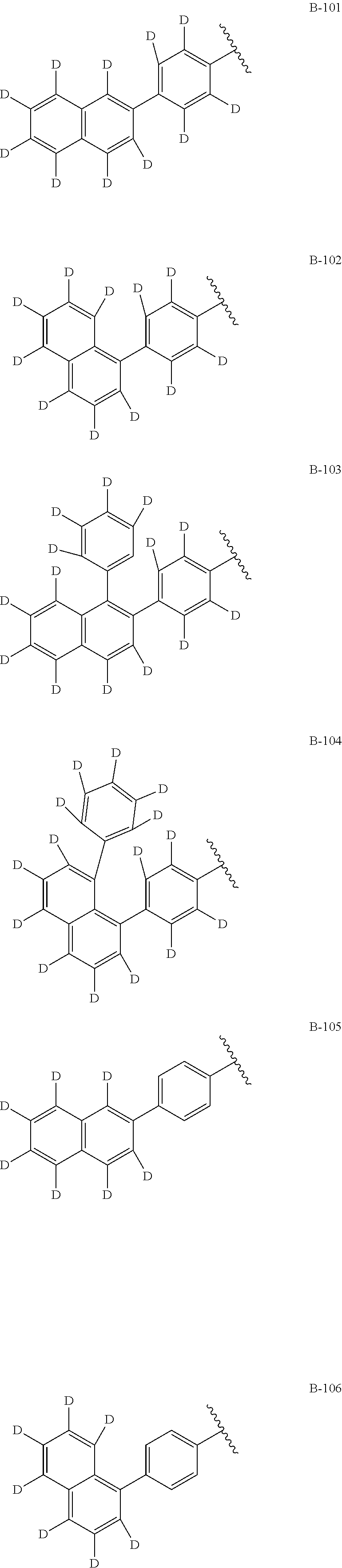

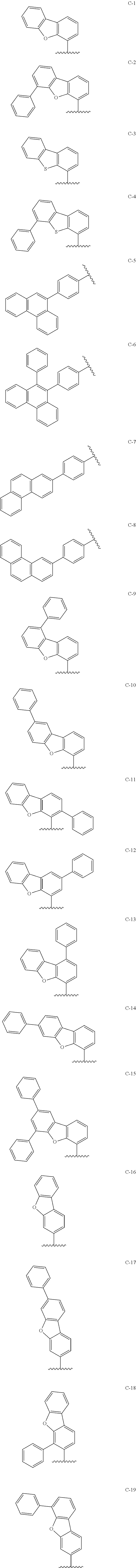

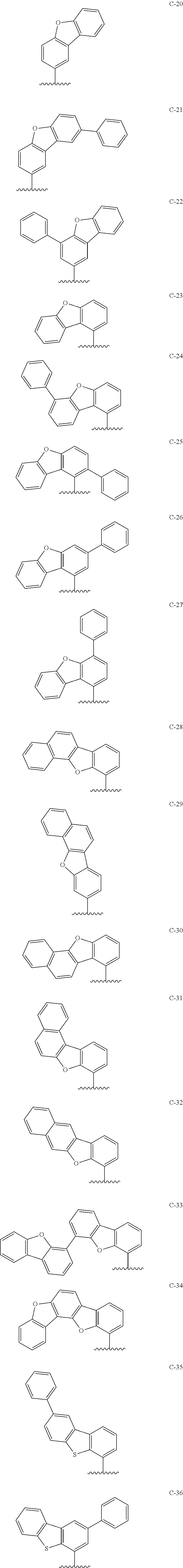

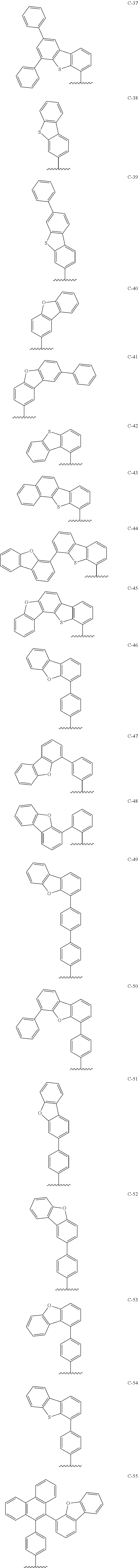

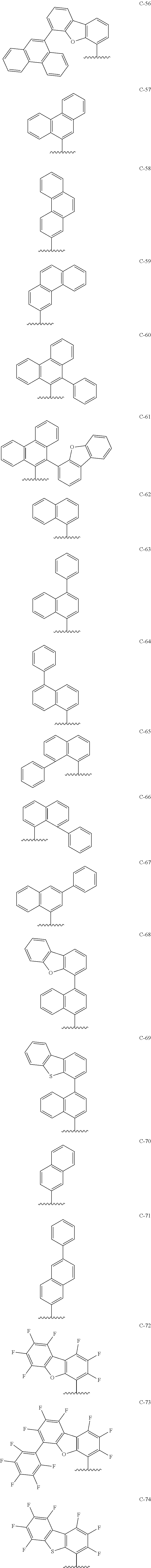

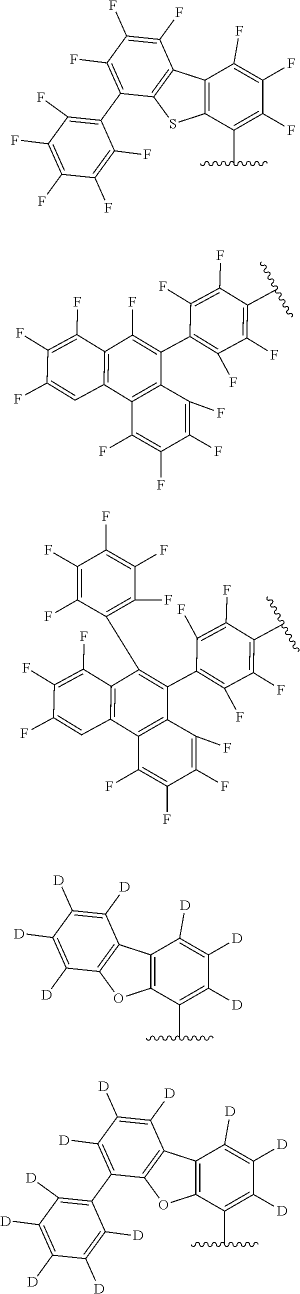 [SMILES: *c1c(F)c(F)c(F)c2c1sc1c(-c3c(F)c(F)c(F)c(F)c3F)c(F)c(F)c(F)c12 ; *c1c(F)c(F)c(-c2c(F)c3c(F)c(F)c(F)cc3c3c(F)c(F)c(F)c(F)c23)c(F)c1F ; *c1c(F)c(F)c(-c2c(-c3c(F)c(F)c(F)c(F)c3F)c3c(F)c(F)c(F)cc3c3c(F)c(F)c(F)c(F)c23)c(F)c1F ; *c1c([2H])c([2H])c([2H])c2c1oc1c([2H])c([2H])c([2H])c([2H])c12 ; *c1c([2H])c([2H])c([2H])c2c1oc1c(-c3c([2H])c([2H])c([2H])c([2H])c3[2H])c([2H])c([2H])c([2H])c12]

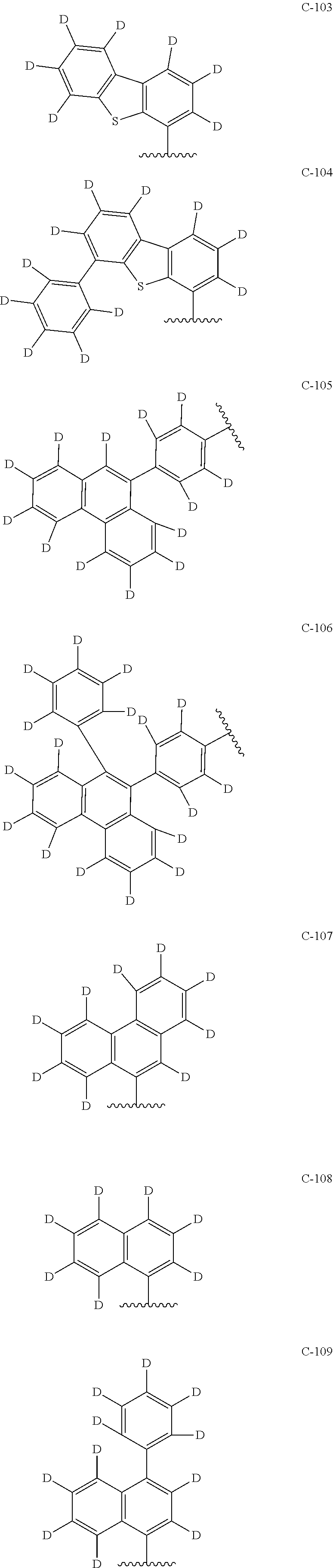

9. A light emitting device comprising:
a first electrode;
a second electrode disposed on the first electrode;
an emission layer disposed between the first electrode and the second electrode; and
a hole transport region disposed between the emission layer and the first electrode and comprising an amine compound represented by Formula E: 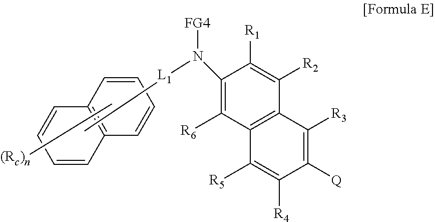
wherein in Formula E,
n is an integer from 0 to 7,
L₁ is a substituted or unsubstituted arylene group having 6 to 30 ring-forming carbon atoms, or a substituted or unsubstituted heteroarylene group having 2 to 30 ring-forming carbon atoms,
at least one of R₁ to R₆ is a substituted or unsubstituted phenyl group, a substituted or unsubstituted dibenzofuran group, or a substituted or unsubstituted dibenzothiophene group,
the remainder of R₁ to R₆ are each independently a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group,
Q is a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group,
R_{c} is a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group, and
FG4 is a group represented by one of Formula F-1 to Formula F-3: 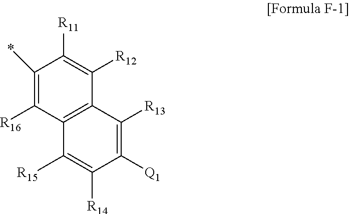
wherein in Formula F-1,
at least one of R₁₁ to R₁₆ is a substituted or unsubstituted phenyl group, a substituted or unsubstituted dibenzofuran group, or a substituted or unsubstituted dibenzothiophene group,
the remainder of R₁₁ to R₁₆ are each independently a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group, and
Q₁ is a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group, 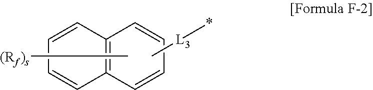
wherein in Formula F2,
s is an integer from 0 to 7,
L₃ is a substituted or unsubstituted arylene group having 6 to 30 ring-forming carbon atoms, or a substituted or unsubstituted heteroarylene group having 2 to 30 ring-forming carbon atoms, and
R_{f} is a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group, and 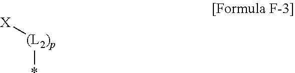
wherein in Formula F-3,
p is 0 or 1,
L₂ is a substituted or unsubstituted arylene group having 6 to 30 ring-forming carbon atoms, or a substituted or unsubstituted heteroarylene group having 2 to 30 ring-forming carbon atoms, and
X is a group represented by one of Formula D-1 to Formula D-3: 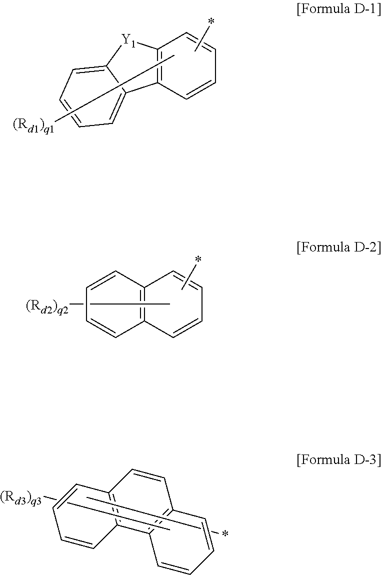
wherein in Formula D-1,
Y₁ is O or S, and
wherein in Formula D-1 to Formula D-3,
q₁ and q₂ are each independently an integer from 0 to 7,
q₃ is an integer from 0 to 9, and
R_{d1} to R_{d3} are each independently a deuterium atom, a halogen atom, a substituted or unsubstituted aryl group having 6 to 30 ring-forming carbon atoms, a substituted or unsubstituted heteroaryl group having 2 to 30 ring-forming carbon atoms, a substituted or unsubstituted alkenyl group 2 to 10 carbon atoms, a substituted or unsubstituted oxy group, or a substituted or unsubstituted thio group, or are bonded to an adjacent group to form a ring.

10. The light emitting device of claim 9, wherein in Formula E,
at least one of R₁ to R₆ is an unsubstituted phenyl group.

11. The light emitting device of claim 9, wherein the amine compound represented by Formula E is represented by one of Formula G-1 to Formula G-3: 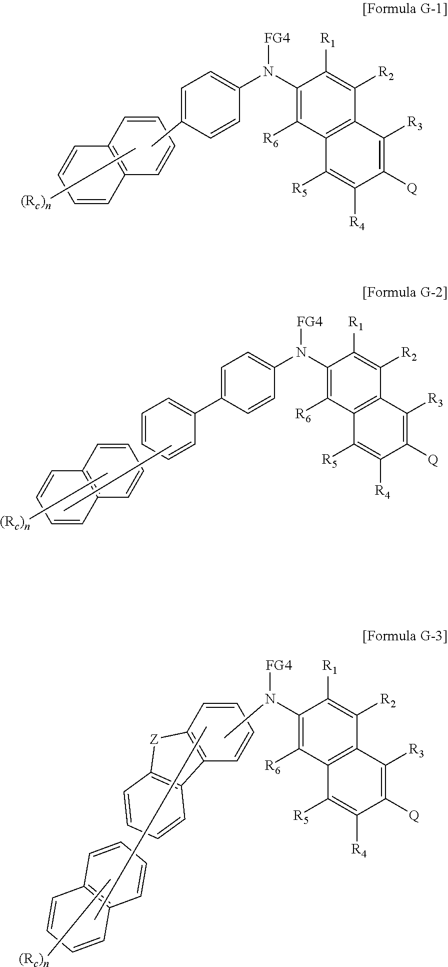
wherein in Formula G-3,
Z is C(R_{z1})(R_{z2}), N(R_{z3}), O, or S, and
R_{z1} to R_{z3} are each independently a substituted or unsubstituted phenyl group, and
wherein in Formula G-1 to Formula G-3,
n, FG4, R_{c}, Q, and R₁ to R₆ are the same as defined in Formula E.

12. The light emitting device of claim 11, wherein the amine compound represented by Formula G-3 is represented by one of Formula GG-1 to Formula GG-3: 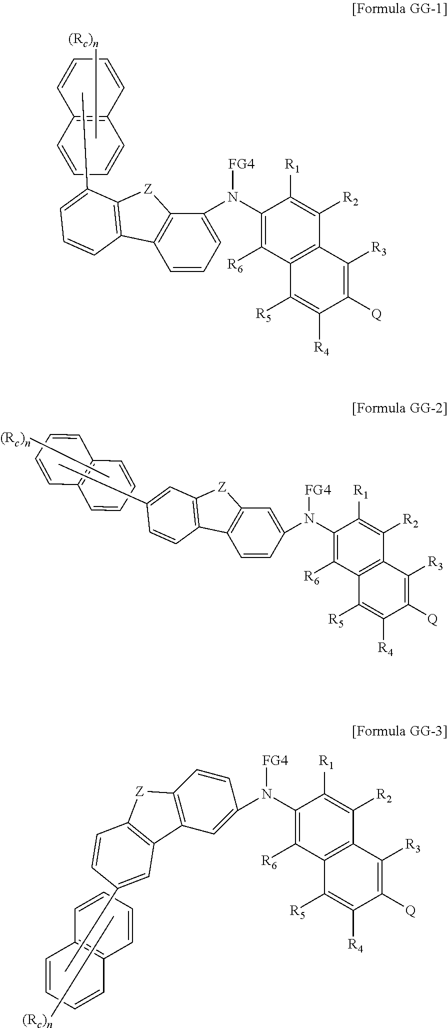
wherein in Formula GG-1 to Formula GG-3,
Z is the same as defined in Formula G-3, and
n, FG4, R_{c}, Q, and R₁ to R₆ are the same as defined in Formula E.

13. The light emitting device of claim 9, wherein in Formula F-3,
L₂ is a substituted or unsubstituted divalent phenylene group, or a substituted or unsubstituted divalent biphenyl group.

14. The light emitting device of claim 9, wherein the amine compound represented by Formula E comprises:
a first substituent represented by one of Formulae A-1 to A-75;
a second substituent represented by one of Formulae B-1 to B-99 and B-101 to B-106; and
a third substituent represented by one of Formulae A-1 to A-75, B-1 to B-99, B-101 to B-106, C-1 to C-77, and C-101 to C-109:

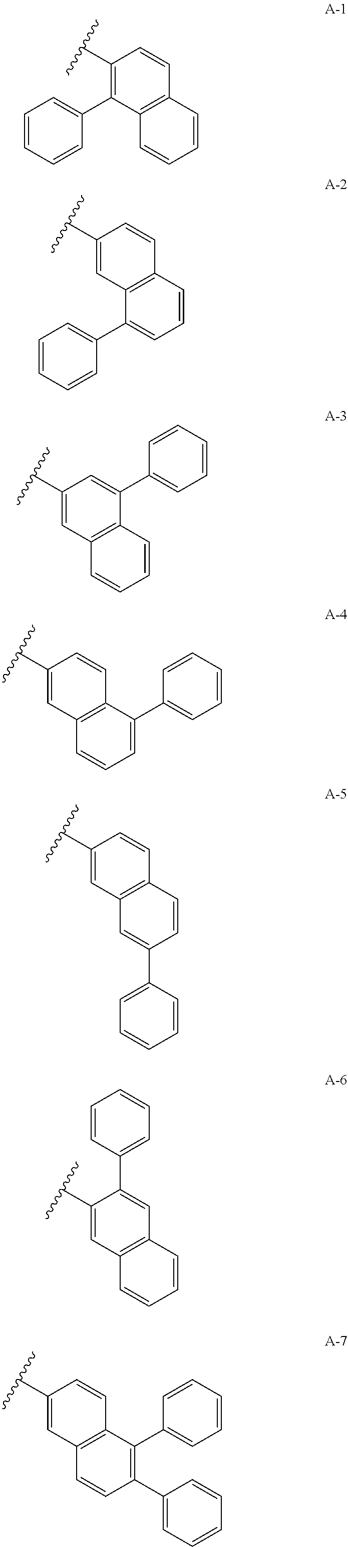

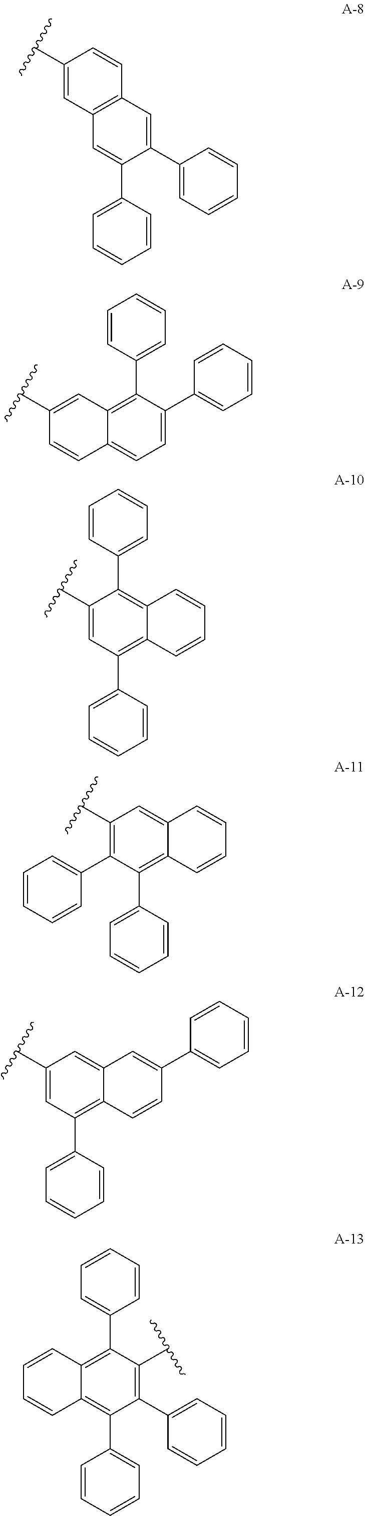

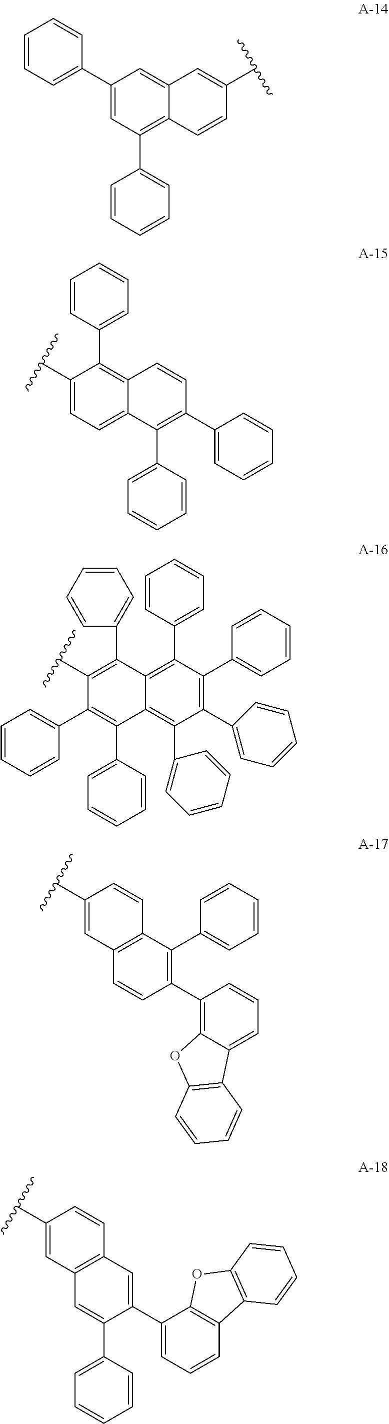

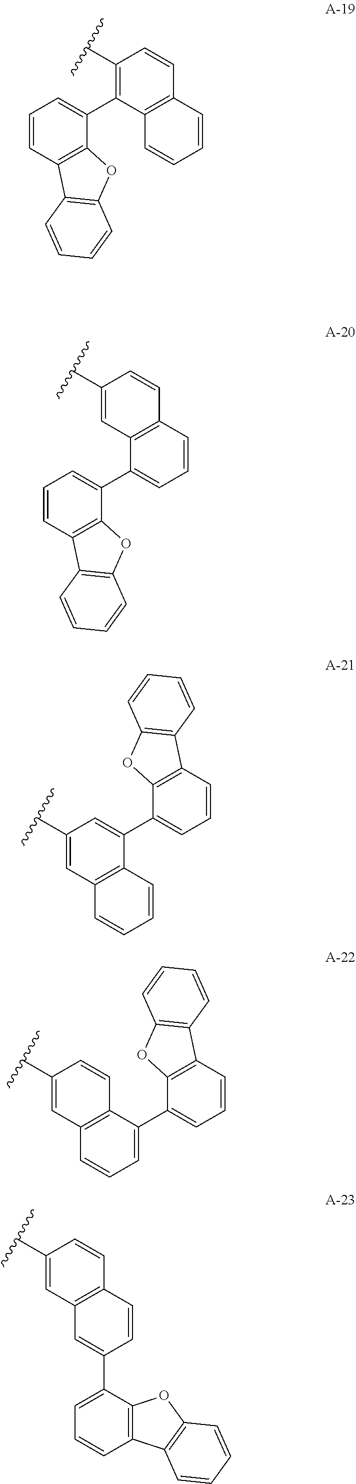 [SMILES: *c1ccc2ccccc2c1-c1cccc2c1oc1ccccc12 ; *c1ccc2cccc(-c3cccc4c3oc3ccccc34)c2c1 ; *c1cc(-c2cccc3c2oc2ccccc23)c2ccccc2c1 ; *c1ccc2c(-c3cccc4c3oc3ccccc34)cccc2c1 ; *c1ccc2ccc(-c3cccc4c3oc3ccccc34)cc2c1]

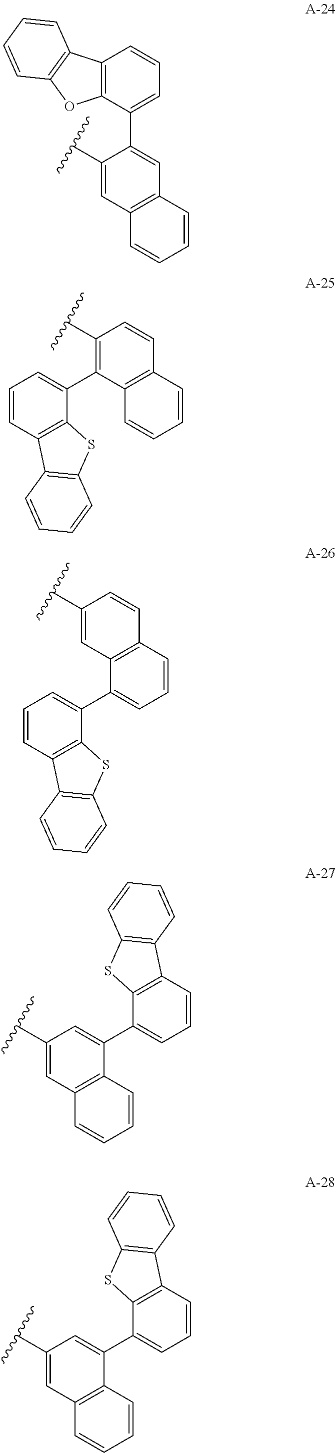 [SMILES: *c1cc2ccccc2cc1-c1cccc2c1oc1ccccc12 ; *c1ccc2ccccc2c1-c1cccc2c1sc1ccccc12 ; *c1ccc2cccc(-c3cccc4c3sc3ccccc34)c2c1 ; *c1cc(-c2cccc3c2sc2ccccc23)c2ccccc2c1 ; *c1cc(-c2cccc3c2sc2ccccc23)c2ccccc2c1]

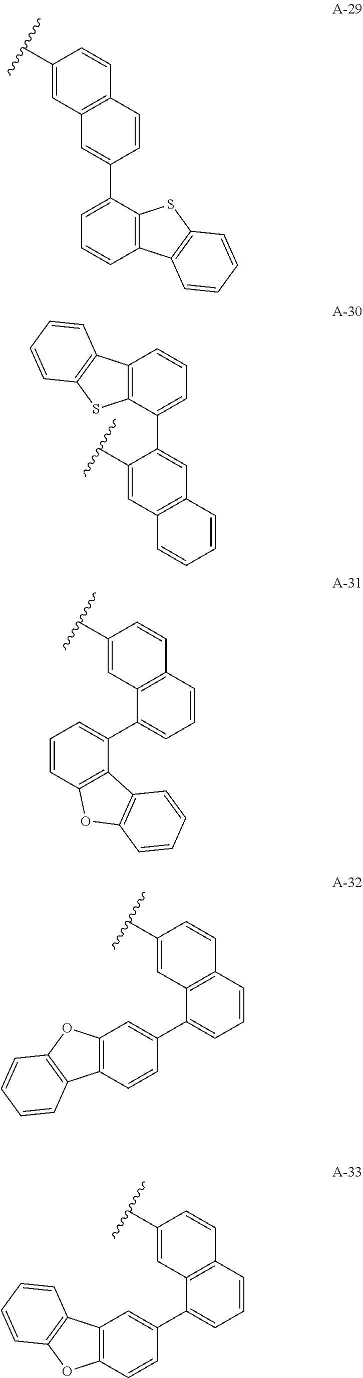 [SMILES: *c1ccc2ccc(-c3cccc4c3sc3ccccc34)cc2c1 ; *c1cc2ccccc2cc1-c1cccc2c1sc1ccccc12 ; *c1ccc2cccc(-c3cccc4oc5ccccc5c34)c2c1 ; *c1ccc2cccc(-c3ccc4c(c3)oc3ccccc34)c2c1 ; *c1ccc2cccc(-c3ccc4oc5ccccc5c4c3)c2c1]

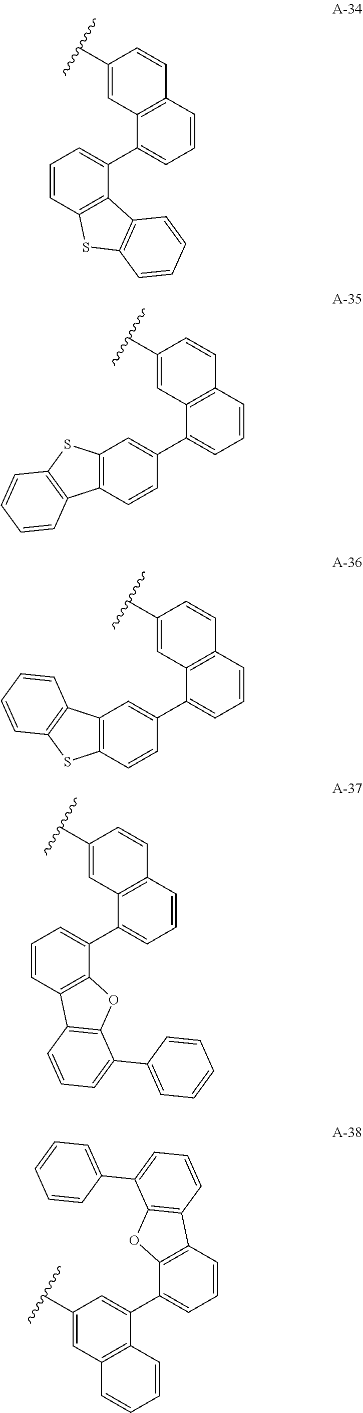 [SMILES: *c1ccc2cccc(-c3cccc4sc5ccccc5c34)c2c1 ; *c1ccc2cccc(-c3ccc4c(c3)sc3ccccc34)c2c1 ; *c1ccc2cccc(-c3ccc4sc5ccccc5c4c3)c2c1 ; *c1ccc2cccc(-c3cccc4c3oc3c(-c5ccccc5)cccc34)c2c1 ; *c1cc(-c2cccc3c2oc2c(-c4ccccc4)cccc23)c2ccccc2c1]

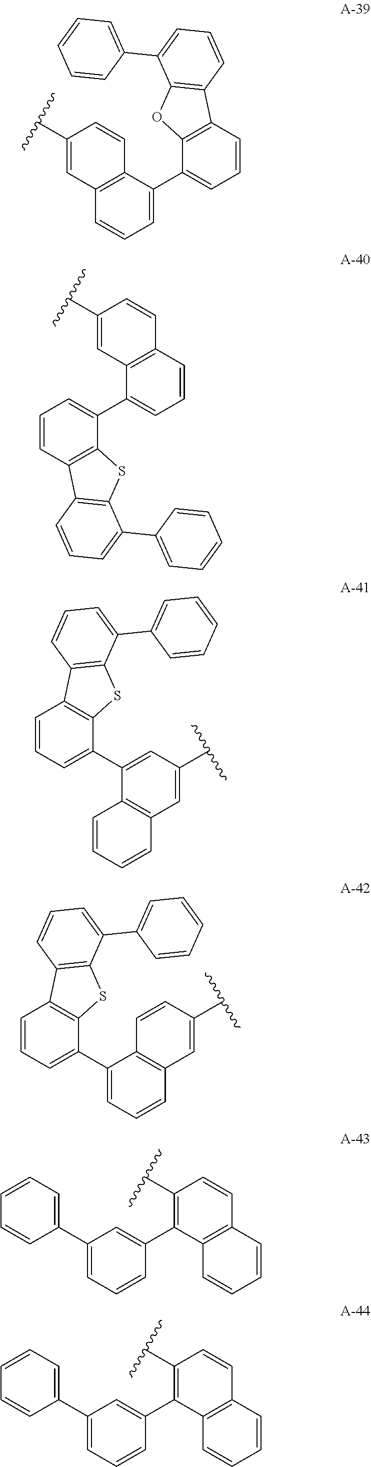

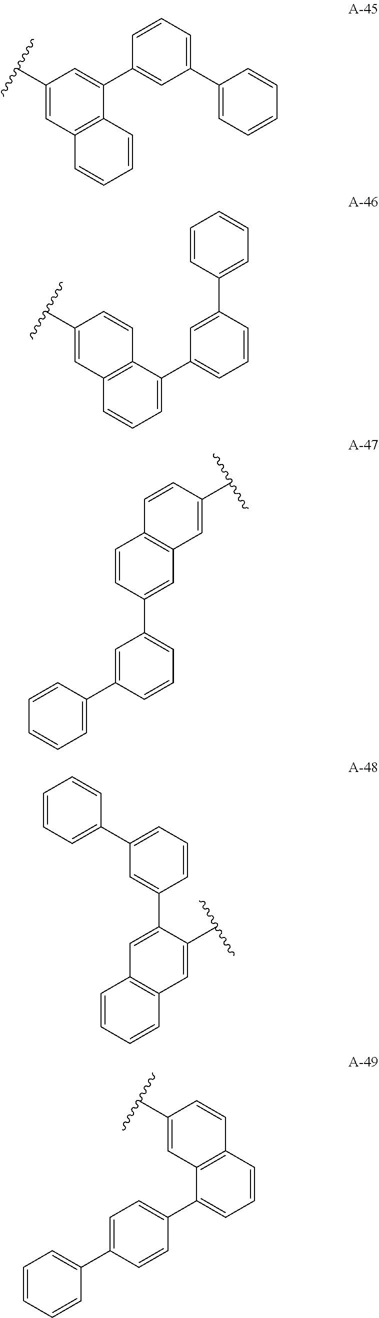

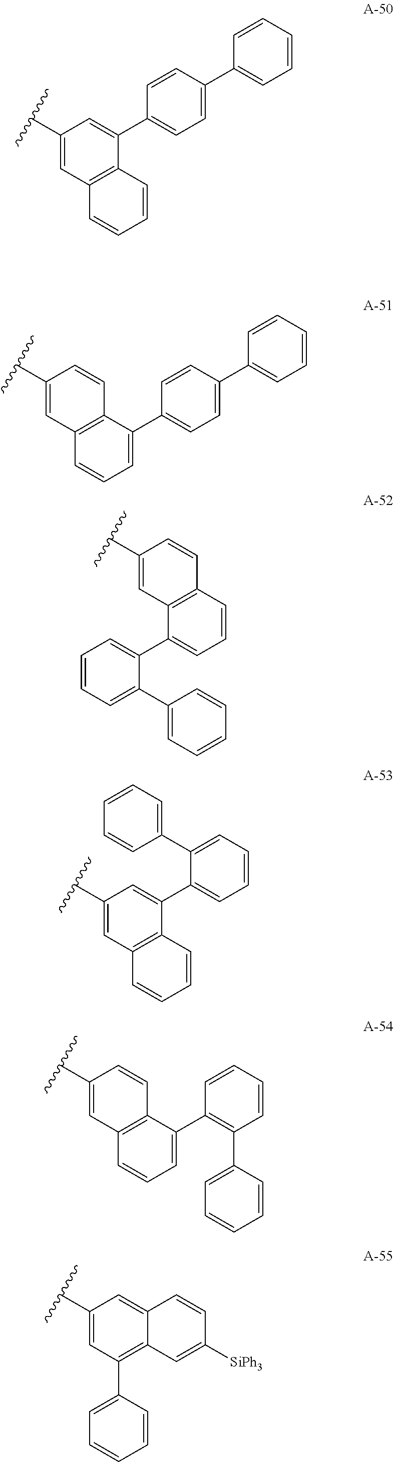

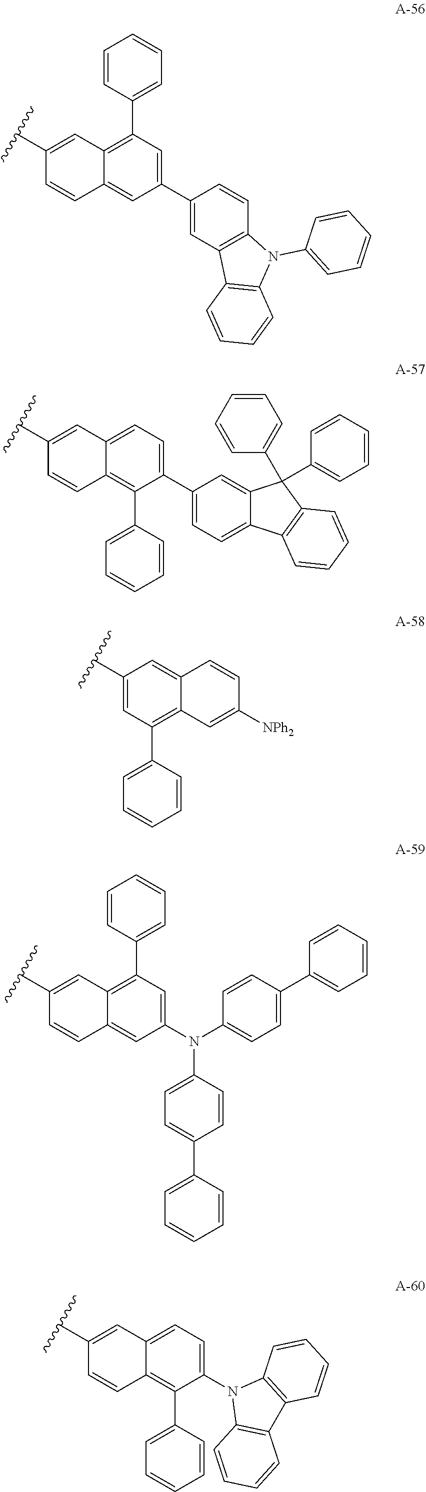

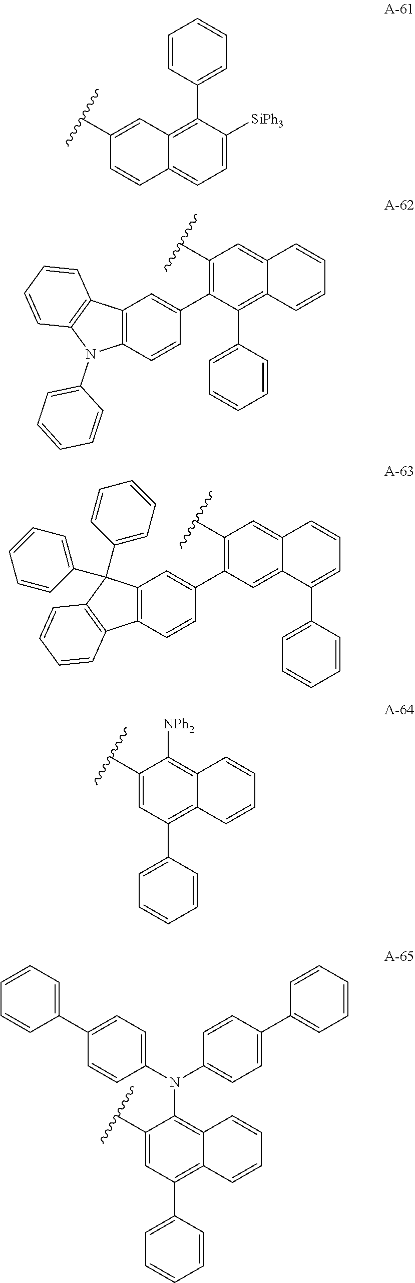

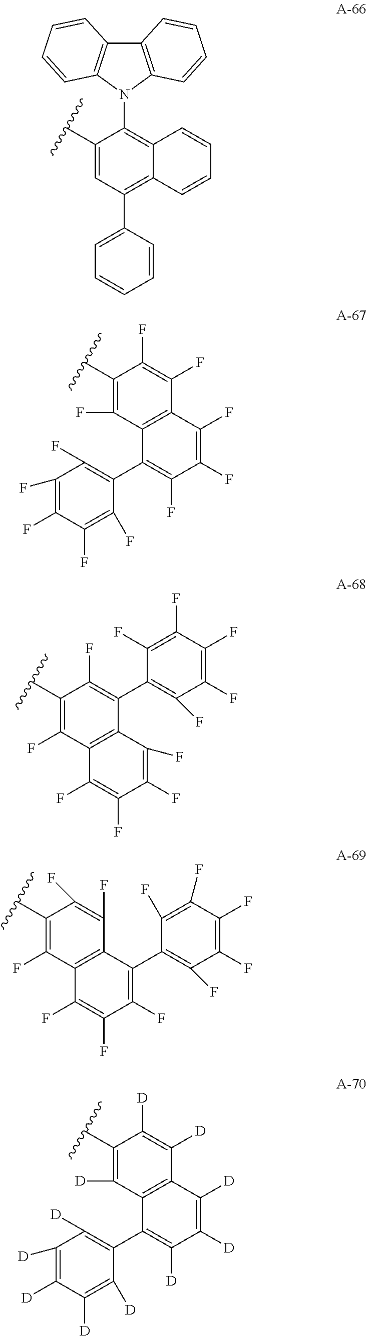

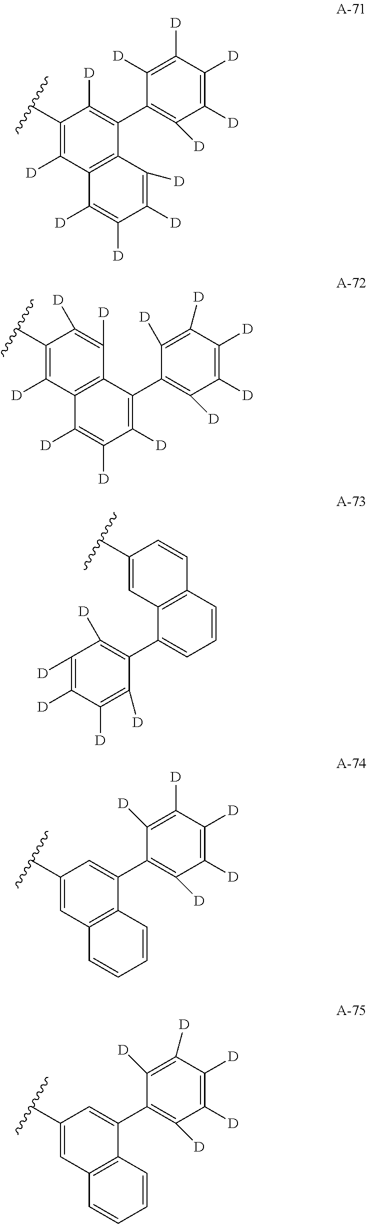 [SMILES: *c1c([2H])c(-c2c([2H])c([2H])c([2H])c([2H])c2[2H])c2c([2H])c([2H])c([2H])c([2H])c2c1[2H] ; *c1c([2H])c([2H])c2c(-c3c([2H])c([2H])c([2H])c([2H])c3[2H])c([2H])c([2H])c([2H])c2c1[2H] ; *c1ccc2cccc(-c3c([2H])c([2H])c([2H])c([2H])c3[2H])c2c1 ; *c1cc(-c2c([2H])c([2H])c([2H])c([2H])c2[2H])c2ccccc2c1 ; *c1cc(-c2c([2H])c([2H])c([2H])c([2H])c2[2H])c2ccccc2c1]

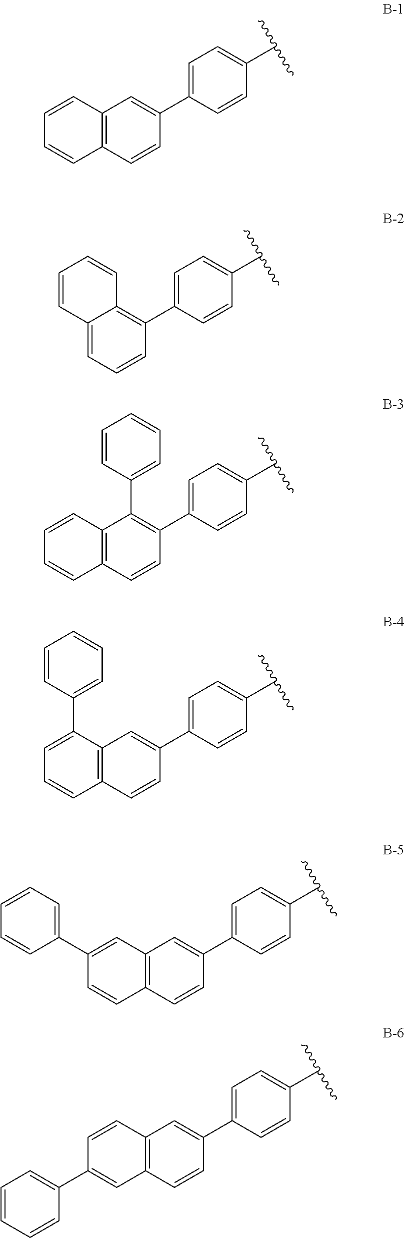

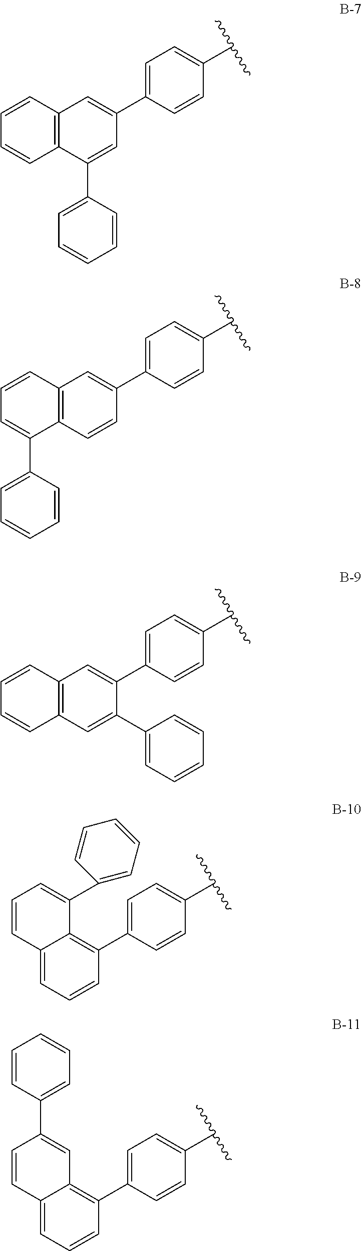

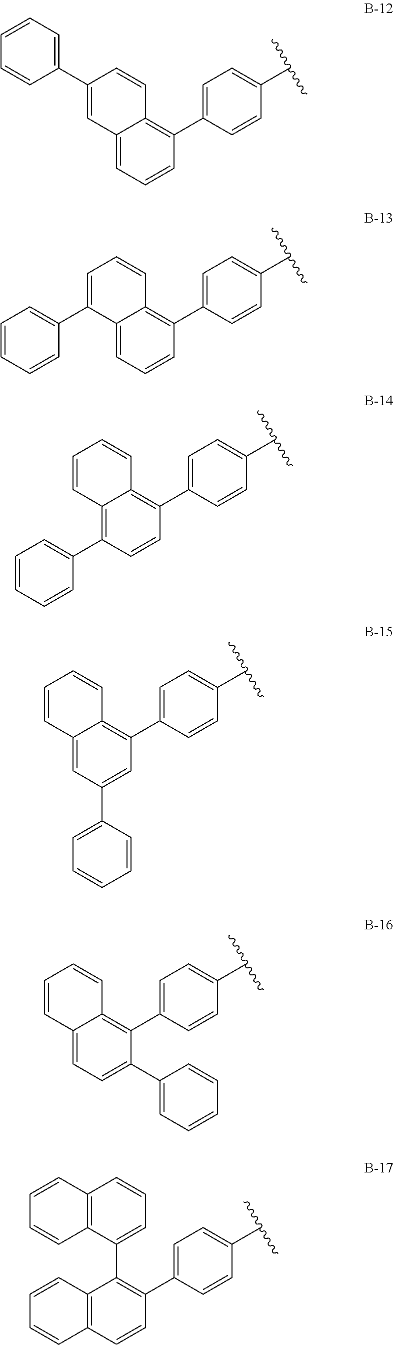 [SMILES: *c1ccc(-c2cccc3cc(-c4ccccc4)ccc23)cc1 ; *c1ccc(-c2cccc3c(-c4ccccc4)cccc23)cc1 ; *c1ccc(-c2ccc(-c3ccccc3)c3ccccc23)cc1 ; *c1ccc(-c2cc(-c3ccccc3)cc3ccccc23)cc1 ; *c1ccc(-c2c(-c3ccccc3)ccc3ccccc23)cc1 ; *c1ccc(-c2ccc3ccccc3c2-c2cccc3ccccc23)cc1]

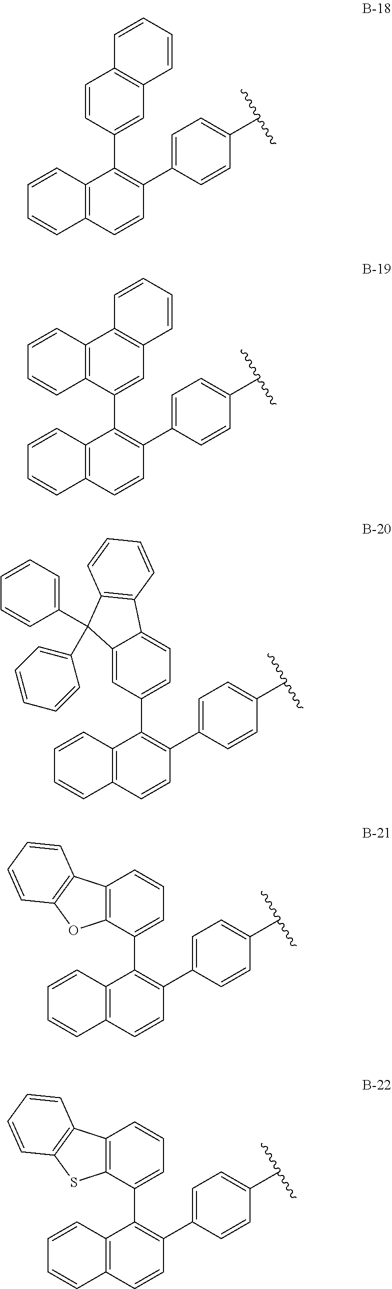

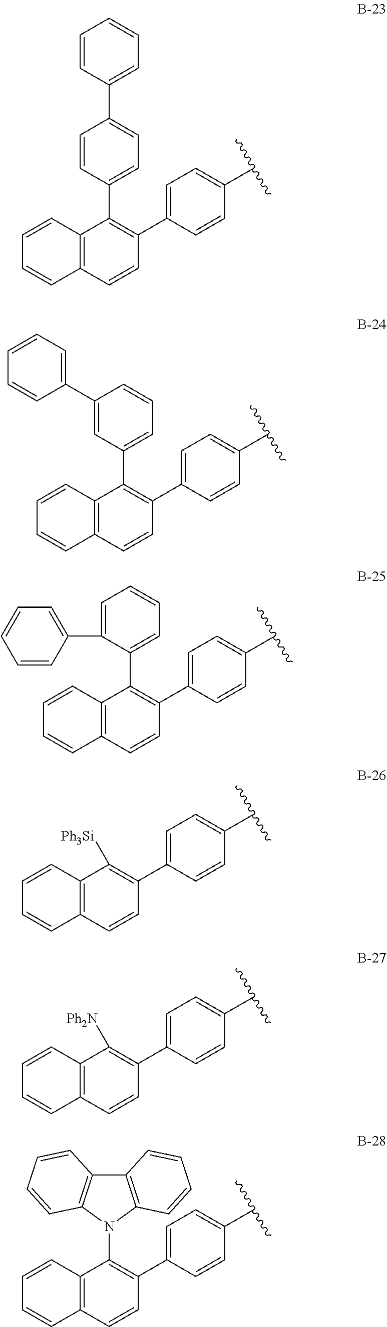

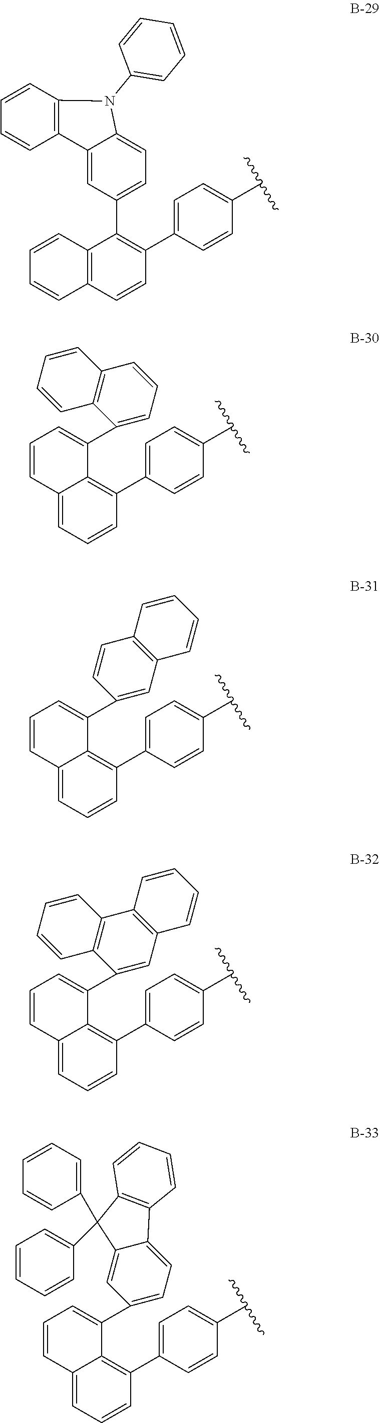 [SMILES: *c1ccc(-c2ccc3ccccc3c2-c2ccc3c(c2)c2ccccc2n3-c2ccccc2)cc1 ; *c1ccc(-c2cccc3cccc(-c4cccc5ccccc45)c23)cc1 ; *c1ccc(-c2cccc3cccc(-c4ccc5ccccc5c4)c23)cc1 ; *c1ccc(-c2cccc3cccc(-c4cc5ccccc5c5ccccc45)c23)cc1 ; *c1ccc(-c2cccc3cccc(-c4ccc5c(c4)C(c4ccccc4)(c4ccccc4)c4ccccc4-5)c23)cc1]

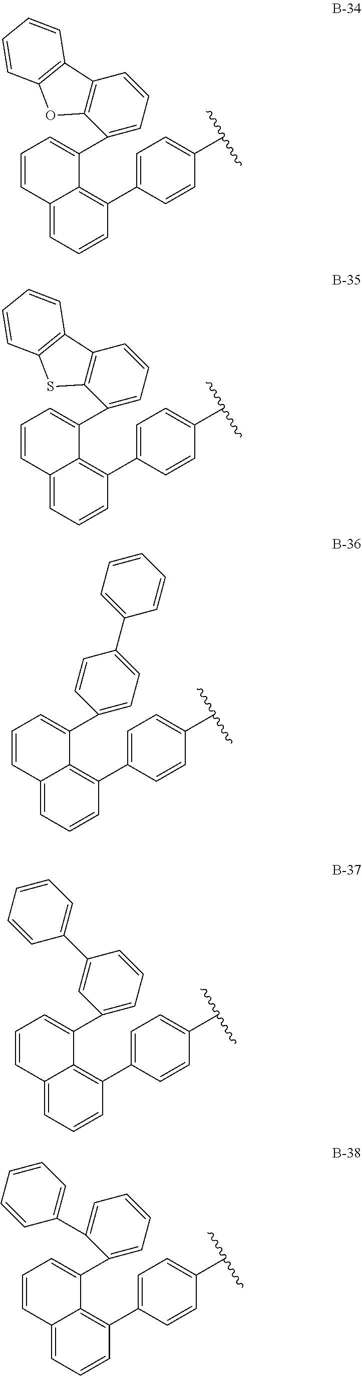

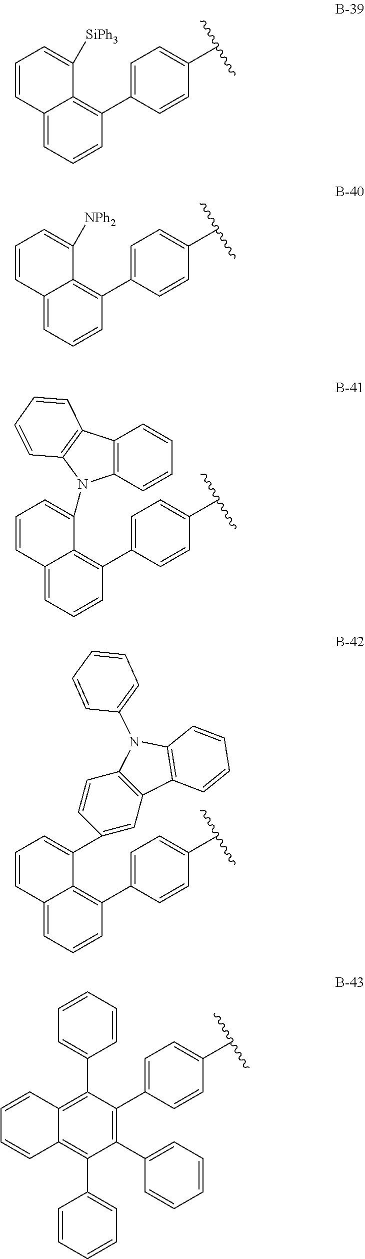

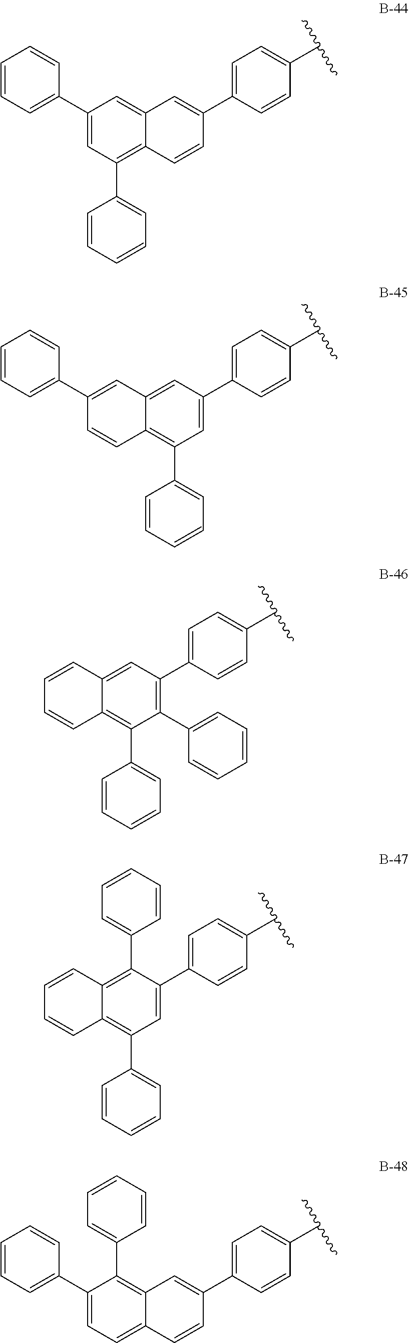

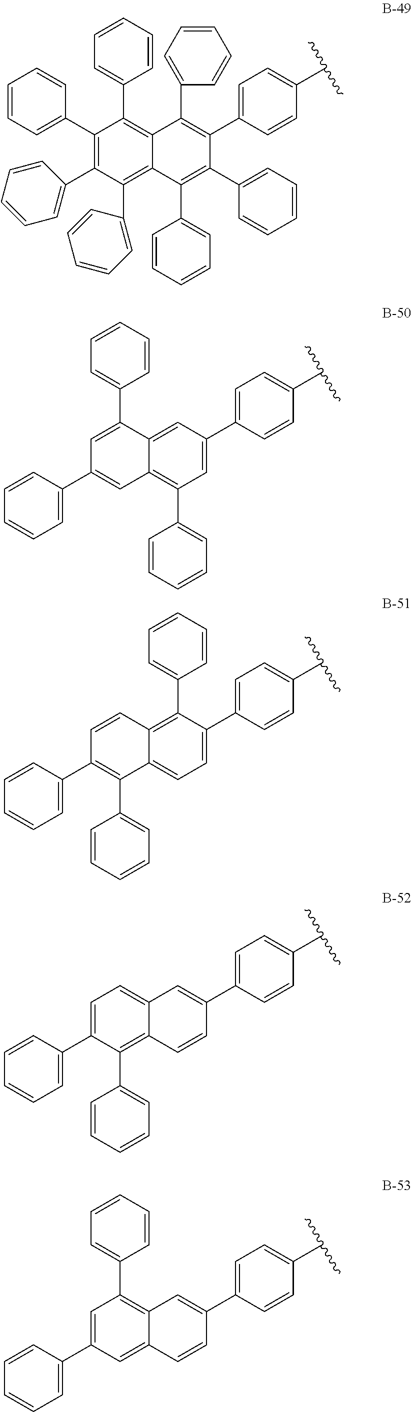

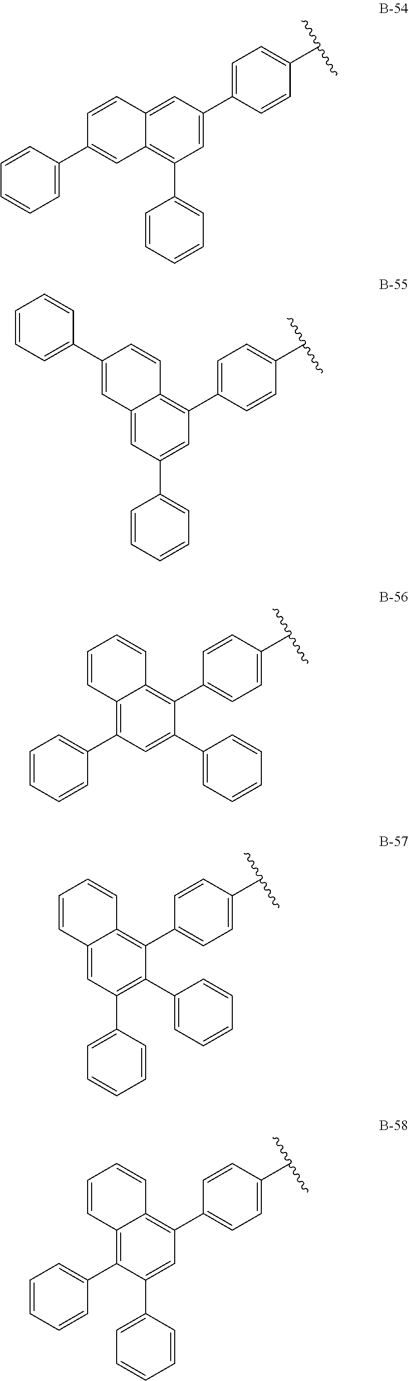 [SMILES: *c1ccc(-c2cc(-c3ccccc3)c3cc(-c4ccccc4)ccc3c2)cc1 ; *c1ccc(-c2cc(-c3ccccc3)cc3cc(-c4ccccc4)ccc23)cc1 ; *c1ccc(-c2c(-c3ccccc3)cc(-c3ccccc3)c3ccccc23)cc1 ; *c1ccc(-c2c(-c3ccccc3)c(-c3ccccc3)cc3ccccc23)cc1 ; *c1ccc(-c2cc(-c3ccccc3)c(-c3ccccc3)c3ccccc23)cc1]

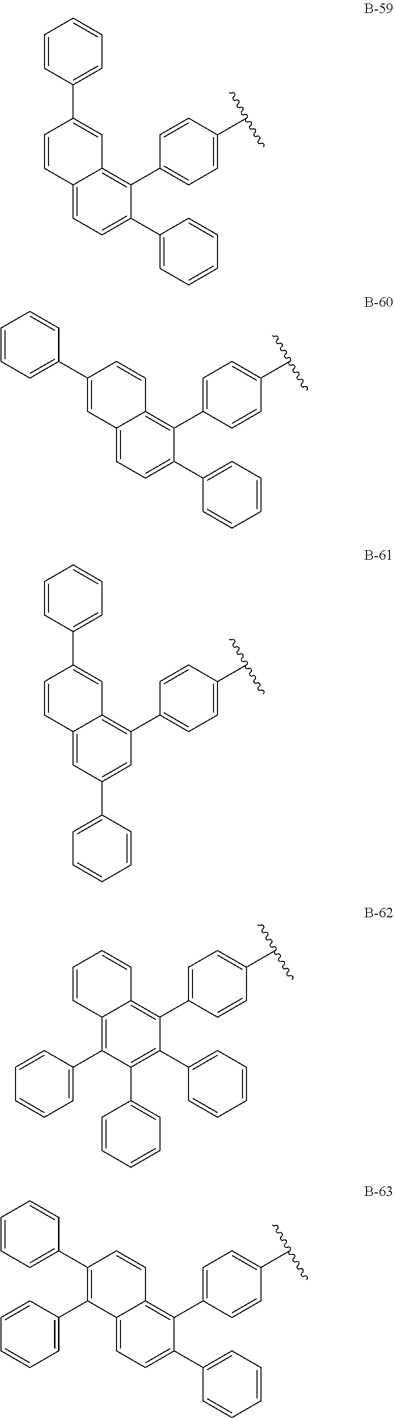 [SMILES: *c1ccc(-c2c(-c3ccccc3)ccc3ccc(-c4ccccc4)cc23)cc1 ; *c1ccc(-c2c(-c3ccccc3)ccc3cc(-c4ccccc4)ccc23)cc1 ; *c1ccc(-c2cc(-c3ccccc3)cc3ccc(-c4ccccc4)cc23)cc1 ; *c1ccc(-c2c(-c3ccccc3)c(-c3ccccc3)c(-c3ccccc3)c3ccccc23)cc1 ; *c1ccc(-c2c(-c3ccccc3)ccc3c(-c4ccccc4)c(-c4ccccc4)ccc23)cc1]

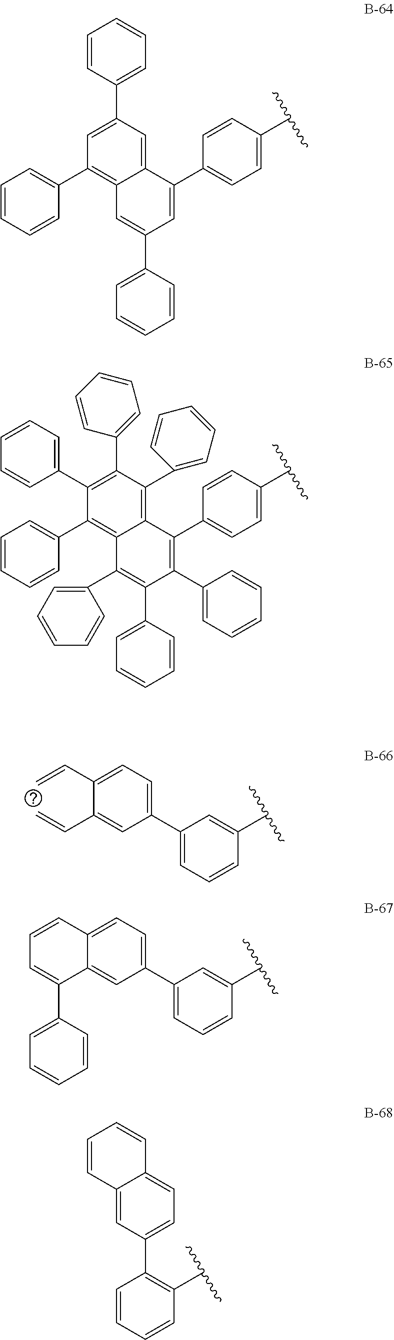 [SMILES: *c1ccc(-c2cc(-c3ccccc3)cc3c(-c4ccccc4)cc(-c4ccccc4)cc23)cc1 ; *c1ccc(-c2c(-c3ccccc3)c(-c3ccccc3)c(-c3ccccc3)c3c(-c4ccccc4)c(-c4ccccc4)c(-c4ccccc4)c(-c4ccccc4)c23)cc1 ; *c1cccc(-c2ccc(C=C)c(C=C)c2)c1 ; *c1cccc(-c2ccc3cccc(-c4ccccc4)c3c2)c1 ; *c1ccccc1-c1ccc2ccccc2c1]

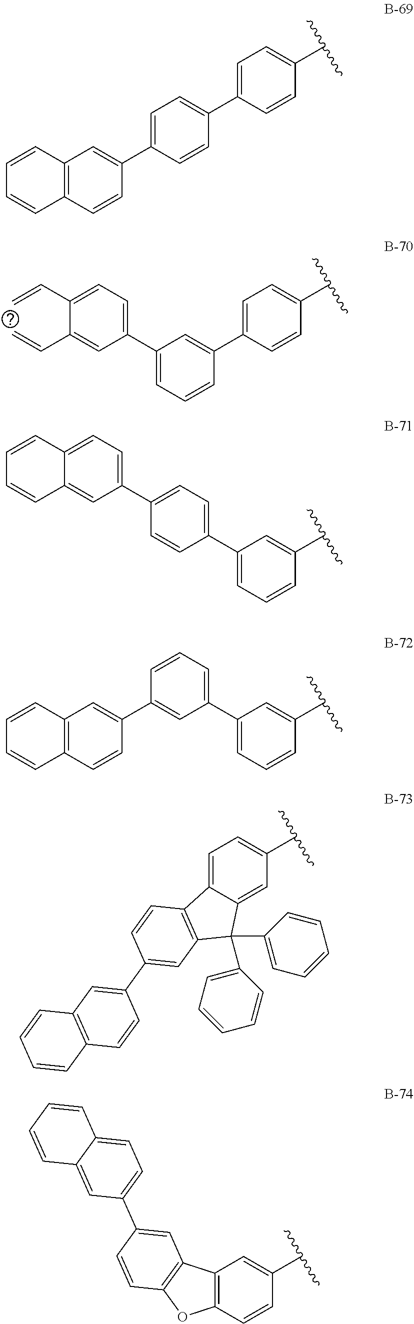

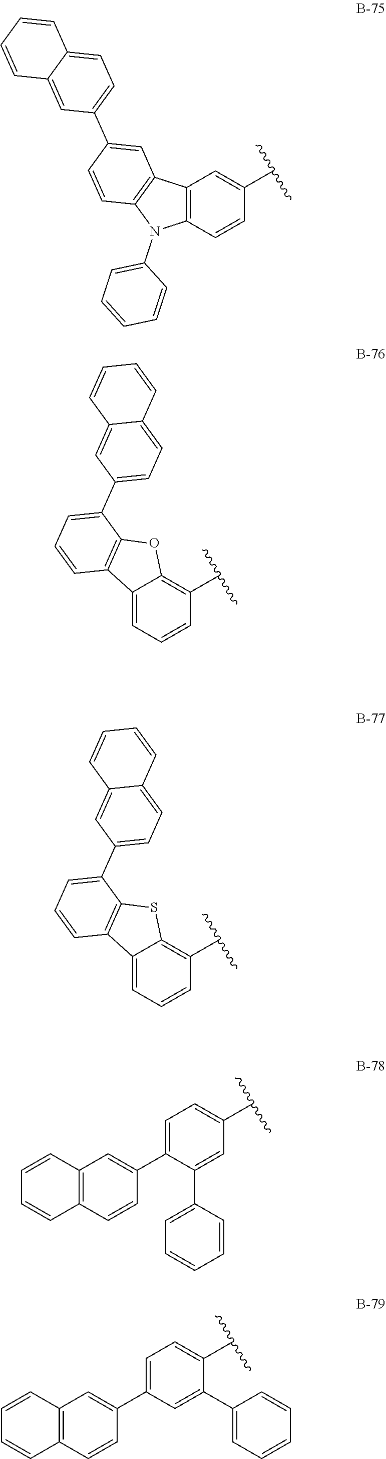

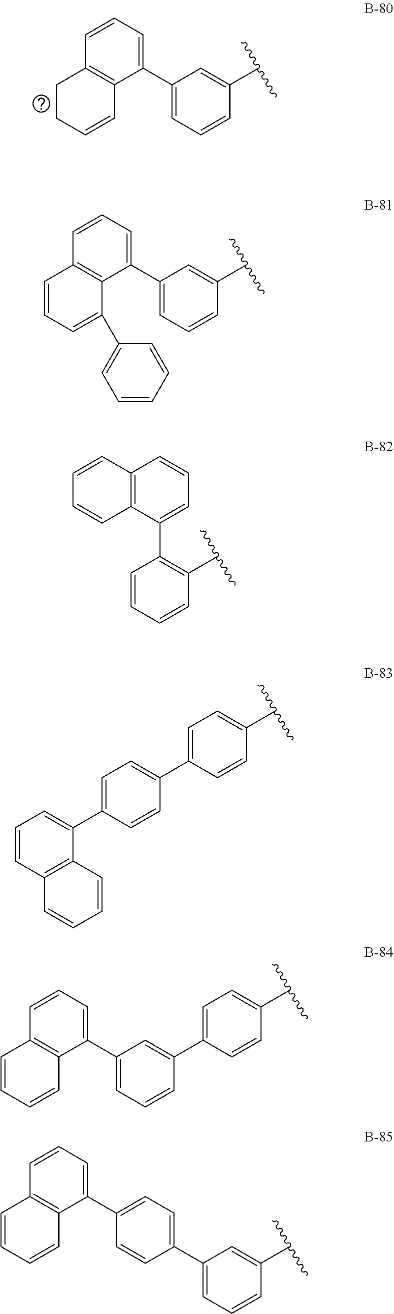 [SMILES: *c1cccc(-c2cccc3c2C=CCC3)c1 ; *c1cccc(-c2cccc3cccc(-c4ccccc4)c23)c1 ; *c1ccccc1-c1cccc2ccccc12 ; *c1ccc(-c2ccc(-c3cccc4ccccc34)cc2)cc1 ; *c1ccc(-c2cccc(-c3cccc4ccccc34)c2)cc1 ; *c1cccc(-c2ccc(-c3cccc4ccccc34)cc2)c1]

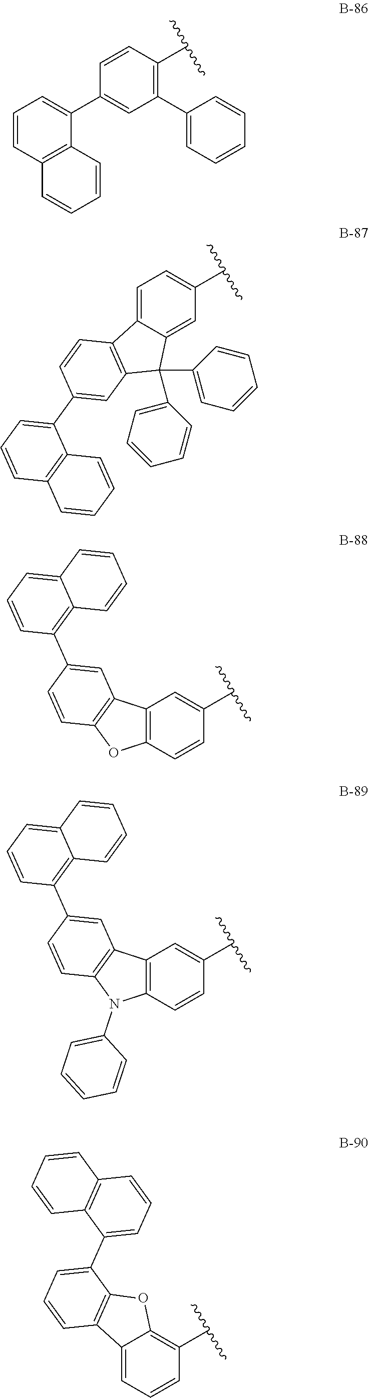

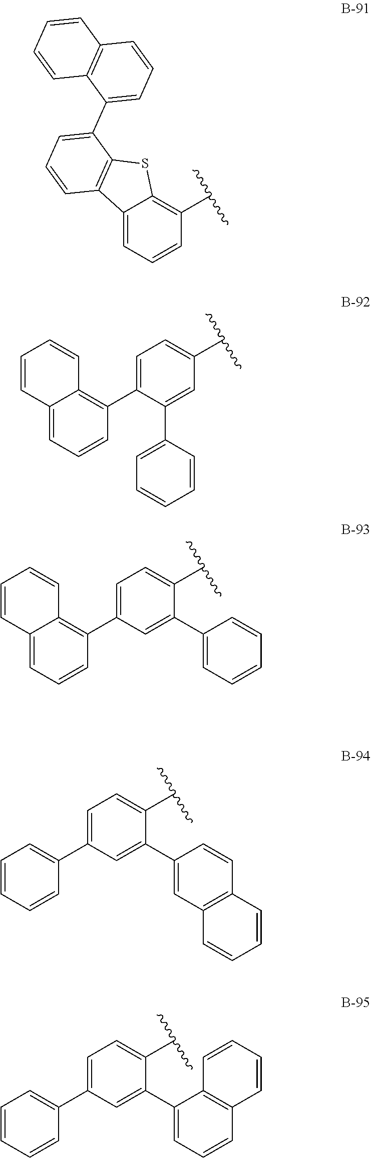 [SMILES: *c1cccc2c1sc1c(-c3cccc4ccccc34)cccc12 ; *c1ccc(-c2cccc3ccccc23)c(-c2ccccc2)c1 ; *c1ccc(-c2cccc3ccccc23)cc1-c1ccccc1 ; *c1ccc(-c2ccccc2)cc1-c1ccc2ccccc2c1 ; *c1ccc(-c2ccccc2)cc1-c1cccc2ccccc12]

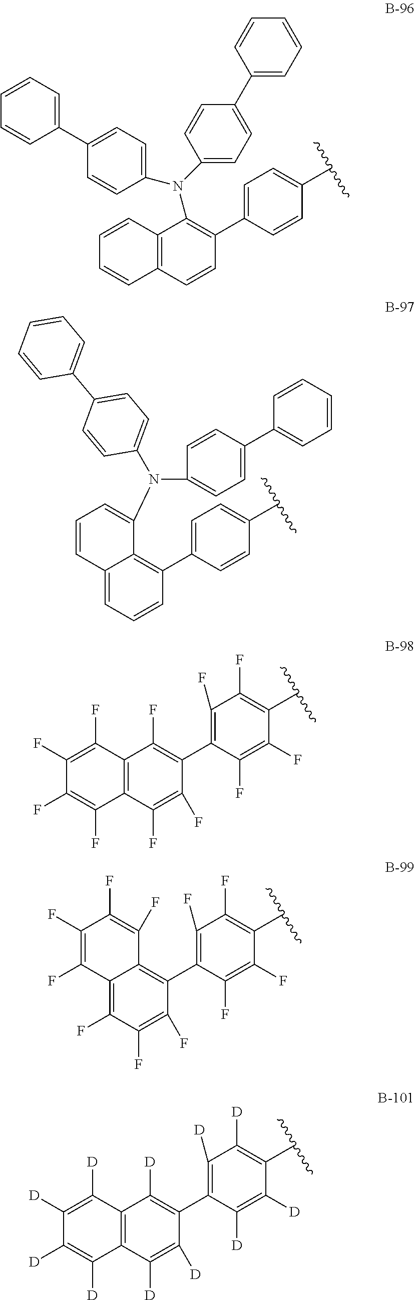

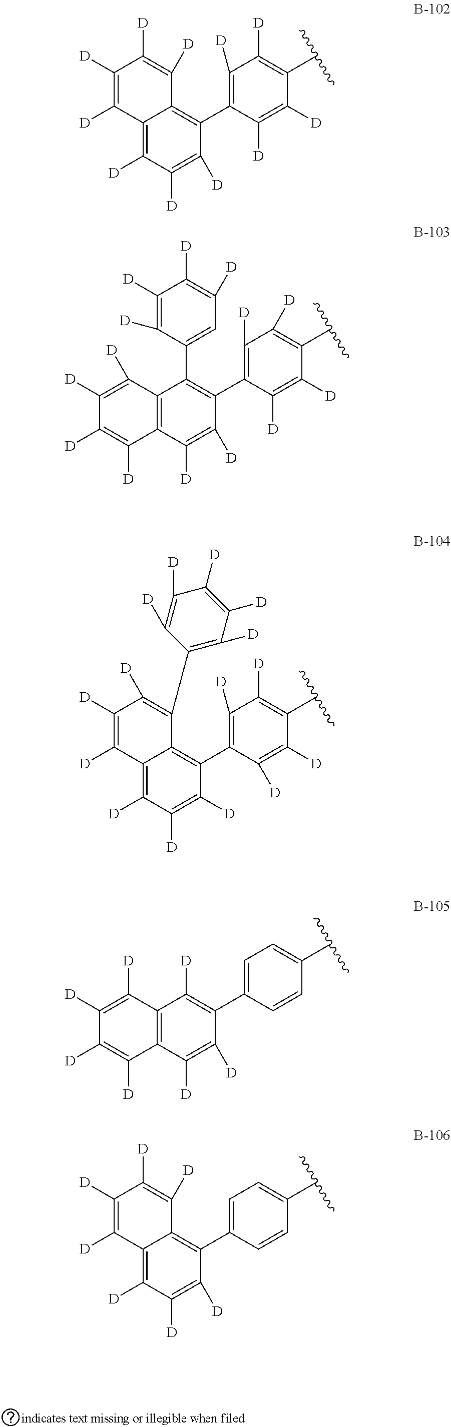

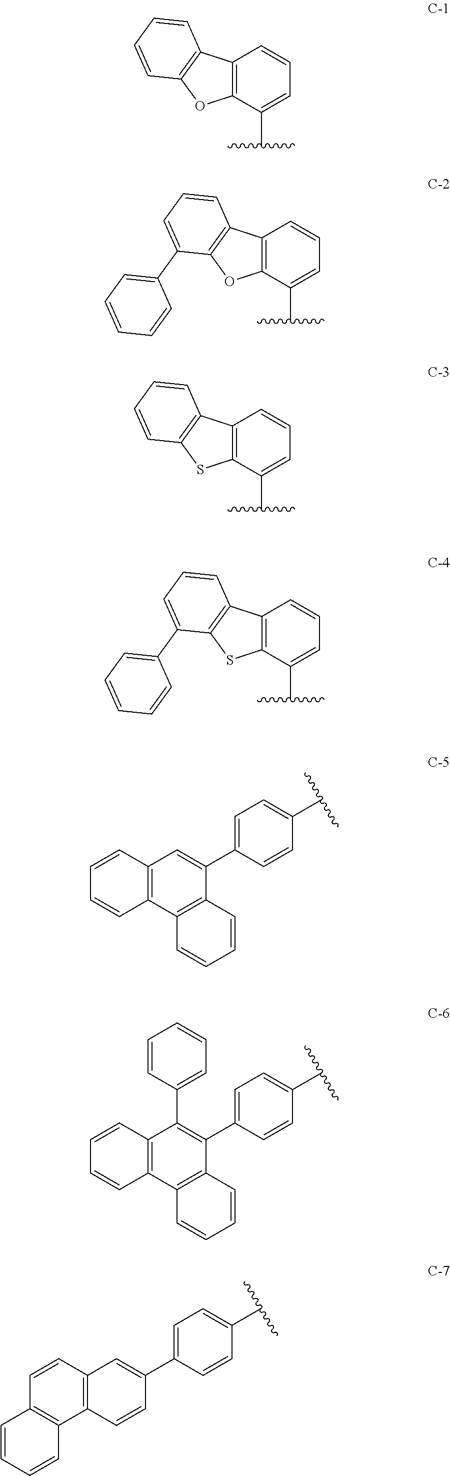

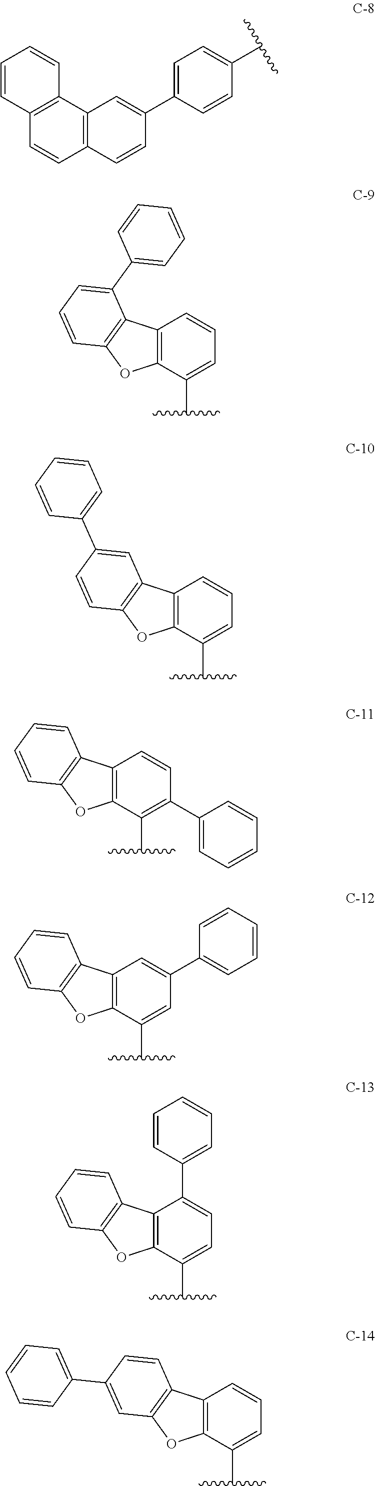

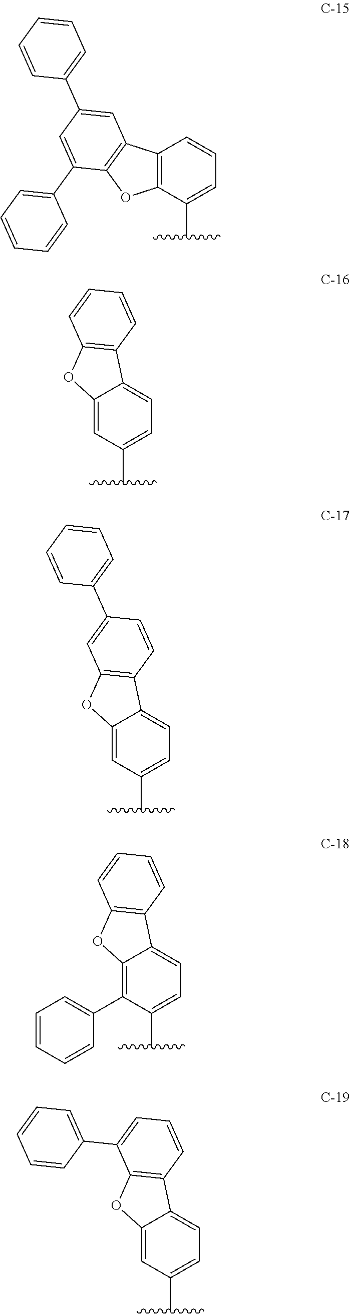 [SMILES: *c1cccc2c1oc1c(-c3ccccc3)cc(-c3ccccc3)cc12 ; *c1ccc2c(c1)oc1ccccc12 ; *c1ccc2c(c1)oc1cc(-c3ccccc3)ccc12 ; *c1ccc2c(oc3ccccc32)c1-c1ccccc1 ; *c1ccc2c(c1)oc1c(-c3ccccc3)cccc12]

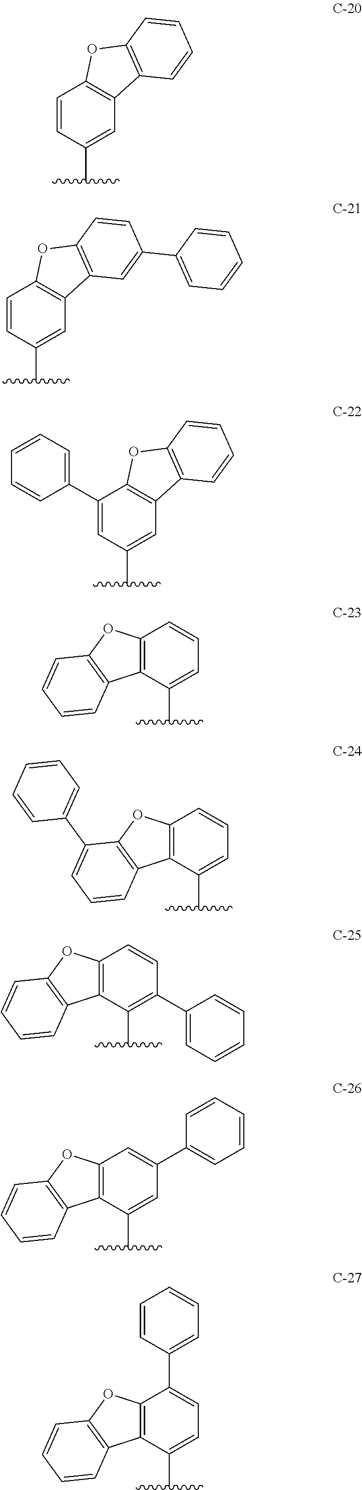

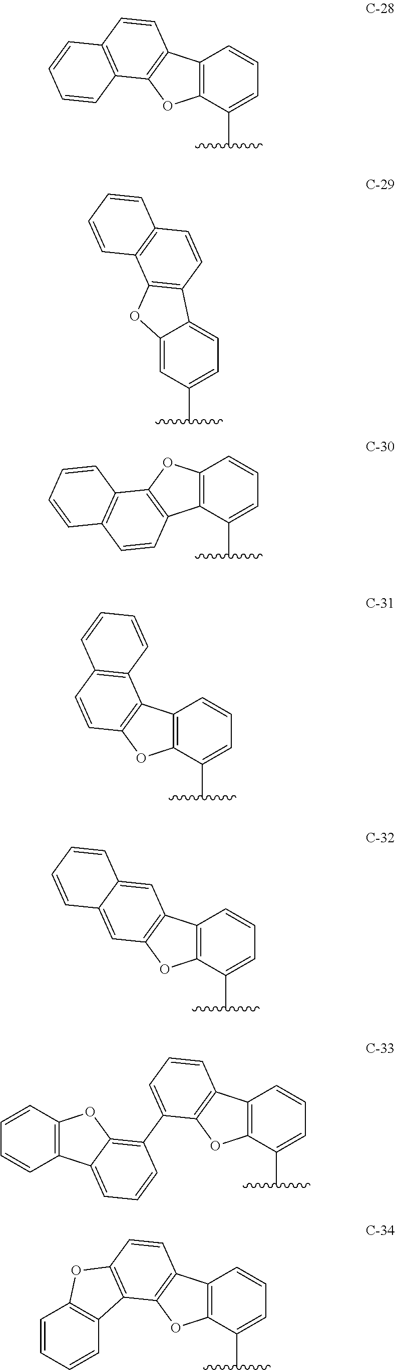

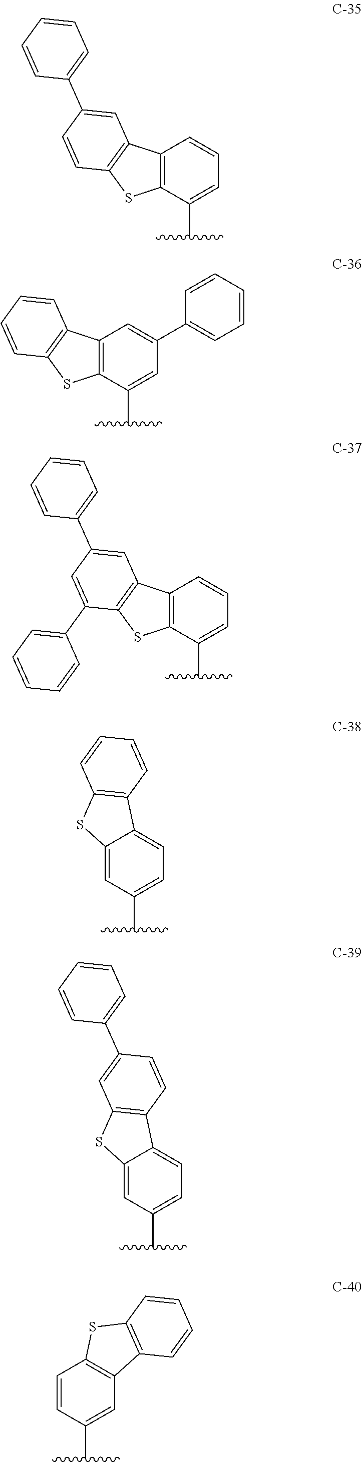

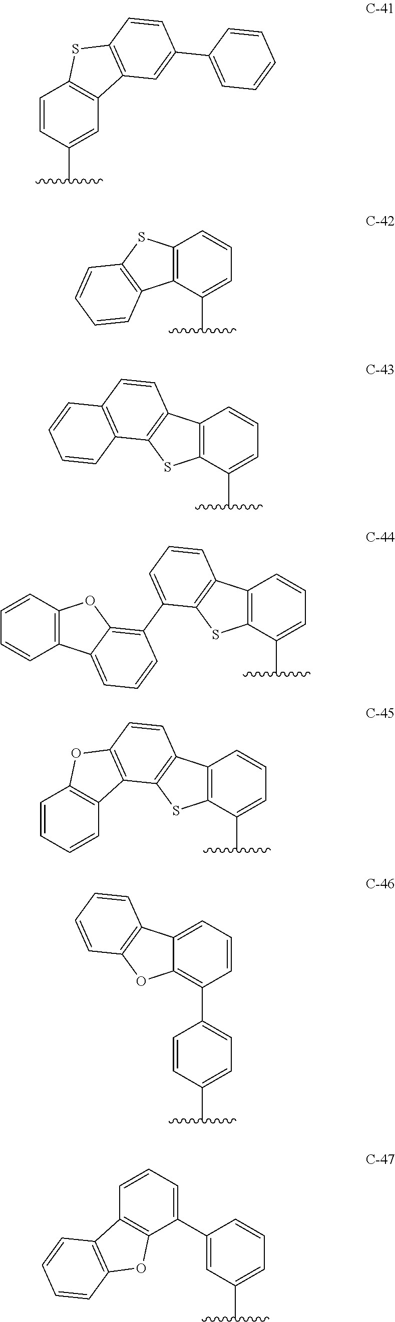

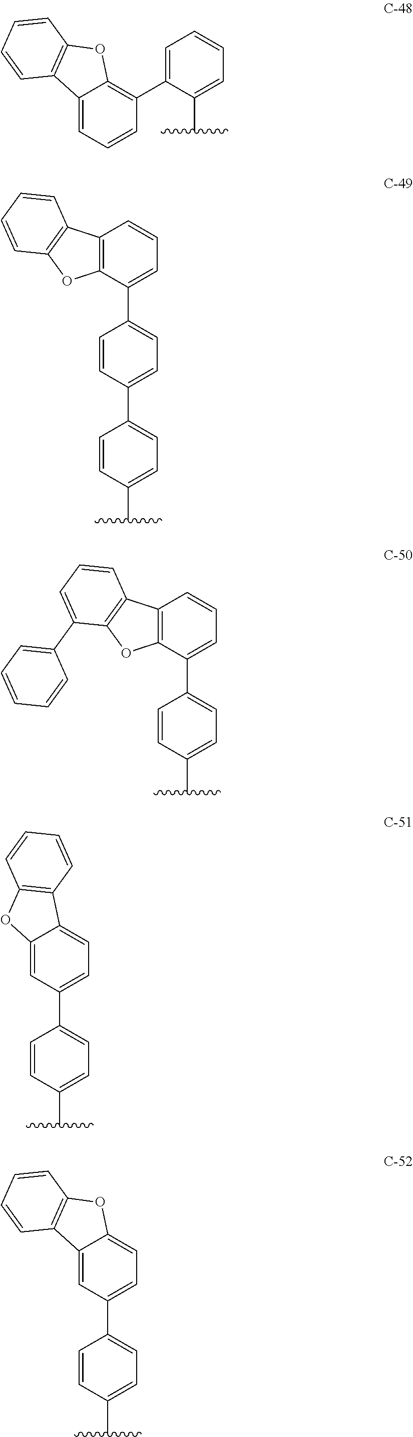 [SMILES: *c1ccccc1-c1cccc2c1oc1ccccc12 ; *c1ccc(-c2ccc(-c3cccc4c3oc3ccccc34)cc2)cc1 ; *c1ccc(-c2cccc3c2oc2c(-c4ccccc4)cccc23)cc1 ; *c1ccc(-c2ccc3c(c2)oc2ccccc23)cc1 ; *c1ccc(-c2ccc3oc4ccccc4c3c2)cc1]

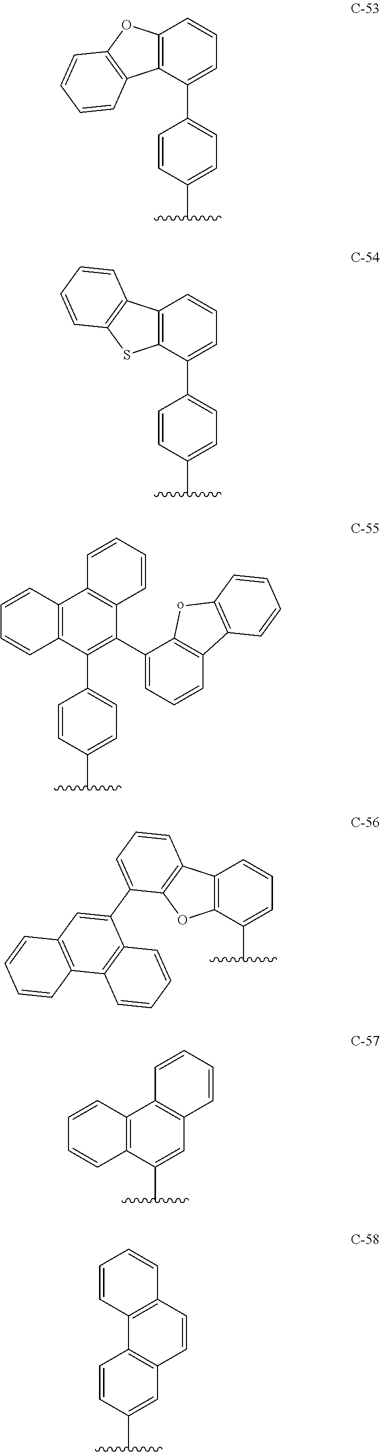

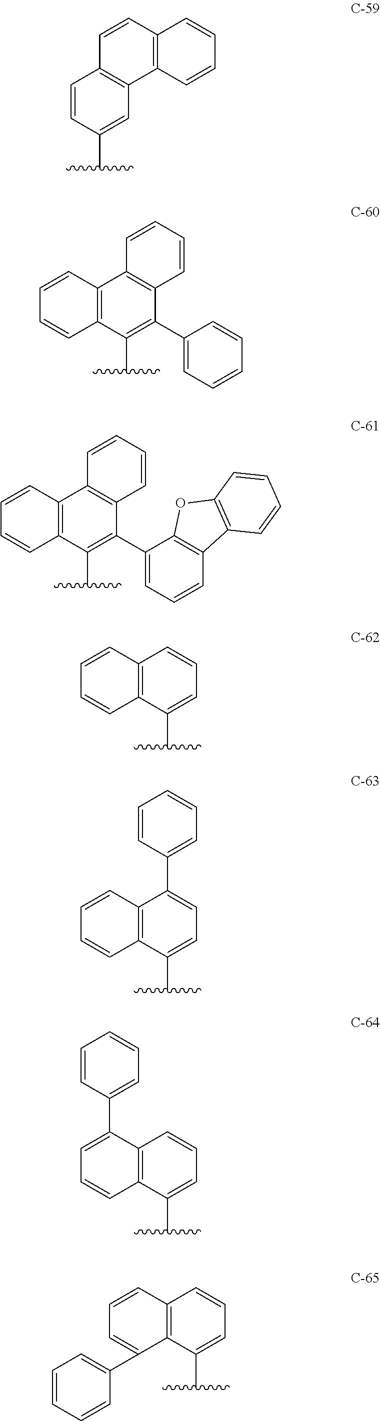

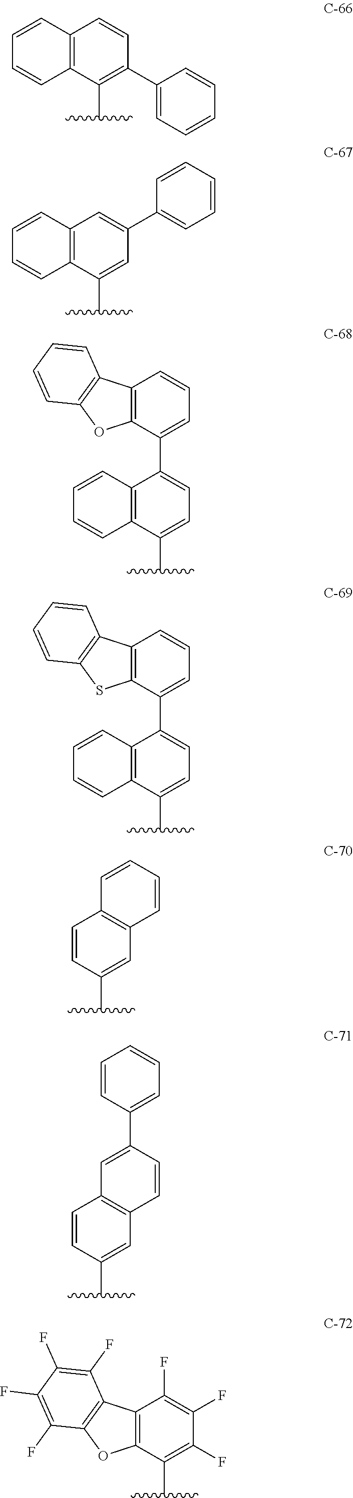

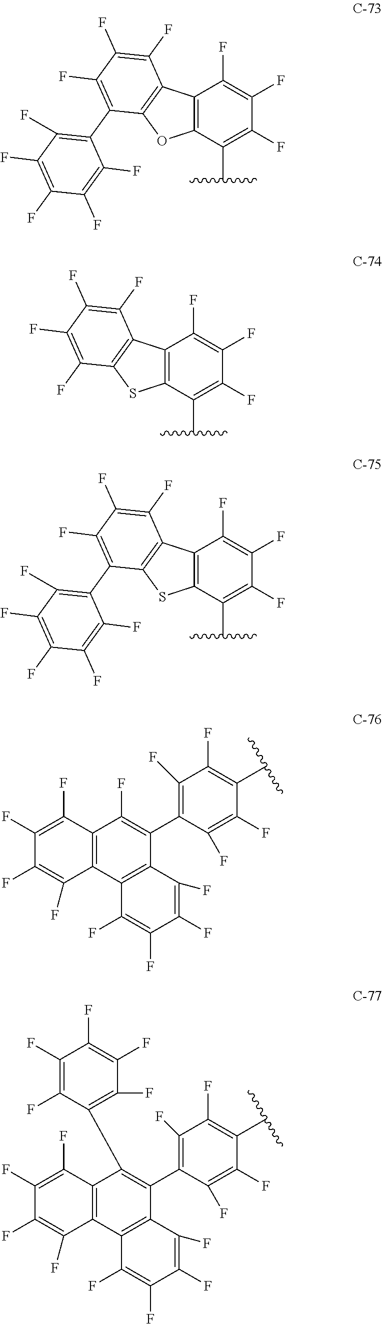 [SMILES: *c1c(F)c(F)c(F)c2c1oc1c(-c3c(F)c(F)c(F)c(F)c3F)c(F)c(F)c(F)c12 ; *c1c(F)c(F)c(F)c2c1sc1c(F)c(F)c(F)c(F)c12 ; *c1c(F)c(F)c(F)c2c1sc1c(-c3c(F)c(F)c(F)c(F)c3F)c(F)c(F)c(F)c12 ; *c1c(F)c(F)c(-c2c(F)c3c(F)c(F)c(F)c(F)c3c3c(F)c(F)c(F)c(F)c23)c(F)c1F ; *c1c(F)c(F)c(-c2c(-c3c(F)c(F)c(F)c(F)c3F)c3c(F)c(F)c(F)c(F)c3c3c(F)c(F)c(F)c(F)c23)c(F)c1F]

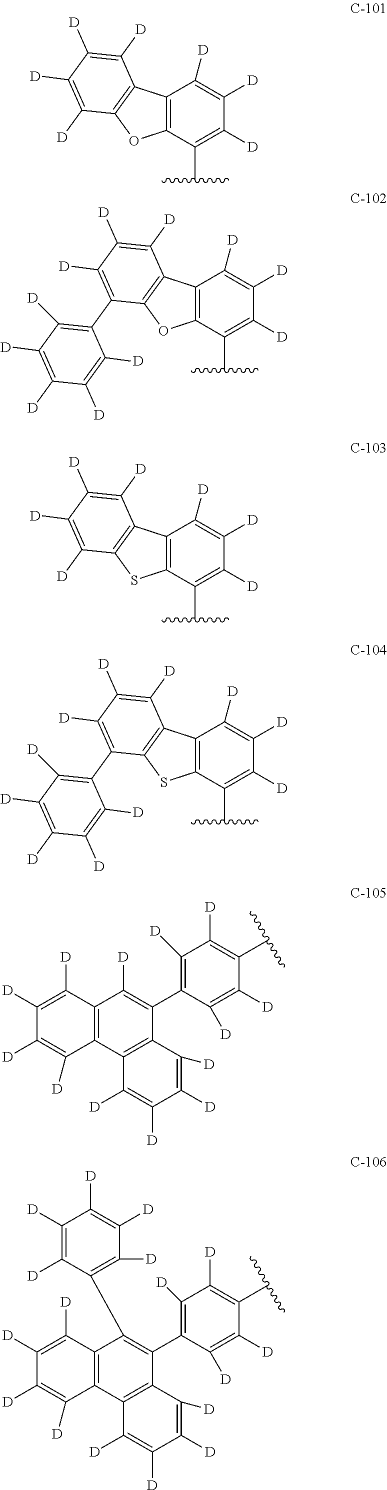

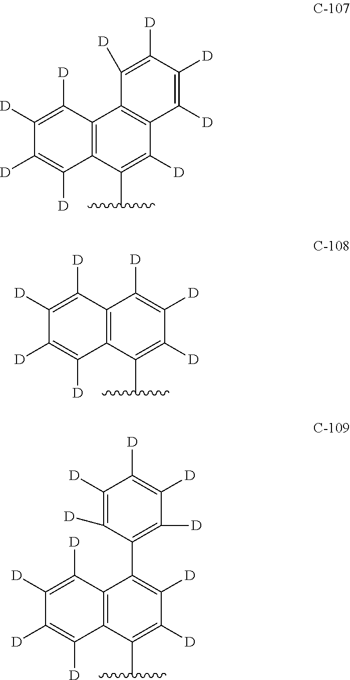 [SMILES: *c1c([2H])c2c([2H])c([2H])c([2H])c([2H])c2c2c([2H])c([2H])c([2H])c([2H])c12 ; *c1c([2H])c([2H])c([2H])c2c([2H])c([2H])c([2H])c([2H])c12 ; *c1c([2H])c([2H])c(-c2c([2H])c([2H])c([2H])c([2H])c2[2H])c2c([2H])c([2H])c([2H])c([2H])c12]

15. An amine compound represented by Formula 1: 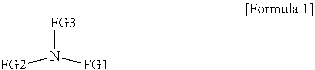
wherein in Formula 1,
FG1 is a group represented by Formula A,
FG2 is a group represented by Formula B, and
FG3 is a group represented by one of Formula A to Formula C: 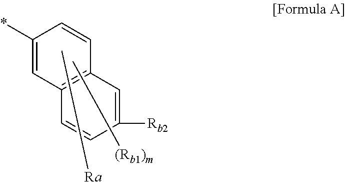
wherein in Formula A,
m is an integer from 1 to 5,
Rₐ is a substituted or unsubstituted phenyl group, a substituted or unsubstituted dibenzofuran group, or a substituted or unsubstituted dibenzothiophene group, and
R_{b1} and R_{b2} are each independently a hydrogen atom, a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group, 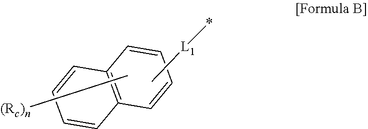
wherein in Formula B,
n is an integer from 0 to 7,
L₁ is a substituted or unsubstituted arylene group having 6 to 30 ring-forming carbon atoms, or a substituted or unsubstituted heteroarylene group having 2 to 30 ring-forming carbon atoms,
R_{c} is a deuterium atom, a halogen atom, a substituted or unsubstituted amine group, a substituted or unsubstituted silyl group, a substituted or unsubstituted phenyl group, a substituted or unsubstituted dibenzofuran group, a substituted or unsubstituted dibenzothiophene group, a substituted or unsubstituted carbazole group, or a substituted or unsubstituted fluorene group, and 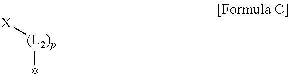
wherein in Formula C,
p is 0 or 1,
L₂ is a substituted or unsubstituted arylene group having 6 to 30 ring-forming carbon atoms, or a substituted or unsubstituted heteroarylene group having 2 to 30 ring-forming carbon atoms, and
X is a group represented by one of Formula D-1 to Formula D-3, 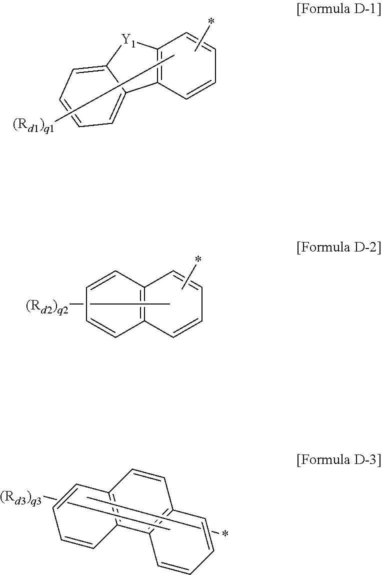
wherein in Formula D-1,
Y₁ is O or S, and
wherein in Formula D-1 to Formula D-3,
q₁ and q₂ are each independently an integer from 0 to 7,
q₃ is an integer from 0 to 9, and
R_{d1} to R_{d3} are each independently a deuterium atom, a halogen atom, a substituted or unsubstituted aryl group having 6 to 30 ring-forming carbon atoms, a substituted or unsubstituted heteroaryl group having 2 to 30 ring-forming carbon atoms, a substituted or unsubstituted alkenyl group 2 to 10 carbon atoms, a substituted or unsubstituted oxy group, or a substituted or unsubstituted thio group, or are bonded to an adjacent group to form a ring.

16. The amine compound of claim 15, wherein the group represented by Formula A is represented by Formula AA-1: 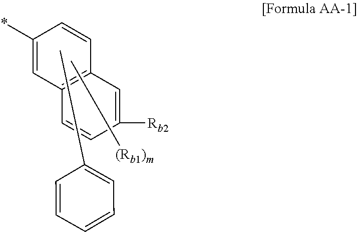
wherein in Formula AA-1,
m, R_{b1}, and R_{b2} are the same as defined in Formula A.

17. The amine compound of claim 15, wherein the group represented by Formula B is represented by one of Formula BB-1 to Formula BB-3: 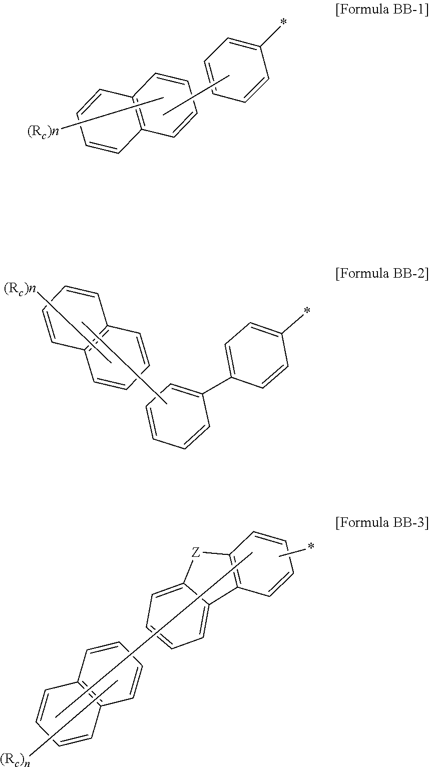
wherein in Formula BB-3,
Z is C(R_{z1})(R_{z2}), N(R_{z3}), O, or S, and
R_{z1} to R_{z3} are each independently a substituted or unsubstituted phenyl group, and
wherein in Formula BB-1 to Formula BB-3,
n and R_{c} are the same as defined Formula B.

18. The amine compound of claim 17, wherein the group represented by Formula BB-3 is represented by one of Formula BBB-1 to Formula BBB-3: 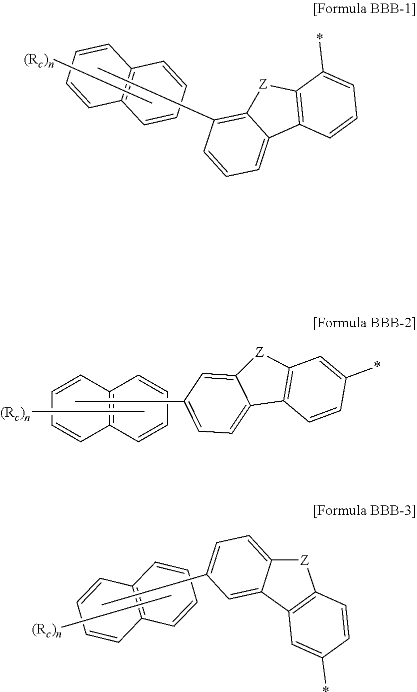
wherein in Formula BBB-1 to Formula BBB-3,
n and R_{c} are the same as defined in Formula B, and
Z is the same as defined in Formula BB-3.

19. The amine compound of claim 15, wherein the group represented by Formula C is represented by one of Formula CC-1 to Formula CC-3: 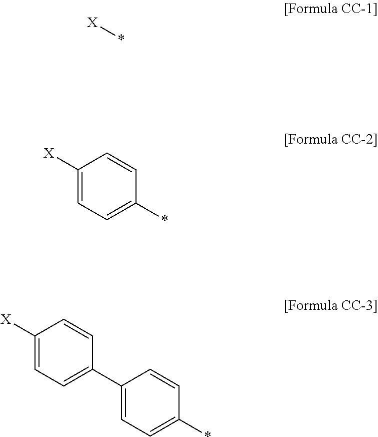
wherein in Formula CC-1 to Formula CC-3,
X is the same as defined in Formula C.

20. The amine compound of claim 15, wherein in Formula 1,
FG1 is a group represented by one of Formulae A-1 to A-75,
FG2 is a group represented by one of Formulae B-1 to B-99 and B-101 to B-106, and
FG3 is a group represented by one of Formulae A-1 to A-75, B-1 to B-99, B-101 to B-106, C-1 to C-77, and C-101 to C-109: 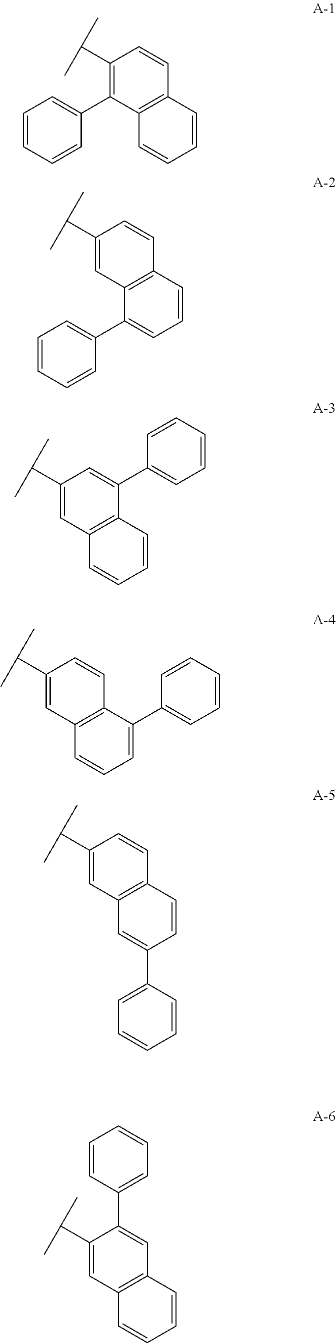 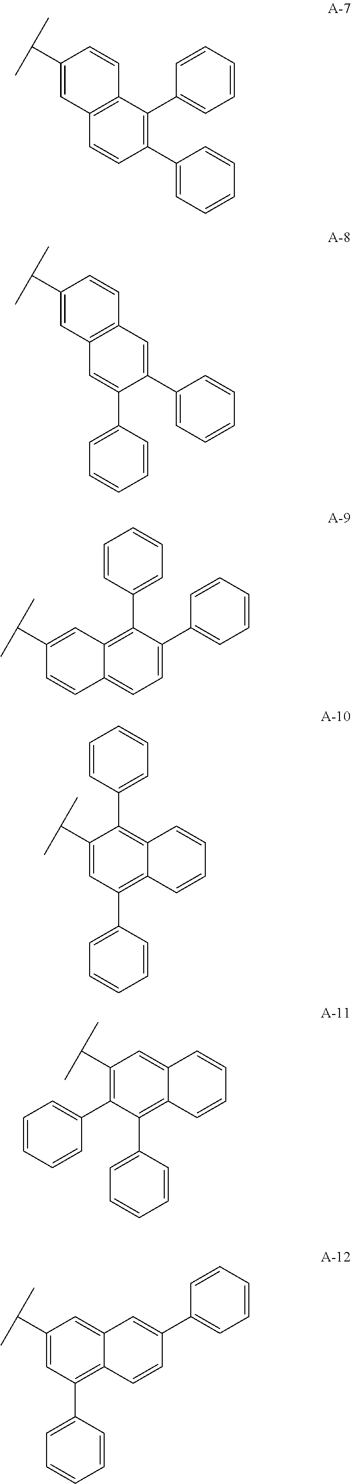 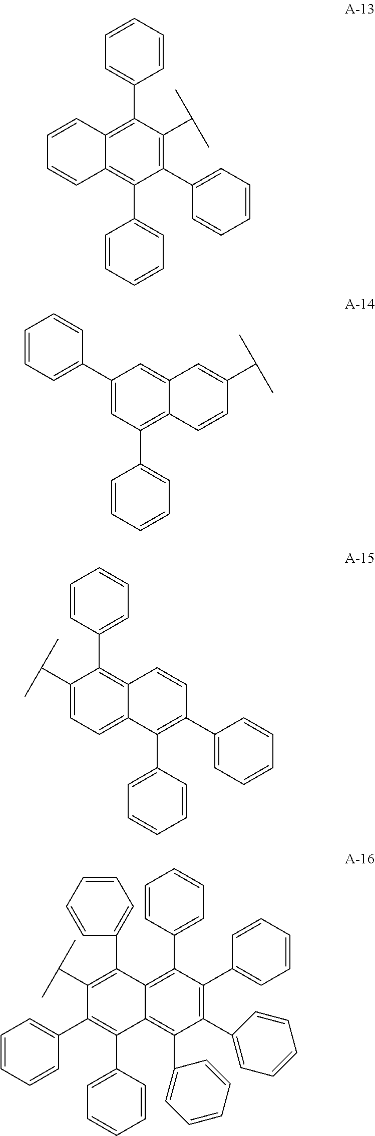 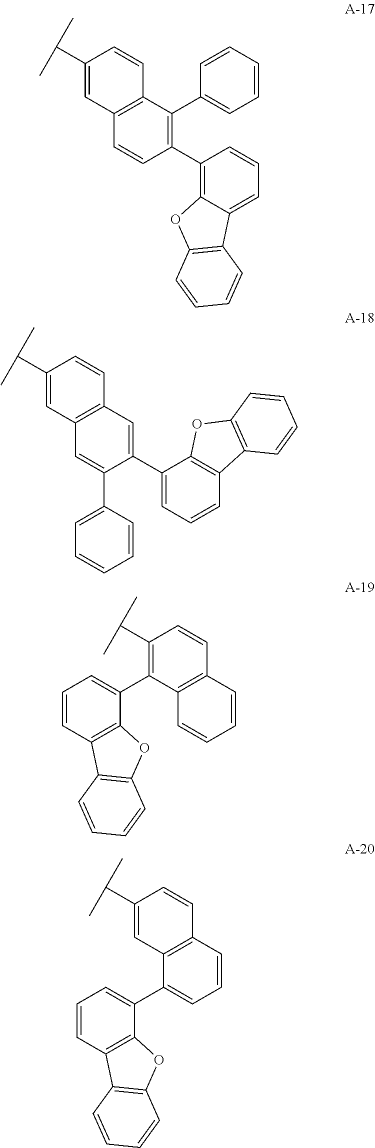 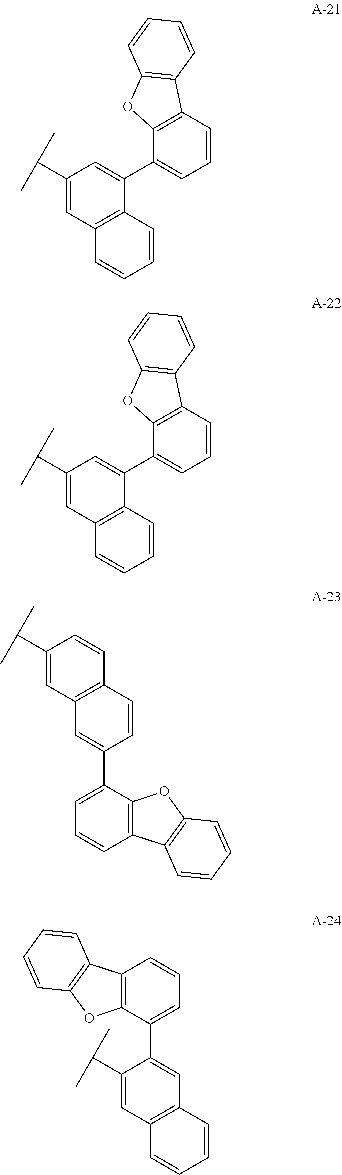 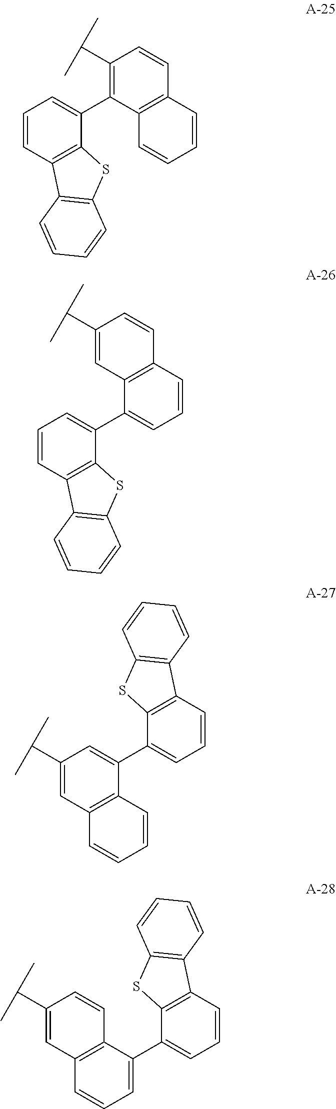 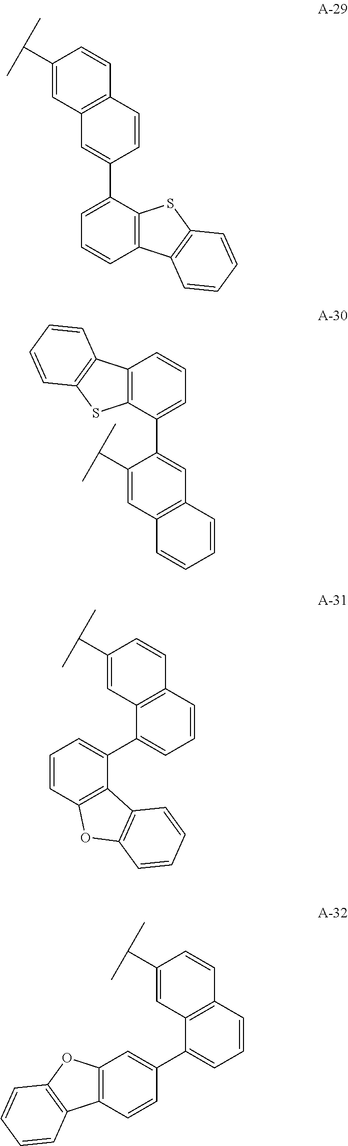 [SMILES: CC(C)c1ccc2ccc(-c3cccc4c3sc3ccccc34)cc2c1 ; CC(C)c1cc2ccccc2cc1-c1cccc2c1sc1ccccc12 ; CC(C)c1ccc2cccc(-c3cccc4oc5ccccc5c34)c2c1 ; CC(C)c1ccc2cccc(-c3ccc4c(c3)oc3ccccc34)c2c1] 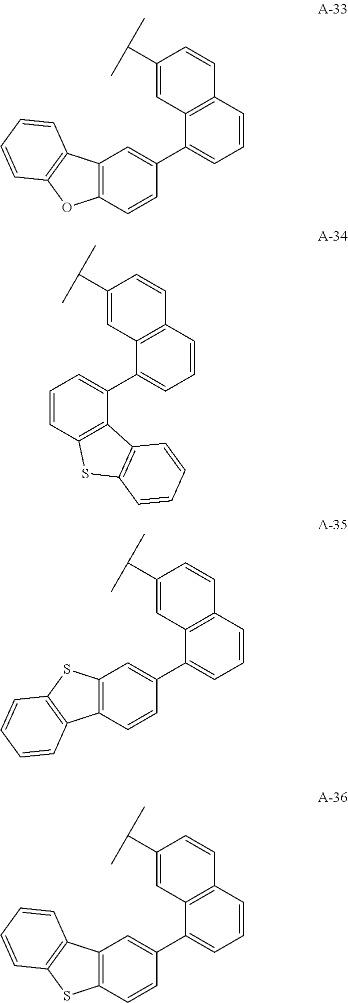 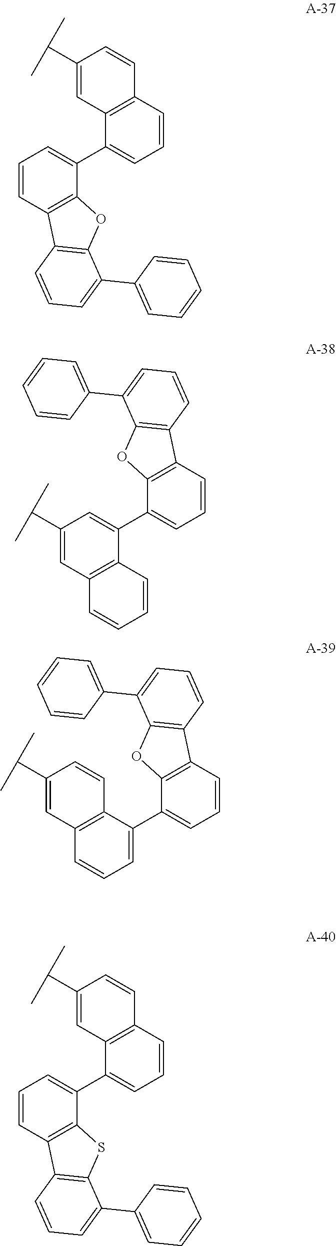 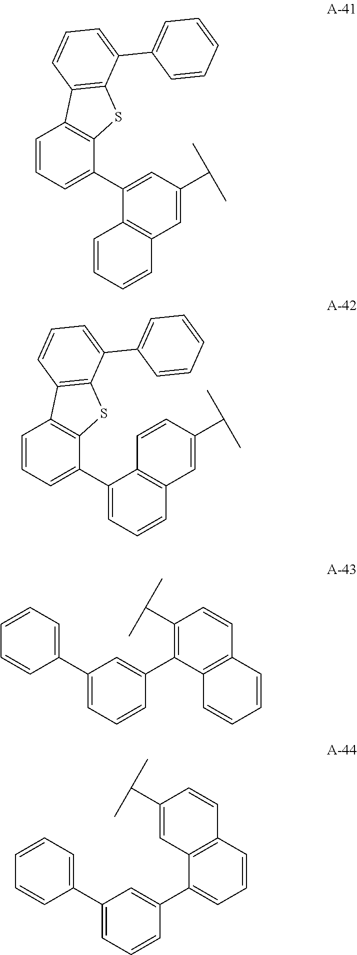 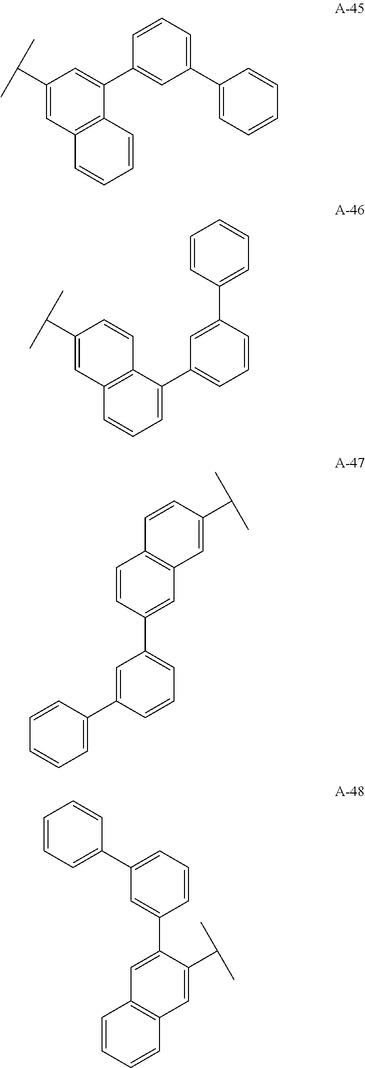 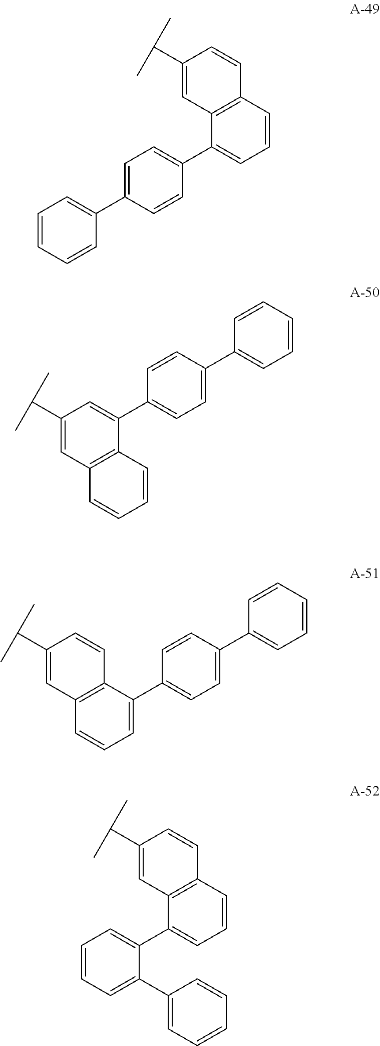 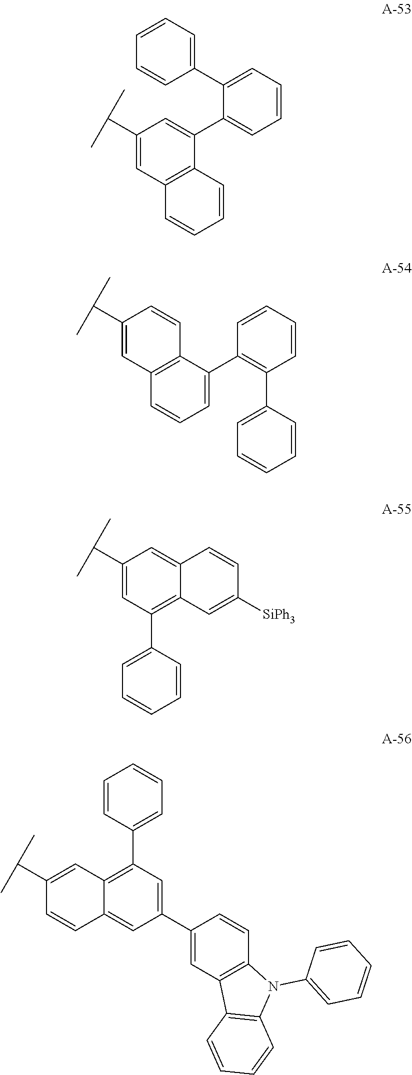 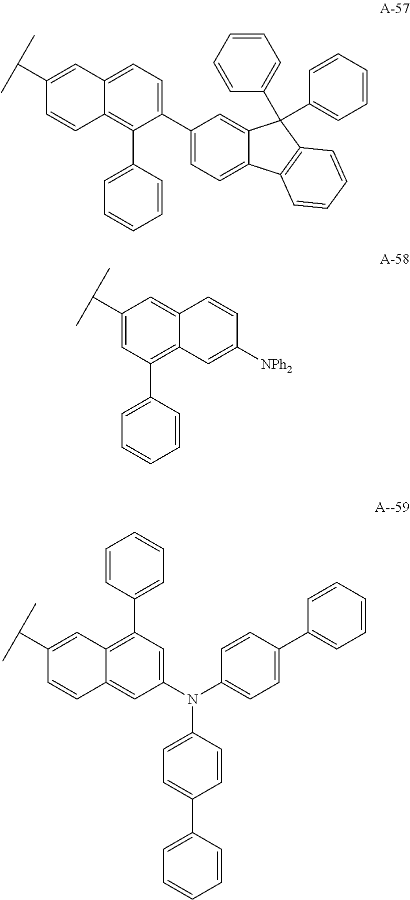 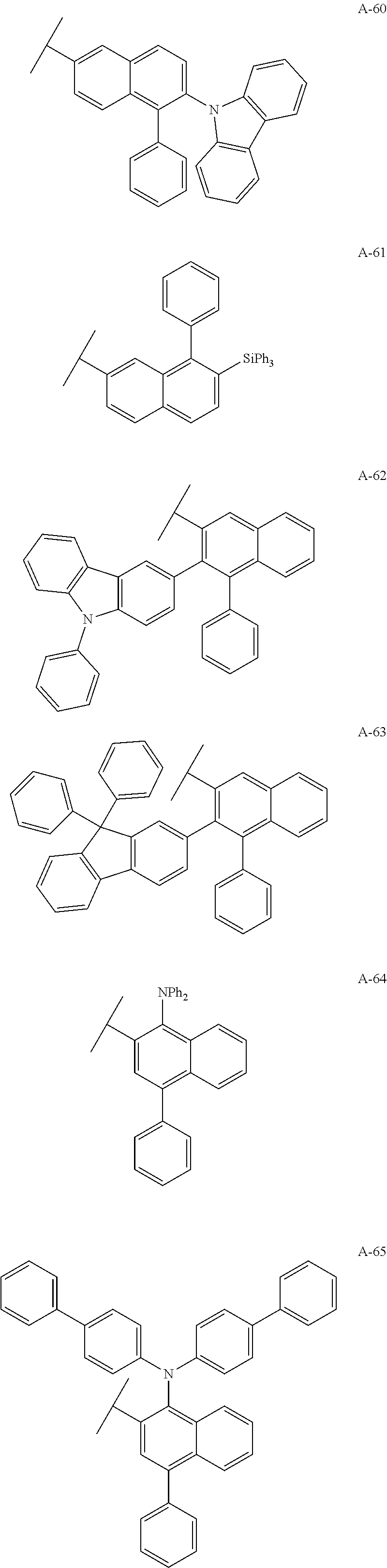 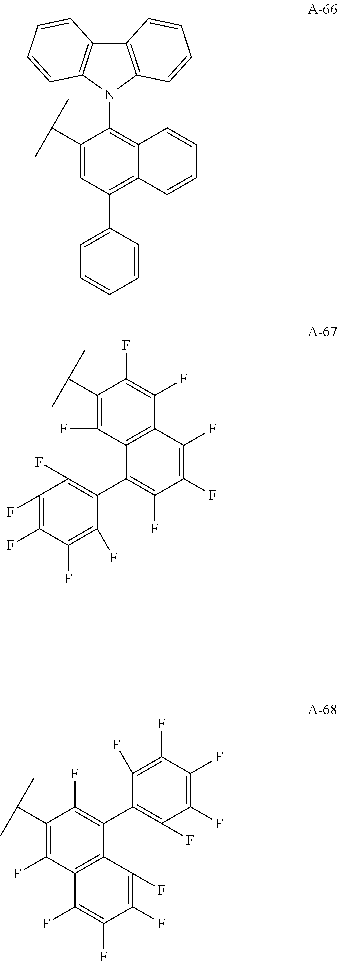 [SMILES: CC(C)c1cc(-c2ccccc2)c2ccccc2c1-n1c2ccccc2c2ccccc21 ; CC(C)c1c(F)c(F)c2c(F)c(F)c(F)c(-c3c(F)c(F)c(F)c(F)c3F)c2c1F ; CC(C)c1c(F)c(-c2c(F)c(F)c(F)c(F)c2F)c2c(F)c(F)c(F)c(F)c2c1F] 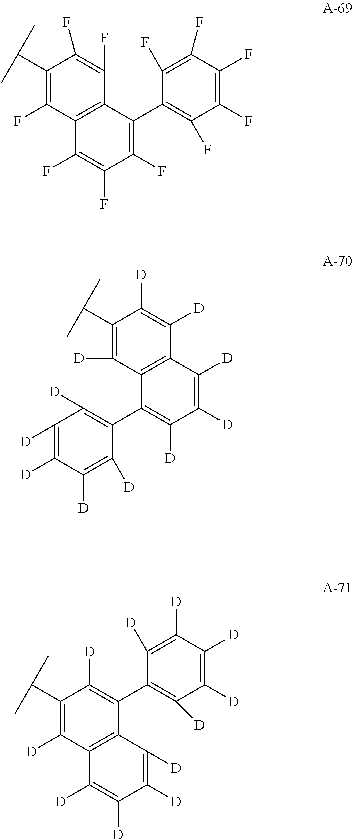 [SMILES: CC(C)c1c(F)c(F)c2c(-c3c(F)c(F)c(F)c(F)c3F)c(F)c(F)c(F)c2c1F ; [2H]c1c([2H])c([2H])c(-c2c([2H])c([2H])c([2H])c3c([2H])c([2H])c(C(C)C)c([2H])c23)c([2H])c1[2H] ; [2H]c1c([2H])c([2H])c(-c2c([2H])c(C(C)C)c([2H])c3c([2H])c([2H])c([2H])c([2H])c23)c([2H])c1[2H]] 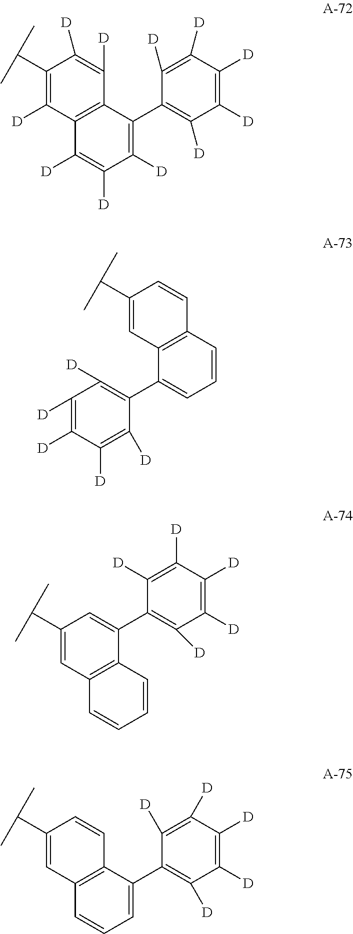 [SMILES: [2H]c1c([2H])c([2H])c(-c2c([2H])c([2H])c([2H])c3c([2H])c(C(C)C)c([2H])c([2H])c23)c([2H])c1[2H] ; [2H]c1c([2H])c([2H])c(-c2cccc3ccc(C(C)C)cc23)c([2H])c1[2H] ; [2H]c1c([2H])c([2H])c(-c2cc(C(C)C)cc3ccccc23)c([2H])c1[2H] ; [2H]c1c([2H])c([2H])c(-c2cccc3cc(C(C)C)ccc23)c([2H])c1[2H]] 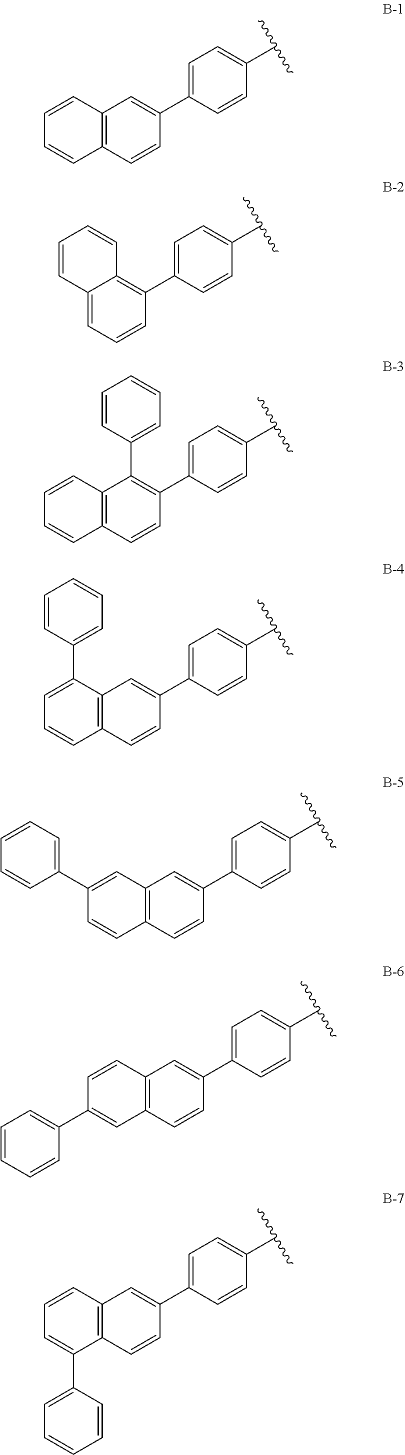 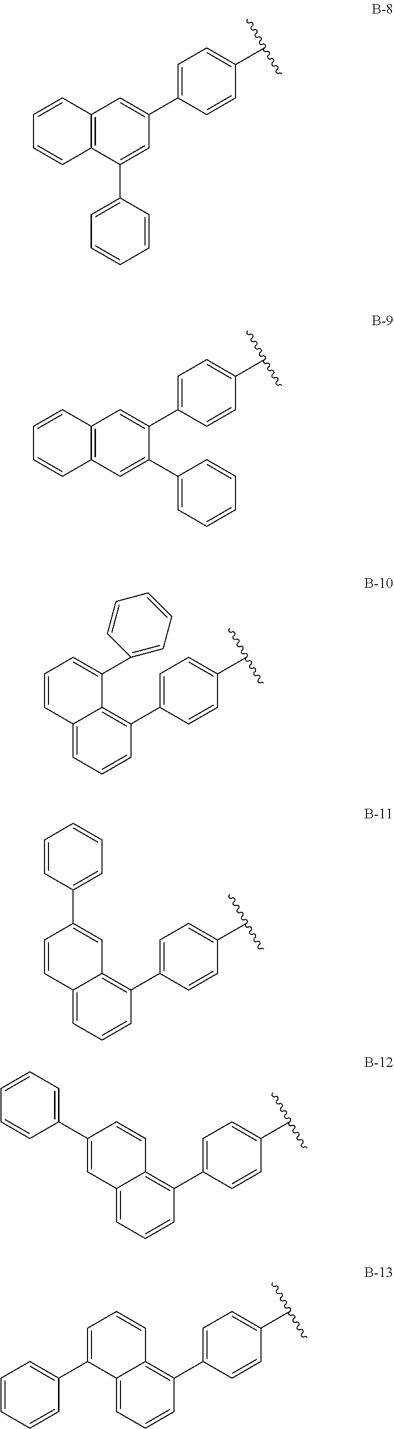 [SMILES: *c1ccc(-c2cc(-c3ccccc3)c3ccccc3c2)cc1 ; *c1ccc(-c2cc3ccccc3cc2-c2ccccc2)cc1 ; *c1ccc(-c2cccc3cccc(-c4ccccc4)c23)cc1 ; *c1ccc(-c2cccc3ccc(-c4ccccc4)cc23)cc1 ; *c1ccc(-c2cccc3cc(-c4ccccc4)ccc23)cc1 ; *c1ccc(-c2cccc3c(-c4ccccc4)cccc23)cc1] 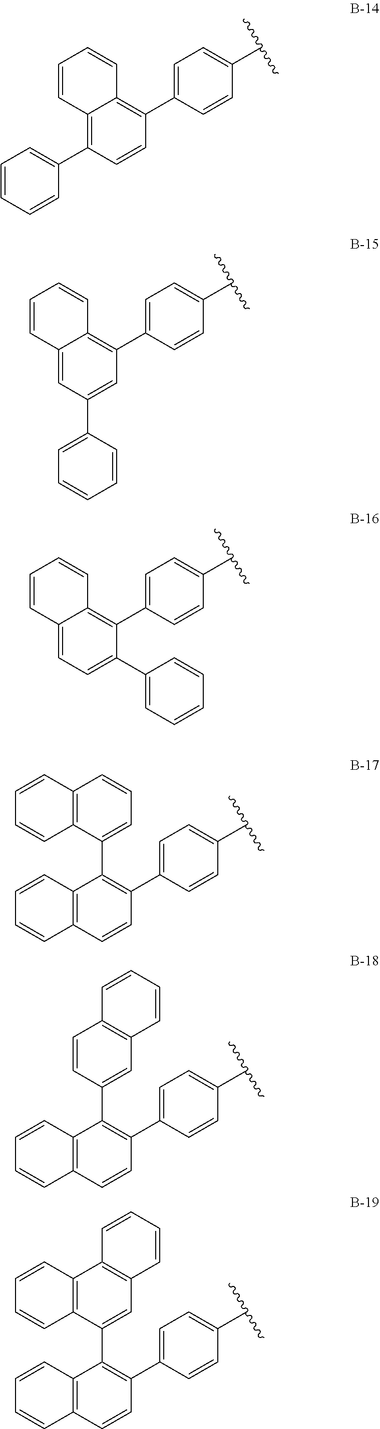 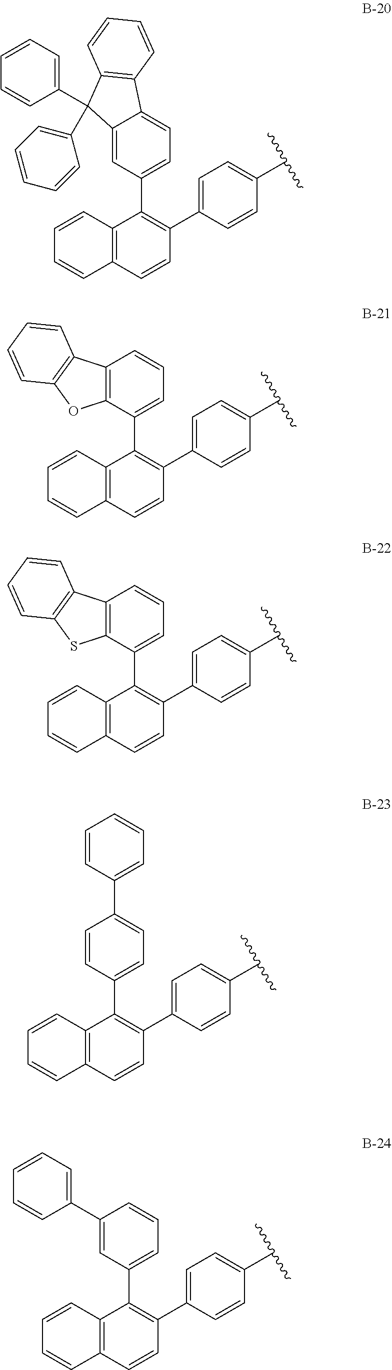 [SMILES: *c1ccc(-c2ccc3ccccc3c2-c2ccc3c(c2)C(c2ccccc2)(c2ccccc2)c2ccccc2-3)cc1 ; *c1ccc(-c2ccc3ccccc3c2-c2cccc3c2oc2ccccc23)cc1 ; *c1ccc(-c2ccc3ccccc3c2-c2cccc3c2sc2ccccc23)cc1 ; *c1ccc(-c2ccc3ccccc3c2-c2ccc(-c3ccccc3)cc2)cc1 ; *c1ccc(-c2ccc3ccccc3c2-c2cccc(-c3ccccc3)c2)cc1] 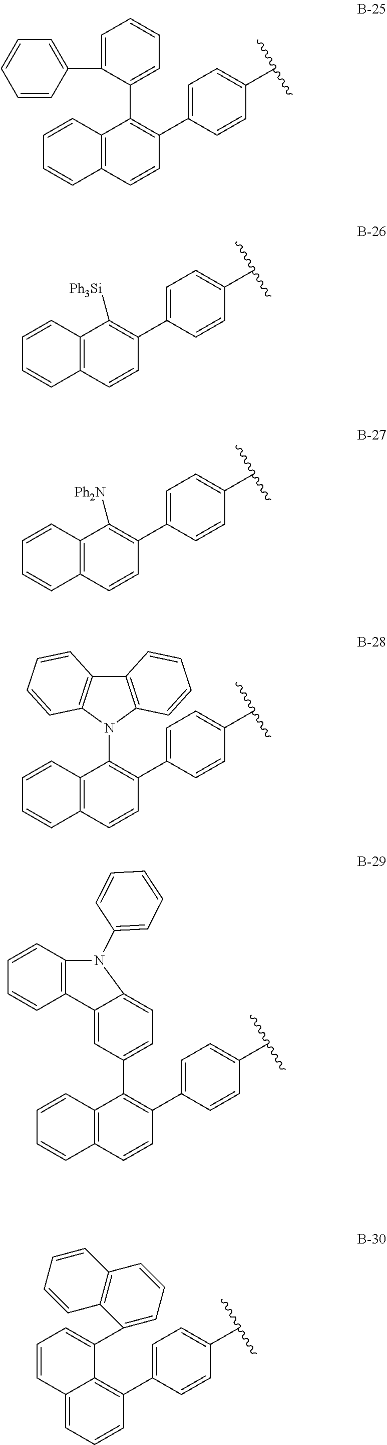 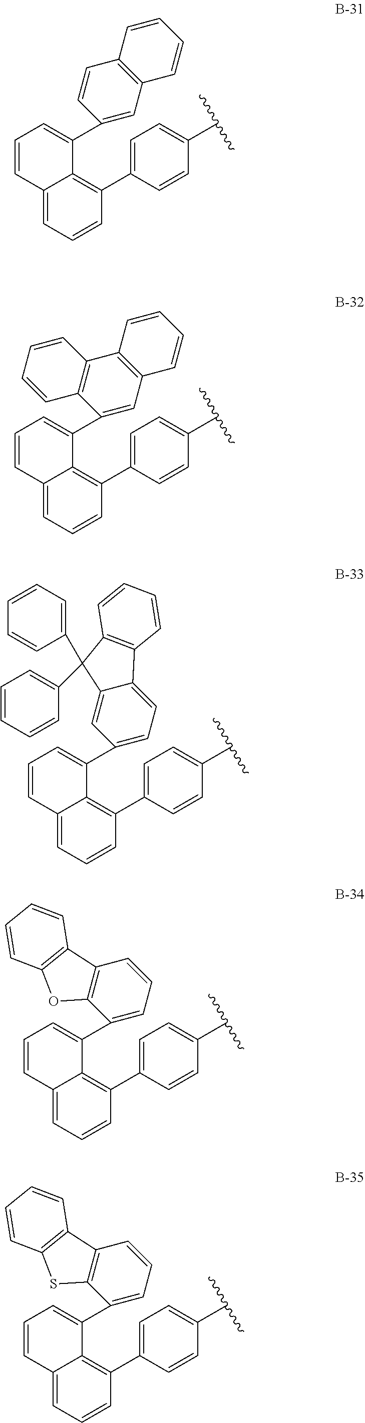 [SMILES: *c1ccc(-c2cccc3cccc(-c4ccc5ccccc5c4)c23)cc1 ; *c1ccc(-c2cccc3cccc(-c4cc5ccccc5c5ccccc45)c23)cc1 ; *c1ccc(-c2cccc3cccc(-c4ccc5c(c4)C(c4ccccc4)(c4ccccc4)c4ccccc4-5)c23)cc1 ; *c1ccc(-c2cccc3cccc(-c4cccc5c4oc4ccccc45)c23)cc1 ; *c1ccc(-c2cccc3cccc(-c4cccc5c4sc4ccccc45)c23)cc1] 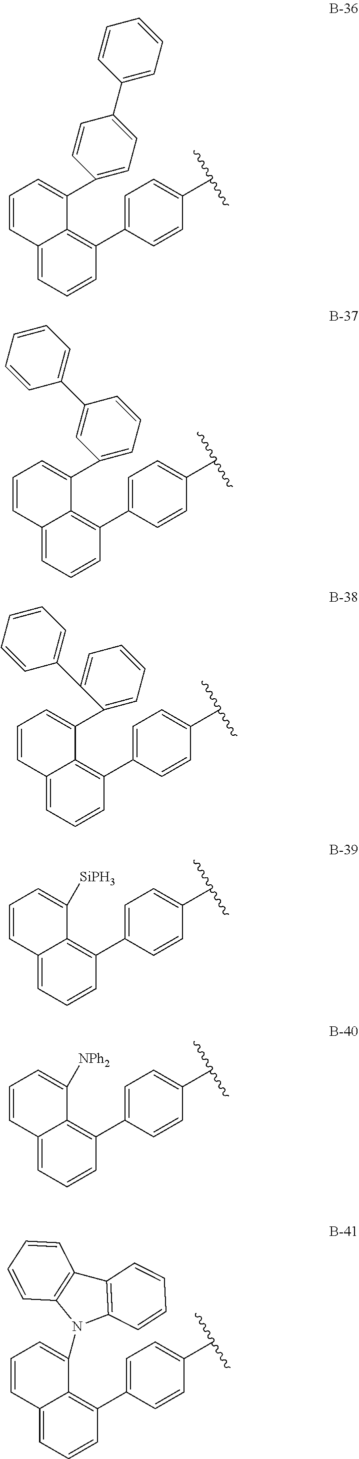 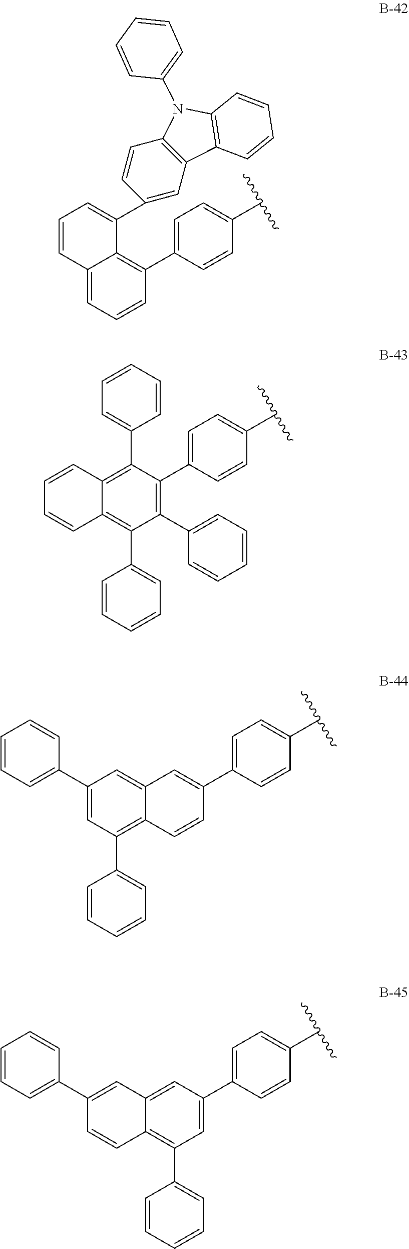 [SMILES: *c1ccc(-c2cccc3cccc(-c4ccc5c(c4)c4ccccc4n5-c4ccccc4)c23)cc1 ; *c1ccc(-c2c(-c3ccccc3)c(-c3ccccc3)c3ccccc3c2-c2ccccc2)cc1 ; *c1ccc(-c2ccc3c(-c4ccccc4)cc(-c4ccccc4)cc3c2)cc1 ; *c1ccc(-c2cc(-c3ccccc3)c3ccc(-c4ccccc4)cc3c2)cc1] 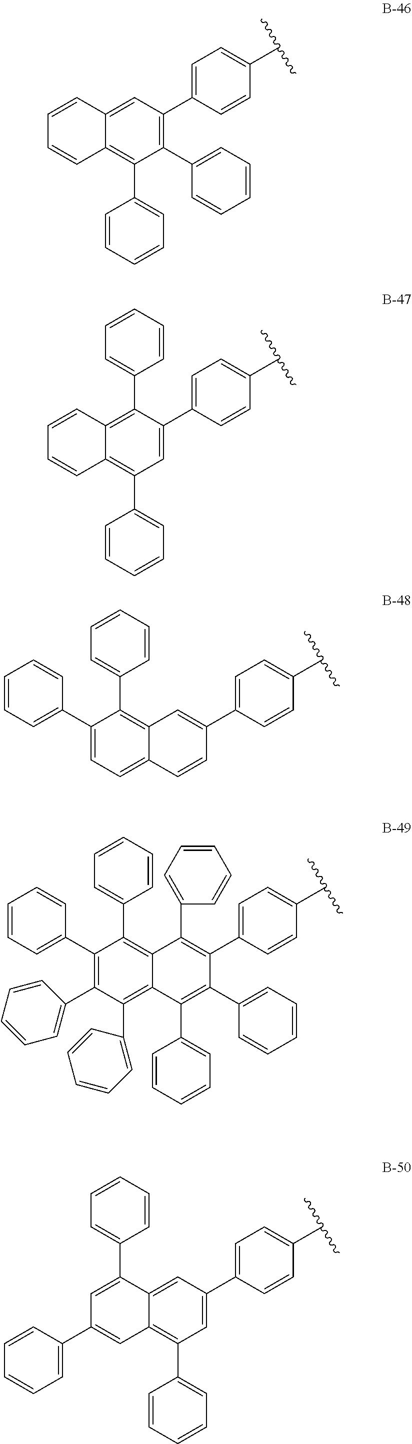 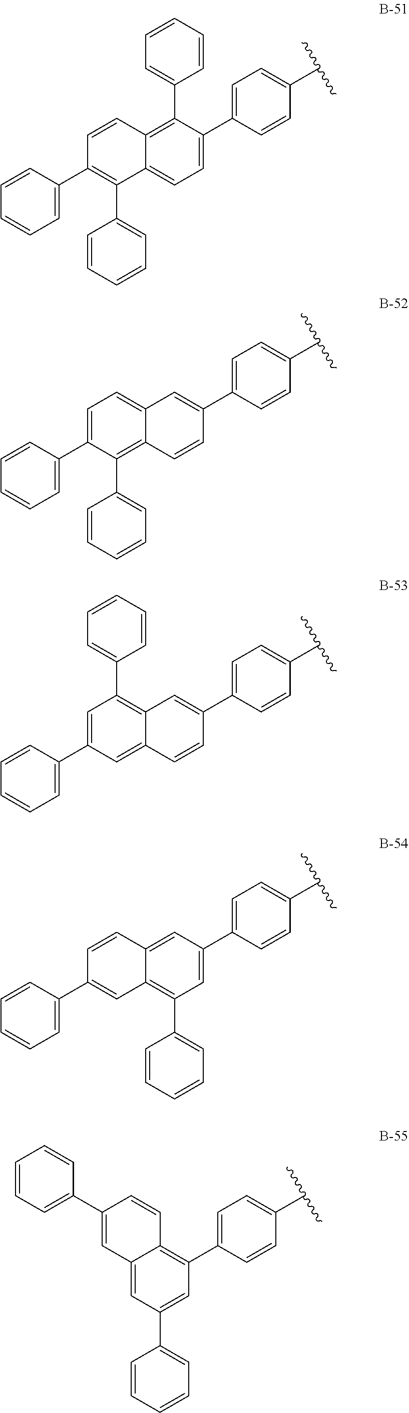 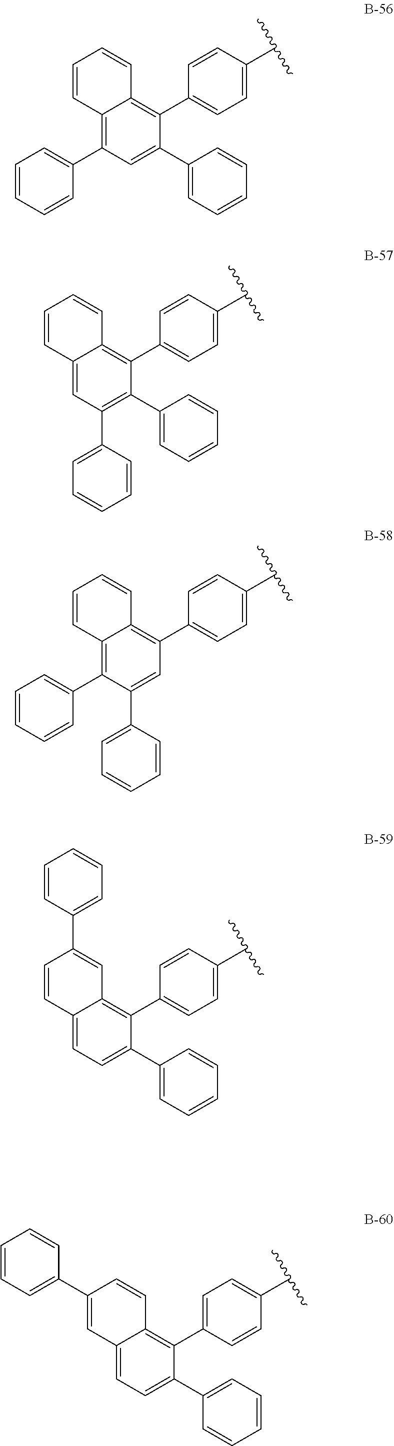 [SMILES: *c1ccc(-c2c(-c3ccccc3)cc(-c3ccccc3)c3ccccc23)cc1 ; *c1ccc(-c2c(-c3ccccc3)c(-c3ccccc3)cc3ccccc23)cc1 ; *c1ccc(-c2cc(-c3ccccc3)c(-c3ccccc3)c3ccccc23)cc1 ; *c1ccc(-c2c(-c3ccccc3)ccc3ccc(-c4ccccc4)cc23)cc1 ; *c1ccc(-c2c(-c3ccccc3)ccc3cc(-c4ccccc4)ccc23)cc1] 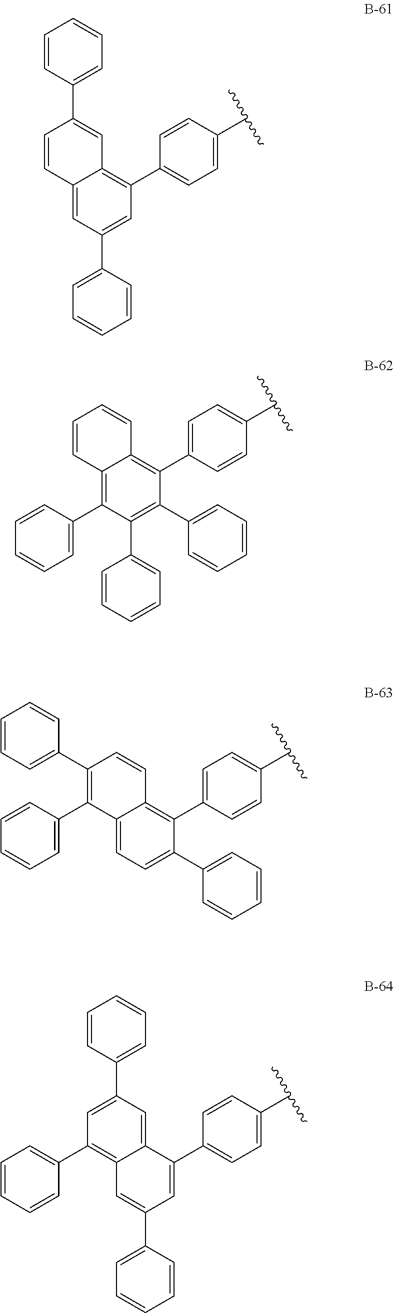 [SMILES: *c1ccc(-c2cc(-c3ccccc3)cc3ccc(-c4ccccc4)cc23)cc1 ; *c1ccc(-c2c(-c3ccccc3)c(-c3ccccc3)c(-c3ccccc3)c3ccccc23)cc1 ; *c1ccc(-c2c(-c3ccccc3)ccc3c(-c4ccccc4)c(-c4ccccc4)ccc23)cc1 ; *c1ccc(-c2cc(-c3ccccc3)cc3c(-c4ccccc4)cc(-c4ccccc4)cc23)cc1] 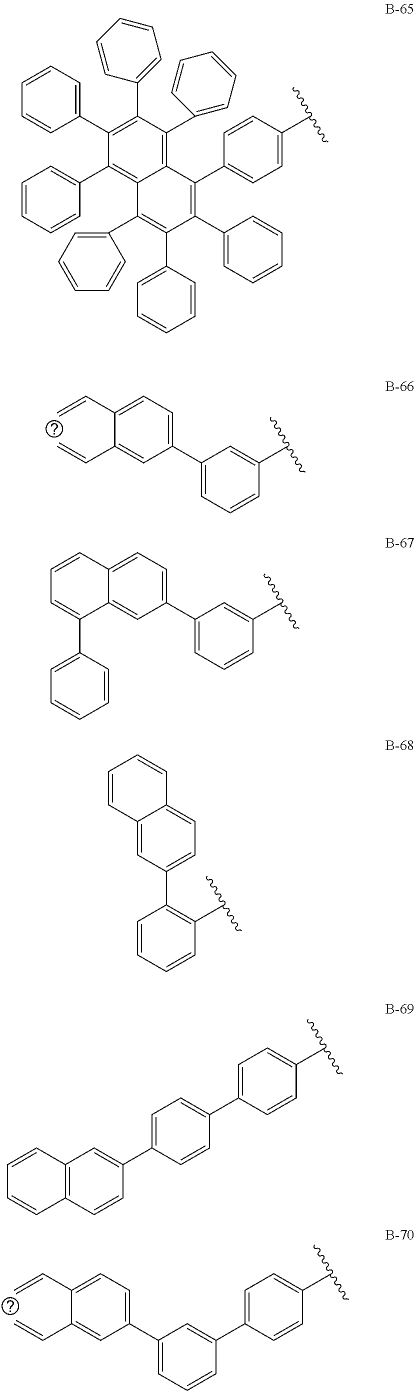 [SMILES: *c1ccc(-c2c(-c3ccccc3)c(-c3ccccc3)c(-c3ccccc3)c3c(-c4ccccc4)c(-c4ccccc4)c(-c4ccccc4)c(-c4ccccc4)c23)cc1 ; *c1cccc(-c2ccc(C=C)c(C=C)c2)c1 ; *c1cccc(-c2ccc3cccc(-c4ccccc4)c3c2)c1 ; *c1ccccc1-c1ccc2ccccc2c1 ; *c1ccc(-c2ccc(-c3ccc4ccccc4c3)cc2)cc1 ; *c1ccc(-c2cccc(-c3ccc(C=C)c(C=C)c3)c2)cc1] 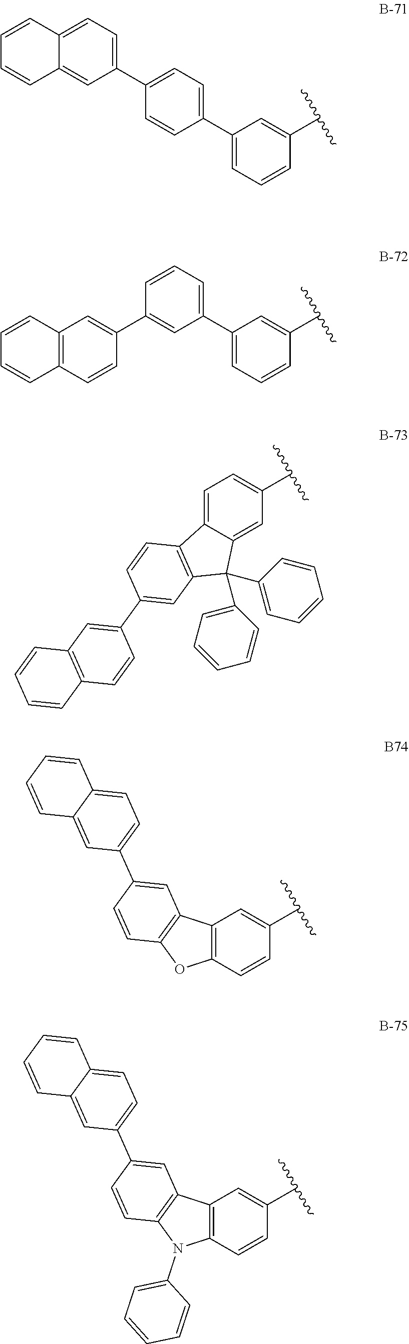 [SMILES: *c1cccc(-c2ccc(-c3ccc4ccccc4c3)cc2)c1 ; *c1cccc(-c2cccc(-c3ccc4ccccc4c3)c2)c1 ; *c1ccc2c(c1)C(c1ccccc1)(c1ccccc1)c1cc(-c3ccc4ccccc4c3)ccc1-2 ; *c1ccc2oc3ccc(-c4ccc5ccccc5c4)cc3c2c1 ; *c1ccc2c(c1)c1cc(-c3ccc4ccccc4c3)ccc1n2-c1ccccc1] 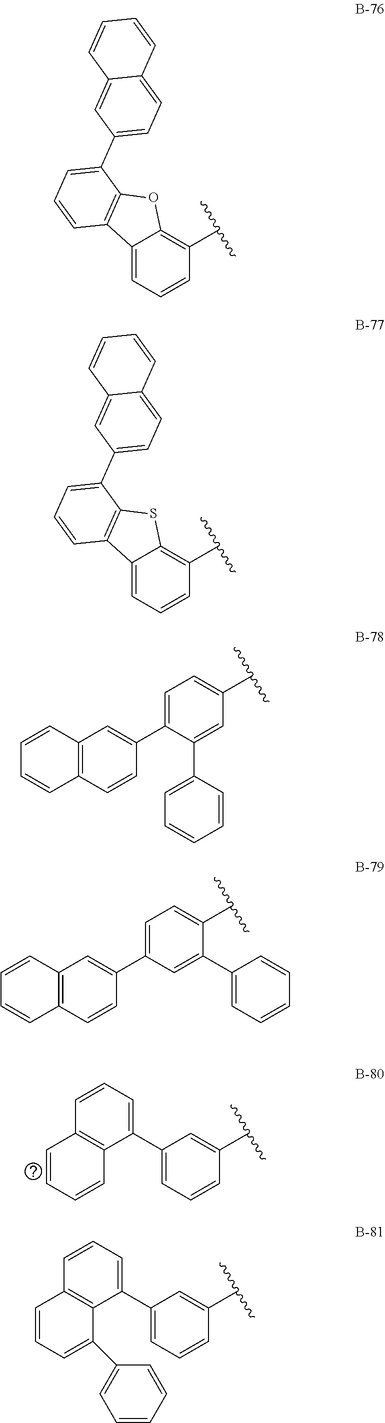 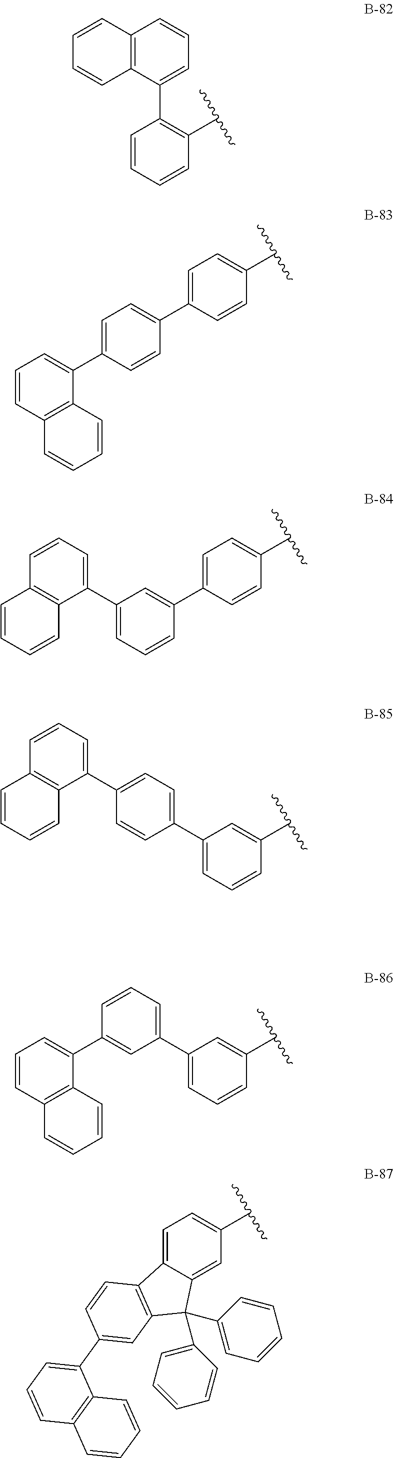 [SMILES: *c1ccccc1-c1cccc2ccccc12 ; *c1ccc(-c2ccc(-c3cccc4ccccc34)cc2)cc1 ; *c1ccc(-c2cccc(-c3cccc4ccccc34)c2)cc1 ; *c1cccc(-c2ccc(-c3cccc4ccccc34)cc2)c1 ; *c1cccc(-c2cccc(-c3cccc4ccccc34)c2)c1 ; *c1ccc2c(c1)C(c1ccccc1)(c1ccccc1)c1cc(-c3cccc4ccccc34)ccc1-2] 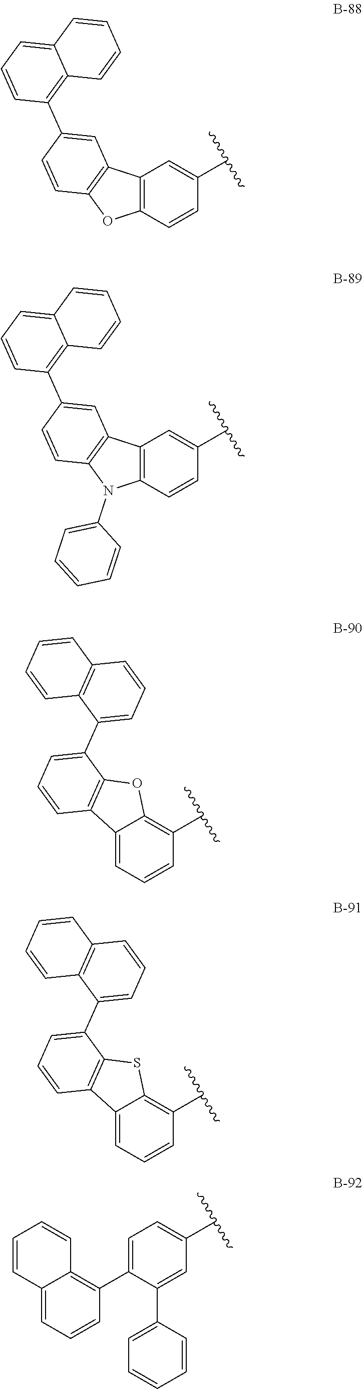 [SMILES: *c1ccc2oc3ccc(-c4cccc5ccccc45)cc3c2c1 ; *c1ccc2c(c1)c1cc(-c3cccc4ccccc34)ccc1n2-c1ccccc1 ; *c1cccc2c1oc1c(-c3cccc4ccccc34)cccc12 ; *c1cccc2c1sc1c(-c3cccc4ccccc34)cccc12 ; *c1ccc(-c2cccc3ccccc23)c(-c2ccccc2)c1] 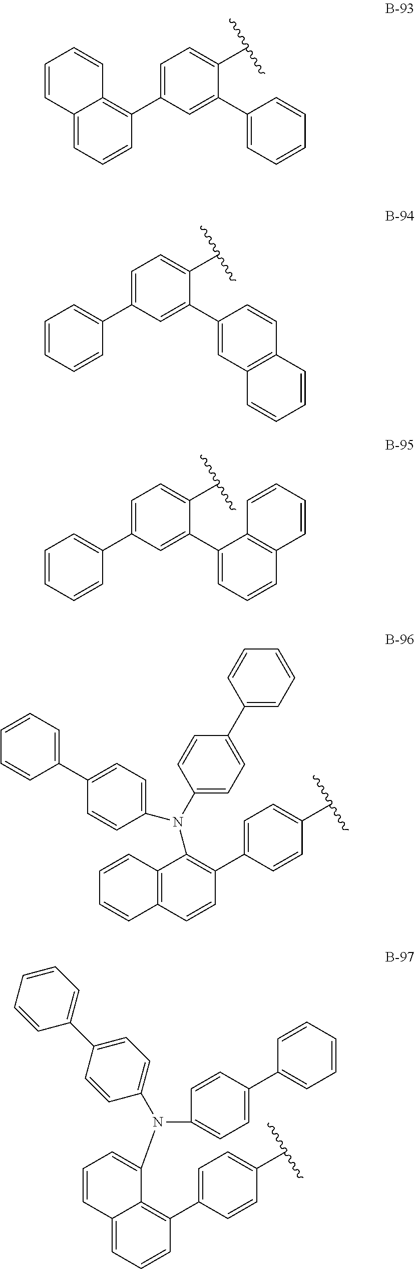 [SMILES: *c1ccc(-c2cccc3ccccc23)cc1-c1ccccc1 ; *c1ccc(-c2ccccc2)cc1-c1ccc2ccccc2c1 ; *c1ccc(-c2ccccc2)cc1-c1cccc2ccccc12 ; *c1ccc(-c2ccc3ccccc3c2N(c2ccc(-c3ccccc3)cc2)c2ccc(-c3ccccc3)cc2)cc1 ; *c1ccc(-c2cccc3cccc(N(c4ccc(-c5ccccc5)cc4)c4ccc(-c5ccccc5)cc4)c23)cc1] 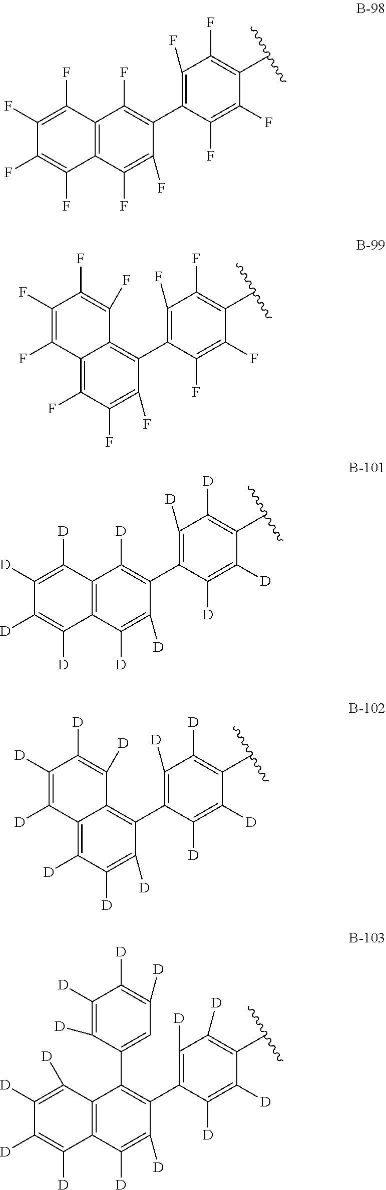 [SMILES: *c1c(F)c(F)c(-c2c(F)c(F)c3c(F)c(F)c(F)c(F)c3c2F)c(F)c1F ; *c1c(F)c(F)c(-c2c(F)c(F)c(F)c3c(F)c(F)c(F)c(F)c23)c(F)c1F ; *c1c([2H])c([2H])c(-c2c([2H])c([2H])c3c([2H])c([2H])c([2H])c([2H])c3c2[2H])c([2H])c1[2H] ; *c1c([2H])c([2H])c(-c2c([2H])c([2H])c([2H])c3c([2H])c([2H])c([2H])c([2H])c23)c([2H])c1[2H] ; *c1c([2H])c([2H])c(-c2c([2H])c([2H])c3c([2H])c([2H])c([2H])c([2H])c3c2-c2cc([2H])c([2H])c([2H])c2[2H])c([2H])c1[2H]] 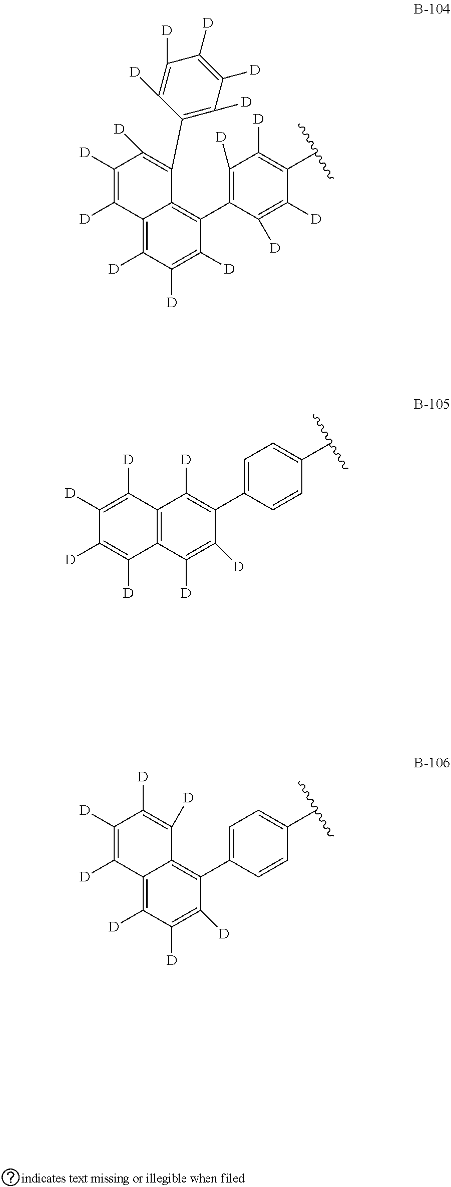 [SMILES: *c1c([2H])c([2H])c(-c2c([2H])c([2H])c([2H])c3c([2H])c([2H])c([2H])c(-c4c([2H])c([2H])c([2H])c([2H])c4[2H])c23)c([2H])c1[2H] ; *c1ccc(-c2c([2H])c([2H])c3c([2H])c([2H])c([2H])c([2H])c3c2[2H])cc1 ; *c1ccc(-c2c([2H])c([2H])c([2H])c3c([2H])c([2H])c([2H])c([2H])c23)cc1] 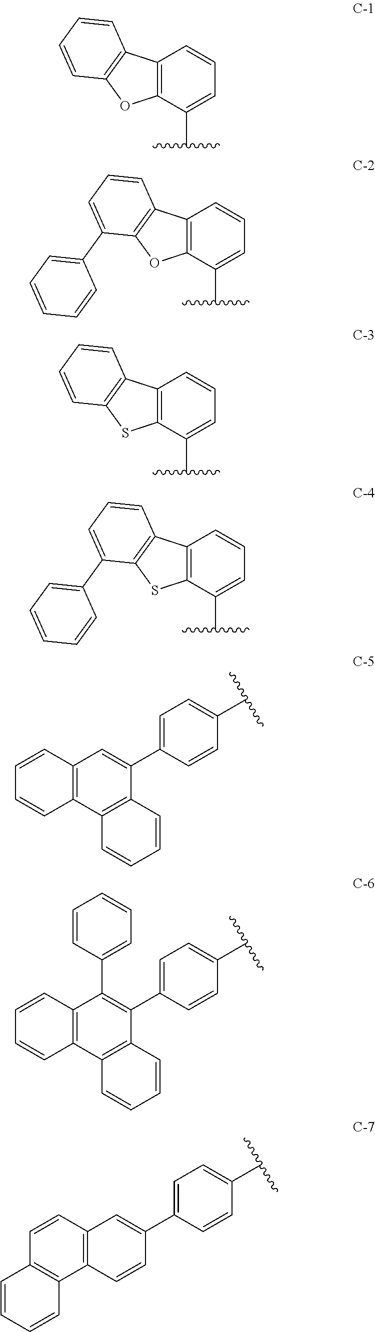 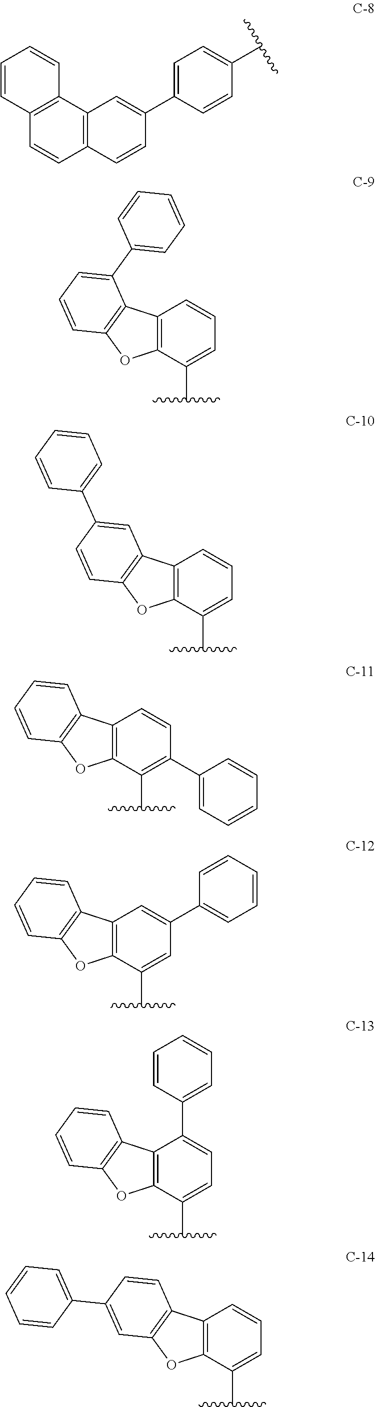 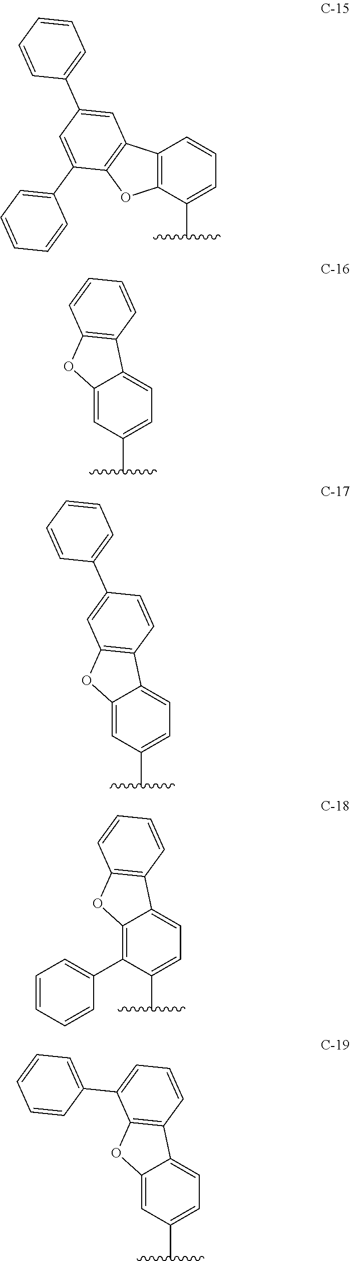 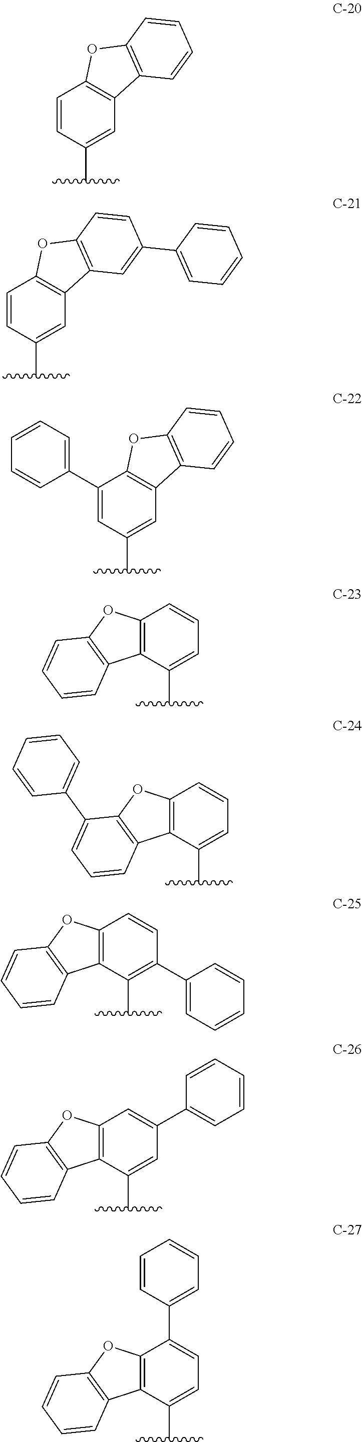 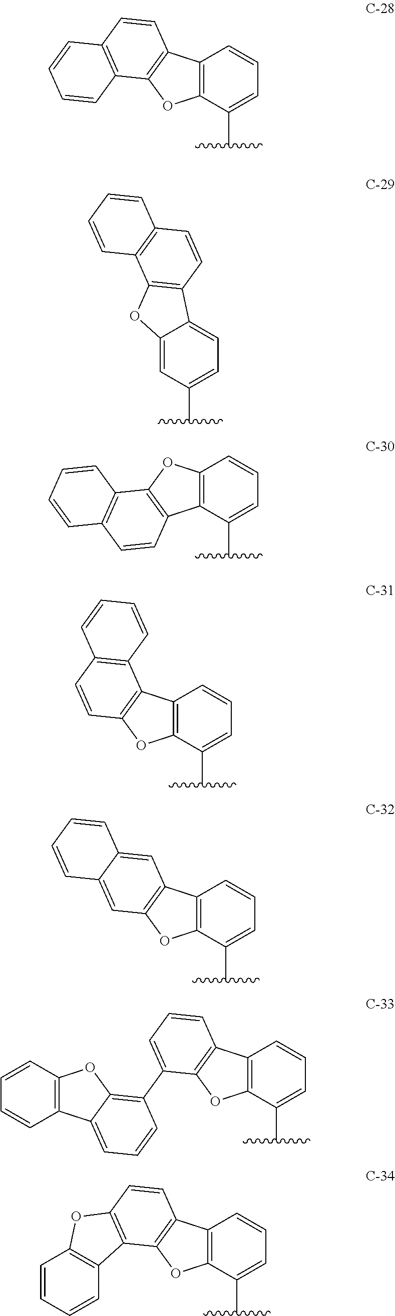 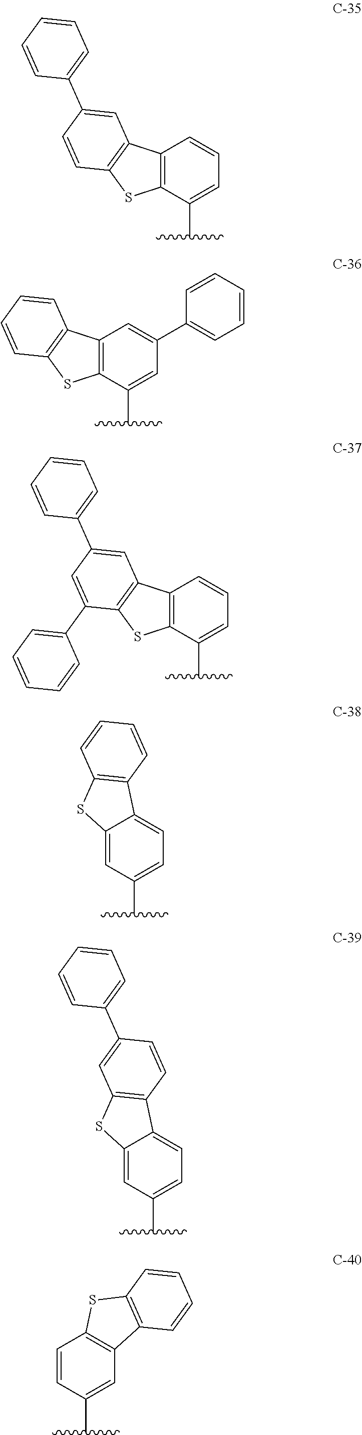 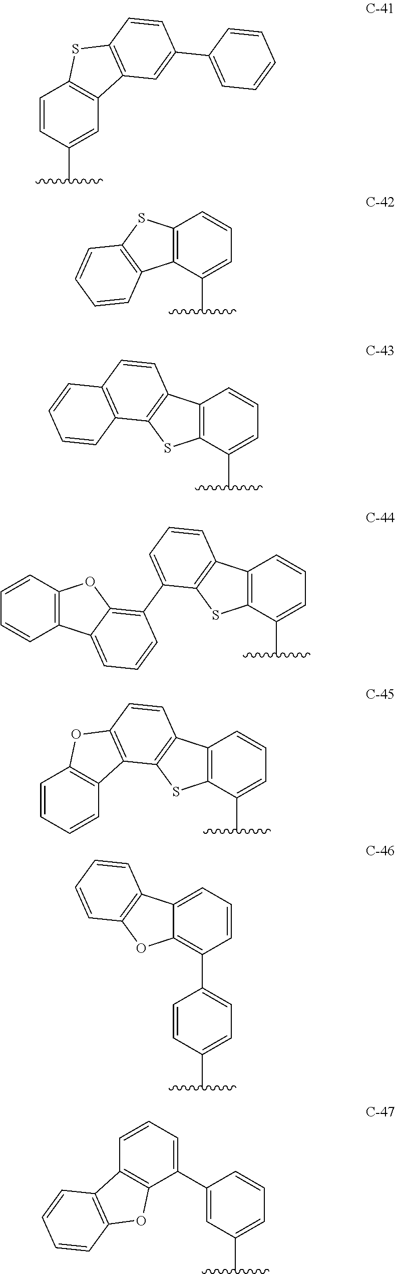 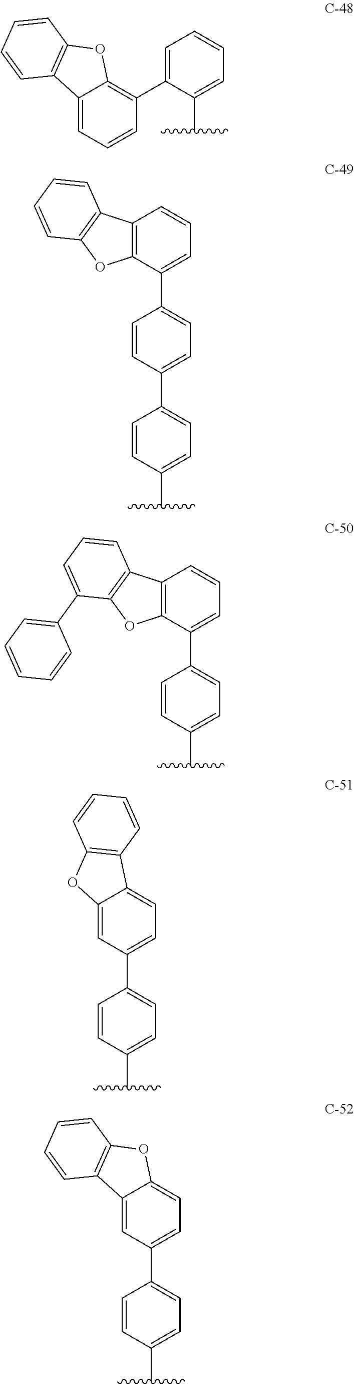 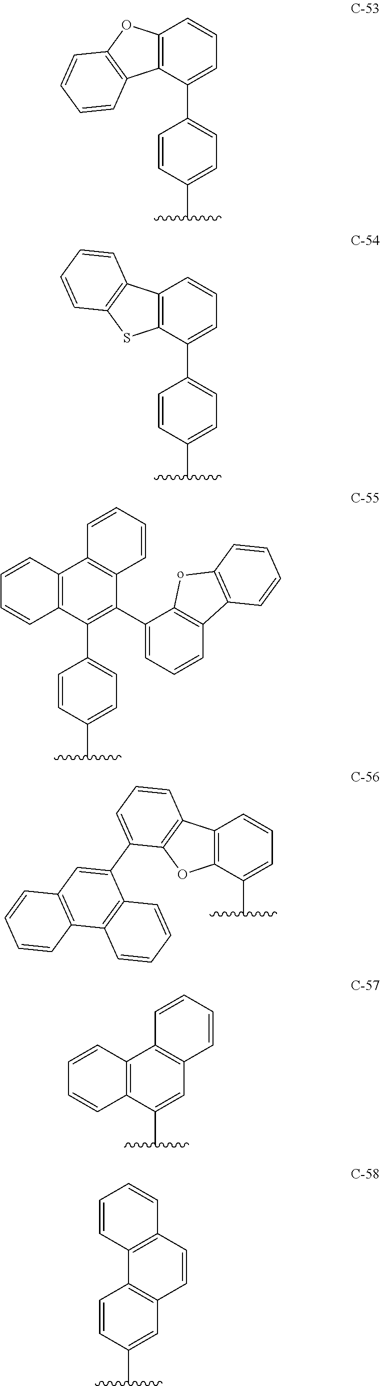 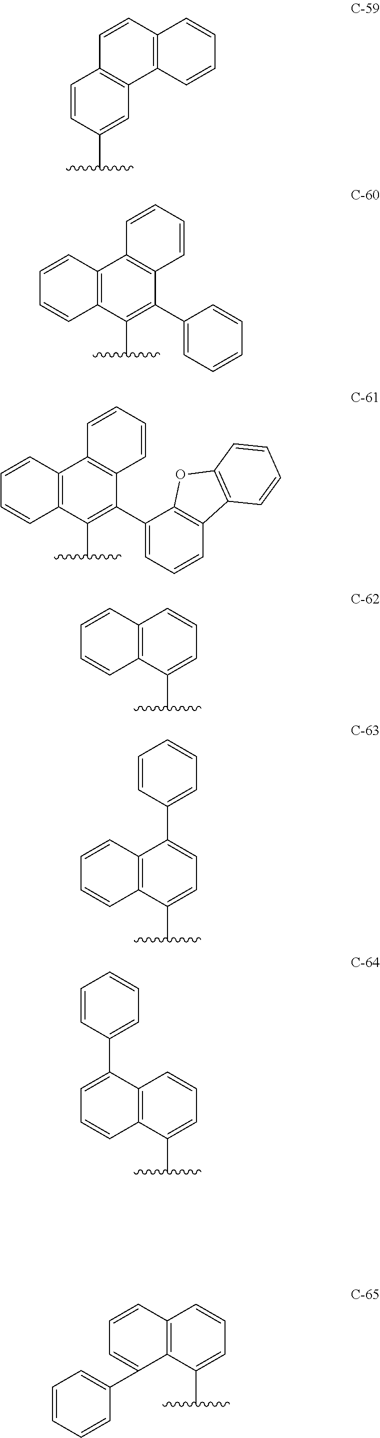 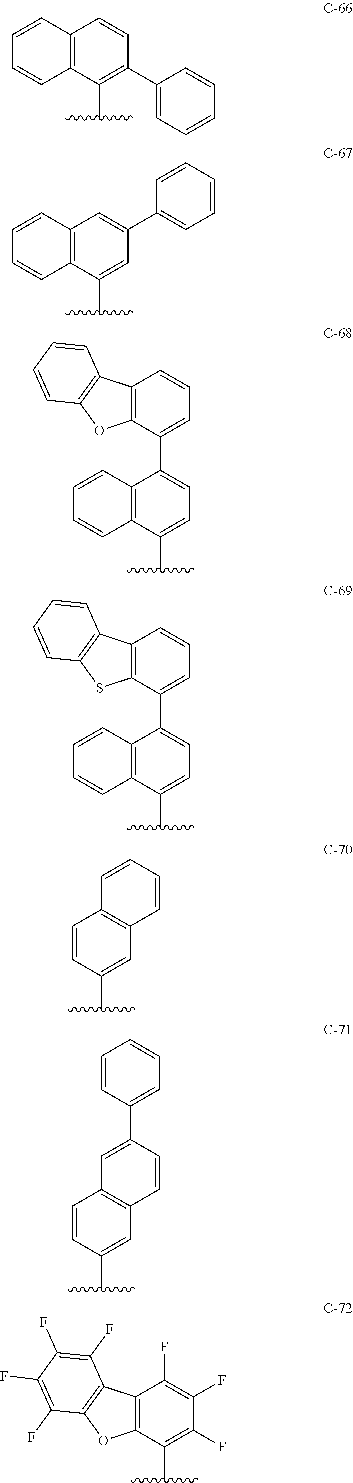 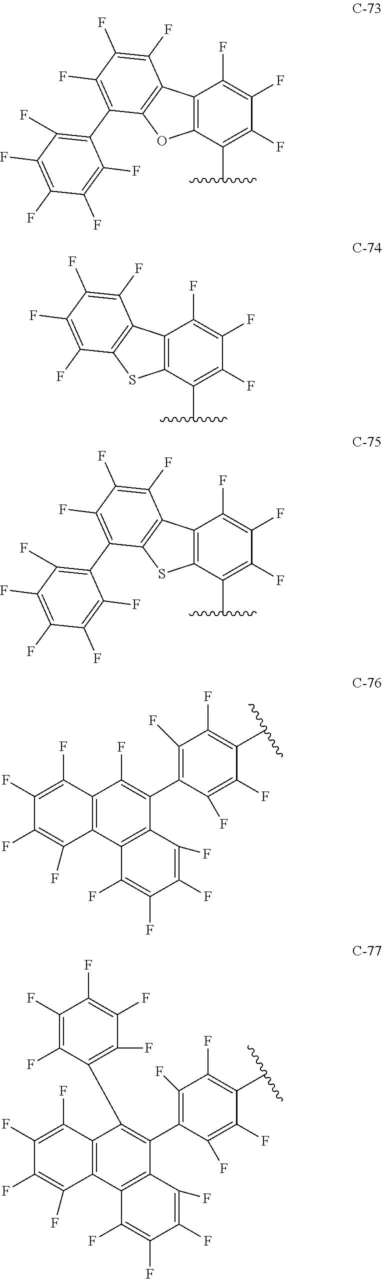 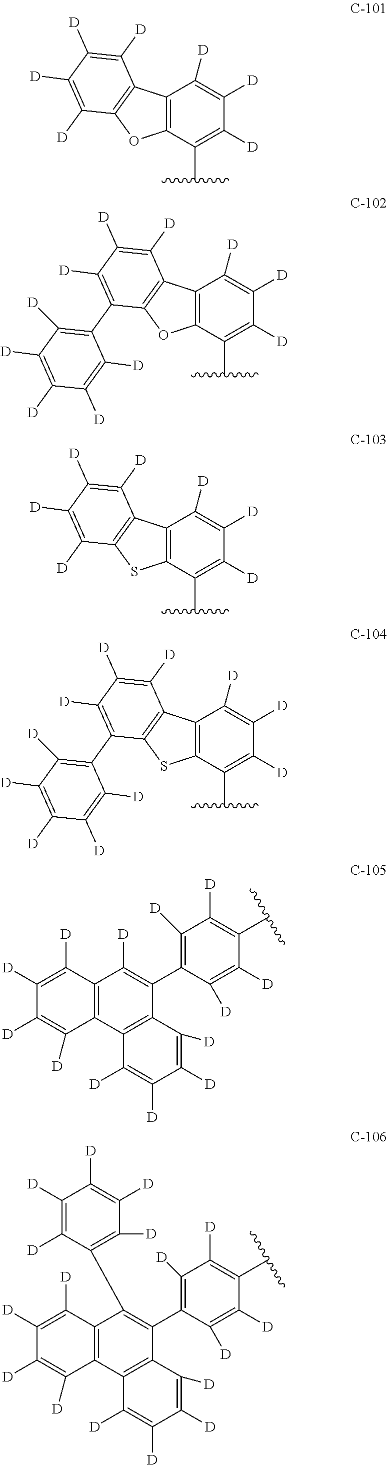 [chemical structure]
